(19) United States
(12) Patent Application Publication
(10) Pub. No.: US 2026/0226034 A1
(43) Pub. Date: Aug. 6, 2026

(54) SULFONE-1H-PYRROLE-2-CARBOXAMIDE INHIBITORS OF SARS-COV-2 NSP14 METHYLTRANSFERASE AND DERIVATIVES THEREOF

(71) Applicant: The Rockefeller University, New York, NY (US)

(72) Inventors: Thomas TUSCHL, New York, NY (US); Aitor GARCIA, New York, NY (US); Cindy MEYER, New York, NY (US); J. Fraser GLICKMAN, New York, NY (US); Michael W. MILLER, Scotch Plains, NJ (US); Nigel John LIVERTON, Harleysville, PA (US); Stacia KARGMAN; Robert Walter MYERS, New York, NY (US); David John HUGGINS, New York, NY (US)

(73) Assignee: The Rockefeller University, New York, NY (US)

(21) Appl. No.: 19/150,636

(22) PCT Filed: Jan. 24, 2024
(86) PCT No.: PCT/US2024/012717

§ 371 Date: Jul. 24, 2025

Related U.S. Application Data
(60) Provisional application No. 63/481,429, filed on Jan. 25, 2023.

(51) Int. Cl. C07D 405/14; A61K 31/4025; A61K 31/4155; A61K 31/416; A61K 31/4178; A61K 31/4184; A61K 31/4192; A61K 31/422; A61K 31/437; A61K 31/4375; A61K 31/4439; A61K 31/4709; A61K 31/4725; A61K 31/497; A61K 31/501; A61K 31/502; A61K 31/506; A61K 31/517; A61K 31/52; A61P 31/14; C07D 401/12; C07D 401/14; C07D 403/12; C07D 403/14; C07D 405/12; C07D 413/14; C07D 471/04; C07D 473/00; C07D 487/04

(52) U.S. Cl. CPC C07D 405/14; A61K 31/4025; A61K 31/4155; A61K 31/416; A61K 31/4178; A61K 31/4184; A61K 31/4192; A61K 31/422; A61K 31/437; A61K 31/4375; A61K 31/4439; A61K 31/4709; A61K 31/4725; A61K 31/497; A61K 31/501; A61K 31/502; A61K 31/506; A61K 31/517; A61K 31/52; A61P 31/14; C07D 401/12; C07D 401/14; C07D 403/12; C07D 403/14; C07D 405/12; C07D 413/14; C07D 471/04; C07D 473/00; C07D 487/04

(57) ABSTRACT

Methods and compositions for treating SARS-CoV-2 and COVID-19 are disclosed. Sulfone-1H-pyrrole-2-carboxamides of the following formula

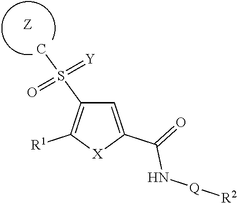

inhibit the SARS-CoV-2 PLpro/NSP3 protein and are therefore useful for treating SARS-CoV-2 and COVID-19.

### CROSS REFERENCE TO RELATED APPLICATIONS

This application claims priority to U.S. Provisional Application No. 63/481,429, filed Jan. 25, 2023, the entire contents of which are hereby incorporated by reference as if fully set forth herein.

### TECHNICAL FIELD

The present application relates generally to compounds that inhibit the NSP14 methyltransferase enzyme found in coronaviruses, including SARS-CoV-2. Therefore, these compounds are useful for treating SARS-CoV-2 and its associated disease, COVID-19. The compounds are generally sulfone-1H-pyrrole-2-carboxamides and derivatives thereof.

### BACKGROUND

Severe Acute Respiratory Syndrome Coronavirus 2 (SARS-CoV-2) is a species of coronavirus whereby infected patients exhibit COVID-19. The SARS-CoV-2 NSP14 protein is a methyltransferase enzyme that methylates the guanosine cap of newly synthesized RNAs at position N7 using S-adenosyl-L-methionine (SAM) as methyl donor forming the cap-0 structure m7GpppA-RNA and releasing S-adenosyl-L-homocysteine (SAH) as byproduct. The RNA cap facilitates translation of viral proteins, protection from exonucleases, and evasion of the host immune response. NSP14 is essential for SARS-CoV-2 replication and can therefore be used as a target to develop treatments for COVID-19.

### SUMMARY

The disclosure is directed to compounds, pharmaceutical compositions and methods for inhibiting SARS-CoV-2 methyltransferase and thereby treating SARS-CoV-2 and COVID-19.

In an aspect, the disclosure relates to compounds of formula (I):

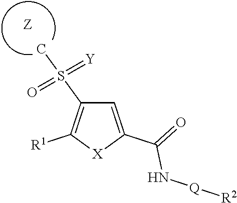

wherein
X is chosen from N(R^{a}) and S;
R^{a} is chosen from (C₁-C₆)hydrocarbon, (C₁-C₆)oxaalkyl, and hydrogen, wherein said (C₁-C₆)hydrocarbon and said (C₁-C₆)oxaalkyl is optionally substituted with one or more hydrogen, alkyl, alkoxy, hydroxyl, oxo, carbonyl, halogen, amino, cyano, or aminoacyl alkyl;
R¹ is chosen from methyl and ethyl; or
R^{a} and R¹ taken together with the atoms to which they are attached form a 5- or 6-member heterocycle optionally substituted with methyl; and
Y is chosen from O and N(R^{b});
R^{b} is chosen from hydrogen, (C₁-C₆)hydrocarbon, and hydroxyalkyl;
Z is chosen from aryl or heteroaryl, wherein said aryl and said heteroaryl is optionally substituted with one or more (C₁-C₆)alkyl, cyano, halogen, haloalkyl, cyanoalkyl, alkoxyalkyl, alkylsulfonyl, oxo, carbonyl, alkylcarbonyl, carboxamide, alkoxy, acetamide, cycloalkyl, hydroxyalkyl, or amino;
Q is chosen from direct bond, (C₁-C₆)hydrocarbon, (C₁-C₆)heteroalkyl, heterocycle, NH,

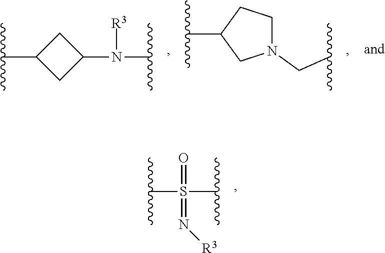

wherein said (C₁-C₆)hydrocarbon, said (C₁-C₆)heteroalkyl, and said heterocycle, is optionally substituted with one or more (C₁-C₁₀)hydrocarbyl, methylazetidine, oxaalkyl, hydroxyalkyl, halogen, oxo, hydroxyl, or haloalkyl;
R³ is chosen from hydrogen, alkyl, and haloalkyl;
R² is chosen from HetA, CarbA, —O-HetA, N(R^{c})₂, and C₁₃ hydrocarbon, said HetA and said CarbA is optionally substituted with one or more HetB, CarbB, halogen, alkoxy, oxaalkyl, cyano, COOH, hydroxyl, oxo, carbonyl, N(R^{c})₂, pyrrolidinylmethyl, azaalkyl, alkyl, thioalkyl, haloalkyl, alkoxylalkyl, haloalkylalkoxy, dimethylaminoalkyl, oxotetrahydrofuran, alkylsulfonyl, dimethylaminoalkylalkoxy, alkylsulfonylalkylalkoxy, —O-CarbB, or —O-HetB,
wherein said HetB, said alkoxy, said CarbB, said oxaalkyl, said azaalkyl, and said thioalkyl, is optionally substituted with one or more halogen, hydroxyl, (C₁-C₆)alkoxy, (C₁-C₆)hydrocarbyl, COOH, cyano, C(O)OC(CH₃)₂, N(R^{c})₂, (C₁-C₆)alkoxy(C₁-C₆)alkyl, (C₁-C₆)alkyl, oxo, carbonyl, C(O)CH₃, or alkylsulfonylcarboxamide,
wherein R^{c} is independently chosen in each instance from hydrogen, hydrocarbyl, haloalkyl, cyano, cyanoalkyl, alkoxyalkyl, carbonylhaloalkyl, oxaalkyl, carbonylalkyl, alkylsulfonyl, and HetA, said HetA optionally substituted with one or more halogen, and
wherein HetA is a 3- to 12-member heterocycle, HetB is a 3- to 11-member heterocycle, CarbA is a 6- to 10-member carbocycle, and CarbB is a 3- to 5-member carbocycle.

In an aspect, the disclosure relates to a method of inhibiting SARS-CoV-2 NSP14 protein in a patient comprising administering a compound as disclosed herein.

In an aspect, the disclosure relates to a method of treating Covid-19 in a patient comprising administering a compound as disclosed herein.

In an aspect, the disclosure relates to a pharmaceutical formulation comprising a pharmaceutically acceptable carrier and a compound as disclosed herein.

### DETAILED DESCRIPTION

In an aspect, the disclosure relates to compounds of formula (I):

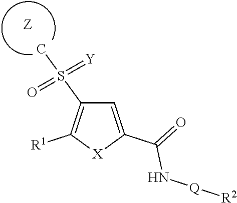

wherein
X is chosen from N(R^{a}) and S;
R^{a} is chosen from (C₁-C₆)hydrocarbon, (C₁-C₆)oxaalkyl, and hydrogen, wherein said (C₁-C₆)hydrocarbon and said (C₁-C₆)oxaalkyl is optionally substituted with one or more hydrogen, alkyl, alkoxy, hydroxyl, oxo, carbonyl, halogen, amino, cyano, or aminoacyl alkyl;
R¹ is chosen from methyl and ethyl; or
R^{a} and R¹ taken together with the atoms to which they are attached form a 5- or 6-member heterocycle optionally substituted with methyl; and
Y is chosen from O and N(R^{b});
R^{b} is chosen from hydrogen, (C₁-C₆)hydrocarbon, and hydroxyalkyl;
Z is chosen from aryl or heteroaryl, wherein said aryl and said heteroaryl is optionally substituted with one or more (C₁-C₆)alkyl, cyano, halogen, haloalkyl, cyanoalkyl, alkoxyalkyl, alkylsulfonyl, oxo, carbonyl, alkylcarbonyl, carboxamide, alkoxy, acetamide, cycloalkyl, hydroxyalkyl, or amino;
Q is chosen from direct bond, (C₁-C₆)hydrocarbon, (C₁-C₆)heteroalkyl, heterocycle, NH,

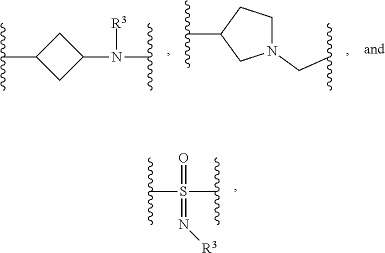

wherein said (C₁-C₆)hydrocarbon, said (C₁-C₆)heteroalkyl, and said heterocycle, is optionally substituted with one or more (C₁-C₁₀)hydrocarbyl, methylazetidine, oxaalkyl, hydroxyalkyl, halogen, oxo, hydroxyl, or haloalkyl;
R³ is chosen from hydrogen, alkyl, and haloalkyl;
R² is chosen from HetA, CarbA, —O-HetA, N(R^{c})₂, and C₁₃ hydrocarbon, said HetA and said CarbA is optionally substituted with one or more HetB, CarbB, halogen, alkoxy, oxaalkyl, cyano, COOH, hydroxyl, oxo, carbonyl, N(R^{c})₂, pyrrolidinylmethyl, azaalkyl, alkyl, thioalkyl, haloalkyl, alkoxylalkyl, haloalkylalkoxy, dimethylaminoalkyl, oxotetrahydrofuran, alkylsulfonyl, dimethylaminoalkylalkoxy, alkylsulfonylalkylalkoxy, —O-CarbB, or —O-HetB,
wherein said HetB, said alkoxy, said CarbB, said oxaalkyl, said azaalkyl, and said thioalkyl, is optionally substituted with one or more halogen, hydroxyl, (C₁-C₆)alkoxy, (C₁-C₆)hydrocarbyl, COOH, cyano, C(O)OC(CH₃)₂, N(R^{c})₂, (C₁-C₆)alkoxy(C₁-C₆)alkyl, (C₁-C₆)alkyl, oxo, carbonyl, C(O)CH₃, or alkylsulfonylcarboxamide,
wherein R^{c} is independently chosen in each instance from hydrogen, hydrocarbyl, haloalkyl, cyano, cyanoalkyl, alkoxyalkyl, carbonylhaloalkyl, oxaalkyl, carbonylalkyl, alkylsulfonyl, and HetA, said HetA optionally substituted with one or more halogen, and
wherein HetA is a 3- to 12-member heterocycle, HetB is a 3- to 11-member heterocycle, CarbA is a 6- to 10-member carbocycle, and CarbB is a 3- to 5-member carbocycle.

It has been found that compounds of formula (I) are useful for inhibiting the SARS-CoV-2 methyltransferase enzyme NSP14.

In some embodiments, X is S. In some embodiments, X is N(R^{a}). In some embodiments, R^{a} is (C₁-C₆)hydrocarbon optionally substituted with one or more hydrogen, alkyl (e.g., methyl, ethyl, and the like), alkoxy (e.g., methoxy, ethoxy, and the like), hydroxyl (i.e., OH), oxo (═O), carbonyl, halogen (e.g., F, Cl, and the like), amino (e.g., NH₂ and the like), cyano, or aminoacyl alkyl (e.g., NH—C(O)—CH₃ and the like), or any combination thereof. In some embodiments, R^{a} is (C₁-C₆)oxaalkyl optionally substituted with one or more hydrogen, alkyl (e.g., methyl, ethyl, and the like), alkoxy (e.g., methoxy, ethoxy, and the like), hydroxyl (i.e., OH), oxo (═O), carbonyl, halogen (e.g., F, Cl, and the like), amino (e.g., NH₂ and the like), cyano, or aminoacyl alkyl (e.g., NH—C(O)—CH₃ and the like), or any combination thereof. In some embodiments, R^{a} may be (C₁-C₃)hydrocarbon optionally substituted with one or more methyl, hydroxyl, methoxy, fluoro, oxo, NH₂, ethyl, cyano, or NH—C(O)—CH₃. In some embodiments, R^{a} may be (C₁-C₄)oxaalkyl optionally substituted with one or more methyl, hydroxyl, methoxy, fluoro, oxo, NH₂, ethyl, cyano, or NH—C(O)—CH₃. In some embodiments, R^{a} is (C₁-C₃)hydrocarbon. In some embodiments, R^{a} is methyl. In some embodiments, R^{a} is hydrogen.

In some embodiments, R¹ is alkyl (e.g., methyl, ethyl, and the like). In some embodiments, R¹ is methyl. In some embodiments, R¹ is ethyl.

In some embodiments, R^{a} and R¹ taken together with the atoms to which they are attached form a 5- or 6-member heterocycle optionally substituted with alkyl (e.g., methyl, ethyl, and the like). In some embodiments, R^{a} and R¹ taken together with the atoms to which they are attached form a 5- or 6-member heterocycle optionally substituted with methyl. In some embodiments, R^{a} and R¹ taken together with the atoms to which they are attached form an optionally substituted 5-member heterocycle. In some embodiments, R^{a} and R¹ taken together with the atoms to which they are attached form pyrrolidine. In some embodiments, R^{a} and R¹ taken together with the atoms to which they are attached form an optionally substituted 6-member heterocycle. In some embodiments, R^{a} and R¹ taken together with the atoms to which they are attached form optionally substituted piperazine. In some embodiments, R^{a} and R¹ taken together with the atoms to which they are attached form piperazine substituted with methyl.

In some embodiments, Y is O. In some embodiments, Y is N(R^{b}). In some embodiments, R^{b} is hydrogen. In some embodiments, R^{b} is (C₁-C₆)alkyl (e.g., methyl, ethyl, and the like). In some embodiments, R^{b} is methyl. In some embodiments, R^{b} is hydroxyalkyl, e.g., hydroxymethyl, hydroxyethyl, and the like).

In some embodiments, Z is aryl optionally substituted with one or more alkyl (e.g., methyl, ethyl, and the like), cyano, halogen (e.g., F, Cl, and the like), haloalkyl (e.g., fluoromethyl, difluoromethyl, trifluoromethyl, fluoroethyl, and the like), cyanoalkyl (e.g., cyanomethyl, cyanoethyl, and the like), alkoxyalkyl (e.g., methoxymethyl, methoxyethyl, ethoxymethyl, ethoxyethyl, and the like), alkylsulfonyl (e.g., methylsulfonyl, ethylsulfonyl, and the like), oxo (═O), carbonyl, alkylcarbonyl, carboxamide, alkoxy (e.g., methoxy, ethoxy, and the like), acetamide, cycloalkyl, hydroxyalkyl, or amino (e.g., NH₂ and the like). In some embodiments, Z is heteroaryl optionally substituted with one or more alkyl (e.g., methyl, ethyl, and the like), cyano, halogen (e.g., F, Cl, and the like), haloalkyl (e.g., fluoromethyl, difluoromethyl, trifluoromethyl, fluoroethyl, and the like), cyanoalkyl (e.g., cyanomethyl, cyanoethyl, and the like), alkoxyalkyl (e.g., methoxymethyl, methoxyethyl, ethoxymethy, ethoxyethyl, and the like), alkylsulfonyl (e.g., methylsulfonyl, ethylsulfonyl, and the like), oxo (═O), carbonyl, alkylcarbonyl, carboxamide, alkoxy (e.g., methoxy, ethoxy, and the like), acetamide, cycloalkyl, hydroxyalkyl, or amino (e.g., NH₂ and the like). In some embodiments, Z is a 5- or 6-member nitrogen-containing monocycle optionally substituted with one or more methyl, C(O)CH₃, haloalkyl, ethyl, cyano, cyanomethyl, methoxy, or methoxymethyl. In some embodiments, Z is a fused nitrogen-containing bicycle optionally substituted with one or more methyl, cyano, halogen, haloalkyl, methylsulfonyl, NH₂, oxo, cycloalkyl, hydroxyalkyl, or carbonyl. In some embodiments, Z is a 5- or 6-membered aryl optionally substituted with carboxamide, acetamide, or cycloalkyl.

In some embodiments, Q is direct bond. In some embodiments, Q is (C₁-C₆)hydrocarbon (e.g., methyl, ethyl, propyl, isopropyl, cyclopropyl, and the like) optionally substituted with one or more (C₁-C₁₀)hydrocarbyl, methylazetidine, oxaalkyl, hydroxyalkyl, halogen, oxo, hydroxyl, or haloalkyl. In some embodiments, Q is (C₁-C₆)heteroalkyl (e.g., (C₁-C₆)oxaalkyl, (C₁-C₆)azaalkyl, (C₁-C₆)thioalkyl, and the like) optionally substituted with one or more (C₁-C₁₀)hydrocarbyl, methylazetidine, oxaalkyl, hydroxyalkyl, halogen, oxo, hydroxyl, or haloalkyl. In some embodiments, Q is heterocycle optionally substituted with one or more (C₁-C₁₀)hydrocarbyl or methylazetidine. In some embodiments, Q is

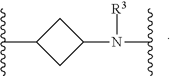

In some embodiments, Q is

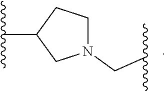 [SMILES: *CN1CCC(*)C1]

In some embodiments, Q is

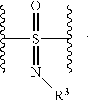

In some embodiments, Q may be CH₂, CH₂CH₂, CH₂-azetidine, or CH₂ substituted with one or more (C₁-C₁₀)alkyl.

In some embodiments, R³ is hydrogen. In some embodiments, R³ is alkyl (e.g., methyl, ethyl, etc.). In some embodiments, R³ is haloalkyl (e.g., fluoromethyl, difluoromethyl, trifluoromethyl, fluoroethyl, chloromethyl, etc.).

In some embodiments, R² may be HetA optionally substituted with one or more HetB, CarbB, halogen, alkoxy, oxaalkyl, cyano, COOH, hydroxyl, oxo, carbonyl, N(R^{c})₂, pyrrolidinylmethyl, azaalkyl, alkyl, thioalkyl, haloalkyl, alkoxylalkyl, haloalkylalkoxy, dimethylaminoalkyl, oxotetrahydrofuran, alkylsulfonyl, dimethylaminoalkylalkoxy, alkylsulfonylalkylalkoxy, —O-CarbB, or —O-HetB. In some embodiments, R² may be CarbA optionally substituted with one or more HetB, CarbB, halogen, alkoxy, oxaalkyl, cyano, COOH, hydroxyl, oxo, carbonyl, N(R^{c})₂, pyrrolidinylmethyl, azaalkyl, alkyl, hiaalkyl, haloalkyl, alkoxylalkyl, haloalkylalkoxy, dimethylaminoalkyl, oxotetrahydrofuran, alkylsulfonyl, dimethylaminoalkylalkoxy, alkylsulfonylalkylalkoxy, —O-CarbB, or —O-HetB. In some embodiments, R² is —O-HetA. In some embodiments, R² is N(R^{c})₂. In some embodiments, R² is (C₁₃)hydrocarbon. In some embodiments, HetB is optionally substituted with one or more halogen, hydroxyl, (C₁-C₆)alkoxy, (C₁-C₆)hydrocarbyl, COOH, cyano, C(O)OC(CH₃)₂, N(R^{c})₂, (C₁-C₆)alkoxy(C₁-C₆)alkyl, (C₁-C₆)alkyl, oxo, carbonyl, C(O)CH₃, or alkylsulfonylcarboxamide. In some embodiments, alkoxy is optionally substituted with one or more halogen, hydroxyl, (C₁-C₆)alkoxy, (C₁-C₆)hydrocarbyl, COOH, cyano, C(O)OC(CH₃)₂, N(R^{c})₂, (C₁-C₆)alkoxy(C₁-C₆)alkyl, (C₁-C₆)alkyl, oxo, carbonyl, C(O)CH₃, or alkylsulfonylcarboxamide. In some embodiments, CarbB is optionally substituted with one or more halogen, hydroxyl, (C₁-C₆)alkoxy, (C₁-C₆)hydrocarbyl, COOH, cyano, C(O)OC(CH₃)₂, N(R^{c})₂, (C₁-C₆)alkoxy(C₁-C₆)alkyl, (C₁-C₆)alkyl, oxo, carbonyl, C(O)CH₃, or alkylsulfonylcarboxamide. In some embodiments, R^{c} is independently chosen in each instance from hydrogen, hydrocarbyl (e.g., methyl, ethyl, propyl, isopropyl, cyclopropyl, and the like), carbonylhaloalkyl, oxaalkyl, carbonylalkyl, alkylsulfonyl, and HetA, said HetA optionally substituted with one or more halogen. In some embodiments, HetA is a 3- to 12-member heterocycle. In some embodiments, HetB is a 3- to 11-member heterocycle. In some embodiments, CarbA is a 6- to 10-member carbocycle. In some embodiments, CarbB is a 3- to 5-member carbocycle.

In some embodiments, R² is heteroaryl or aryl, optionally substituted with one or more 3- to 11-member heterocycle, halogen, (C₁-C₁₀)alkoxy, (C₁-C₁₀)hydrocarbyl, (C₁-C₁₀)oxaalkyl, cyano, COOH, hydroxyl, oxo, carbonyl, N(R^{c})₂, pyrrolidinylmethyl, (C₁-C₁₀)azaalkyl, difluoromethyl, or (C₁-C₁₀)thioalkyl; wherein said 3- to 11-member heterocycle, said (C₁-C₁₀)alkoxy, said (C₁-C₁₀)hydrocarbyl, said (C₁-C₁₀)oxaalkyl, said (C₁-C₁₀)azaalkyl, and said (C₁-C₁₀)thioalkyl, are each independently optionally substituted with one or more halogen, hydroxyl, (C₁-C₆)alkoxy, (C₁-C₆)hydrocarbyl, COOH, cyano, C(O)OC(CH₃)₂, or N(R^{c})₂; and wherein R^{c} in each instance is independently chosen from hydrogen, (C₁-C₁₀)hydrocarbyl, C(O)haloalkyl, oxo(C₁-C₆)alkyl, and alkylsulfonyl.

In some embodiments, R² is 6:5 or 6:6 bicyclic heterocycle, 5- to 10-member heterocycle, or phenyl; (i) wherein the phenyl is optionally substituted with one or more haloalkyl, alkoxyalkyl, cyano, COOH, alkoxy, or haloalkylalkoxy; (ii) the 6:5 or 6:6 bicyclic heterocycle or the 5- to 10-member heterocycle is optionally substituted with one or more 3- to 10-member heterocycle, (C₁-C₆)alkyl, dimethylaminemethyl, pyrrolidinylmethyl, oxo, carbonyl, halogen, hydroxyl, N(R^{d})₂, oxotetrahydrofuran, alkylsulfonyl, (C₁-C₆)hydrocarbon, cyano, COOH, or (C₁-C₃)alkoxy, (iia) wherein R^{d} in each instance is independently chosen from hydrogen, (C₁-C₆)hydrocarbon, and (C₁-C₆)oxaalkyl; (iib) wherein the 3- to 10-member heterocycle is optionally substituted with one or more alkoxyalkyl, alkyl, hydroxyl, oxo, carbonyl, or C(O)CH₃; and (iic) wherein the (C₁-C₆)hydrocarbon is optionally substituted with one or more halogen.

In an aspect, the disclosure relates to a method of inhibiting coronavirus NSP14 protein with a compound described above. In one embodiment, the coronavirus is SARS-CoV-2.

In an aspect, the disclosure relates to a method of treating a Covid-19 patient with a compound described above. In one embodiment, the disclosure relates to treating the disease associated with coronavirus generally.

In an aspect, the disclosure relates to a pharmaceutical formulation comprising a pharmaceutically acceptable carrier and a compound described above.

### Abbreviations and Definitions

Unless defined otherwise, all technical and scientific terms used herein have the same meaning as is commonly understood by one of ordinary skill in the art to which this disclosure belongs. A comprehensive list of abbreviations utilized by organic chemists (i.e., persons of ordinary skill in the art) appears in the first issue of each volume of the Journal of Organic Chemistry. The list, which is typically presented in a table entitled “Standard List of Abbreviations” is incorporated herein by reference. In the event that there is a plurality of definitions for terms cited herein, those in this section prevail unless otherwise stated.

As used herein, the terms “comprising” and “including,” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

A “patient” or “subject,” as used herein, includes both humans and other animals, particularly mammals. Thus, the methods are applicable to both human therapy and veterinary applications. In some embodiments, the patient is a mammal, for example, a primate. In some embodiments, the patient is a human.

Throughout this specification the terms and substituents retain their definitions.

Unless otherwise specified herein, “hydrocarbon” or hydrocarbyl (as a substituent), means any substituent comprised of hydrogen and carbon as the only elemental constituents. If not otherwise limited, (C₁-Cₙ)hydrocarbon, wherein n may be any integer from 1 to 20 or higher, is intended to include alkyl, cycloalkyl, polycycloalkyl, alkenyl, alkynyl, aryl, and combinations thereof. Non-limiting examples of a hydrocarbon include cyclopropylmethyl, benzyl, phenethyl, cyclohexylmethyl, adamantyl, camphoryl, naphthylethyl, etc. Hydrocarbyl refers to any substituent comprised of hydrogen and carbon as the only elemental constituents. Aliphatic hydrocarbons are hydrocarbons that are not aromatic; they may be saturated or unsaturated, cyclic, linear, or branched. Non-limiting examples of aliphatic hydrocarbons include isopropyl, 2-butenyl, 2-butynyl, cyclopentyl, norbornyl, etc. Non-limiting examples of aromatic hydrocarbons include benzene (phenyl), naphthalene (naphthyl), anthracene, etc. For example, (C₁-C₁₀)hydrocarbon includes all combination therein, i.e., (C₁-C₂)hydrocarbon, (C₁-C₃)hydrocarbon, (C₁-C₄)hydrocarbon, (C₁-C₅)hydrocarbon, (C₁-C₆)hydrocarbon, (C₁-C₇)hydrocarbon, (C₁-C₅)hydrocarbon, (C₁-C₉)hydrocarbon, (C₁-C₁₀)hydrocarbon, (C₂-C₃)hydrocarbon, (C₂-C₄)hydrocarbon, (C₂-C₅)hydrocarbon, (C₂-C₆)hydrocarbon, (C₂-C₇)hydrocarbon, (C₂-C₈)hydrocarbon, (C₂-C₉)hydrocarbon, (C₂-C₁₀)hydrocarbon, (C₃-C₄)hydrocarbon, (C₃-C₅)hydrocarbon, (C₃-C₆)hydrocarbon, (C₃-C₇)hydrocarbon, (C₃-C₈)hydrocarbon, (C₃-C₉)hydrocarbon, (C₃-C₁₀)hydrocarbon, (C₄-C₅)hydrocarbon, (C₄-C₆)hydrocarbon, (C₄-C₇)hydrocarbon, (C₄-C₈)hydrocarbon, (C₄-C₉)hydrocarbon, (C₄-C₁₀)hydrocarbon, (C₅-C₆)hydrocarbon, (C₅-C₇)hydrocarbon, (C₅-C₈)hydrocarbon, (C₅-C₉)hydrocarbon, (C₅-C₁₀)hydrocarbon, (C₆-C₇)hydrocarbon, (C₆-C₈)hydrocarbon, (C₆-C₉)hydrocarbon, (C₆-C₁₀)hydrocarbon, (C₇-C₈)hydrocarbon, (C₇-C₉)hydrocarbon, (C₇—C₁₀)hydrocarbon, (C₈-C₉)hydrocarbon, (C₅-C₁₀)hydrocarbon, (C₉-C₁₀)hydrocarbon, (C₁)hydrocarbon, (C₂)hydrocarbon, (C₃)hydrocarbon, (C₄)hydrocarbon, (C₅)hydrocarbon, (C₆)hydrocarbon, (C₇)hydrocarbon, (C₈)hydrocarbon, (C₉)hydrocarbon, and (C₁₀)hydrocarbon.

Unless otherwise specified, alkyl (or alkylene) is intended to include linear or branched hydrocarbon structures. Unless otherwise specified, alkyl refers to alkyl groups from 1 to 20 or higher carbon atoms, in some instances 1 to 10 carbon atoms, in some instances 1 to 6 carbon atoms, in some instances 1 to 4 carbon atoms, and in some instances 1 to 3 carbon atoms. Non-limiting examples of alkyl groups include methyl, ethyl, propyl, isopropyl, cyclopropyl, n-butyl, s-butyl, t-butyl, and the like. (C₁-C₁₀)alkyl includes all combinations therein, i.e., (C₁-C₂)alkyl, (C₁-C₃)alkyl, (C₁-C₄)alkyl, (C₁-C₅)alkyl, (C₁-C₆)alkyl, (C₁-C₇)alkyl, (C₁-C₅)alkyl, (C₁-C₉)alkyl, (C₂-C₃)alkyl, (C₂-C₄)alkyl, (C₂-C₅)alkyl, (C₂-C₆)alkyl, (C₂-C₇)alkyl, (C₂-C₈)alkyl, (C₂-C₉)alkyl, (C₂-C₁₀)alkyl, (C₃-C₄)alkyl, (C₃-C₅)alkyl, (C₃-C₆)alkyl, (C₃-C₇)alkyl, (C₃-C₈)alkyl, (C₃-C₉)alkyl, (C₃-C₁₀)alkyl, (C₄-C₅)alkyl, (C₄-C₆)alkyl, (C₄-C₇)alkyl, (C₄-C₈)alkyl, (C₄-C₉)alkyl, (C₄-C₁₀)alkyl, (C₅-C₆)alkyl, (C₅-C₇)alkyl, (C₅-C₈)alkyl, (C₅-C₉)alkyl, (C₅-C₁₀)alkyl, (C₆-C₇)alkyl, (C₆-C₈)alkyl, (C₆-C₉)alkyl, (C₆-C₁₀)alkyl, (C₇-C₈)alkyl, (C₇-C₉)alkyl, (C₇-C₁₀)alkyl, (C₈-C₉)alkyl, (C₅-C₁₀)alkyl, (C₉-C₁₀)alkyl, (C₁)alkyl, (C₂)alkyl, (C₃)alkyl, (C₄)alkyl, (C₅)alkyl, (C₆)alkyl, (C₇)alkyl, (C₈)alkyl, (C₉)alkyl, and (C₁₀)alkyl.

Unless otherwise specified, oxaalkyl is intended to include alkyl residues in which one or more carbons (and their associated hydrogens) have been replaced by oxygen. Non-limiting examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, 196, but without the restriction of 127(a)], i.e., it refers to compounds in which the oxygen is bonded via a single bond to its adjacent atoms (forming ether bonds); it does not refer to doubly bonded oxygen, as would be found in carbonyl groups. (C₁-C₁₀)oxaalkyl includes all combinations therein, i.e., (C₁-C₂)oxaalkyl, (C₁-C₃)oxaalkyl, (C₁-C₄)oxaalkyl, (C₁-C₅)oxaalkyl, (C₁-C₆)oxaalkyl, (C₁-C₇)oxaalkyl, (C₁-C₈)oxaalkyl, (C₁-C₉)oxaalkyl, (C₂-C₃)oxaalkyl, (C₂-C₄)oxaalkyl, (C₂-C₅)oxaalkyl, (C₂-C₆)oxaalkyl, (C₂-C₇)oxaalkyl, (C₂-C₈)oxaalkyl, (C₂-C₉)oxaalkyl, (C₂-C₁₀)oxaalkyl, (C₃-C₄)oxaalkyl, (C₃-C₅)oxaalkyl, (C₃-C₆)oxaalkyl, (C₃-C₇)oxaalkyl, (C₃-C₈)oxaalkyl, (C₃-C₉)oxaalkyl, (C₃-C₁₀)oxaalkyl, (C₄-C₅)oxaalkyl, (C₄-C₆)oxaalkyl, (C₄-C₇)oxaalkyl, (C₄-C₈)oxaalkyl, (C₄-C₉)oxaalkyl, (C₄-C₁₀)oxaalkyl, (C₅-C₆)oxaalkyl, (C₅-C₇)oxaalkyl, (C₅-C₈)oxaalkyl, (C₅-C₉)oxaalkyl, (C₅-C₁₀)oxaalkyl, (C₆-C₇)oxaalkyl, (C₆-C₈)oxaalkyl, (C₆-C₉)oxaalkyl, (C₆-C₁₀)oxaalkyl, (C₇-C₈)oxaalkyl, (C₇-C₉)oxaalkyl, (C₇-C₁₀)oxaalkyl, (C₈-C₉)oxaalkyl, (C₅-C₁₀)oxaalkyl, (C₉-C₁₀)oxaalkyl, (C₁)oxaalkyl, (C₂)oxaalkyl, (C₃)oxaalkyl, (C₄)oxaalkyl, (C₅)oxaalkyl, (C₆)oxaalkyl, (C₇)oxaalkyl, (C₈)oxaalkyl, (C₉)oxaalkyl, and (C₁₀)oxaalkyl.

Unless otherwise specified, azaalkyl is intended to include alkyl residues in which one or more carbons (and their associated hydrogens) have been replaced by nitrogen. Non-limiting examples include ethylaminoethyl and the like. (C₁-C₁₀)azaalkyl includes all combinations therein, i.e., (C₁-C₂)azaalkyl, (C₁-C₃)azaalkyl, (C₁-C₄)azaalkyl, (C₁-C₅)azaalkyl, (C₁-C₆)azaalkyl, (C₁-C₇)azaalkyl, (C₁-C₈)azaalkyl, (C₁-C₉)azaalkyl, (C₂-C₃)azaalkyl, (C₂-C₄)azaalkyl, (C₂-C₅)azaalkyl, (C₂-C₆)azaalkyl, (C₂-C₇)azaalkyl, (C₂-C₈)azaalkyl, (C₂-C₉)azaalkyl, (C₂-C₁₀)azaalkyl, (C₃-C₄)azaalkyl, (C₃-C₅)azaalkyl, (C₃-C₆)azaalkyl, (C₃-C₇)azaalkyl, (C₃-C₈)azaalkyl, (C₃-C₉)azaalkyl, (C₃-C₁₀)azaalkyl, (C₄-C₅)azaalkyl, (C₄-C₆)azaalkyl, (C₄-C₇)azaalkyl, (C₄-C₈)azaalkyl, (C₄-C₉)azaalkyl, (C₄-C₁₀)azaalkyl, (C₅-C₆)azaalkyl, (C₅-C₇)azaalkyl, (C₅-C₈)azaalkyl, (C₅-C₉)azaalkyl, (C₅-C₁₀)azaalkyl, (C₆-C₇)azaalkyl, (C₆-C₈)azaalkyl, (C₆-C₉)azaalkyl, (C₆-C₁₀)azaalkyl, (C₇-C₈)azaalkyl, (C₇-C₉)azaalkyl, (C₇-C₁₀)azaalkyl, (C₈-C₉)azaalkyl, (C₅-C₁₀)azaalkyl, (C₉-C₁₀)azaalkyl, (C₁)azaalkyl, (C₂)azaalkyl, (C₃)azaalkyl, (C₄)azaalkyl, (C₅)azaalkyl, (C₆)azaalkyl, (C₇)azaalkyl, (C₈)azaalkyl, (C₉)azaalkyl, and (C₁₀)azaalkyl.

Unless otherwise specified, thioalkyl is intended to include alkyl residues in which one or more carbons (and their associated hydrogens) have been replaced by sulfur. Non-limiting examples include methylthiopropyl and the like. (C₁-C₁₀)thioalkyl includes all combinations therein, i.e., (C₁-C₂)thioalkyl, (C₁-C₃)thioalkyl, (C₁-C₄)thioalkyl, (C₁-C₅)thioalkyl, (C₁-C₆)thioalkyl, (C₁-C₇)thioalkyl, (C₁-C₈)thioalkyl, (C₁-C₉)thioalkyl, (C₂-C₃)thioalkyl, (C₂-C₄)thioalkyl, (C₂-C₅)thioalkyl, (C₂-C₆)thioalkyl, (C₂-C₇)thioalkyl, (C₂-C₈)thioalkyl, (C₂-C₉)thioalkyl, (C₂-C₁₀)thioalkyl, (C₃-C₄)thioalkyl, (C₃-C₅)thioalkyl, (C₃-C₆)thioalkyl, (C₃-C₇)thioalkyl, (C₃-C₈)thioalkyl, (C₃-C₉)thioalkyl, (C₃-C₁₀)thioalkyl, (C₄-C₅)thioalkyl, (C₄-C₆)thioalkyl, (C₄-C₇)thioalkyl, (C₄-C₈)thioalkyl, (C₄-C₉)thioalkyl, (C₄-C₁₀)thioalkyl, (C₅-C₆)thioalkyl, (C₅-C₇)thioalkyl, (C₅-C₈)thioalkyl, (C₅-C₉)thioalkyl, (C₅-C₁₀)thioalkyl, (C₆-C₇)thioalkyl, (C₆-C₈)thioalkyl, (C₆-C₉)thioalkyl, (C₆-C₁₀)thioalkyl, (C₇-C₈)thioalkyl, (C₇-C₉)thioalkyl, (C₇-C₁₀)thioalkyl, (C₈-C₉)thioalkyl, (C₅-C₁₀)thioalkyl, (C₉-C₁₀)thioalkyl, (C₁)thioalkyl, (C₂)thioalkyl, (C₃)thioalkyl, (C₄)thioalkyl, (C₅)thioalkyl, (C₆)thioalkyl, (C₇)thioalkyl, (C₈)thioalkyl, (C₉)thioalkyl, and (C₁₀)thioalkyl.

Unless otherwise specified herein, “carbocycle” is intended to include ring systems in which the ring atoms are all carbon but of any oxidation state. If not otherwise limited, “carbocycle” is intended to include both non-aromatic and aromatic systems. In addition, unless otherwise specified herein, “carbocycle” is intended to include monocycles, bicycles, spirocycles, and polycycles. In a non-limiting example, (C₃-C₁₀)carbocycle may refer to cyclopropane, cyclohexane, benzene, phenyl, cyclopentadiene, cyclohexene, norbornane, decalin, naphthalene, indane, and the like. (C₃-C₁₀)carbocycle includes all combinations therein, i.e., (C₃-C₄)carbocycle, (C₃-C₅)carbocycle, (C₃-C₆)carbocycle, (C₃-C₇)carbocycle, (C₃-C₈)carbocycle, (C₃-C₉)carbocycle, (C₃-C₁₀)carbocycle, (C₄-C₅)carbocycle, (C₄-C₆)carbocycle, (C₄-C₇)carbocycle, (C₄-C₈)carbocycle, (C₄-C₉)carbocycle, (C₄-C₁₀)carbocycle, (C₅-C₆)carbocycle, (C₅-C₇)carbocycle, (C₅-C₈)carbocycle, (C₅-C₉)carbocycle, (C₅-C₁₀)carbocycle, (C₆-C₇)carbocycle, (C₆-C₈)carbocycle, (C₆-C₉)carbocycle, (C₆-C₁₀)carbocycle, (C₇-C₈)carbocycle, (C₇-C₉)carbocycle, (C₇-C₁₀)carbocycle, (C₈-C₉)carbocycle, (C₅-C₁₀)carbocycle, (C₉-C₁₀)carbocycle, (C₃)carbocycle, (C₄)carbocycle, (C₅)carbocycle, (C₆)carbocycle, (C₇)carbocycle, (C₈)carbocycle, (C₉)carbocycle, and (C₁₀)carbocycle.

Unless otherwise specified herein, “cycloalkyl” is a subset of hydrocarbyl and is intended to include cyclic hydrocarbon structures. If not otherwise limited, “cycloalkyl” may include cyclic alkyl groups of from 3 to 8 carbon atoms or from 3 to 6 carbon atoms. Non-limiting examples of cycloalkyl include cy-propyl, cy-butyl, cy-pentyl, norbornyl, and the like. (C₃-C₁₀)cycloalkyl includes all combinations therein, i.e., (C₃-C₄)cycloalkyl, (C₃-C₅)cycloalkyl, (C₃-C₆)cycloalkyl, (C₃-C₇)cycloalkyl, (C₃-C₈)cycloalkyl, (C₃-C₉)cycloalkyl, (C₃-C₁₀)cycloalkyl, (C₄-C₅)cycloalkyl, (C₄-C₆)cycloalkyl, (C₄-C₇)cycloalkyl, (C₄-C₈)cycloalkyl, (C₄-C₉)cycloalkyl, (C₄-C₁₀)cycloalkyl, (C₅-C₆)cycloalkyl, (C₅-C₇)cycloalkyl, (C₅-C₈)cycloalkyl, (C₅-C₉)cycloalkyl, (C₅-C₁₀)cycloalkyl, (C₆-C₇)cycloalkyl, (C₆-C₈)cycloalkyl, (C₆-C₉)cycloalkyl, (C₆-C₁₀)cycloalkyl, (C₇-C₈)cycloalkyl, (C₇-C₉)cycloalkyl, (C₇-C₁₀)cycloalkyl, (C₈-C₉)cycloalkyl, (C₅-C₁₀)cycloalkyl, (C₉-C₁₀)cycloalkyl, (C₃)cycloalkyl, (C₄)cycloalkyl, (C₅)cycloalkyl, (C₆)cycloalkyl, (C₇)cycloalkyl, (C₈)cycloalkyl, (C₉)cycloalkyl, and (C₁₀)cycloalkyl.

Heterocycle means a cycloalkyl or aryl carbocycle residue in which from 1 to 4 carbon atoms (and their associated hydrogens) is replaced by a heteroatom selected from the group consisting of N, O and S. The nitrogen and sulfur heteroatoms may optionally be oxidized, and the nitrogen heteroatom may optionally be quaternized. Unless otherwise specified, a heterocycle may be non-aromatic or aromatic. Heterocycle, if not otherwise limited, refers to monocycles, bicycles, spirocycles, and polycycles. Accordingly, 3- to 11-member heterocycle includes all combinations therein, e.g., 3-member heterocycle, 4-member heterocycle, 5-member heterocycle, 6-member heterocycle, 7-member heterocycle, 8-member heterocycle, 9-member heterocycle, 10-member heterocycle, 11-member heterocycle, 5:5 bicyclic heterocycle, 5:6 bicyclic heterocycle, 6:6 bicyclic heterocycle, 6:7 bicyclic heterocycle, 5:3 spiro heterocycle, and the like. Non-limiting examples of heterocycles that fall within the scope of the disclosure include pyrrolidine, pyrazole, pyrazine, pyrrole, indole, indazole, indoline, indolin-2-one, dihydroindene, dihydrocyclopentapyridine, dihydrofuropyridine, imidazole, quinoline, dihydroquinolinone, tetrahydroquinoline, isoquinoline, tetrahydroisoquinoline, benzofuran, dihydrobenzofuran, benzodioxan, benzodioxole (commonly referred to as methylenedioxyphenyl, when occurring as a substituent), benzoimidazole, dihydrobenzodioxepine, dihydropyrrolotriazole, triazole, tetrazole, morpholine, thiazole, pyridine, triazolopyridine, pyrazolopyridine, dihydropyridooxazine, imidazopyridine, pyridazine, pyrimidine, thiophene, furan, oxazole, oxazoline, oxadiazole, isoxazole, dioxane, chromane, tetrahydrofuran and the like.

Cycloazaalkyl is a subset of heterocycle in which at least 1 carbon atom (and its associated hydrogens) is replaced by N. Non-limiting examples of cycloaazaalkyl include azetidine, pyrrolidine, pyrazole, pyrrole, piperidine, pyridazine, pyrimidine and the like. (C₃-C₁₀)cycloazaalkyl includes all combinations therein, i.e., (C₃-C₄)cycloazaalkyl, (C₃-C₅)cycloazaalkyl, (C₃-C₆)cycloazaalkyl, (C₃-C₇)cycloazaalkyl, (C₃-C₈)cycloazaalkyl, (C₃-C₉)cycloazaalkyl, (C₃-C₁₀)cycloazaalkyl, (C₄-C₅)cycloazaalkyl, (C₄-C₆)cycloazaalkyl, (C₄-C₇)cycloazaalkyl, (C₄-C₈)cycloazaalkyl, (C₄-C₉)cycloazaalkyl, (C₄-C₁₀)cycloazaalkyl, (C₅-C₆)cycloazaalkyl, (C₅-C₇)cycloazaalkyl, (C₅-C₈)cycloazaalkyl, (C₅-C₉)cycloazaalkyl, (C₅-C₁₀)cycloazaalkyl, (C₆-C₇)cycloazaalkyl, (C₆-C₈)cycloazaalkyl, (C₆-C₉)cycloazaalkyl, (C₆-C₁₀)cycloazaalkyl, (C₇-C₈)cycloazaalkyl, (C₇-C₉)cycloazaalkyl, (C₇-C₁₀)cycloazaalkyl, (C₈-C₉)cycloazaalkyl, (C₅-C₁₀)cycloazaalkyl, (C₉-C₁₀)cycloazaalkyl, (C₃)cycloazaalkyl, (C₄)cycloazaalkyl, (C₅)cycloazaalkyl, (C₆)cycloazaalkyl, (C₇)cycloazaalkyl, (C₈)cycloazaalkyl, (C₉)cycloazaalkyl, and (C₁₀)cycloazaalkyl.

Cyclooxaalkyl is a subset of heterocycle in which at least 1 carbon atom (and its associated hydrogens) is replaced by O. Non-limiting examples of cyclooxaalkyl include furan, morpholine, chromane, oxazole and the like. (C₃-C₁₀)cyclooxaalkyl includes all combinations therein, i.e., (C₃-C₄)cyclooxaalkyl, (C₃-C₅)cyclooxaalkyl, (C₃-C₆)cyclooxaalkyl, (C₃-C₇)cyclooxaalkyl, (C₃-C₈)cyclooxaalkyl, (C₃-C₉)cyclooxaalkyl, (C₃-C₁₀)cyclooxaalkyl, (C₄-C₅)cyclooxaalkyl, (C₄-C₆)cyclooxaalkyl, (C₄-C₇)cyclooxaalkyl, (C₄-C₈)cyclooxaalkyl, (C₄-C₉)cyclooxaalkyl, (C₄-C₁₀)cyclooxaalkyl, (C₅-C₆)cyclooxaalkyl, (C₅-C₇)cyclooxaalkyl, (C₅-C₈)cyclooxaalkyl, (C₅-C₉)cyclooxaalkyl, (C₅-C₁₀)cyclooxaalkyl, (C₆-C₇)cyclooxaalkyl, (C₆-C₈)cyclooxaalkyl, (C₆-C₉)cyclooxaalkyl, (C₆-C₁₀)cyclooxaalkyl, (C₇-C₈)cyclooxaalkyl, (C₇-C₉)cyclooxaalkyl, (C₇-C₁₀)cyclooxaalkyl, (C₈-C₉)cyclooxaalkyl, (C₅-C₁₀)cyclooxaalkyl, (C₉-C₁₀)cyclooxaalkyl, (C₃)cyclooxaalkyl, (C₄)cyclooxaalkyl, (C₅)cyclooxaalkyl, (C₆)cyclooxaalkyl, (C₇)cyclooxaalkyl, (C₈)cyclooxaalkyl, (C₉)cyclooxaalkyl, and (C₁₀)cyclooxaalkyl.

Cyclothioalkyl is a subset of heterocycle in which at least 1 carbon atom (and its associated hydrogens) is replaced by S. Non-limiting examples of cyclothioalkyl include thiophene and the like. (C₃-C₁₀)cyclothioalkyl includes all combinations therein, i.e., (C₃-C₄)cyclothioalkyl, (C₃-C₅)cyclothioalkyl, (C₃-C₆)cyclothioalkyl, (C₃-C₇)cyclothioalkyl, (C₃-C₈)cyclothioalkyl, (C₃-C₉)cyclothioalkyl, (C₃-C₁₀)cyclothioalkyl, (C₄-C₅)cyclothioalkyl, (C₄-C₆)cyclothioalkyl, (C₄-C₇)cyclothioalkyl, (C₄-C₈)cyclothioalkyl, (C₄-C₉)cyclothioalkyl, (C₄-C₁₀)cyclothioalkyl, (C₅-C₆)cyclothioalkyl, (C₅-C₇)cyclothioalkyl, (C₅-C₈)cyclothioalkyl, (C₅-C₉)cyclothioalkyl, (C₅-C₁₀)cyclothioalkyl, (C₆-C₇)cyclothioalkyl, (C₆-C₈)cyclothioalkyl, (C₆-C₉)cyclothioalkyl, (C₆-C₁₀)cyclothioalkyl, (C₇-C₈)cyclothioalkyl, (C₇-C₉)cyclothioalkyl, (C₇-C₁₀)cyclothioalkyl, (C₈-C₉)cyclothioalkyl, (C₅-C₁₀)cyclothioalkyl, (C₉-C₁₀)cyclothioalkyl, (C₃)cyclothioalkyl, (C₄)cyclothioalkyl, (C₅)cyclothioalkyl, (C₆)cyclothioalkyl, (C₇)cyclothioalkyl, (C₈)cyclothioalkyl, (C₉)cyclothioalkyl, and (C₁₀)cyclothioalkyl.

Heteroaryl is a subset of heterocycle in which the heterocycle is aromatic. In some instances, the heteroaryl contains 4, 5, 6, or 7 ring members. In some instances, the heteroaryl is bicyclic and contains 8, 9, 10, or 11 total ring members. Non-limiting examples include furan, benzofuran, isobenzofuran, pyrrole, indole, isoindole, thiophene, benzothiophene, imidazole, benzimidazole, purine, pyrazole, indazole, oxazole, benzoxazole, isoxazole, benzisoxazole, thiazole, benzothiazole, triazole, tetrazole, pyridine, quinoline, isoquinoline, pyrazine, quinoxaline, acridine, pyrimidine, quinazoline, pyridazine, cinnoline, phthalazine, and triazine. Non-limiting examples of heterocyclyl residues additionally include piperazinyl, 2-oxopiperazinyl, 2-oxopiperidinyl, 2-oxo-pyrrolidinyl, 2-oxoazepinyl, azepinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

Aryl and heteroaryl mean (i) a phenyl group (or benzene) or a monocyclic 5- or 6-member heteroaromatic ring containing 1-4 heteroatoms selected from O, N, or S; (ii) a bicyclic 9- or 11-member aromatic or heteroaromatic ring system containing 0-4 heteroatoms selected from O, N, or S; or (iii) a tricyclic 13- or 14-member aromatic or heteroaromatic ring system containing 0-5 heteroatoms selected from O, N, or S. Non-limiting examples of the aromatic 6 to 14-member carbocyclic rings include benzene, naphthalene, indane, tetralin, and fluorene. Non-limiting examples of the 5- to 10-member aromatic heterocyclic rings include imidazole, pyridine, indole, thiophene, benzopyranone, thiazole, furan, benzimidazole, quinoline, isoquinoline, quinoxaline, pyrimidine, pyrazine, tetrazole and pyrazole. As used herein aryl and heteroaryl refer to residues in which one or more rings are aromatic, but not all need be.

Unless otherwise specified herein, “alkoxy” or “alkoxyl” refers to groups of from 1 to 20 carbon atoms, from 1 to 10 carbon atoms, or from 1 to 6 carbon atoms of a straight, branched, or cyclic configuration, and combinations thereof, attached to the parent structure through an oxygen. Non-limiting examples include methoxy, ethoxy, propoxy, isopropoxy cyclopropyloxy, cyclohexyloxy, methylenedioxy, ethylenedioxy, and the like. (C₁-C₁₀)alkoxy includes all combinations therein, i.e., (C₁-C₂)alkoxy, (C₁-C₃)alkoxy, (C₁-C₄)alkoxy, (C₁-C₅)alkoxy, (C₁-C₆)alkoxy, (C₁-C₇)alkoxy, (C₁-C₈)alkoxy, (C₁-C₉)alkoxy, (C₂-C₃)alkoxy, (C₂-C₄)alkoxy, (C₂-C₅)alkoxy, (C₂-C₆)alkoxy, (C₂-C₇)alkoxy, (C₂-C₈)alkoxy, (C₂-C₉)alkoxy, (C₂-C₁₀)alkoxy, (C₃-C₄)alkoxy, (C₃-C₅)alkoxy, (C₃-C₆)alkoxy, (C₃-C₇)alkoxy, (C₃-C₈)alkoxy, (C₃-C₉)alkoxy, (C₃-C₁₀)alkoxy, (C₄-C₅)alkoxy, (C₄-C₆)alkoxy, (C₄-C₇)alkoxy, (C₄-C₈)alkoxy, (C₄-C₉)alkoxy, (C₄-C₁₀)alkoxy, (C₅-C₆)alkoxy, (C₅-C₇)alkoxy, (C₅-C₈)alkoxy, (C₅-C₉)alkoxy, (C₅-C₁₀)alkoxy, (C₆-C₇)alkoxy, (C₆-C₈)alkoxy, (C₆-C₉)alkoxy, (C₆-C₁₀)alkoxy, (C₇-C₈)alkoxy, (C₇-C₉)alkoxy, (C₇-C₁₀)alkoxy, (C₈-C₉)alkoxy, (C₈-C₁₀)alkoxy, (C₉-C₁₀)alkoxy, (C₁)alkoxy, (C₂)alkoxy, (C₃)alkoxy, (C₄)alkoxy, (C₅)alkoxy, (C₆)alkoxy, (C₇)alkoxy, (C₈)alkoxy, (C₉)alkoxy, and (C₁₀)alkoxy.

Unless otherwise specified, acyl refers to formyl and to groups of 1, 2, 3, 4, 5, 6, 7 or 8 carbon atoms of a straight, branched, or cyclic configuration, saturated or unsaturated, and aromatic, and combinations thereof, attached to the parent structure through a carbonyl functionality. One or more carbons in the acyl residue may be replaced by nitrogen, oxygen or sulfur as long as the point of attachment to the parent remains at the carbonyl. Non-limiting examples include acetyl, benzoyl, propionyl, isobutyryl, t-butoxycarbonyl, benzyloxycarbonyl, and the like. The double bonded oxygen, when referred to as a substituent itself is called “oxo”.

Arylalkyl refers to a substituent in which an aryl residue is attached to the parent structure through alkyl. Non-limiting examples include benzyl, phenethyl and the like. Heteroarylalkyl refers to a substituent in which a heteroaryl residue is attached to the parent structure through alkyl. In one embodiment, the alkyl group of an arylalkyl or a heteroarylalkyl is an alkyl group of from 1 to 6 carbons. Non-limiting examples include pyridinylmethyl, pyrimidinylethyl, and the like.

An oxygen heterocycle is a heterocycle containing at least one oxygen in the ring; it may contain additional oxygens, as well as other heteroatoms. A sulphur heterocycle is a heterocycle containing at least one sulphur in the ring; it may contain additional sulphurs, as well as other heteroatoms. Oxygen heteroaryl is a subset of oxygen heterocycle; non-limiting examples include furan and oxazole. Sulphur heteroaryl is a subset of sulphur heterocycle; non-limiting examples include thiophene and thiazine. A nitrogen heterocycle is a heterocycle containing at least one nitrogen in the ring; it may contain additional nitrogens, as well as other heteroatoms. Non-limiting examples include piperidine, piperazine, morpholine, pyrrolidine and thiomorpholine. Nitrogen heteroaryl is a subset of nitrogen heterocycle; non-limiting examples include pyridine, pyrrole and thiazole.

The term “halogen” means fluorine, chlorine, bromine, or iodine atoms. In one embodiment, halogen may be a fluorine or chlorine atom.

The terms “haloalkyl” and “haloalkoxy” mean alkyl or alkoxy, respectively, substituted with one or more halogen atoms. The terms “alkylcarbonyl” and “alkoxycarbonyl” mean —C(═O)alkyl or —C(═O)alkoxy, respectively.

As used herein, the term “optionally substituted” may be used interchangeably with “unsubstituted or substituted”. The term “substituted” refers to the replacement of one or more hydrogen atoms in a specified group with a specified radical. For example, unless otherwise specified, substituted alkyl, aryl, cycloalkyl, heterocyclyl, etc. refer to alkyl, aryl, cycloalkyl, or heterocyclyl, etc., wherein one or more H atoms in each residue are independently replaced with halogen (e.g., fluoro, chloro, etc.), haloalkyl (e.g., difluoromethyl, trifluoromethyl, etc.), alkyl (e.g., methyl, etc.), acyl, alkoxyalkyl, carbonyl (also interchangeably written as ═O), phenyl, heteroaryl, benzenesulfonyl, hydroxy, haloalkoxy, oxaalkyl, azaalkyl, cycloazaalkyl, 5:3 spiro heterocycle, azabicyclo 3.1.0 hexane, azabicyclo 2.2.1 heptane, thioalkyl, cyclothioalkyl, carboxy, alkoxycarbonyl [—C(═O)O-alkyl], aminocarbonyl (also known as carboxamido) [—C(═O)NH₂], alkylaminocarbonyl [—C(═O)NH-alkyl], alkylcarbonylamino [—NH—C(═O)-alkyl], alkoxycarbonylamino [HNC(═O)O-alkyl], cyano, acetoxy, nitro, amino, alkylamino, alkylaminoalkyl, cycloalkylaminoalkyl, dialkylamino, dialkylaminoalkyl, dialkylaminoalkoxy, heterocyclylalkoxy, arylalkyl, (cycloalkyl)alkyl, heterocyclyl, heterocyclylalkyl, alkylaminoalkyl, heterocyclylaminoalkyl, heterocyclylalkylaminoalkyl, cycloalkylaminoalkyl, cycloalkylalkylaminoalkyl, arylaminoalkyl, arylalkylaminoalkyl, (alkyl)(aryl)aminoalkyl, mercapto, alkylthio, sulfoxide, sulfone, sulfonylamino, alkylsulfonyl, alkylsulfinyl, acylaminoalkyl, acylaminoalkoxy, acylamino, aryl, benzyl, heterocyclyl, heterocyclylalkyl, phenoxy, benzyloxy, heteroaryloxy, hydroxyimino, alkoxyimino, oxaalkyl, aminosulfonyl, trityl, amidino, guanidino, ureido, (C₁₋₆)hydrocarbyl, SO₂alkyl, SO₂NH₂, SO₂NHalkyl, benzyloxyphenyl, or benzyloxy. “Oxo” is also included among the substituents referred to in “optionally substituted”; it will be appreciated by persons of skill in the art that, because oxo is a divalent radical, there are circumstances in which it will not be appropriate as a substituent (e.g., on phenyl). In one embodiment, 1, 2, or 3 hydrogen atoms are replaced with a specified radical. In the case of alkyl and cycloalkyl, more than three hydrogen atoms can be replaced by fluorine; indeed, all available hydrogen atoms could be replaced by fluorine. In some embodiments, substituents are halogen, haloalkyl, alkyl, acyl, hydroxyalkyl, hydroxy, alkoxy, haloalkoxy, aminocarbonyl oxaalkyl, carboxy, cyano, acetoxy, nitro, amino, alkylamino, dialkylamino, alkylthio, alkylsulfinyl, alkylsulfonyl, alkylsulfonylamino, arylsulfonyl, arylsulfonylamino, and benzyloxy.

It is understood that any alkyl, alkenyl, alkynyl, cycloalkyl and cycloalkenyl moiety described herein can also be an aliphatic group, an alicyclic group or a heterocyclic group. An “aliphatic group” is non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted. It is understood that aliphatic groups may be used in place of the alkyl, alkenyl, alkynyl, alkylene, alkenylene, and alkynylene groups described herein.

Substituents Rⁿ are generally defined when introduced and retain that definition throughout the specification and in all independent claims.

The terms “subject” or “subject in need thereof” are used interchangeably herein. These terms refer to a patient who has been diagnosed with the underlying disorder to be treated. The subject may currently be experiencing symptoms associated with the disorder or may have experienced symptoms in the past. Additionally, a “subject in need thereof” may be a patient at risk of developing a particular disease, or to a patient reporting one or more of the physiological systems of a disease, even though a diagnosis of this disease may not have been made.

As used herein, the terms “treatment” or “treating” are used interchangeably. These terms refer to an approach for obtaining beneficial or desired results including, but not limited to, therapeutic benefit. Therapeutic benefit includes eradication or amelioration of the underlying disorder being treated; it also includes the eradication or amelioration of one or more of the symptoms associated with the underlying disorder such that an improvement is observed in the patient, notwithstanding that the patient may still be afflicted with the underlying disorder.

Preparation of compounds can involve the protection and deprotection of various chemical groups. The need for protection and deprotection, and the selection of appropriate protecting groups, can be readily determined by one skilled in the art. Suitable groups for that purpose are discussed in standard textbooks in the field of chemistry, such as Protective Groups in Organic Synthesis by T. W. Greene and P. G. M. Wuts [John Wiley & Sons, New York, 1999], in Protecting Group Chemistry, 1^{st} Ed., Oxford University Press, 2000; and in March's Advanced Organic chemistry: Reactions, Mechanisms, and Structure, 5^{th} Ed., Wiley-Interscience Publication, 2001.

As used herein, and as would be understood by the person of skill in the art, the recitation of “a compound”—unless expressly further limited—is intended to include salts of that compound. In a particular embodiment, the term “compound of formula” refers to the compound or a pharmaceutically acceptable salt thereof.

The term “pharmaceutically acceptable salt” refers to salts prepared from pharmaceutically acceptable non-toxic acids or bases including inorganic acids and bases and organic acids and bases. When the compounds of the present disclosure are basic, salts may be prepared from pharmaceutically acceptable non-toxic acids including inorganic and organic acids. Suitable pharmaceutically acceptable acid addition salts for the compounds of the present disclosure include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic side chain, suitable pharmaceutically acceptable base addition salts for the compounds of the present disclosure include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

Also provided herein is a pharmaceutical composition comprising a compound disclosed above, or a pharmaceutically acceptable salt thereof, together with one or more pharmaceutically carriers thereof and optionally one or more other therapeutic ingredients. The carrier(s) must be “acceptable” in the sense of being compatible with the other ingredients of the formulation and not deleterious to the recipient thereof.

The formulations include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association a compound of formula I or a pharmaceutically acceptable salt thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present disclosure suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

Unless otherwise stated or depicted, structures depicted herein are also meant to include all stereoisomeric (e.g., enantiomeric, diastereomeric, and cis-trans isomeric) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and cis-trans isomeric (or conformational) mixtures of the present compounds are within the scope of the disclosure. Unless otherwise stated, all tautomeric forms of the compounds of the disclosure are within the scope of the disclosure.

Additionally, unless otherwise stated, structures depicted herein are also meant to include compounds that differ only in the presence of one or more isotopically enriched atoms. Radioisotopes of hydrogen, carbon, phosphorous, fluorine, and chlorine include ²H, ³H, ¹³C, ¹⁴C, ¹⁵N, ³⁵, ¹⁸F, and ³⁶Cl, respectively. Compounds that contain those radioisotopes and/or other radioisotopes of other atoms are within the scope of this disclosure. Such compounds are useful, for example, as analytical tools or probes in biological assays. Radiolabeled compounds of the present disclosure can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent.

For the purpose of the present disclosure, a “pure” or “substantially pure” enantiomer is intended to mean that the enantiomer is at least 95% f the configuration shown and 5% r less of other enantiomers. Similarly, a “pure” or “substantially pure” diastereomer is intended to mean that the diastereomer is at least 95% f the relative configuration shown and 5% r less of other diastereomers.

### General Synthetic Schemes

A general procedure for the preparation of substituted pyrroles such as Formula I is outlined in Scheme A.1. Treatment of a suitably substituted halo-aryl/heteroaryl a.a with ethyl 3-mercaptopropanoate under Pd(0) catalysis provides the thio-propanoate substituted aryl/heteroaryl a.b. Treatment of a.b with appropriate bases (e.g. potassium tert-butoxide) provides the aryl/heteroaryl thiol a.c. The thiol a.c can be converted into the thio-substituted pyrrole a.e by sequential treatment of sulfuryl chloride (SO₂CO₂) followed by the appropriately substituted heteroaryl ester a.d. The thio-substituted esters a.e can be converted into sulfone-ester a.f by using appropriate oxidizing conditions (e.g. m-CPBA or Oxone). The ester in a.f can be hydrolyzed under basic, aqueous conditions (e.g. LiOH or NaOH, water) to provide the corresponding acid a.g. The acid moiety in a.g can be reacted with appropriately substituted amines (H₂N-Q-Ar) using amide coupling conditions (e.g. HATU/DIPEA) to provide examples of Formula I.

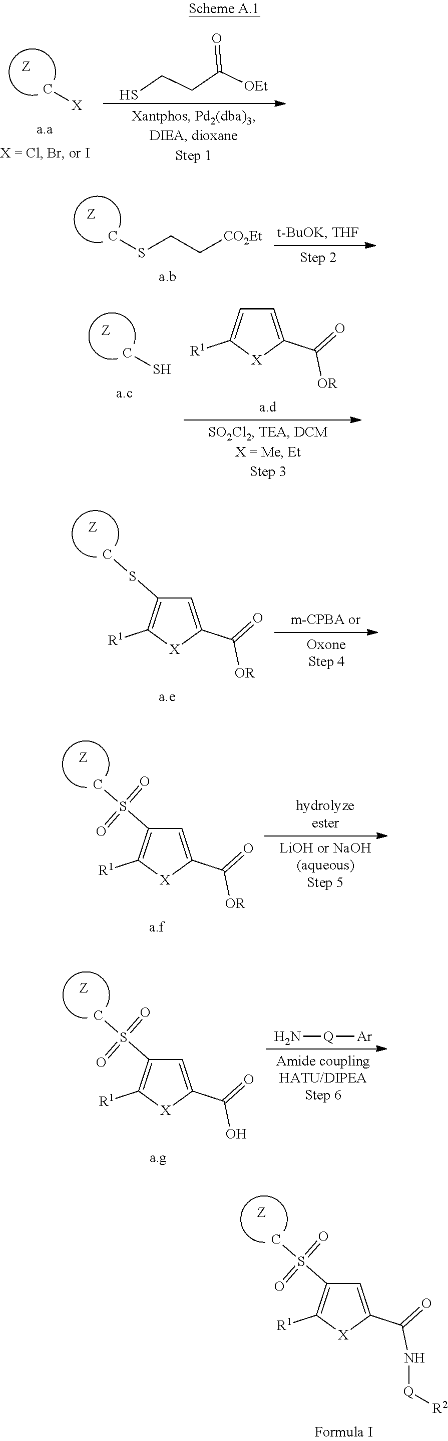

Amines (NH₂-Q-Ar) used for the preparation of examples of Formula I can be prepared according to conditions in Scheme B.1. An appropriately substituted cyano-substituted heteroaryl intermediate such as B.a can be reacted with amines (B.b) to provide amino-nitrile substituted heteroaryl intermediates such as B.c (bases such as TEA, DIPEA, or Cs₂CO₃ in an appropriate solvent). The cyano group in B.c can be reduced using various conditions (e.g. a. LAH, Raney-Ni, H₂, or Pd/C, H₂) to provide amines NH₂-Q-Ar used for making examples of Formula I. Alternatively, the cyano heteroaryl intermediates B.c can be converted into the Boc protected amines B.c.1 using appropriate conditions (NiCl₂-6H₂O/NaBH₄/Boc₂O/MeOH). The Boc group in B.c.1 can be removed using acidic conditions (e.g. TFA or HCl) to provide the amines B.d (NH₂-Q-Ar).

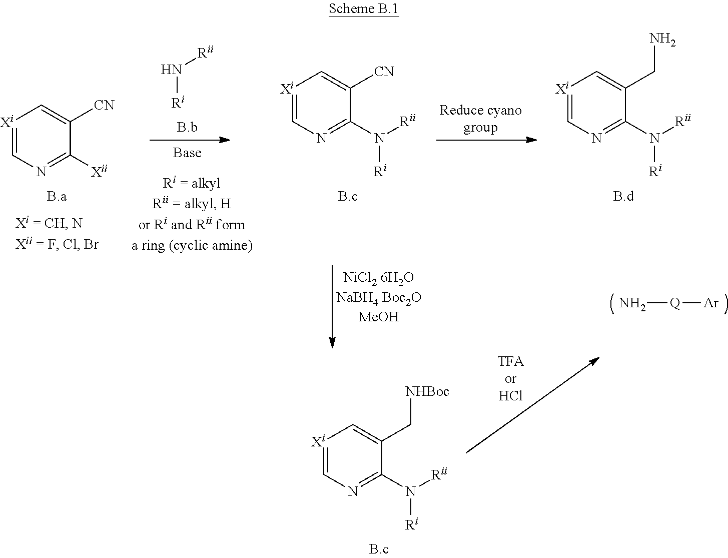

Amines (NH₂-Q-Ar) used for the preparation of examples of Formula I can be prepared according to conditions in Scheme C.1. Appropriately substituted halo-indazoles C.a can be converted into the corresponding cyano-indazole C.b. using conditions to those skilled in the art (e.g. Zn, Zn(CN)₂, Pd₂(dba)₃-DPPF). Cyano-indazoles C.b can be converted into amines C.c (NH2-Q-Ar) using standard conditions (e.g. H₂, Ni, NH₃-MeOH or H₂, Pd/C, MeOH).

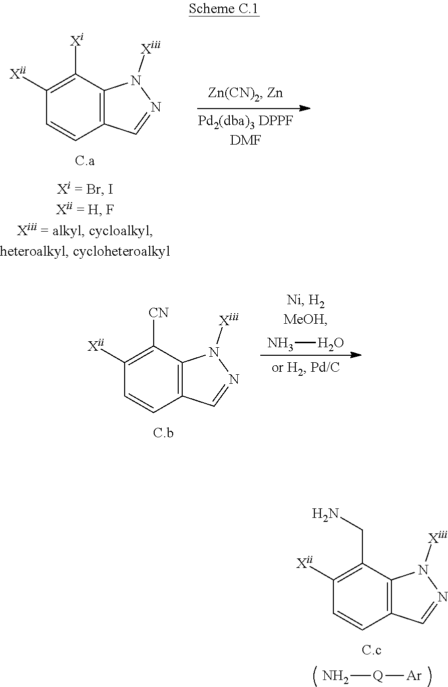

Amines (NH₂-Q-Ar) used for the preparation of examples of Formula I can be prepared according to conditions in Scheme D.1. Appropriately substituted ortho-fluoro,nitro aryl precursors such as D.a can be converted into the amino substituted intermediates such as D.b. via displacement of fluorine with an appropriate amine (H₂NXⁱⁱⁱ). The nitro group in D.b. can be reduced to provide the diamino intermediate D.c (e.g. Fe/NH₄Cl/EtOH). The diamino intermediate D.c can be converted into halo-benzimidazoles such as D.d (e.g. TsOH/CH(OMe)₃). Halo-benzimidazoles D.d can be converted into the corresponding cyano-substituted benzimidazoles D.e (e.g. Zn/Zn(CN)₂/Pd₂(dba)₃-DPPF/DMF). Cyano-substituted benzimidazoles D.e can be converted into amino-methyl substituted benzimidazoles D.f (NH₂-Q-Ar) via hydrogenation of the cyano group (e.g. H₂/Raney Ni/MeOH/NH₃-H₂O).

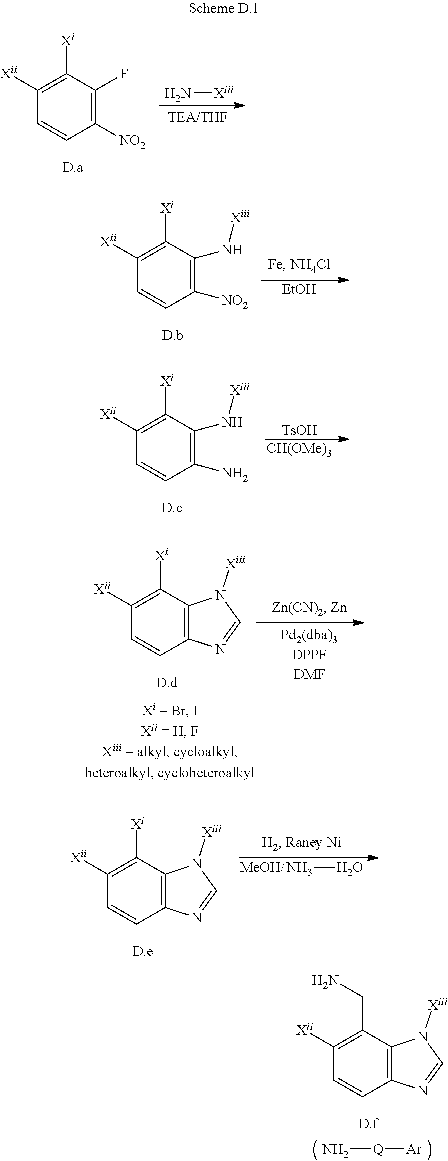

Indazole-pyrrole carboxamide examples can be prepared according to the steps outlined in Scheme E.1. An appropriately substituted halo-indazole e.a can be converted into the SEM protected indazole e.b. (in some cases a mixture of SEM protected indazole intermediates e.b is obtained which can be utilized without need for separation). Treatment of suitably substituted SEM-protected indazoles e.b with ethyl 3-mercaptopropanoate under Pd(0) catalysis provides thio-propanoate substituted indazoles. The formed thio-propanoate substituted indazoles can be treated with appropriate bases (e.g. potassium tert-butoxide) to form indazole thiols e.c. The SEM-protected thiols e.c can be converted into the thio-substituted pyrrole carboxy-esters e.e by sequential treatment of sulfuryl chloride (SO₂CO₂) followed by the appropriately substituted pyrrole ester e.d. The thio-substituted pyrrole carboxy-esters e.e can be converted into the corresponding sulfones e.f via oxidation (e.g. m-CPBA or Oxone). The ester moiety in e.f can be converted into the corresponding amide analogs e.g. via sequential hydrolysis of the ester to the acid (e.g. LiOH or MeOH in water/MeOH) followed by amide coupling (e.g. H₂N-Q-Ar/HATU/DIPEA). The SEM group in e.g. can be removed (e.g. TFA/DCM or HCl/MeOH) to furnish examples of Formula I.

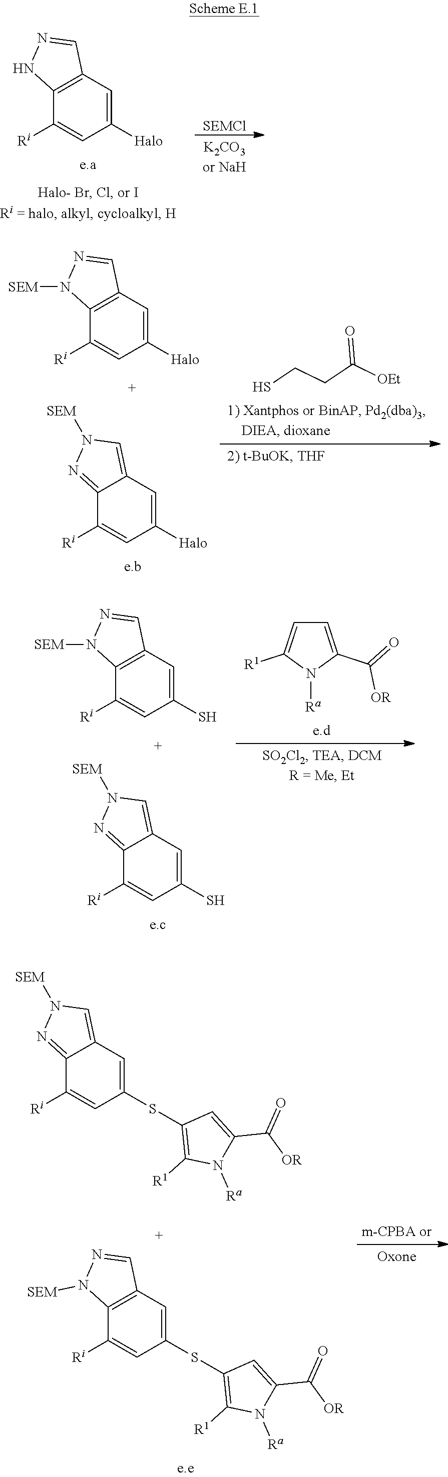

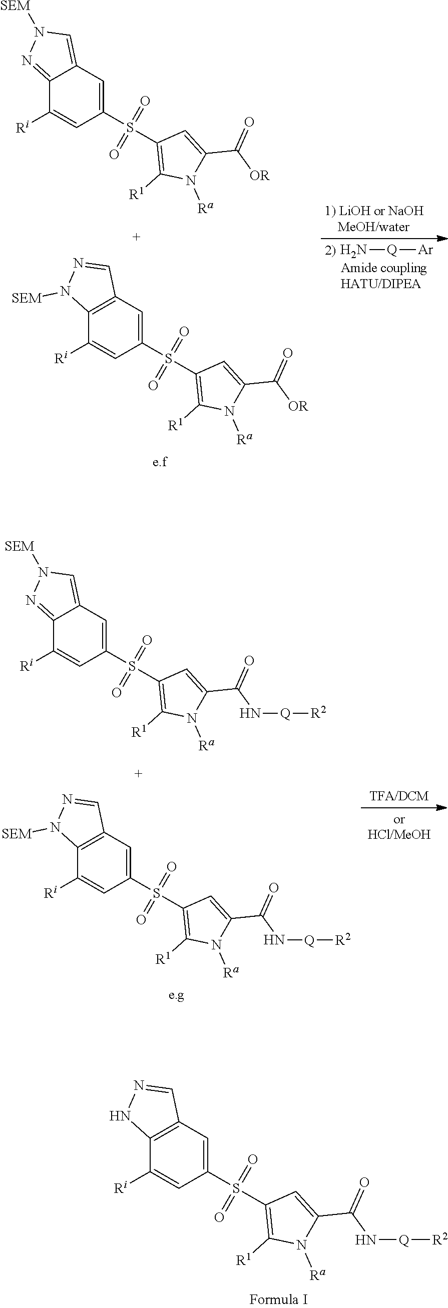

Indazole-pyrrole carboxamide analogs can be prepared according to the steps outlined in Scheme F.1. The SEM protected thiol f.a cab be prepared from the corresponding halo-indazole in a similar fashion to that described above in Scheme E.1 (Steps 1-3). The thio-substituted pyrrole f.c. can be prepared in a similar fashion to that described in Step 4 of Scheme E.1 using the appropriate pyrrole-ester fb and thiol f.a. The pyrrole NH in f.c can be converted into the corresponding Ra-substituted pyrrole intermediate f.d using the appropriate conditions (e.g. R^{a}=alkyl/hydroxy alkyl: NaH/X—R^{a} or R^{a}=cyclopropyl: Cu(OAc)₂/R^{a}—B(OH)₂). The substituted pyrrole-ester f.d can be converted into examples of Formula I using conditions outlined in steps 5-8 of Scheme E.1.

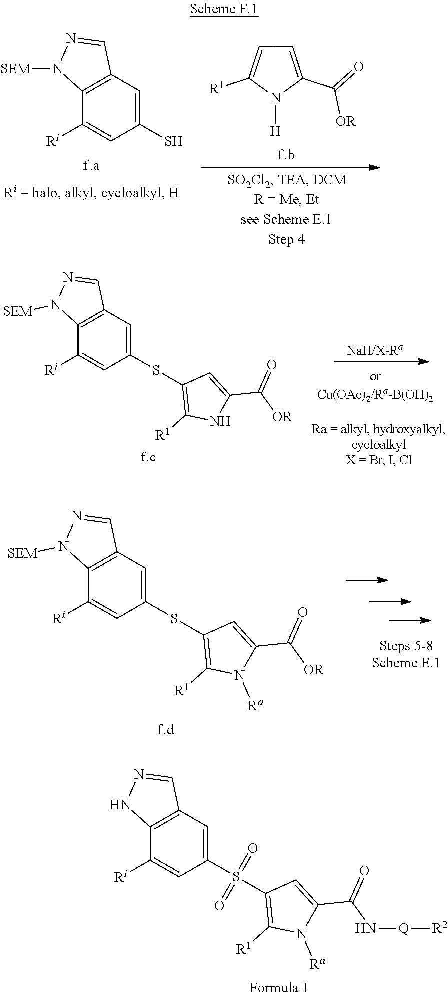

N-Aryl-Amino-cycloheteroalkyl intermediates g.d can be prepared according to Scheme G.1 (H₂N-Q-R₂). Halo-aryl/heteroaryl substrates g.a can be converted into the corresponding Boc protected N-aryl/heteroaryl intermediates g.c using an appropriate base/solvent (e.g. K₂CO₃/DMF) or Pd(0) catalyzed Buchwald conditions (e.g. Pd₂dba₃/Xantphos or BINAP/Cs₂CO₃) and Boc protected cycloheteroalkyl amine g.b. The Boc group in g.c can be removed (e.g. HCl or TFA) to provide the amino-heteroalkyl intermediate g.d (e.g. NH₂-Q-R²).

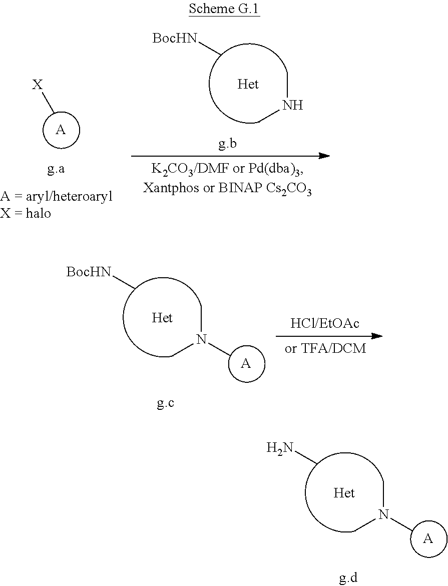

The preparation of sulfoximine derived examples is depicted in Scheme H.1. The intermediate f.d can be converted in the sulfoximine intermediate h.a by known methods (e.g. PhI(OAc)₂, H₂NCO₂NII₄). The sulfoximine NH in h.a can be derivatized to furnish the alkyl substituted sulfoximine h.b (e.g. NaH/X—R^{b}) or (Cu(OAc)₂/R^{b}—B(OH)₂). The ester in h.b. can be hydrolyzed to the corresponding acid h.c. The intermediate h.c can be converted into examples of Formula I using conditions outlined in Steps 7 and 8 of Scheme E.1.

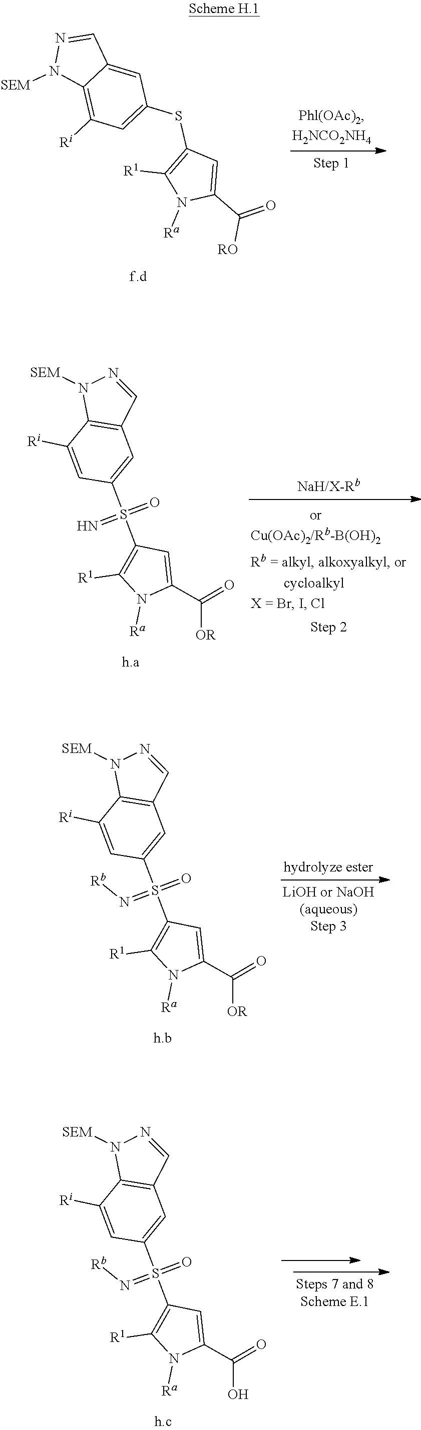

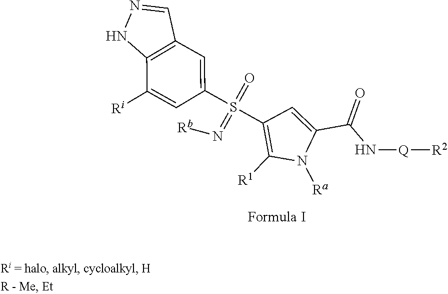

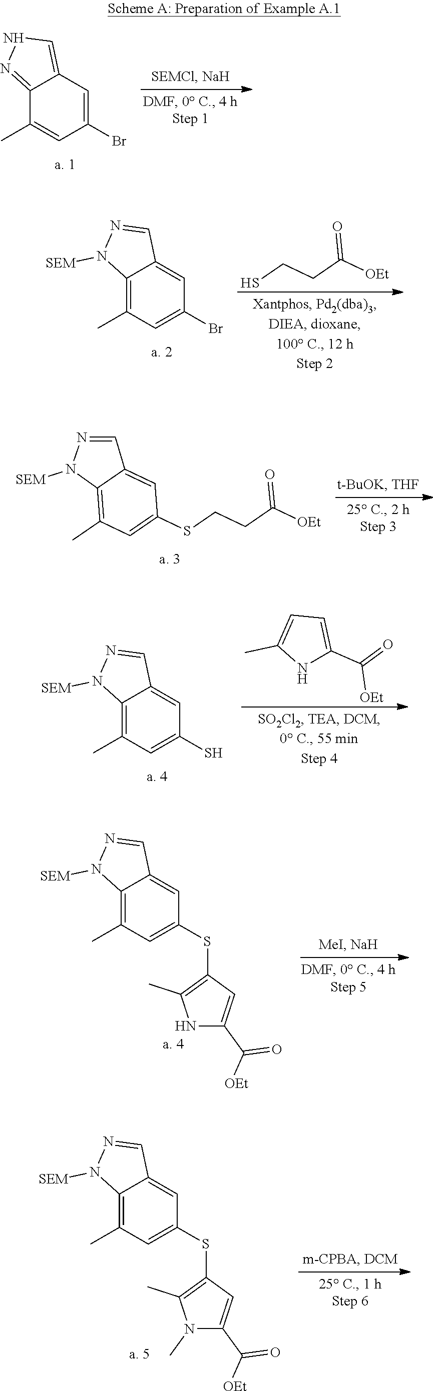

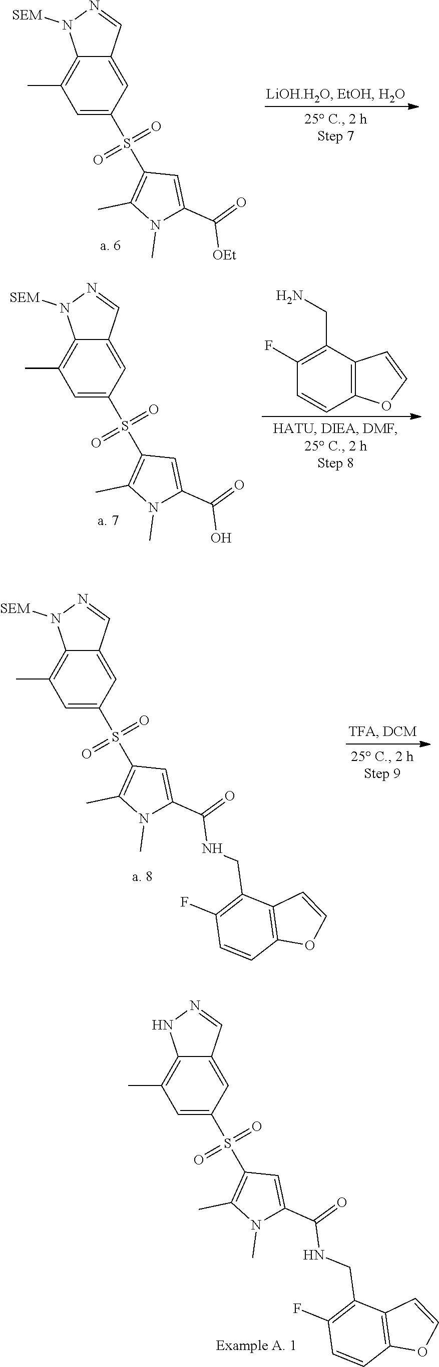 [SMILES: CCOC(=O)c1cc(S(=O)(=O)c2cc(C)c3c(cnn3COCC[Si](C)(C)C)c2)c(C)n1C ; Cc1cc(S(=O)(=O)c2cc(C(=O)O)n(C)c2C)cc2cnn(COCC[Si](C)(C)C)c12 ; NCc1c(F)ccc2occc12 ; Cc1cc(S(=O)(=O)c2cc(C(=O)NCc3c(F)ccc4occc34)n(C)c2C)cc2cnn(COCC[Si](C)(C)C)c12 ; Cc1cc(S(=O)(=O)c2cc(C(=O)NCc3c(F)ccc4occc34)n(C)c2C)cc2cn[nH]c12]

### EXPERIMENTAL SECTION

Unless otherwise noted, all reactions are magnetically stirred.

Unless otherwise noted, when ethyl acetate, hexanes, dichloromethane, 2-propanol, and methanol are used in the experiments below, they are Fisher Optima grade solvents.

Unless otherwise noted, when diethyl ether is used in the experiments described below, it is Fisher ACS certified material and is stabilized with BHT.

Unless otherwise noted, “concentrated to dryness” or “removal of the solvent” means evaporating the solvent from a solution or mixture using a rotary evaporator.

Unless otherwise noted, flash chromatography is carried out on an Isco, Analogix, or Biotage automated chromatography system using a commercially available cartridge as the column. Columns are usually filled with silica gel as the stationary phase.

Unless otherwise noted, preparative HPLC (or prep-HPLC) is carried out with commercial columns in a reverse phase manner (the stationary phase is hydrophobic). Typical solvent mixtures include A (water) and B (organic i.e., acetonitrile, methanol, etc.). Additives can also be used in the solvent mixture such as HCl, NH₄HCO₃, and formic acid.

Abbreviations used in the experimental section may include the following:

| | |
|---|---|
| ACN = | Acetonitrile |
| 1H = | proton |
| AcOH = | Acetic acid |
| Aq = | aqueous |
| BINAP = | ([1,1′-Binaphthalene]-2,2′-diyl)bis(diphenylphosphane) |
| Bn = | Benzyl |
| Boc = | tert-butoxycarbonyl |
| Boc2O = | BOC anhydride |
| BOP = | benzotriazol-1-yloxytris (dimethylamino)phosphonium hexafluorophosphate |
| C (or ° C.) = | Degrees Celsius |
| Cbz = | Benzyloxycarbonyl |
| DAST = | diethyl(trifluorosulfido)amine |
| dba = | Dibenzylideneacetone |
| DBU = | 1,8-diazabicyclo[5.4.0]undec-7-ene |
| DCM = | Dichloromethane |
| DIPEA, DIEA = | Disopropylethylamine |
| DME = | 1,2-Dimethoxyethane |
| DMEDA = | N,N′-Dimethylethane-1,2-diamine |
| DMF = | N,N-Dimethylmethanamide |
| DMSO = | Dimethyl sulfoxide |
| DPPA = | Diphenoxyphosphoryl azide |
| dppf = | 1,1′-(bis-diphenylphosphino) ferrocene |
| Eq = | Equivalents |
| EtOAc = | Ethyl acetate |
| EtOH = | Ethanol |
| g = | grams |
| H, hr = | hours |
| HATU = | 1-[Bis(dimethylamino)methylene]-1H-1,2,3-triazolo[4,5-b]pyridinium 3-oxide hexafluorophosphate |
| HOBt = | 1-hydroxybenzatriazole |
| HPLC = | High pressure liquid chromatography |
| LAH = | Lithium aluminum hydride |
| LDA = | Lithium diisopropylamide |
| LiHMDS = | lithium hexamethyldisilazide |
| M = | Molar |
| mCPBA = | meta-Chloroperoxy benzoic acid |
| Me = | Methyl |
| MeCN = | Acetonitrile |
| Mel = | Methyl iodide |
| MeOH = | Methanol |
| mg = | Milligrams |
| MHz = | Megahertz |
| min = | Minutes |
| mL (or ml) = | Milliliter |
| mmol = | millimolar |
| MS = | Mass spectroscopy |
| MsCl = | Methanesulfonyl chloride |
| MW = | microwave |
| N = | Normal |
| NBS = | N-bromosuccinimide |
| NCS = | N-Chlorosuccinimide |
| NMP = | 1-methyl-2-pyrrolidone |
| NMR = | Nuclear Magnetic Resonance |
| pin = | pinacol |
| PTLC = | Preparative thin layer chromatography |
| RT or rt = | Room temperature |
| Sat, sat. or sat′d = | saturated |
| SEM = | 2-(Trimethylsilyl)ethoxy-methyl |
| SEMCl = | 2-Chloromethoxyethyl) trimethylsilane |
| SFC = | Supercritical fluid chromatography |
| SiO2 = | Silica gel |
| TBAF = | Tetrabutylammonium fluoride |
| tBoc = | tert-butoxycarbonyl |
| TBS = | tert-butyldimethyl silyl |
| t-Bu = | tert-butyl |
| TEA = | Triethyl amine |
| TFA = | Trifluoroacetic acid |
| THF = | tetrahydrofuran |
| TLC = | Thin layer chromatography |
| Tol. = | toluene |
| TsCl = | para-Toluenesulfonyl chloride |
| TsOH = | para-toluene sulfonic acid |
| Xantphos = | (9,9-Dimethyl-9H-xanthene-4,5-diyl)bis(diphenylphosphane) |
| Zhan catalyst = | Dichloro(1,3-bis(2,4,6-trimethylphenyl)-2-imidazolidinylidene)((5- |
| | ((dimethylamino)sulfonyl)-2-(1-methylethoxy-O)phenyl)methylene-C)ruthenium(II) |

### Syntheses

### Preparation of (1-cyclopropyl-6-fluoro-indazol-7-yl)methanamine

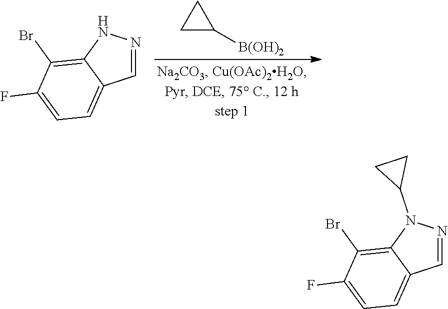 [SMILES: Fc1ccc2cn[nH]c2c1Br ; OB(O)C1CC1 ; Fc1ccc2cnn(C3CC3)c2c1Br]

A mixture of 7-bromo-6-fluoro-1H-indazole (0.800 g, 3.72 mmol, 1 eq), cyclopropylboronic acid (384 mg, 4.46 mmol, 1.2 eq), Na₂CO₃ (789 mg, 7.44 mmol, 2 eq), Cu(OAc)₂·H₂O (743 mg, 3.72 mmol, 1 eq) and pyridine (294 mg, 3.72 mmol, 1 eq) in DCE (15 mL) was degassed and purged with N₂ (3×). The mixture was stirred at 75° C. for 12 h under a N₂ atmosphere. The mixture was diluted with H₂O (10 mL), and the mixture was extracted with ethyl acetate (15 mL×3). The combined organic layers were washed with brine (20 mL), dried over Na₂SO₄, and filtered. The filtrate was concentrated under reduced pressure to give a residue. The residue was purified by column chromatography (SiO₂, petroleum ether/ethyl acetate=1/0 to 3/1) to furnish 7-bromo-1-cyclopropyl-6-fluoro-indazole. ¹H NMR: (400 MHz, DMSO-d₆) δ 8.08 (s, 1H), 7.83-7.77 (m, 1H), 7.18 (br t, J=8.9 Hz, 1H), 4.17-3.95 (m, 1H), 1.28 (br s, 2H), 1.17 (br d, J=6.4 Hz, 2H).

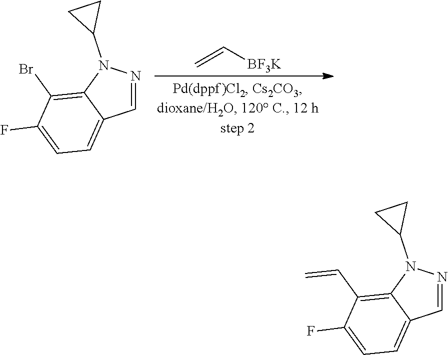

A mixture of 7-bromo-1-cyclopropyl-6-fluoro-indazole (0.200 g, 0.784 mmol, 1 eq), potassium vinyltrifluoroboronate (126 mg, 0.941 mmol, 1.2 eq), Pd(dppf)Cl₂ (57 mg, 0.078 mmol, 0.1 eq), Cs₂CO₃ (511 mg, 1.57 mmol, 2 eq) in dioxane (2 mL) and H₂O (0.2 mL) was degassed and purged with N₂ (3×). The mixture was stirred at 120° C. for 12 h under a N₂ atmosphere. The mixture was diluted with H₂O (10 mL) and extracted with ethyl acetate (15 mL×3). The combined organic layers were washed with brine (20 mL), dried over Na₂SO₄, and filtered. The filtrate was concentrated under reduced pressure to give a residue. The residue was purified by column chromatography (SiO₂, petroleum ether/ethyl acetate=1/0 to 4/1) to furnish 1-cyclopropyl-6-fluoro-7-vinyl-indazole.

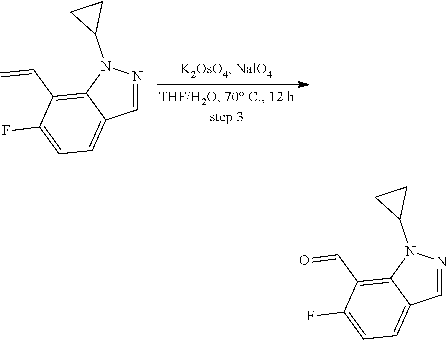

A mixture of 1-cyclopropyl-6-fluoro-7-vinyl-indazole (0.100 g, 0.494 mmol, 1 eq), sodium periodate (529 mg, 2.47 mmol, 5 eq), potassium osmate dihydrate (15 mg, 0.040 mmol, 0.08 eq) in THE (2 mL) and H₂O (0.4 mL) was degassed and purged with N₂ (3×). The mixture was stirred at 70° C. for 12 h under N₂ atmosphere. The mixture was diluted with H₂O (10 mL), and the mixture was extracted with EtOAc (15 mL×3). The combined organic layers were washed with brine (20 mL), dried over Na₂SO₄, and filtered. The filtrate was concentrated under reduced pressure to give a residue. The residue was purified by column chromatography (SiO₂, petroleum ether/ethyl acetate=1/0 to 3/1) to furnish 1-cyclopropyl-6-fluoro-indazole-7-carbaldehyde. 1H NMR: (400 MHz, DMSO-d₆) δ 10.83 (s, 1H), 8.19 (s, 1H), 8.12 (dd, J=4.9, 8.7 Hz, 1H), 7.17 (dd, J=8.7, 11.5 Hz, 1H), 4.15-4.07 (m, 1H), 1.17 (t, J=7.1 Hz, 4H).

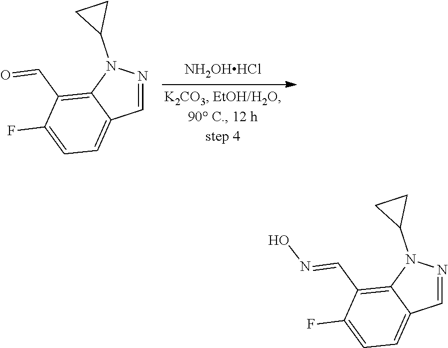

A mixture of 1-cyclopropyl-6-fluoro-indazole-7-carbaldehyde (40 mg, 0.020 mmol, 1 eq), hydroxylamine hydrochloride (44 mg, 0.63 mmol, 3.2 eq), K₂CO₃ (41 mg, 0.29 mmol, 1.5 eq) in EtOH (1 mL) and H₂O (1 mL) was degassed and purged with N₂ (3×). The mixture was stirred at 90° C. for 12 h under a N₂ atmosphere. The reaction mixture was concentrated under reduced pressure to remove EtOH. The residue was diluted with H₂O (5 mL), and the mixture was extracted with ethyl acetate (5 mL×3). The combined organic layers were washed with brine (5 mL×3), dried over Na₂SO₄, and filtered. The filtrate was concentrated under reduced pressure to furnish (7E)-1-cyclopropyl-6-fluoro-indazole-7-carbaldehyde oxime. LCMS: (M+H⁺):220.2.

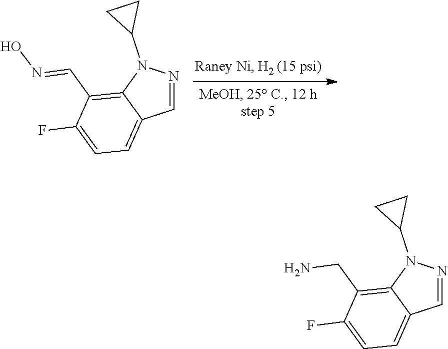 [SMILES: O/N=C/c1c(F)ccc2cnn(C3CC3)c12 ; NCc1c(F)ccc2cnn(C3CC3)c12]

To a solution of (7E)-1-cyclopropyl-6-fluoro-indazole-7-carbaldehyde oxime (23 mg, 0.11 mmol, 1 eq) in MeOH (1 mL) was added Raney-Ni (23 mg). The suspension was degassed and purged with H₂ (3×). The mixture was stirred under H₂ (15 Psi) at 25° C. for 12 h. The reaction mixture was filtered and concentrated under reduced pressure to furnish (1-cyclopropyl-6-fluoro-indazol-7-yl)methanamine.

### Preparation of (6-fluoro-1-methyl-1H-indazol-7-yl)methanamine

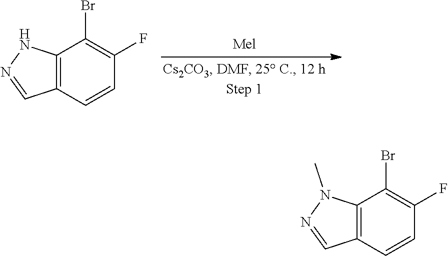

To a solution of 7-bromo-6-fluoro-1H-indazole (0.450 g, 2.09 mmol, 1 eq) in DMF (10 mL) was added Cs₂CO₃ (1.36 g, 4.19 mmol, 2 eq) and Mel (327 mg, 2.30 mmol, 1.1 eq) at 25° C. The mixture was stirred at 25° C. for 12 h. The reaction mixture was quenched by addition water (10 mL), and the mixture was extracted with ethyl acetate (10 mL×3). The combined organic layers were washed with brine (5 mL×3), dried over anhydrous sodium sulfate, and filtered. The filtrate was concentrated under reduced pressure to give a residue. The residue was purified by column chromatography (SiO₂, petroleum ether/ethyl acetate=100/0 to 30/1) to furnish 7-bromo-6-fluoro-1-methyl-1H-indazole. LCMS: (M+H⁺): 229.0.

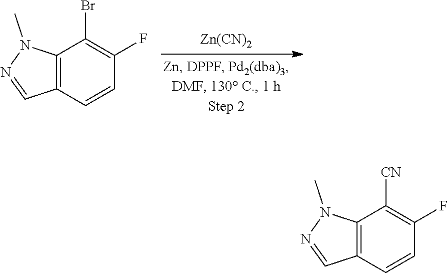

A mixture of 7-bromo-6-fluoro-1-methyl-1H-indazole (650 mg, 2.84 mmol, 1 eq), dicyanozinc (667 mg, 5.68 mmol, 2 eq), Pd₂(dba)₃ (260 mg, 0.284 mmol, 0.1 eq), dppf (315 mg, 0.568 mmol, 0.2 eq) and Zn (371 mg, 5.68 mmol, 2 eq) in DMF (10 mL) was degassed and purged with N₂ (3×). The mixture was stirred at 130° C. for 1 h under microwave irradiation. The reaction mixture was poured into H₂O (30 mL). The mixture was extracted with EtOAc (10 mL×3). The combined organic layers were washed with brine (50 mL), dried over Na₂SO₄, and filtered. The filtrate was concentrated under reduced pressure. The residue was purified by flash silica gel chromatography (ISCO®; 12 g SepaFlash® Silica Flash Column, eluent of 6~10% ethyl acetate/petroleum ether gradient at 50 mL/min) to furnish 6-fluoro-1-methyl-1H-indazole-7-carbonitrile.

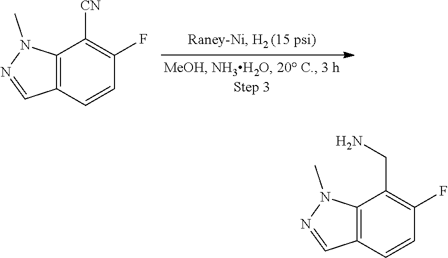

To a solution of 6-fluoro-1-methyl-1H-indazole-7-carbonitrile (200 mg, 1.14 mmol, 1 eq) and NH₃·H₂O (1 mL) in MeOH (2 mL) was added Raney-Ni (200 mg) under a N₂ atmosphere. The suspension was degassed and purged with H₂ (3×). The mixture was stirred under H₂ (15 Psi) at 20° C. for 3 h. The mixture was filtered, and the filtrate was concentrated to furnish (6-fluoro-1-methyl-1H-indazol-7-yl)methanamine. LCMS: (M-NH₂+): 163.2.

### Preparation of (1-cyclopropylindazol-7-yl)methanamine

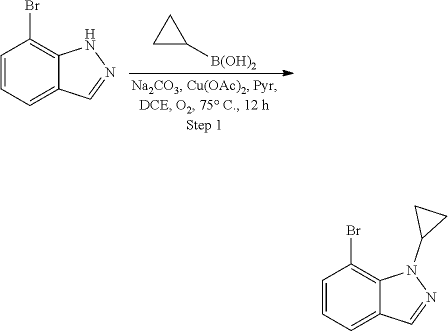 [SMILES: Brc1cccc2cn[nH]c12 ; OB(O)C1CC1 ; Brc1cccc2cnn(C3CC3)c12]

A mixture of 7-bromo-1H-indazole (1.00 g, 5.08 mmol, 1 eq), cyclopropylboronic acid (523 mg, 6.09 mmol, 1.2 eq), Na₂CO₃ (1.08 g, 10.2 mmol, 2 eq), Cu(OAc)₂·H₂O (1.01 g, 5.08 mmol, 1.01 mL, 1 eq) and pyridine (401 mg, 5.08 mmol, 1 eq) in DCE (100 mL) was degassed and purged with O₂ for (3×). The mixture was stirred at 75° C. for 12 h under O₂ (15 Psi) atmosphere. The reaction mixture was filtered and concentrated under reduced pressure. The residue was purified by flash silica gel chromatography (ISCO®; 12 g SepaFlash® Silica Flash Column, gradient elution of 2% ethyl acetate/petroleum ether at 50 mL/min) to furnish 7-bromo-1-cyclopropyl-indazole. ¹H NMR: (400 MHz, DMSO-d6) δ 8.08 (s, 1H), 7.77 (d, J=8.0 Hz, 1H), 7.64 (d, J=7.4 Hz, 1H), 7.06 (t, J=7.7 Hz, 1H), 4.09 (tt, J=3.7, 7.1 Hz, 1H), 1.31-1.24 (m, 2H), 1.20-1.13 (m, 2H).

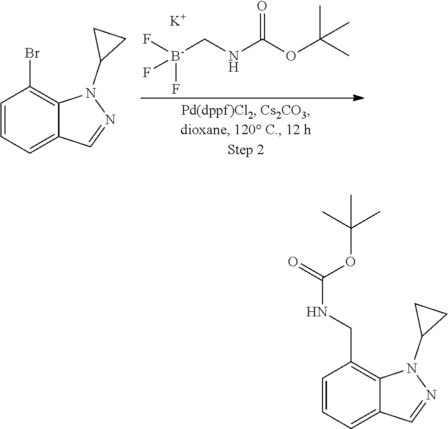 [SMILES: Brc1cccc2cnn(C3CC3)c12 ; CC(C)(C)OC(=O)NC[B-](F)(F)F ; [K+] ; CC(C)(C)OC(=O)NCc1cccc2cnn(C3CC3)c12]

A mixture of 7-bromo-1-cyclopropyl-indazole (520 mg, 2.19 mmol, 1 eq), potassium (tert-butoxycarbonylamino)methyl-trifluoro-boranide (624 mg, 2.63 mmol, 1.2 eq), Pd(dppf)Cl₂ (160 mg, 0.219 mmol, 0.1 eq), Cs₂CO₃ (1.43 g, 4.39 mmol, 2 eq) in dioxane (26 mL) and H₂O (1.7 mL) was degassed and purged with N₂ (3×). The mixture was stirred at 120° C. for 12 h under a N₂ atmosphere. The reaction mixture was concentrated under reduced pressure to remove solvent. The residue was diluted with H₂O (50 mL), and the mixture was extracted with EtOAc (20 mL×3). The combined organic layers were washed with brine (50 mL), dried over Na₂SO₄, and filtered. The filtrate was concentrated under reduced pressure. The residue was purified by flash silica gel chromatography (ISCO®; 20 g SepaFlash® Silica Flash Column, elution of 2% ethyl acetate/petroleum ether at 40 mL/min) to furnish tert-butyl N-[(1-cyclopropylindazol-7-yl)methyl]carbamate. LCMS: (M+H⁺).288.2; ¹H NMR: (400 MHz, DMSO-d6) δ 7.96 (s, 1H), 7.61 (d, J=7.9 Hz, 1H), 7.48 (br s, 1H), 7.23 (br d, J=6.8 Hz, 1H), 7.10 (t, J=7.5 Hz, 1H), 4.88 (br d, J=5.6 Hz, 2H), 4.06-3.97 (m, 1H), 1.44-1.35 (m, 9H), 1.27-1.22 (m, 2H), 1.16-1.10 (m, 2H).

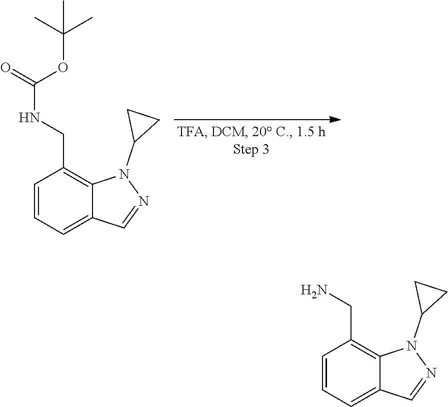 [SMILES: CC(C)(C)OC(=O)NCc1cccc2cnn(C3CC3)c12 ; NCc1cccc2cnn(C3CC3)c12]

A mixture of tert-butyl N-[(1-cyclopropylindazol-7-yl)methyl]carbamate (460 mg, 1.60 mmol, 1 eq) in DCM (9 mL) and TFA (3 mL) was degassed and purged with N₂ (3×). The mixture was stirred at 20° C. for 1.5 hr under a N₂ atmosphere. The reaction mixture was concentrated under reduced pressure to remove solvent to furnish (1-cyclopropylindazol-7-yl)methanamine. LCMS: (M-NH₂⁺):171.2.

### Preparation of (1-cyclopropyl-6-fluoro-1H-benzo[d]imidazol-7-yl)methanamine

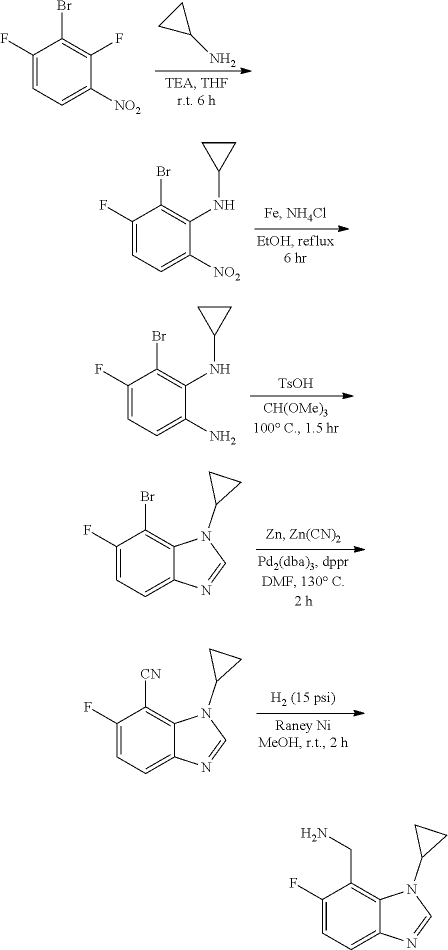

(1-Cyclopropyl-6-fluoro-1H-benzo[d]imidazol-7-yl)methanamine was prepared as depicted above using conditions previously outlined for related amines.

### Preparation of (1s,3s)-N¹-(5-fluoropyrimidin-2-yl)cyclobutane-1,3-diamine

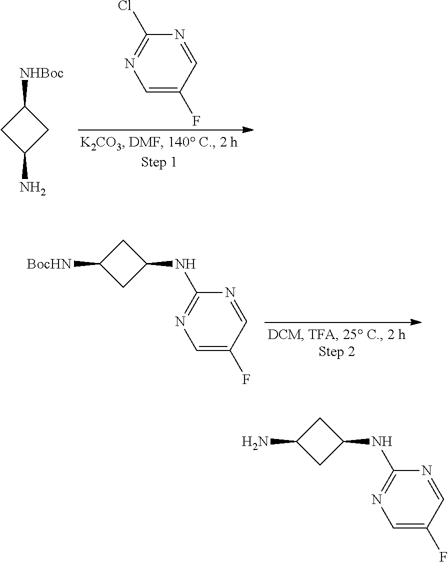 [SMILES: CC(C)(C)OC(=O)N[C@H]1C[C@@H](N)C1 ; Fc1cnc(Cl)nc1 ; CC(C)(C)OC(=O)N[C@H]1C[C@@H](Nc2ncc(F)cn2)C1 ; N[C@H]1C[C@@H](Nc2ncc(F)cn2)C1]

(1s,3s)-N¹-(5-Fluoropyrimidin-2-yl)cyclobutane-1,3-diamine was prepared using conditions like those outlined for other amine intermediates.

### Preparation of (1s,3s)-N¹-(5-fluoropyrimidin-2-yl)-N-methylcyclobutane-1,3-diamine

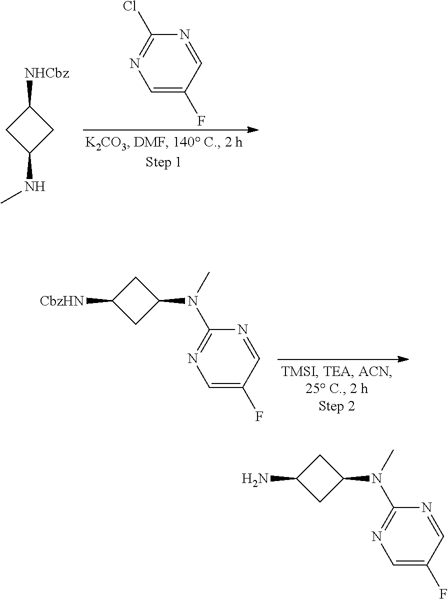 [SMILES: CN[C@H]1C[C@@H](NC(=O)OCc2ccccc2)C1 ; Fc1cnc(Cl)nc1 ; CN(c1ncc(F)cn1)[C@H]1C[C@@H](NC(=O)OCc2ccccc2)C1 ; CN(c1ncc(F)cn1)[C@H]1C[C@@H](N)C1]

(1s,3s)-N¹-(5-fluoropyrimidin-2-yl)-N-methylcyclobutane-1,3-diamine was prepared using conditions like those outlined for other amine intermediates.

### Preparation of racemic-1-(5-fluoropyrimidin-2-yl)-3-(methoxymethyl)pyrrolidin-3-amine

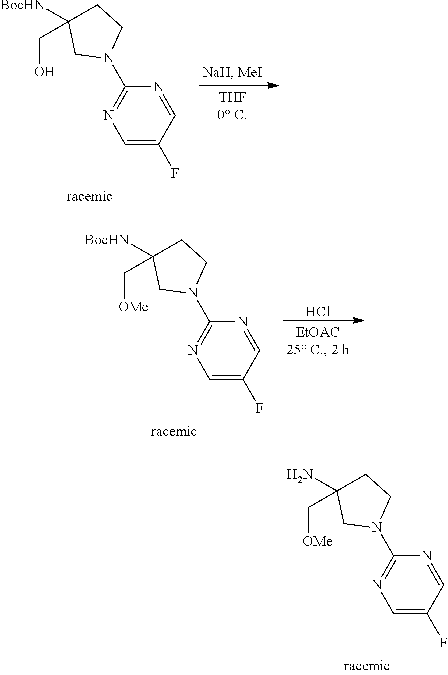 [SMILES: CC(C)(C)OC(=O)NC1(CO)CCN(c2ncc(F)cn2)C1 ; COCC1(NC(=O)OC(C)(C)C)CCN(c2ncc(F)cn2)C1 ; Cl ; COCC1(N)CCN(c2ncc(F)cn2)C1]

The preparation of racemic-1-(5-fluoropyrimidin-2-yl)-3-(methoxymethyl)pyrrolidin-3-amine from tert-butyl (1-(5-fluoropyrimidin-2-yl)-3-(hydroxymethyl)pyrrolidin-3-yl)carbamate is depicted in the above scheme.

### Preparation of (R)-1-(5-fluoropyrimidin-2-yl)pyrrolidin-3-amine

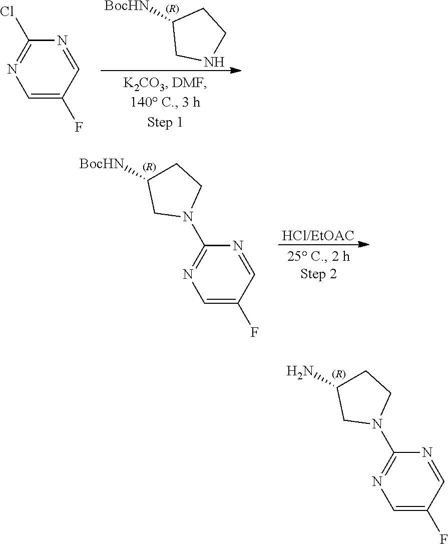

A mixture of 2-chloro-5-fluoro-pyrimidine (1.42 g, 10.7 mmol, 1.33 mL, 1 eq), tert-butyl N-[(3R)-pyrrolidin-3-yl]carbamate (2.00 g, 10.7 mmol, 1 eq), K₂CO₃ (2.97 g, 21.5 mmol, 2 eq) in DMF (10 mL) was degassed and purged with N₂ (3×). The mixture was stirred at 140° C. for 3 h under a N₂ atmosphere. The reaction was poured into water (30 mL), and the mixture was extracted with ethyl acetate (20 mL×3). The combined organic phase was washed with brine (30 mL), dried with anhydrous Na₂SO₄, and filtered. The filtrate was concentrated under vacuum to furnish tert-butyl N-[(3R)-1-(5-fluoropyrimidin-2-yl)pyrrolidin-3-yl]carbamate as a yellow solid. The material was used into the next step without further purification.

A mixture of tert-butyl N-[(3R)-1-(5-fluoropyrimidin-2-yl)pyrrolidin-3-yl]carbamate (2.90 g, 10.3 mmol, 1 eq) in HCl/EtOAc (20 mL) was degassed and purged with N₂ (3×). The mixture was stirred at 25° C. for 2 h under a N₂ atmosphere. The reaction mixture was concentrated under reduced pressure to furnish (R)-1-(5-fluoropyrimidin-2-yl)pyrrolidin-3-amine.

The following primary amines can be prepared in a similar fashion to that shown above for the preparation of (R)-1-(5-fluoropyrimidin-2-yl)pyrrolidin-3-amine using the appropriate reagents and conditions.

| Protected | Het/Ar halide | Primary |
|---|---|---|
| Amine | Conditions | Amine |
| 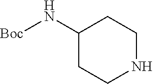 [SMILES: CC(C)(C)OC(=O)NC1CCNCC1] | 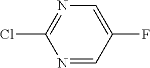 K₂CO₃, DMF, 140° C., 3 h | 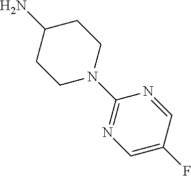 [SMILES: NC1CCN(c2ncc(F)cn2)CC1] |
| 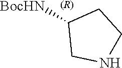 | 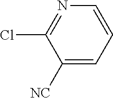 K₂CO₃, DMF, 140° C., 3 h | 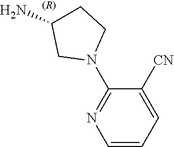 |
| 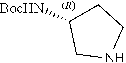 | 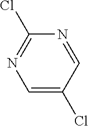 K₂CO₃, DMF, 40° C., 12 h | 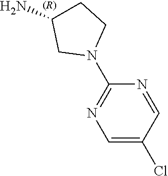 |
| 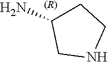 | 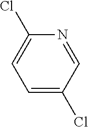 K₂CO₃, DMF, 90° C., 12 h | 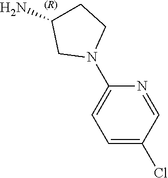 |
| 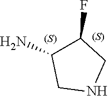 | 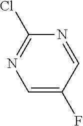 TEA, DMA, 130° C., 3 h | 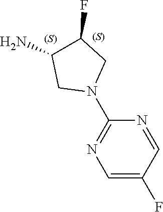 |
| 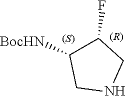 | 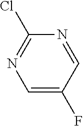 K₂CO₃, DMF, 140° C., 3 h | 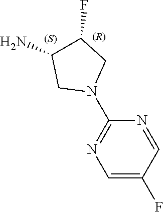 |
| 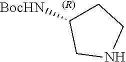 | 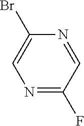 Pd₂(dba)₃, BINAP, Cs₂CO₃, | 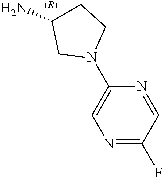 |
| | dioxane, | |
| | 100° C., 4 h | |
| 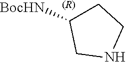 | 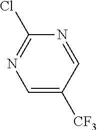 [SMILES: FC(F)(F)c1cnc(Cl)nc1] K₂CO₃, DMF, 120° C., 12 h | 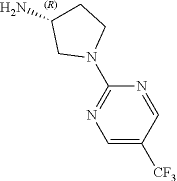 |
| 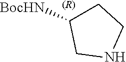 | 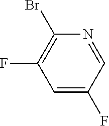 Pd₂(dba)₃, BINAP, Cs₂CO₃, | 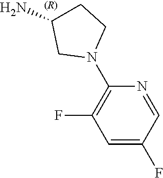 |
| | dioxane, | |
| | 110° C., 12 h | |
| 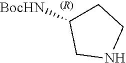 | 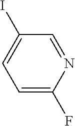 Pd₂(dba)₃, Xantphos, DIEA, dioxane, | 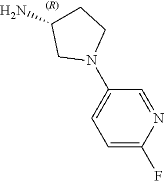 |
| | 100° C., 12 h | |
| 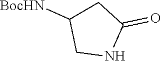 [SMILES: CC(C)(C)OC(=O)NC1CNC(=O)C1] racemic | 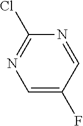 Pd₂(dba)₃, Xantphos, Cs₂CO₃, | 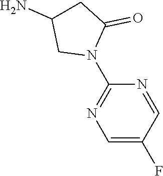 [SMILES: NC1CC(=O)N(c2ncc(F)cn2)C1] |
| | dioxane, 100° C., | racemic |
| | 12 h | |
| 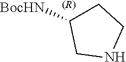 | 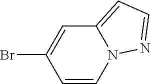 Pd₂(dba)₃, BINAP, Cs₂CO₃, dioxane, 100° C., 12 h | 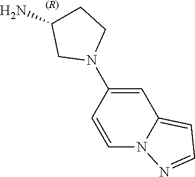 |
| 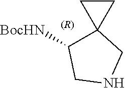 | 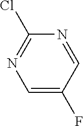 K₂CO₃, DMF, 140° C., 2 h | 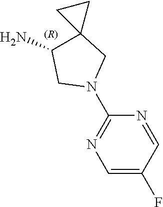 |
| 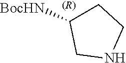 | 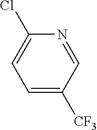 [SMILES: FC(F)(F)c1ccc(Cl)nc1] K₂CO₃, DMF, 140° C., 2 h | 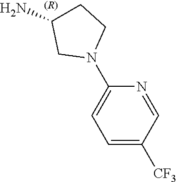 |
| 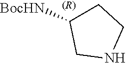 | 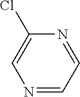 K₂CO₃, DMF, 140° C., 2 h | 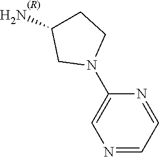 |
| 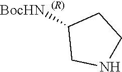 | 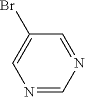 Pd₂(dba)₃, BINAP, Cs₂CO₃, | 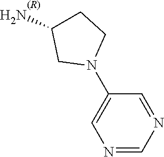 |
| | dioxane, 110° C., | |
| | 12 h | |
| 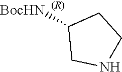 | 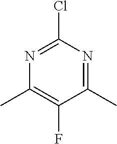 K₂CO₃, DMF, 140° C., 2 h | 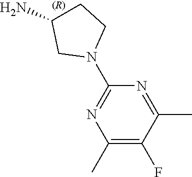 |
| 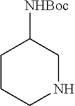 [SMILES: CC(C)(C)OC(=O)NC1CCCNC1] (racemic) | 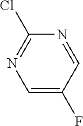 K₂CO₃, DMF, 140° C., 3 h | 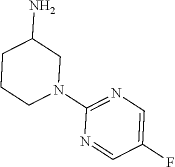 [SMILES: NC1CCCN(c2ncc(F)cn2)C1] |
| | | (racemic) |
| 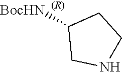 | 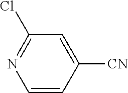 K₂CO₃, DMF, 140° C., 2 h | 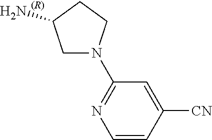 |
| 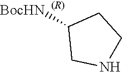 | 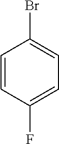 Pd₂(dba)₃, BINAP, | 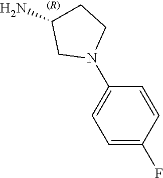 |
| | Cs₂CO₃, | |
| | dioxane, 110° C., | |
| | 12 h | |
| 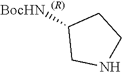 | 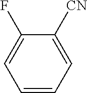 K₂CO₃, DMF, 140° C., 3 h | 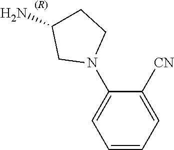 |
| 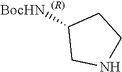 | 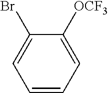 [SMILES: FC(F)(F)Oc1ccccc1Br] Pd₂(dba)₃, BINAP, Cs₂CO₃, | 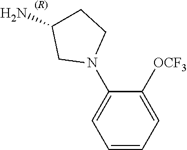 |
| | dioxane, 110° C., | |
| | 4 h | |
| 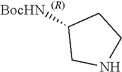 | 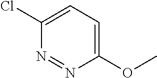 TEA, DMF, 130° C., 12 h | 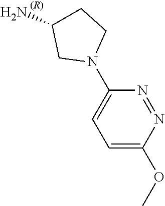 |
| 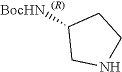 | 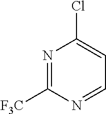 [SMILES: FC(F)(F)c1nccc(Cl)n1] K₂CO₃, DMF, 140° C., 2 h | 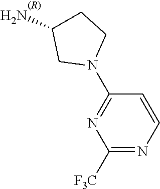 |
| 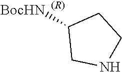 | 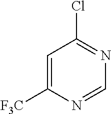 [SMILES: FC(F)(F)c1cc(Cl)ncn1] K₂CO₃, DMF, 140° C., 1 h | 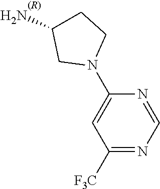 |
| 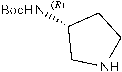 | 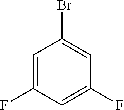 Pd(OAc)₂, BINAP, Cs₂CO₃, toluene, 90° C., | 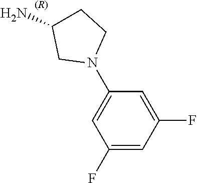 |
| | 4 h | |
| 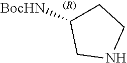 | 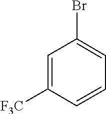 [SMILES: FC(F)(F)c1cccc(Br)c1] Pd₂(dba)₃, BINAP, Cs₂CO₃, | 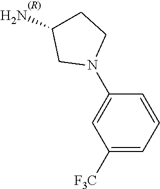 |
| | dioxane, 110° C., | |
| | 12 h | |
| 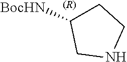 | 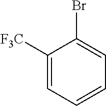 [SMILES: FC(F)(F)c1ccccc1Br] Pd₂(dba)₃, BINAP, | 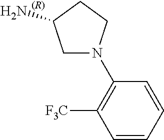 |
| | Cs₂CO₃, | |
| | dioxane, 110° C., | |
| | 4 h | |
| 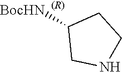 | 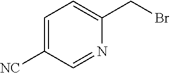 K₂CO₃, DMF, 140° C., 2 h | 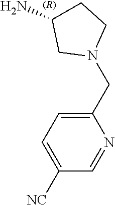 |
| 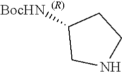 | 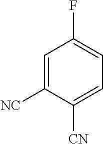 [SMILES: N#Cc1ccc(F)cc1C#N] K₂CO₃, DMF, 80° C., 3 h | 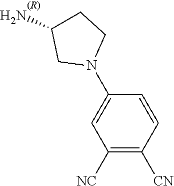 |
| 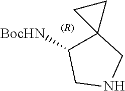 | 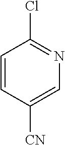 K₂CO₃, DMF, 140° C., 10 h | 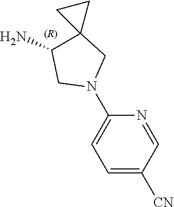 |
| 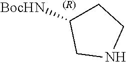 | 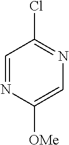 Pd₂(dba)₃, BINAP, | 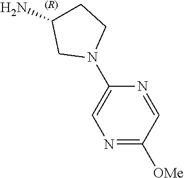 |
| | Cs₂CO₃, | |
| | dioxane, 110° C., | |
| | 12h | |
| 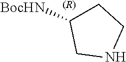 | 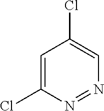 TEA, THF, 20° C., 12 h | 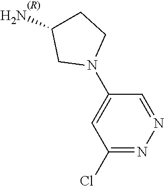 |
| 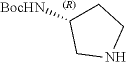 | 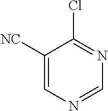 TEA, DMF, 80° C., 2 h | 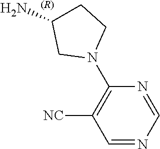 |
| 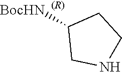 | 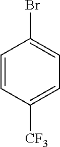 [SMILES: FC(F)(F)c1ccc(Br)cc1] Pd₂(dba)₃, BINAP, | 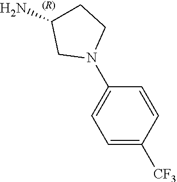 |
| | K₂CO₃, DMF, | |
| | 140° C., 3 h | |
| 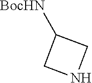 [SMILES: CC(C)(C)OC(=O)NC1CNC1] | 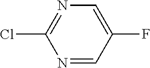 K₂CO₃, DMF, 140° C., 3 h | 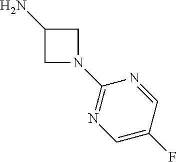 [SMILES: NC1CN(c2ncc(F)cn2)C1] |
| 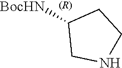 | 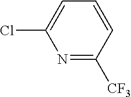 [SMILES: FC(F)(F)c1cccc(Cl)n1] K₂CO₃, DMF, 140° C., 3 h | 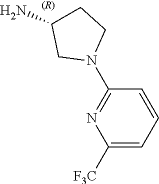 |
| 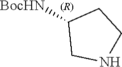 | 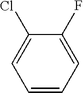 K₂CO₃, DMF, 140° C., 3 h | 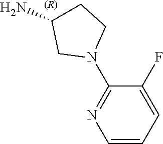 |
| 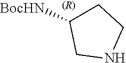 | 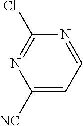 TEA, DMF, 130° C., 3 h Boc | 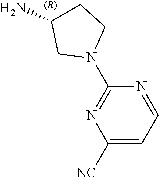 |
| | deprotection | |
| | (TMSOTf, | |
| | 2,6-lutidine, | |
| | DCM) | |
| 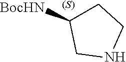 | 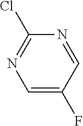 K₂CO₃, DMF, 140° C., 3 h | 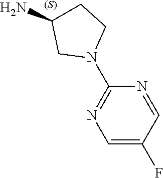 |
| 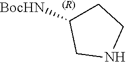 | 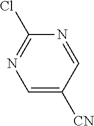 K₂CO₃, DMF, 140° C., 3 h | 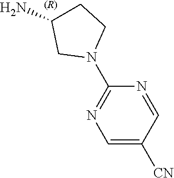 |
| | Boc | |
| | deprotection | |
| | (TMSOTf, | |
| | 2,6-lutidine, | |
| | DCM) | |
| 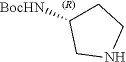 | 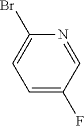 Pd₂(dba)₃, Xantphos, Cs₂CO₃, | 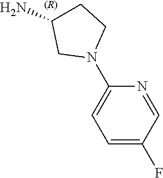 |
| | dioxane, 100° C., | |
| | 16 h | |
| 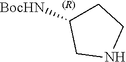 | 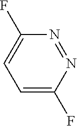 n-PrOH, DIPEA, 130° C., 12 h | 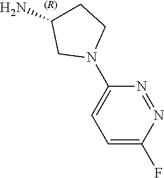 |
| 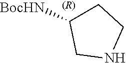 | 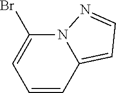 Pd₂(dba)₃, BINAP, Cs₂CO₃, | 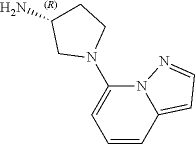 |
| | dioxane, | |
| | 100° C., 12 h | |
| 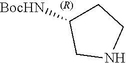 | 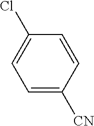 K₂CO₃, DMF, 140° C., 12 h | 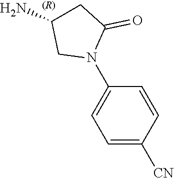 |
| 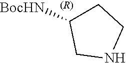 | 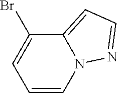 Pd₂(dba)₃, BINAP, Cs₂CO₃, | 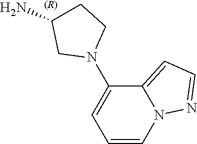 |
| | dioxane, 100° C., | |
| | 12 h | |
| 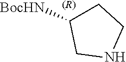 | 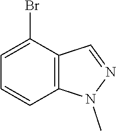 Pd₂(dba)₃, BINAP, Cs₂CO₃, dioxane, 100° C., 12 h | 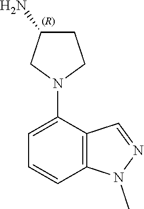 |
| 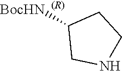 | 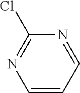 K₂CO₃, DMF, 140° C., 2 h | 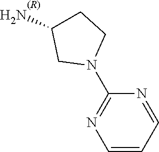 |
| 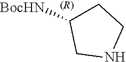 | 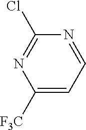 [SMILES: FC(F)(F)c1ccnc(Cl)n1] K₂CO₃, DMF, 140° C., 2 h | 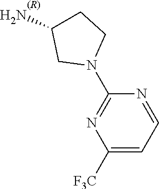 |
| 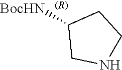 | 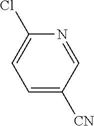 K₂CO₃, DMF, 140° C., 1 h | 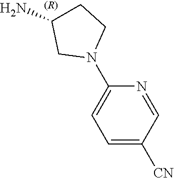 |
| 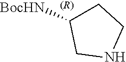 | 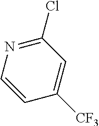 [SMILES: FC(F)(F)c1ccnc(Cl)c1] K₂CO₃, DMF, 140° C., 1 h | 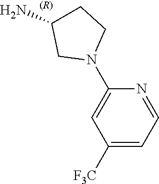 |
| 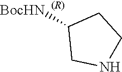 | 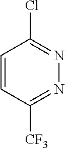 [SMILES: FC(F)(F)c1ccc(Cl)nn1] TEA, DMF, 80° C., 2 h | 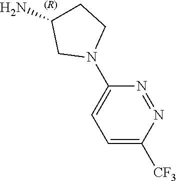 |
| 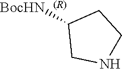 | 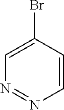 TEA, DMF, 100° C., 12 h | 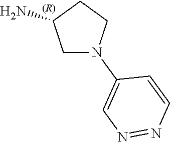 |
| 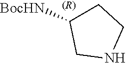 | 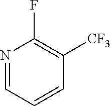 [SMILES: Fc1ncccc1C(F)(F)F] K₂CO₃, DMF, 140° C., | 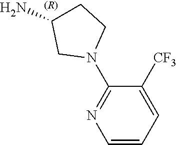 |
| | 2 h | |
| 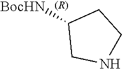 | 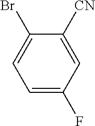 Pd₂(dba)₃, BINAP, Cs₂CO₃, | 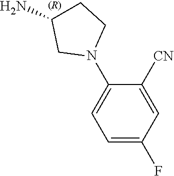 |
| | dioxane, 110° C., | |
| | 12 h | |
| 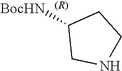 | 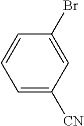 Pd₂(dba)₃, BINAP, Cs₂CO₃, | 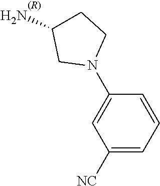 |
| | dioxane, 110° C., | |
| | 12 h | |
| 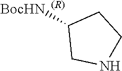 | 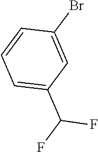 Pd₂(dba)₃, BINAP, Cs₂CO₃, | 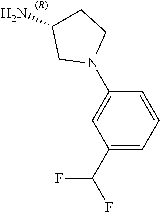 |
| | dioxane, 110° C., | |
| | 12 h | |
| 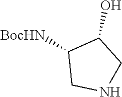 [SMILES: CC(C)(C)OC(=O)N[C@H]1CNC[C@H]1O] | 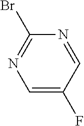 K₂CO₃, DMF, 140° C., 0.5 h | 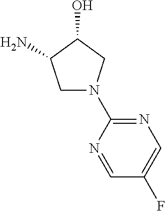 [SMILES: N[C@H]1CN(c2ncc(F)cn2)C[C@H]1O] |
| 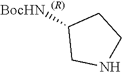 | 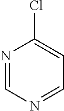 K₂CO3, DMF, 140° C., | 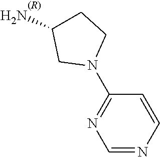 |
| | 1 h | |
| 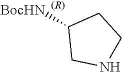 | 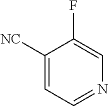 K₂CO₃, DMF, 100° C., | 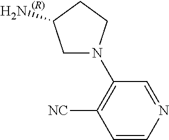 |
| | 6 h | |
| 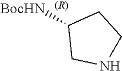 | 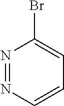 Pd₂(dba)₃, BINAP, Cs₂CO₃, | 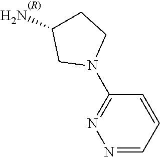 |
| | dioxane, 110° C., | |
| | 12 h | |
| 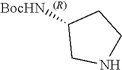 | 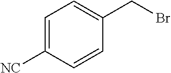 K₂CO₃, DMF, 140° C., 1 h | 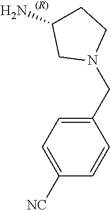 |
| 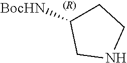 | 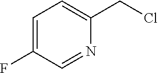 K₂CO₃, DMF, 140° C., 1 h | 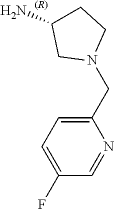 |
| 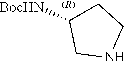 | 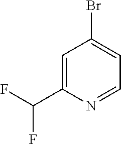 Pd₂(dba)₃, BINAP, Cs₂CO₃, dioxane, 110° C., | 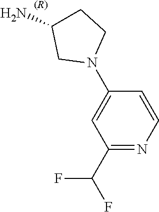 |
| | 12 h | |
| 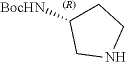 | 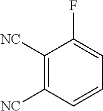 [SMILES: N#Cc1cccc(F)c1C#N] K₂CO₃, DMF, 80° C., 12 h | 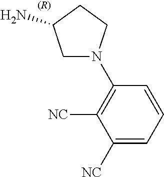 |
| 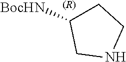 | 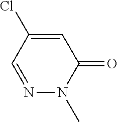 Pd₂(dba)₃, BINAP, K₂CO₃, | 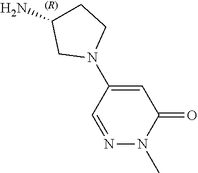 |
| | DMF, 80° C., | |
| | 2 h | |
| 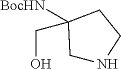 [SMILES: CC(C)(C)OC(=O)NC1(CO)CCNC1] (racemic) | 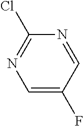 K₂CO₃, DMF, 60° C., 1 h | 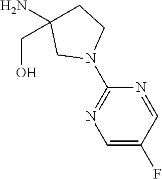 [SMILES: NC1(CO)CCN(c2ncc(F)cn2)C1] |
| | | (racemic) |
| 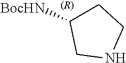 | 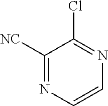 TEA, DMF, 80° C., 2 h | 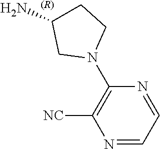 |
| 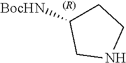 | 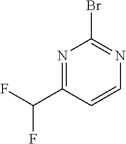 TEA, DMF, 80° C., 3 h | 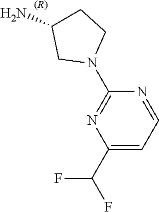 |
| 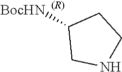 | 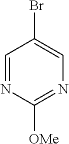 Pd₂(dba)₃, Xantphos, | 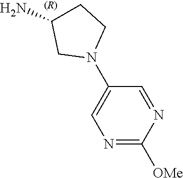 |
| | Cs₂CO₃, | |
| | dioxane, 100° C., | |
| | 12 h | |

### Preparation of (4-fluoro-2-methoxypyridin-3-yl)methanamine and 3-(aminomethyl)-4-fluoropyridin-2(11H)-one

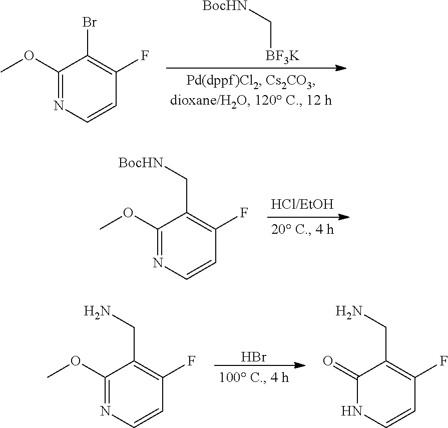

A mixture of 3-bromo-4-fluoro-2-methoxy-pyridine (1.20 g, 5.82 mmol, 1 eq), potassium;(tert-butoxycarbonylamino)methyl-trifluoro-boranuide (1.66 g, 6.99 mmol, 1.2 eq), Cs₂CO₃ (3.80 g, 11.7 mmol, 2 eq), Pd(dppf)Cl₂ (426 mg, 582 mol, 0.1 eq) in dioxane (12 mL) and H₂O (3 mL) was degassed and purged with N₂ (3×). The mixture was stirred at 120° C. for 12 h under a N₂ atmosphere. Water was added (10 ml), and the mixture was extracted with EtOAc (15 mL×3). The combined organic layers were washed with brine (20 mL), dried over Na₂SO₄, and filtered. The filtrate was concentrated under reduced pressure to give a residue. The residue was purified by column chromatography (SiO₂, petroleum ether/ethyl acetate=1/0 to 3/1) to give tert-butyl ((4-fluoro-2-methoxypyridin-3-yl)methyl)carbamate.

A mixture of tert-butyl N-[(4-fluoro-2-methoxy-3-pyridyl)methyl]carbamate (0.140 g, 546 mol, 1 eq) in HCl/EtOAc (1 mL) was degassed and purged with N₂ (3×). The mixture was stirred at 20° C. for 4 h under a N₂ atmosphere. The mixture was concentrated under reduced pressure to furnish (4-fluoro-2-methoxypyridin-3-yl)methanamine.

A mixture of (4-fluoro-2-methoxy-3-pyridyl)methanamine (70 mg, 0.49 mmol, 1 eq) in HBr (0.5 mL) was degassed and purged with N₂ (3×). The mixture was stirred at 100° C. for 4 h under a N₂ atmosphere. Water was added to the reaction (10 mL), and the extracted with EtOAc (15 mL×3). The combined organic layers were washed with brine (20 mL), dried over Na₂SO₄, and filtered. The filtrate was concentrated under reduced pressure to give 3-(aminomethyl)-4-fluoropyridin-2(1H)-one.

Step-by-step preparation of Example A.1

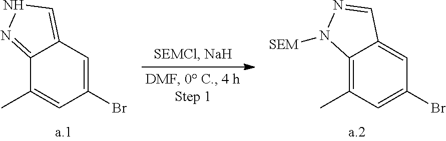 [SMILES: Cc1cc(Br)cc2c[nH]nc12 ; Cc1cc(Br)cc2cnn(COCC[Si](C)(C)C)c12]

To a solution of 5-bromo-7-methyl-2H-indazole (1.00 g, 4.74 mmol, 1 eq) in DMF (10 mL) was added NaH (379 mg, 9.48 mmol, 60% dispersion in oil) in portions at 0° C. After stirring for 30 min, SEMCl (1.58 g, 9.48 mmol, 1.68 mL, 2 eq) was then added, and the solution was stirred for another 3.5 h. The reaction mixture was quenched by addition of H₂O (30 mL) at 0° C. The reaction was further diluted with H₂O (20 mL), and the mixture was extracted with ethyl acetate (30 mL×3). The combined organic layers were washed with brine (20 mL×3), dried over Na₂SO₄, and filtered. The filtrate was concentrated under reduced pressure. The residue was purified by flash silica gel chromatography (ISCO®; 12 g SepaFlash® Silica Flash Column, gradient elution of 0~10% ethyl acetate/petroleum ether at 40 mL/min) to furnish 2-[(5-bromo-7-methyl-indazol-1-yl)methoxy]ethyl-trimethyl-silane.

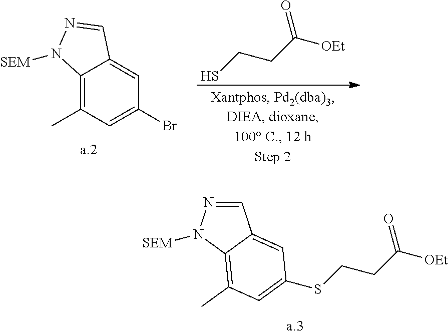 [SMILES: Cc1cc(Br)cc2cnn(COCC[Si](C)(C)C)c12 ; CCOC(=O)CCS ; CCOC(=O)CCSc1cc(C)c2c(cnn2COCC[Si](C)(C)C)c1]

A mixture of 2-[(5-bromo-7-methyl-indazol-1-yl)methoxy]ethyl-trimethyl-silane (900 mg, 2.64 mmol, 1 eq), ethyl 3-sulfanylpropanoate (531 mg, 3.96 mmol, 1.50 eq), DIEA (1.02 g, 7.91 mmol, 1.38 mL, 3 eq), Xantphos (153 mg, 0.264 mmol, 0.1 eq) and Pd₂(dba)₃ (241 mg, 0.264 mmol, 0.1 eq) in dioxane (10 mL) was degassed and purged with N₂ (3×). The mixture was stirred at 100° C. for 12 h under a N₂ atmosphere. The mixture was filtered, and the filtrate was concentrated under reduced pressure to give a residue. The residue was purified by flash silica gel chromatography (ISCO®; 12 g SepaFlash® Silica Flash Column, gradient elution of 0~10% ethyl acetate/petroleum ether at 40 mL/min) to furnish ethyl 3-[7-methyl-1-(2-trimethylsilylethoxymethyl)indazol-5-yl]sulfanylpropanoate. LCMS: (M+H⁺):395.2.

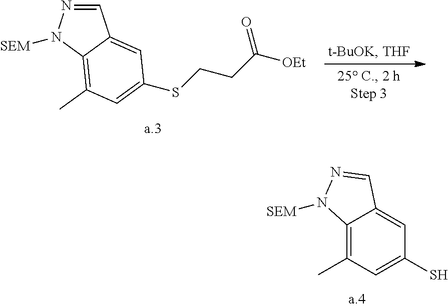 [SMILES: CCOC(=O)CCSc1cc(C)c2c(cnn2COCC[Si](C)(C)C)c1 ; Cc1cc(S)cc2cnn(COCC[Si](C)(C)C)c12]

A mixture of ethyl 3-[7-methyl-1-(2-trimethylsilylethoxymethyl)indazol-5-yl]sulfanylpropanoate (1.30 g, 3.29 mmol, 1 eq), t-BuOK (1.11 g, 9.88 mmol, 3 eq) in THE (20 mL) was degassed and purged with N₂ (3×). The mixture was stirred at 25° C. for 2 h under a N₂ atmosphere. The pH of the reaction mixture was adjusted to 4 by addition of aq. HCl (2 M). The mixture was extracted with EtOAc (30 mL×3). The combined organic layers were washed with brine (100 mL), dried over Na₂SO₄, and filtered. The filtrate was concentrated under reduced pressure to give a residue. The residue was purified by flash silica gel chromatography (ISCO®; 12 g SepaFlash® Silica Flash Column, gradient elution of 0-15% ethyl acetate/petroleum ether at 40 mL/min) to furnish 7-methyl-1-(2-trimethylsilylethoxymethyl)indazole-5-thiol. LCMS: (M+H⁺):295.2; ¹H NMR: (400 MHz, methanol-d4) δ 7.88 (s, 1H), 7.52 (d, J=1.1 Hz, 1H), 7.13 (s, 1H), 5.79 (s, 2H), 3.54-3.45 (m, 2H), 2.69 (s, 3H), 0.81 (t, J=8.0 Hz, 2H), −0.07-0.14 (m, 9H).

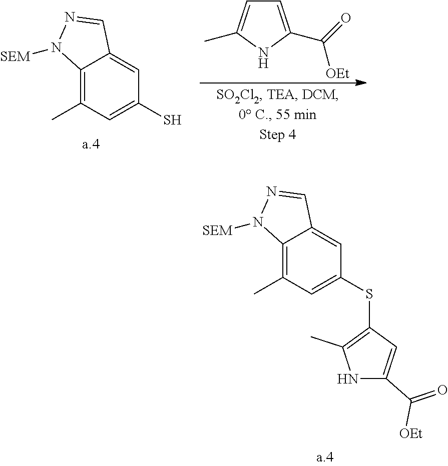 [SMILES: Cc1cc(S)cc2cnn(COCC[Si](C)(C)C)c12 ; CCOC(=O)c1ccc(C)[nH]1 ; CCOC(=O)c1cc(Sc2cc(C)c3c(cnn3COCC[Si](C)(C)C)c2)c(C)[nH]1]

To a solution of 7-methyl-1-(2-trimethylsilylethoxymethyl)indazole-5-thiol (211 mg, 0.718 mmol, 1.1 eq), TEA (3.30 mg, 0.033 mmol, 0.05 eq) and distilled DCM (10 mL), sulfuryl chloride (106 mg, 0.783 mmol, 1.2 eq) was carefully added in portions at 0° C. After stirring for 10 min, ethyl 5-methyl-1H-pyrrole-2-carboxylate (100 mg, 0.653 mmol, 1 eq) was added, and the solution was stirred for another 45 min. The reaction mixture was concentrated under reduced pressure to remove solvent. The residue was purified by flash silica gel chromatography (ISCO®; 12 g SepaFlash® Silica Flash Column, gradient elution of 0~30% ethyl acetate/petroleum ether at 60 mL/min) to furnish ethyl 5-methyl-4-[7-methyl-1-(2-trimethylsilylethoxymethyl)indazol-5-yl]sulfanyl-1H-pyrrole-2-carboxylate. LCMS: (M+H⁺):446.2.

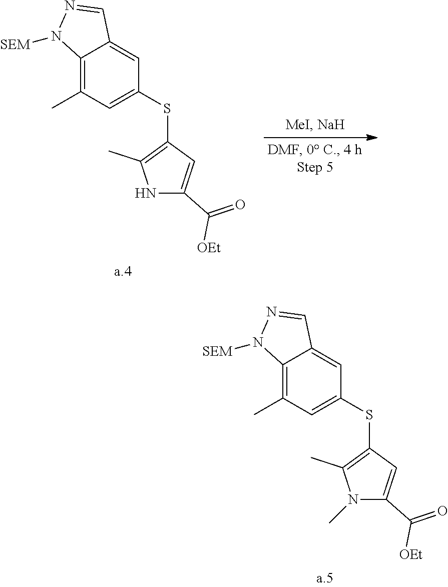 [SMILES: CCOC(=O)c1cc(Sc2cc(C)c3c(cnn3COCC[Si](C)(C)C)c2)c(C)[nH]1 ; CCOC(=O)c1cc(Sc2cc(C)c3c(cnn3COCC[Si](C)(C)C)c2)c(C)n1C]

To a solution of ethyl 5-methyl-4-[7-methyl-1-(2-trimethylsilylethoxymethyl)indazol-5-yl]sulfanyl-1H-pyrrole-2-carboxylate (300 mg, 0.673 mmol, 1 eq) in DMF (10 mL) was added NaH (54 mg, 1.35 mmol, 60% dispersion in oil) in portions at 0° C. The solution was allowed to stir for 30 min. Methyl iodide (191 mg, 1.35 mmol, 2 eq) was then added, and the solution was stirred for another 3.5 h. The reaction mixture was quenched by addition H₂O (30 mL) at 0° C., and then further diluted with additional H₂O (20 mL). The mixture was extracted with ethyl acetate (30 mL×3). The combined organic layers were washed with brine (20 mL×3), dried over Na₂SO₄, and filtered. The filtrate was concentrated under reduced pressure to give a residue. The residue was purified by flash silica gel chromatography (ISCO®; 12 g SepaFlash® Silica Flash Column, gradient elution of 0~10% ethyl acetate/petroleum ether gradient at 40 mL/min) to furnish ethyl 1,5-dimethyl-4-[7-methyl-1-(2-trimethylsilylethoxymethyl)indazol-5-yl]sulfanyl-pyrrole-2-carboxylate. LCMS: (M+H⁺):460.2.

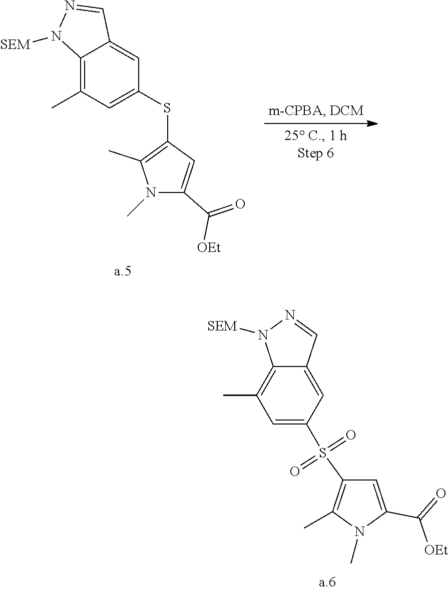 [SMILES: CCOC(=O)c1cc(Sc2cc(C)c3c(cnn3COCC[Si](C)(C)C)c2)c(C)n1C ; CCOC(=O)c1cc(S(=O)(=O)c2cc(C)c3c(cnn3COCC[Si](C)(C)C)c2)c(C)n1C]

To a solution of ethyl 1,5-dimethyl-4-[7-methyl-1-(2-trimethylsilylethoxymethyl)indazol-5-yl]sulfanyl-pyrrole-2-carboxylate (200 mg, 0.435 mmol, 1 eq) in DCM (5 mL) was added m-CPBA (235 mg, 1.09 mmol, 80% purity, 2.5 eq) at 0° C. The mixture was stirred at 25 C for 1 h. The reaction mixture was washed sequentially with aq. Na₂SO₃ (20 mL, 10%) and NaHCO₃ (20 ml, sat.). The organic layer was dried over Na₂SO₄ and filtered. The filtrate was concentrated under reduced pressure to give a residue. The residue was purified by flash silica gel chromatography (ISCO®; 12 g SepaFlash® Silica Flash Column, gradient elution of 0~20% ethyl acetate/petroleum ether at 40 mL/min) to furnish ethyl 1,5-dimethyl-4-[7-methyl-1-(2-trimethylsilylethoxymethyl)indazol-5-yl]sulfonyl-pyrrole-2-carboxylate. LCMS: (M+H⁺):492.3.

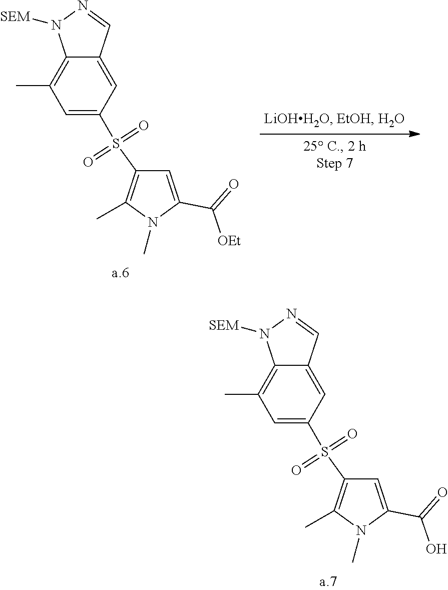 [SMILES: CCOC(=O)c1cc(S(=O)(=O)c2cc(C)c3c(cnn3COCC[Si](C)(C)C)c2)c(C)n1C ; Cc1cc(S(=O)(=O)c2cc(C(=O)O)n(C)c2C)cc2cnn(COCC[Si](C)(C)C)c12]

A mixture of ethyl 1,5-dimethyl-4-[7-methyl-1-(2-trimethylsilylethoxymethyl)indazol-5-yl]sulfonyl-pyrrole-2-carboxylate (120 mg, 0.244 mmol, 1 eq) and LiOH·H₂O (10 mg, 0.24 mmol, 1 eq) in EtOH (5 mL) and H₂O (1 mL) was degassed and purged with N₂ (3×). The mixture was stirred at 25° C. for 2 h under a N₂ atmosphere. The reaction mixture was concentrated under reduced pressure to remove solvent. The residue was diluted with H₂O (3 mL). The pH of the aqueous phase was adjusted to 2 with aq. HCl (2 M). The formed solid was collected and dried under reduced pressure to furnish 1,5-dimethyl-4-[7-methyl-1-(2-trimethylsilylethoxymethyl)indazol-5-yl]sulfonyl-pyrrole-2-carboxylic acid. LCMS: (M+H⁺):464.2.

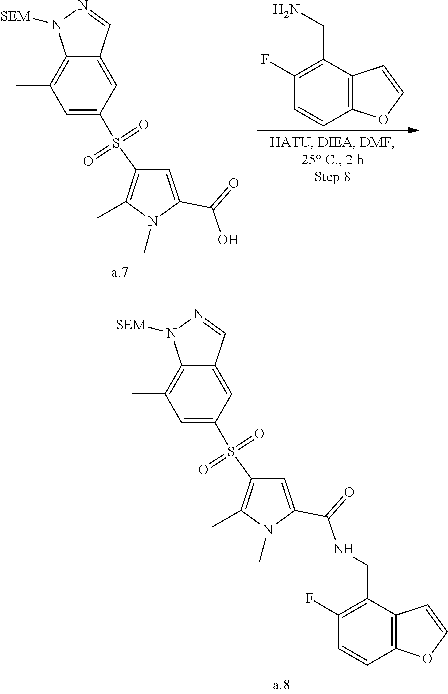 [SMILES: Cc1cc(S(=O)(=O)c2cc(C(=O)O)n(C)c2C)cc2cnn(COCC[Si](C)(C)C)c12 ; NCc1c(F)ccc2occc12 ; Cc1cc(S(=O)(=O)c2cc(C(=O)NCc3c(F)ccc4occc34)n(C)c2C)cc2cnn(COCC[Si](C)(C)C)c12]

A mixture of 1,5-dimethyl-4-[7-methyl-1-(2-trimethylsilylethoxymethyl)indazol-5-yl]sulfonyl-pyrrole-2-carboxylic acid (140 mg, 0.302 mmol, 1 eq), (5-fluorobenzofuran-4-yl)methanamine (50 mg, 0.30 mmol, 1 eq), HATU (138 mg, 0.362 mmol, 1.2 eq), DIEA (117 mg, 0.905 mmol, 3 eq) in DMF (1 mL) was degassed and purged with N₂ (3×). The mixture was stirred at 25° C. for 2 h under a N₂ atmosphere. The mixture was poured into water (20 mL), and the mixture was extracted with ethyl acetate (15 mL×3). The combined organic phase was washed with brine (15 mL), dried with anhydrous Na₂SO₄, and filtered. The filtrate was concentrated under vacuum. The residue was purified by flash silica gel chromatography (ISCO®; 4 g SepaFlash® Silica Flash Column, gradient elution of 0~30% ethyl acetate/petroleum ethergradient at 40 mL/min) to furnish N-[(5-fluorobenzofuran-4-yl)methyl]-1,5-dimethyl-4-[7-methyl-1-(2-trimethylsilylethoxymethyl)indazol-5-yl]sulfonyl-pyrrole-2-carboxamide. LCMS: (M+H⁺):611.3.

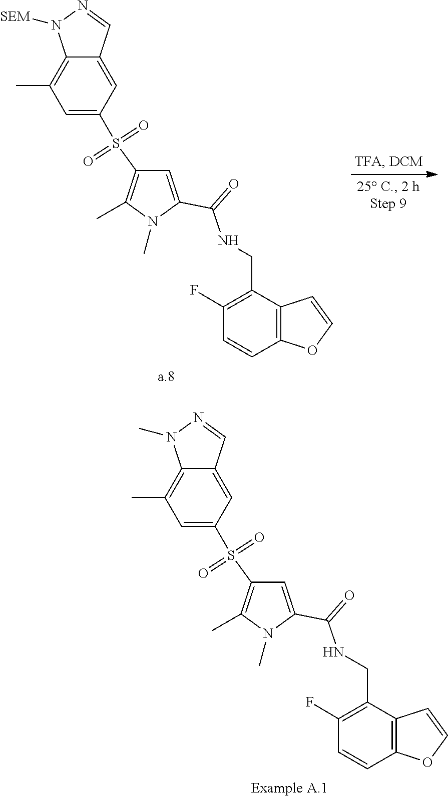 [SMILES: Cc1cc(S(=O)(=O)c2cc(C(=O)NCc3c(F)ccc4occc34)n(C)c2C)cc2cnn(COCC[Si](C)(C)C)c12 ; Cc1cc(S(=O)(=O)c2cc(C(=O)NCc3c(F)ccc4occc34)n(C)c2C)cc2cnn(C)c12]

A mixture of N-[(5-fluorobenzofuran-4-yl)methyl]-1,5-dimethyl-4-[7-methyl-1-(2-trimethylsilylethoxymethyl)indazol-5-yl]sulfonyl-pyrrole-2-carboxamide (100 mg, 0.164 mmol, 1 eq) in TFA (0.5 mL) and DCM (1.5 mL) was degassed and purged with N₂ (3×). The mixture was stirred at 25° C. for 2 h under a N₂ atmosphere. The mixture was poured into water (10 mL), and the mixture was extracted with ethyl acetate (10 mL×3). The combined organic phase was washed with brine (10 mL), dried with anhydrous Na₂SO₄, and filtered. The filtrate was concentrated. The residue was purified by preparative-HPLC (column: Waters Xbridge BEH C18 100*30 mm*10 um;mobile phase: [H₂O(10 mM NH₄HCO₃)-ACN]; gradient:355%-65% B over 8.0 min) to furnish N-[(5-fluorobenzofuran-4-yl)methyl]-1,5-dimethyl-4-[(7-methyl-1H-indazol-5-yl)sulfonyl]pyrrole-2-carboxamide. LCMS: (M+H⁺):481.0; ¹H NMR: (400 MHz, methanol-d4) δ 8.25 (s, 1H), 8.19 (s, 1H), 7.78 (d, J=2.1 Hz, 1H), 7.52 (s, 1H), 7.41 (dd, JH 3.8, 8.9 Hz, 1H), 7.11-6.99 (m, 3H), 4.74 (s, 2H), 3.77 (s, 3H), 2.56 (s, 3H), 2.42 (s, 3H).

The examples in Table 1 were prepared in a similar fashion to Example A.1 in Scheme A using the appropriate reagents in Steps 1 (halide) and Steps 8 (amine).

TABLE 1

| | Halide | Amine | | ¹H NMR |
|---|---|---|---|---|
| Ex. | (Step 1) | (Step 8) | Structure | LCMS |
| A.2 | 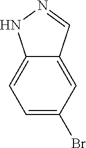 | 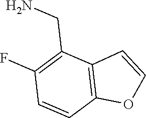 [SMILES: NCc1c(F)ccc2occc12] | 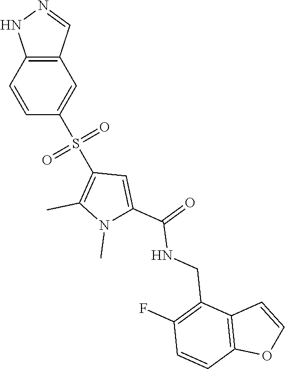 [SMILES: Cc1c(S(=O)(=O)c2ccc3[nH]ncc3c2)cc(C(=O)NCc2c(F)ccc3occc23)n1C] | LCMS (M + H⁺): 467.0 ¹H NMR: (400 MHz, methanol- d4) δ 8.42 (s, 1H), 8.22 (s, 1H), 7.80-7.73 (m, 2H), 7.64 (d, J = 8.8 Hz, 1H), 7.42 (dd, J = 3.7, 9.0 Hz, 1H), 7.11-7.00 (m, 3H), 4.74 (s, 2H), 3.78 (s, 3H), 2.43 (s, 3H) |
| A.3 | 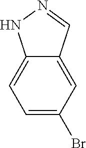 | 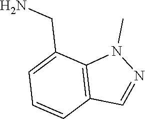 | 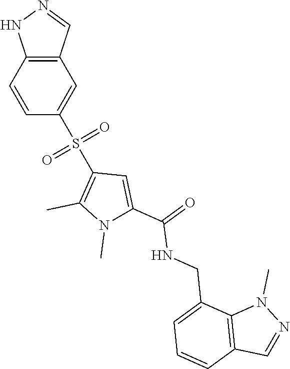 [SMILES: Cc1c(S(=O)(=O)c2ccc3[nH]ncc3c2)cc(C(=O)NCc2cccc3cnn(C)c23)n1C] | LCMS (M + H⁺): 463.1 ¹H NMR: (400 MHz, methanol- d4) δ 8.47-8.40 (m, 1H), 8.26-8.18 (m, 1H), 7.97 (s, 1H), 7.86-7.77 (m, 1H), 7.71-7.59 (m, 2H), 7.33 (br d, J = 7.0 Hz, 1H), 7.20 (s, 1H), 7.10 (t, J = 7.6 Hz, 1H), 4.97 (s, 2H), 4.28 (s, 3H), 3.80 (s, 3H), 2.45 (s, 3H) |
| A.5 | 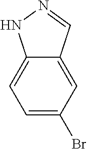 | 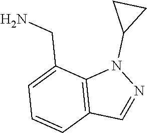 [SMILES: NCc1cccc2cnn(C3CC3)c12] | 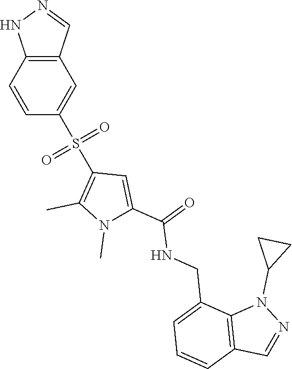 [SMILES: Cc1c(S(=O)(=O)c2ccc3[nH]ncc3c2)cc(C(=O)NCc2cccc3cnn(C4CC4)c23)n1C] | LCMS (M + H⁺): 489.1 ¹H NMR: (400 MHz, DMSO-d6) δ 8.91-8.82 (m, 1H), 8.39 (s, 1H), 8.30 (s, 1H), 7.99 (s, 1H), 7.76- 7.68 (m, 2H), 7.64 (d, J = 7.9 Hz, 1H), 7.34 (s, 1H), 7.26 (d, J = 7.2 Hz, 1H), 7.14-7.05 (m, 1H), 6.11- 6.03 (m, 1H), 5.11 (d, J = 5.5 Hz, 2H), 4.06-4.00 (m, 1H), 3.76 (s, 3H), 2.41 (s, 3H), 1.30-1.24 (m, 2H), 1.16-1.10 (m, 2H) |

TABLE A

| | Halide | Amine | | ¹H NMR |
|---|---|---|---|---|
| Ex. | (Step 1) | (Step 8) | Structure | LCMS |
| A.13 | 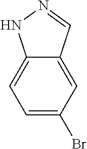 | 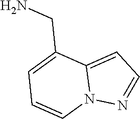 [SMILES: NCc1cccn2nccc12] | 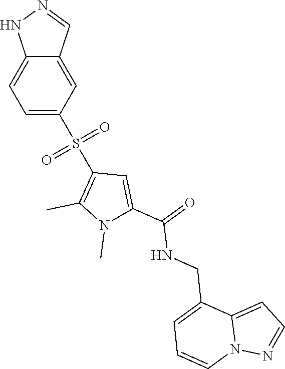 [SMILES: Cc1c(S(=O)(=O)c2ccc3[nH]ncc3c2)cc(C(=O)NCc2cccn3nccc23)n1C] | LCMS (M + H⁺): 449.0; ¹H NMR (400 MHz, DMSO-d6) δ 13.74-13.26 (m, 1H), 8.88 (t, J = 5.8 Hz, 1H), 8.59 (d, J = 7.0 Hz, 1H), 8.38 (s, 1H), 8.30 (s, 1H), 7.99 (d, J = 2.3 Hz, 1H), 7.70 (d, J = 0.9 Hz, 2H), 7.27 (s, 1H), 7.07 (d, J = 6.9 Hz, 1H), 6.85 (t, J = 6.9 Hz, 1H), 6.73-6.69 (m, 1H), 4.56 (d, J = 5.8 Hz, 2H), 3.76 (s, 3H), 2.40 (s, 3H) |
| A.15 | 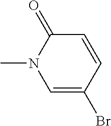 (no SEM protection required) | 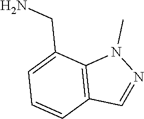 | 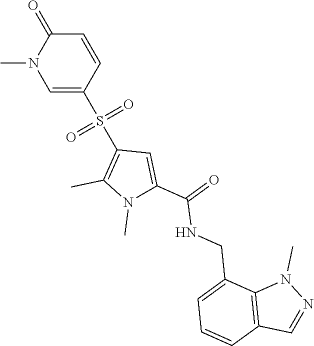 [SMILES: Cc1c(S(=O)(=O)c2ccc(=O)n(C)c2)cc(C(=O)NCc2cccc3cnn(C)c23)n1C] | LCMS (M + H⁺): 454.0; ¹H NMR (400 MHz, DMSO-d6) δ 8.83 (br t, J = 5.3 Hz, 1H), 8.46 (d, J = 2.6 Hz, 1H), 8.03 (s, 1H), 7.67 (d, J = 8.0 Hz, 1H), 7.56 (dd, J = 2.7, 9.6 Hz, 1H), 7.30- 7.22 (m, 2H), 7.08 (t, J = 7.5 Hz, 1H), 6.46 (d, J = 9.6 Hz, 1H), 4.89 (d, J = 5.3 Hz, 2H), 4.25 (s, 3H), 3.79 (s, 3H), 3.49 (s, 3H), 2.41 (s, 3H) |

5-Bromo-3-fluoro-1H-indazole (Scheme Aa) can be converted into the SEM-protected acid in a manner like that described in Scheme Ah (Steps 1-6).

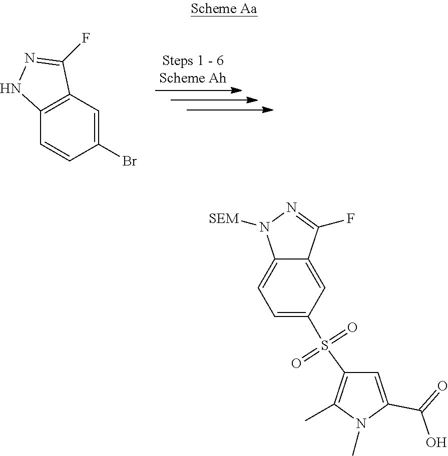 [SMILES: Fc1n[nH]c2ccc(Br)cc12 ; Cc1c(S(=O)(=O)c2ccc3c(c2)c(F)nn3COCC[Si](C)(C)C)cc(C(=O)O)n1C]

The acids in Table Aa can be made in a similar fashion to that described above in Scheme Aa using the appropriate heteroaryl halide.

TABLE Aa

| Heteroaryl halide | Acid |
|---|---|
| 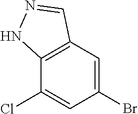 [SMILES: Clc1cc(Br)cc2cn[nH]c12] | 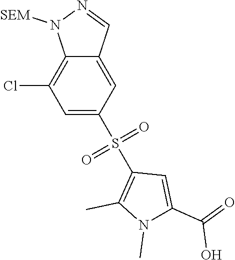 [SMILES: Cc1c(S(=O)(=O)c2cc(Cl)c3c(cnn3COCC[Si](C)(C)C)c2)cc(C(=O)O)n1C] |
| 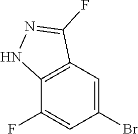 [SMILES: Fc1n[nH]c2c(F)cc(Br)cc12] | 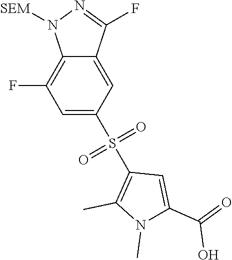 [SMILES: Cc1c(S(=O)(=O)c2cc(F)c3c(c2)c(F)nn3COCC[Si](C)(C)C)cc(C(=O)O)n1C] |
| 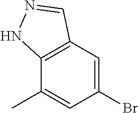 | 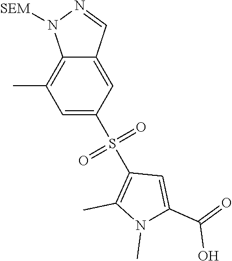 [SMILES: Cc1cc(S(=O)(=O)c2cc(C(=O)O)n(C)c2C)cc2cnn(COCC[Si](C)(C)C)c12] |
| 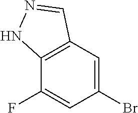 [SMILES: Fc1cc(Br)cc2cn[nH]c12] | 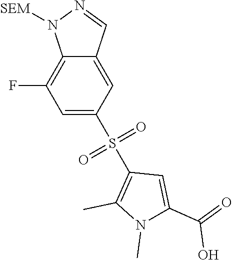 [SMILES: Cc1c(S(=O)(=O)c2cc(F)c3c(cnn3COCC[Si](C)(C)C)c2)cc(C(=O)O)n1C] |
| 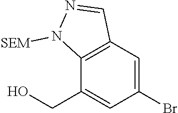 [SMILES: C[Si](C)(C)CCOCn1ncc2cc(Br)cc(CO)c21] (Steps 2-6, Scheme Ah) | 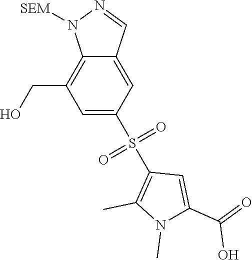 [SMILES: Cc1c(S(=O)(=O)c2cc(CO)c3c(cnn3COCC[Si](C)(C)C)c2)cc(C(=O)O)n1C] |
| 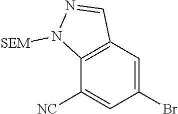 [SMILES: C[Si](C)(C)CCOCn1ncc2cc(Br)cc(C#N)c21] (Steps 2-6, Scheme Ah) | 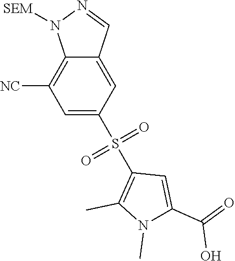 [SMILES: Cc1c(S(=O)(=O)c2cc(C#N)c3c(cnn3COCC[Si](C)(C)C)c2)cc(C(=O)O)n1C] |
| 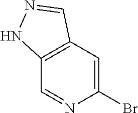 | 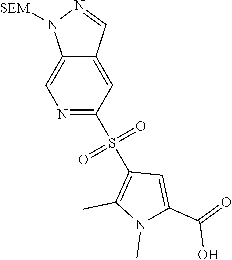 [SMILES: Cc1c(S(=O)(=O)c2cc3cnn(COCC[Si](C)(C)C)c3cn2)cc(C(=O)O)n1C] |
| 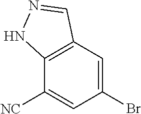 [SMILES: N#Cc1cc(Br)cc2cn[nH]c12] | 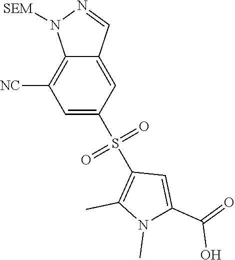 [SMILES: Cc1c(S(=O)(=O)c2cc(C#N)c3c(cnn3COCC[Si](C)(C)C)c2)cc(C(=O)O)n1C] |
| 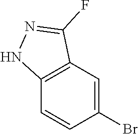 [SMILES: Fc1n[nH]c2ccc(Br)cc12] | 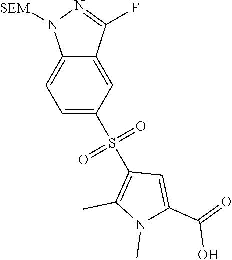 [SMILES: Cc1c(S(=O)(=O)c2ccc3c(c2)c(F)nn3COCC[Si](C)(C)C)cc(C(=O)O)n1C] |
| 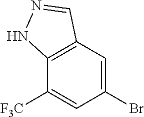 [SMILES: FC(F)(F)c1cc(Br)cc2cn[nH]c12] | 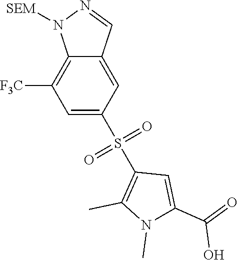 [SMILES: Cc1c(S(=O)(=O)c2cc(C(F)(F)F)c3c(cnn3COCC[Si](C)(C)C)c2)cc(C(=O)O)n1C] |
| 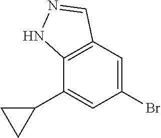 [SMILES: Brc1cc(C2CC2)c2[nH]ncc2c1] | 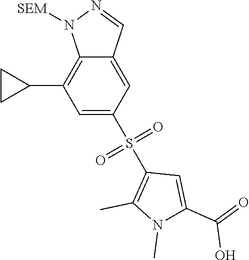 [SMILES: Cc1c(S(=O)(=O)c2cc(C3CC3)c3c(cnn3COCC[Si](C)(C)C)c2)cc(C(=O)O)n1C] |
| 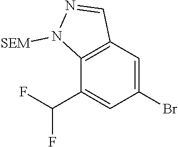 [SMILES: C[Si](C)(C)CCOCn1ncc2cc(Br)cc(C(F)F)c21] (Steps 2-6, Scheme Ah) | 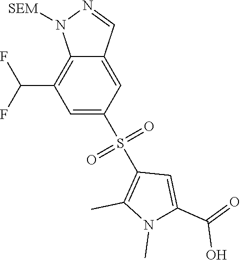 [SMILES: Cc1c(S(=O)(=O)c2cc(C(F)F)c3c(cnn3COCC[Si](C)(C)C)c2)cc(C(=O)O)n1C] |
| 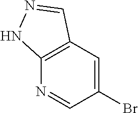 | 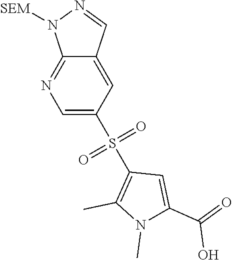 [SMILES: Cc1c(S(=O)(=O)c2cnc3c(cnn3COCC[Si](C)(C)C)c2)cc(C(=O)O)n1C] |
| 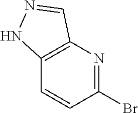 | 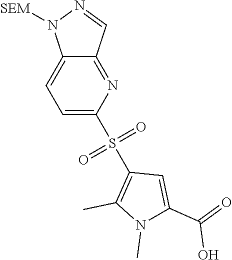 [SMILES: Cc1c(S(=O)(=O)c2ccc3c(cnn3COCC[Si](C)(C)C)n2)cc(C(=O)O)n1C] |

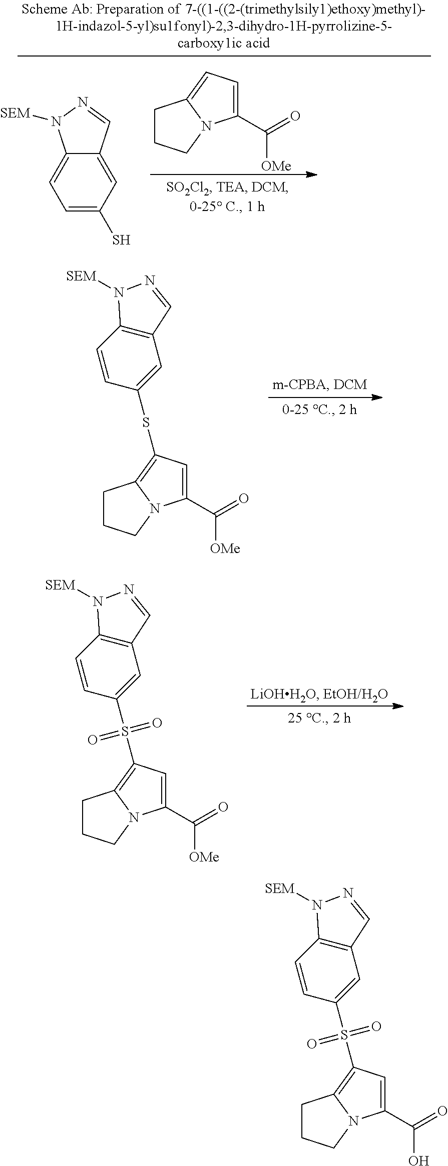

1-((2-(Tri methyl silyl)ethoxy)methyl)-1H-indazole-5-thiol can be converted into the 7-((1-((2-(trimethyl silyl)ethoxy)methyl)-1H-indazol-5-yl)sulfonyl)-2,3-dihydro-1H-pyrrolizine-5-carboxylic acid (Scheme Ab) using conditions like those outlined in Scheme A.

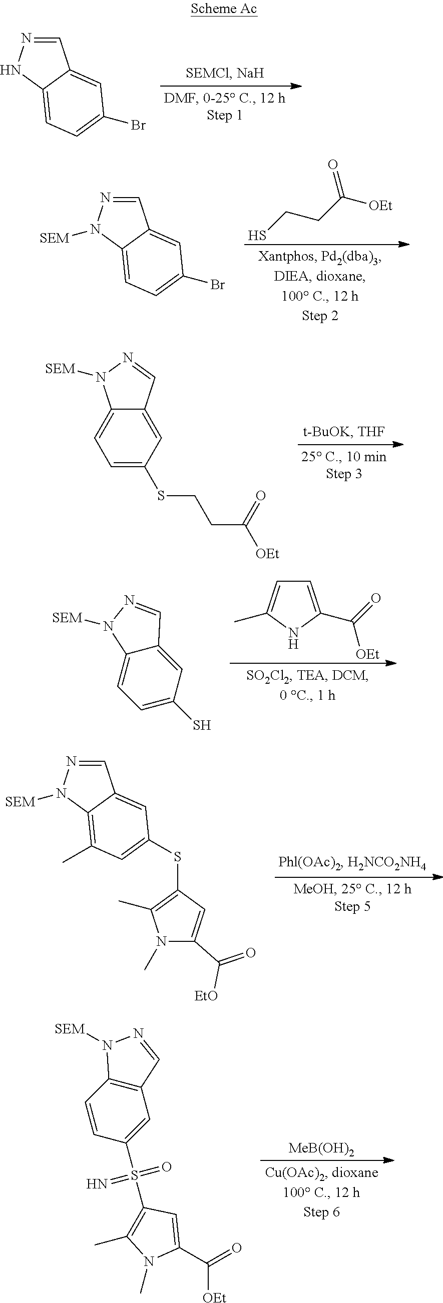

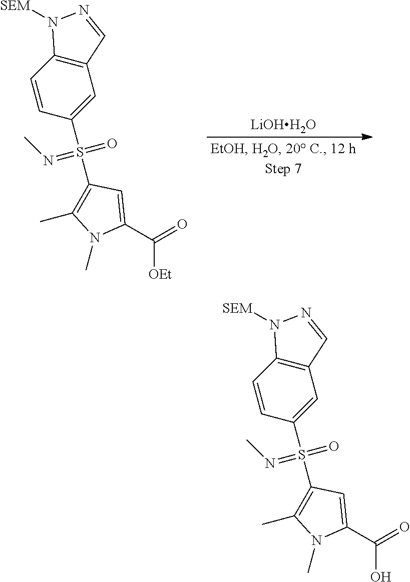 [SMILES: CCOC(=O)c1cc(S(=O)(=NC)c2ccc3c(cnn3COCC[Si](C)(C)C)c2)c(C)n1C ; "LiOH·H2O" ; CN=S(=O)(c1ccc2c(cnn2COCC[Si](C)(C)C)c1)c1cc(C(=O)O)n(C)c1C]

Step 1. To a solution of 5-bromo-1H-indazole (40.0 g, 203 mmol, 1 eq) in DMF (400 mL) was added NaH (16.2 g, 406 mmol, 60 wt % dispersion in oil) and SEM-Cl (40.6 g, 244 mmol, 43.1 mL, 1.2 eq) at 0° C. The mixture was stirred at 25° C. for 12 h. The reaction mixture was quenched with H₂O (1000 mL), and the mixture was extracted with EtOAc (500 mL×3). The combined organic layers were washed with brine (1000 mL), dried over Na₂SO₄, and filtered. The filtrate was concentrated under reduced pressure. The residue was purified by flash silica gel chromatography (ISCO®; 330 g SepaFlash® Silica Flash Column, eluent of 2~3% ethyl acetate/petroleum ether gradient at 100 mL/min) to furnish 2-[(5-bromoindazol-1-yl)methoxy]ethyl-trimethyl-silane.

Step 2. A mixture of 2-[(5-bromoindazol-1-yl)methoxy]ethyl-trimethyl-silane (10.0 g, 30.6 mmol, 1 eq), ethyl 3-sulfanylpropanoate (4.92 g, 36.7 mmol, 1.2 eq), DIPEA (11.9 g, 91.7 mmol, 16.0 mL, 3 eq), Xantphos (2.65 g, 4.58 mmol, 0.15 eq) and Pd₂(dba)₃ (2.80 g, 3.06 mmol, 0.1 eq) in dioxane (100 mL) was degassed and purged with N₂ for 3 times. The mixture was stirred at 100° C. for 12 h under a N₂ atmosphere. The reaction mixture was diluted with H₂O (400 mL), and the mixture was extracted with EtOAc (200 mL×3). The combined organic layers were washed with brine (400 mL), dried over Na₂SO₄, and filtered. The filtrate was concentrated under reduced pressure. The residue was purified by flash silica gel chromatography (ISCO®; 120 g SepaFlash® Silica Flash Column, eluent of 5-15% ethyl acetate/petroleum ether gradient at 100 mL/min) to furnish ethyl 3-[1-(2-trimethylsilylethoxymethyl)indazol-5-yl]sulfanylpropanoate.

Step 3. To a solution of ethyl 3-[1-(2-trimethylsilylethoxymethyl)indazol-5-yl]sulfanylpropanoate (3.00 g, 7.88 mmol, 1 eq) in THE (30 mL) was added t-BuOK (1 M, 23.7 mL, 3 eq). The mixture was stirred at 25° C. for 10 min. The pH of the reaction mixture was adjusted to 4 by addition of aqueous HCl (2 M). The mixture was extracted with EtOAc. The organic layers were dried over Na₂SO₄ and filtered. The filtrate was concentrated under reduced pressure to give a residue. The residue was purified by flash silica gel chromatography (ISCO®; 24 g SepaFlash® Silica Flash Column, eluent of 4-10% ethyl acetate/petroleum ether gradient at 60 mL/min) to furnish 1-(2-trimethylsilylethoxymethyl)indazole-5-thiol.

Step 4. At 0° C., to a solution of 1-(2-trimethylsilylethoxymethyl)indazole-5-thiol (906 mg, 3.23 mmol, 1 eq), TEA (16 mg, 0.16 mmol, 0.05 eq) and distilled DCM (10 mL), sulfuryl chloride (436 mg, 3.23 mmol, 323 μL, 1 eq) was carefully added in portions. After stirring for 10 min at 0° C. ethyl 1,5-dimethylpyrrole-2-carboxylate (540 mg, 3.23 mmol, 1 eq) was added, and the solution was stirred for another 50 min. The reaction mixture was concentrated under reduced pressure to remove solvent. The residue was purified by flash silica gel chromatography (ISCO®; 4 g SepaFlash® Silica Flash Column, eluent of 4-5% ethyl acetate/petroleum ether gradient at 40 mL/min) to furnish ethyl 1,5-dimethyl-4-[1-(2-trimethylsilylethoxymethyl) indazol-5-yl]sulfanyl-pyrrole-2-carboxylate.

Step 5. A mixture of ethyl 1,5-dimethyl-4-[1-(2-trimethylsilylethoxymethyl)indazol-5-yl]sulfanyl-pyrrole-2-carboxylate (1.00 g, 2.24 mmol, 1 eq), [acetoxy(phenyl)-iodanyl]acetate (1.81 g, 5.61 mmol, 2.5 eq), ammonium carbamate (438 mg, 5.61 mmol, 2.5 eq) in MeOH (10 mL) was degassed and purged with N₂ for 3 times. The mixture was stirred at 25° C. for 12 h under a N₂ atmosphere. The reaction mixture was concentrated under reduced pressure to remove the solvent. The residue was purified by flash silica gel chromatography (ISCO®; 12 g SepaFlash® Silica Flash Column, eluent of 30-40% ethyl acetate/petroleum ether gradient at 60 mL/min) to furnish ethyl 1,5-dimethyl-4-[[1-(2-trimethylsilylethoxymethyl)indazol-5-yl]sulfonimidoyl]pyrrole-2-carboxylate.

Step 6. A mixture of ethyl 1,5-dimethyl-4-[[1-(2-trimethylsilylethoxymethyl)indazol-5-yl]sulfonimidoyl]pyrrole-2-carboxylate (400 mg, 839 mol, 1 eq), methylboronic acid (151 mg, 2.52 mmol, 3 eq), Cu(OAc)₂ (229 mg, 1.26 mmol, 1.5 eq) in dioxane (5 mL) was degassed and purged with O₂ (15Psi) (3×). The mixture was stirred at 100° C. for 12 hr under a N₂ atmosphere. The reaction mixture was diluted with H₂O (20 mL). The mixture was extracted with EtOAc (10 mL×3). The combined organic layers were washed with brine (20 mL), dried over Na₂SO₄, and filtered. The filtrate was concentrated under reduced pressure to give a residue. The residue was purified by flash silica gel chromatography (ISCO®; 4 g SepaFlash® Silica Flash Column, eluent of 30-35% ethyl acetate/petroleum ether gradient at 40 mL/min) to furnish ethyl 1,5-dimethyl-4-[N-methyl-S-[1-(2-trimethylsilylethoxymethyl)indazol-5-yl]sulfonimidoyl]pyrrole-2-carboxylate.

Step 7. To a solution of ethyl 1,5-dimethyl-4-[N-methyl-S-[1-(2-trimethylsilylethoxy methyl)indazol-5-yl]sulfonimidoyl]pyrrole-2-carboxylate (170 mg, 346 mol, 1 eq) in EtOH (3 mL) and H₂O (1 mL) was added LiOH·H₂O (29 mg, 690 mol, 2 eq). The mixture was stirred at 25° C. for 12 h. The reaction mixture was concentrated under reduced pressure to remove the solvent. The residue was diluted with H₂O (1 mL), and the pH was adjusted to 3 by addition of aqeuous HCl (2 M). The mixture was extracted with EtOAc (10 mL×3). The combined organic layers were washed with brine (20 mL), dried over Na₂SO₄, and filtered. The filtrate was concentrated under reduced pressure to furnish 1,5-dimethyl-4-[N-methyl-S-[1-(2-trimethylsilylethoxymethyl)indazol-5-yl]sulfonimidoyl]pyrrole-2-carboxylic acid.

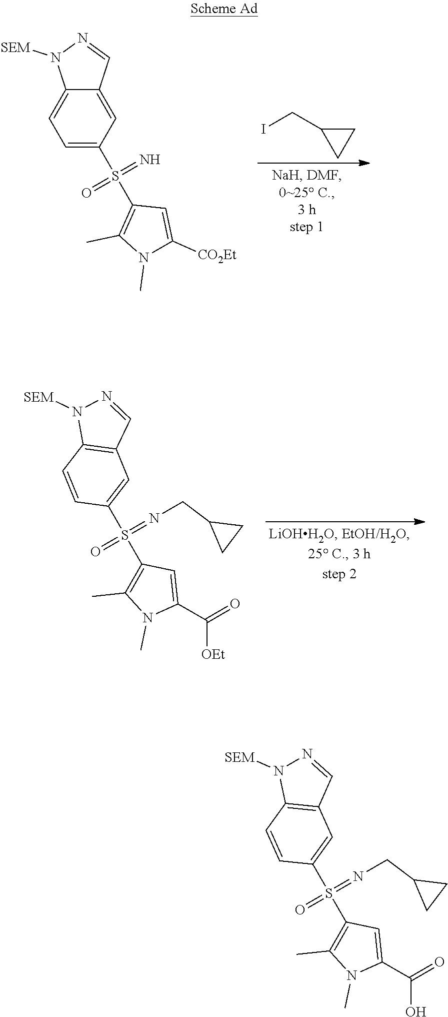 [SMILES: CCOC(=O)c1cc(S(=N)(=O)c2ccc3c(cnn3COCC[Si](C)(C)C)c2)c(C)n1C ; ICC1CC1 ; CCOC(=O)c1cc(S(=O)(=NCC2CC2)c2ccc3c(cnn3COCC[Si](C)(C)C)c2)c(C)n1C ; Cc1c(S(=O)(=NCC2CC2)c2ccc3c(cnn3COCC[Si](C)(C)C)c2)cc(C(=O)O)n1C]

Step 1. To a solution of ethyl 1,5-dimethyl-4-[[1-(2-trimethylsilylethoxymethyl)indazol-5-yl]sulfonimidoyl]pyrrole-2-carboxylate (0.560 g, 1.17 mmol, 1 eq) and iodomethylcyclopropane (235 mg, 1.29 mmol, 1.1 eq) in DMF (5 mL) was added NaH (70 mg, 1.76 mmol, 60 wt % dispersion in oil, 1.5 eq) at 0° C. The mixture was stirred at 25° C. for 3 h. The reaction mixture was quenched with saturated NH₄Cl (aq, 10 mL). The mixture was extracted with EtOAc (10 mL×3). The combined organic layers were washed with brine (10 mL), dried over Na₂SO₄, and filtered. The filtrate was concentrated under reduced pressure to furnish ethyl 4-[N-(cyclopropylmethyl)-S-[1-(2-trimethylsilylethoxymethyl)indazol-5-yl]sulfonimidoyl]-1,5-dimethyl-pyrrole-2-carboxylate.

Step 2. To a solution of ethyl 4-[N-(cyclopropylmethyl)-S-[1-(2-trimethylsilylethoxymethyl)indazol-5-yl]sulfonimidoyl]-1,5-dimethyl-pyrrole-2-carboxylate (470 mg, 886 mol, 1 eq) in EtOH (2 mL) and H₂O (0.5 mL) was added LiOH·H₂O (74 mg, 1.8 mmol, 2 eq). The mixture was stirred at 25° C. for 3 h. The reaction mixture was concentrated under reduced pressure. The mixture was poured into H₂O (4 mL). The pH of the mixture was adjusted to 5 by addition of aqueous HCl(2 N). The mixture was extracted with ethyl acetate (10 mL×3). The combined organic layers were washed with brine (20 mL), dried over Na₂SO₄, and filtered. The filtrate was concentrated under reduced pressure to furnish 4-[N-(cyclopropylmethyl)-S-[1-(2-trimethylsilylethoxymethyl)indazol-5-yl]sulfonimidoyl]-1,5-dimethyl-pyrrole-2-carboxylic acid.

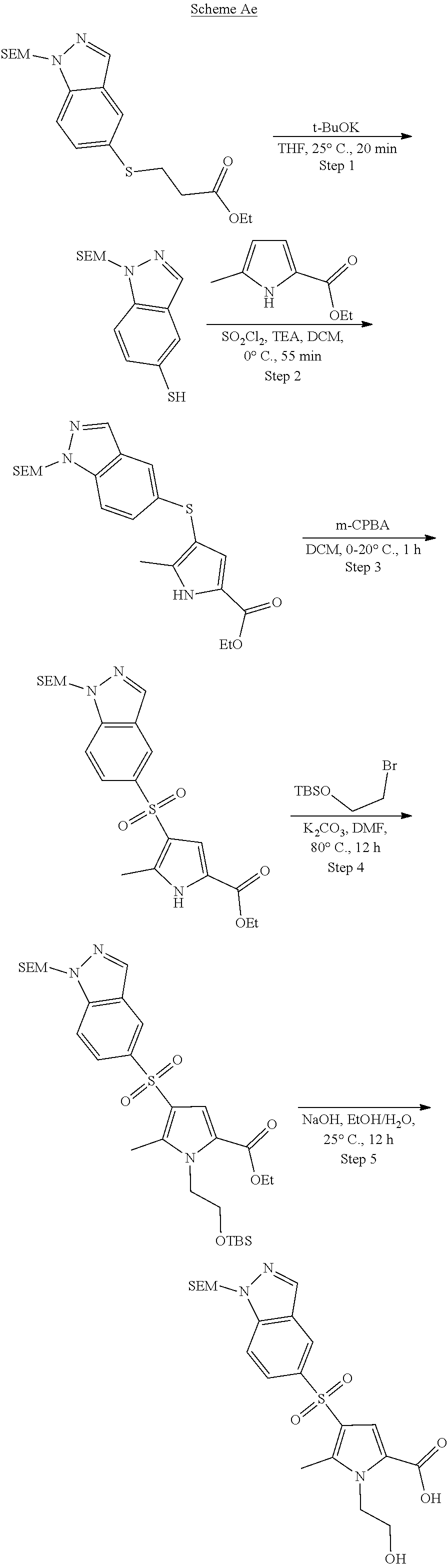

1-(2-Hydroxyethyl)-5-methyl-4-((1-((2-(trimethylsilyl)ethoxy)methyl)-1H-indazol-5-yl)sulfonyl)-1H-pyrrole-2-carboxylic was prepared using similar conditions outlined for other sulfone-acid intermediates using conditions outlined in Scheme Ae.

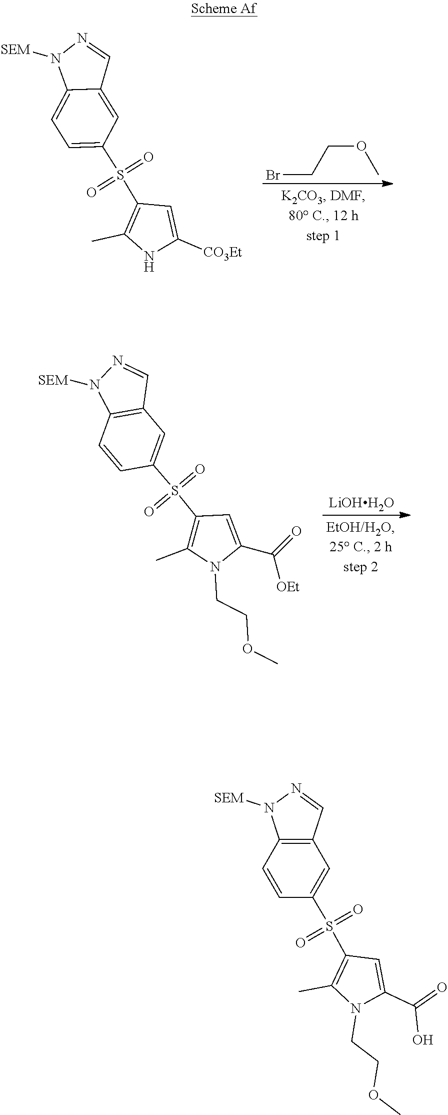

1-(2-Methoxyethyl)-5-methyl-4-((1-((2-(trimethylsilyl)ethoxy)methyl)-1H-indazol-5-yl)sulfonyl)-1H-pyrrole-2-carboxylic acid was prepared using similar conditions outlined for other sulfone-acid intermediates outlined in Scheme Af.

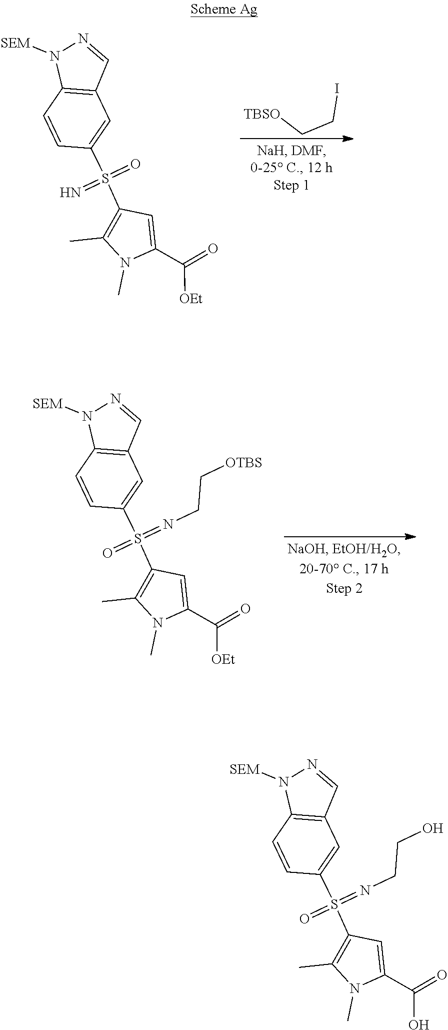 [SMILES: CCOC(=O)c1cc(S(=N)(=O)c2ccc3c(cnn3COCC[Si](C)(C)C)c2)c(C)n1C ; CC(C)(C)[Si](C)(C)OCCI ; CCOC(=O)c1cc(S(=O)(=NCCO[Si](C)(C)C(C)(C)C)c2ccc3c(cnn3COCC[Si](C)(C)C)c2)c(C)n1C ; Cc1c(S(=O)(=NCCO)c2ccc3c(cnn3COCC[Si](C)(C)C)c2)cc(C(=O)O)n1C]

Step 1. To a solution of ethyl 1,5-dimethyl-4-[[1-(2-trimethylsilylethoxymethyl)indazol-5-yl]sulfonimidoyl]pyrrole-2-carboxylate (100 mg, 210 mol, 1 eq) in DMF (2 mL) was added NaH (17 mg, 420 mol, 60 wt % dispersion in oil) at 0° C. tert-Butyl-(2-iodoethoxy)-dimethyl-silane (120 mg, 420 mol, 2 eq) was added, and the mixture was stirred at 25° C. for 12 h. The reaction mixture was quenched with MeOH (0.5 mL), and the mixture was concentrated under reduced pressure. The residue was purified by prep-HPLC (neutral condition, column: Waters Xbridge Prep OBD C18 150×40 mm×10 um; mobile phase: [H₂O(10 mM NH₄HCO₃)-ACN];gradient:45%-85% B over 8.0 min) to furnish ethyl 4-[N-[2-[tert-butyl(dimethyl)silyl]oxyethyl]-S-[1-(2-trimethylsilylethoxymethyl)indazol-5-yl]sulfonimidoyl]-1,5-dimethyl-pyrrole-2-carboxylate.

Step 2. To a solution of 4-[N-[2-[tert-butyl(dimethyl)silyl]oxyethyl]-S-[1-(2-trimethylsilyl ethoxymethyl)indazol-5-yl]sulfonimidoyl]-1,5-dimethyl-pyrrole-2-carboxylic acid (30 mg, 49 mol, 1 eq) in EtOH (1 mL) was added NaOH (4.0 mg, 99 mol, 2 eq) and H₂O (0.3 mL). The mixture was stirred at 20° C. for 12 h. The mixture was heated to 70° C. for 5 h. The reaction mixture was concentrated under reduced pressure to remove solvent. The residue was diluted with H₂O (1 mL), and the pH was adjusted to 3 by addition of aqeuous HCl (2M). The mixture was extracted with EtOAc (10 mL×3). The combined organic layers were washed with brine (20 mL), dried over Na₂SO₄, and filtered. The filtrate was concentrated under reduced pressure to furnish 4-[N-(2-hydroxyethyl)-S-[1-(2-trimethylsilylethoxymethyl)indazol-5-yl]sulfonimidoyl]-1,5-dimethyl-pyrrole-2-carboxylic acid.

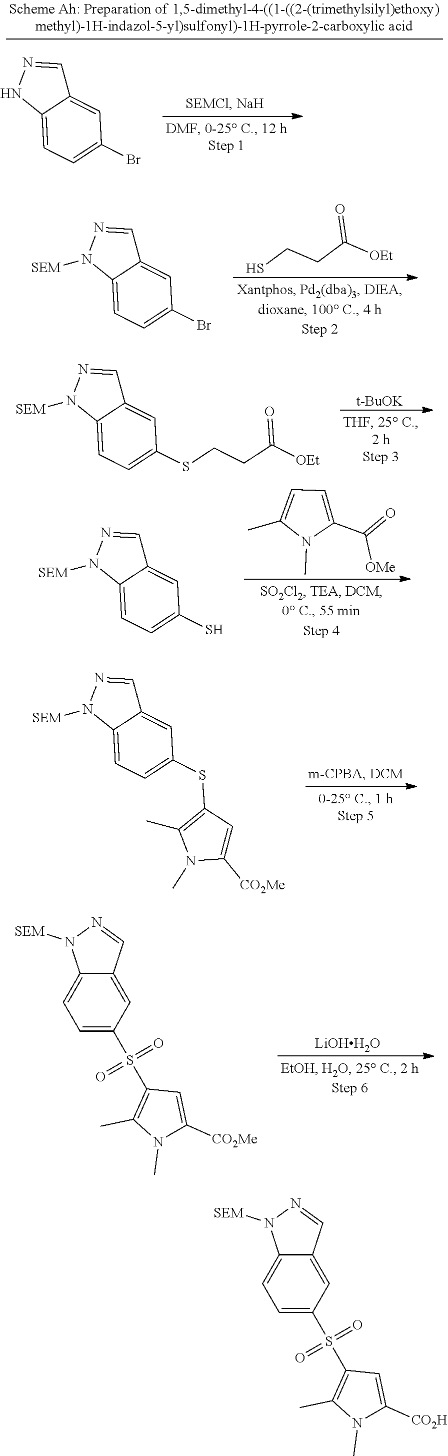

Step 1. At 0° C., to a solution of 5-bromo-1H-indazole (5.00 g, 25.4 mmol, 1 eq) in DMF (50 mL) was added NaH (2.00 g, 50.8 mmol, 60 wt % dispersion in oil) carefully in portions. After stirring the mixture for 1 h, SEM-Cl (8.46 g, 50.8 mmol, 8.98 mL, 2 eq) was added, and the solution was stirred for another 11 h at 25° C. The reaction mixture was quenched by addition H₂O (100 mL) at 0° C. The mixture was extracted with ethyl acetate (50 mL×3). The combined organic layers were washed with brine (50 mL×3), dried over Na₂SO₄, and filtered. The filtrate was concentrated under reduced pressure to give a residue. The residue was purified by flash silica gel chromatography (ISCO®; 40 g SepaFlash® Silica Flash Column, eluent of 0-15% ethyl acetate/petroleum ether gradient at 100 mL/min) to furnish 2-[(5-bromoindazol-1-yl) methoxy]ethyl-trimethyl-silane.

Step 2. A mixture of 2-[(5-bromoindazol-1-yl)methoxy]ethyl-trimethyl-silane (5.20 g, 15.9 mmol, 1 eq), ethyl 3-sulfanylpropanoate (3.20 g, 23.8 mmol, 1.50 eq), DIEA (6.16 g, 47.7 mmol, 8.30 mL, 3 eq), Xantphos (921 mg, 1.59 mmol, 0.1 eq) and Pd₂(dba)₃ (1.45 g, 1.59 mmol, 0.1 eq) in dioxane (50 mL) was degassed and purged with N₂ (3×). The mixture was stirred at 100° C. for 4 h under a N₂ atmosphere. The mixture was filtered, and the filtrate was concentrated under reduced pressure to give a residue. The residue was purified by flash silica gel chromatography (ISCO®; 40 g SepaFlash® Silica Flash Column, eluent of 0~10% ethyl acetate/petroleum ether gradient at 100 mL/min) to furnish ethyl 3-[1-(2-trimethylsilylethoxymethyl)indazol-5-yl]sulfanylpropanoate.

Step 3. A mixture of ethyl 3-[1-(2-trimethylsilylethoxymethyl)indazol-5-yl]sulfanylpropanoate (3.00 g, 7.88 mmol, 1 eq), t-BuOK (2.65 g, 23.7 mmol, 3 eq) in THE (30 mL) was degassed and purged with N₂ (3×). The mixture was stirred at 25° C. for 2 h under a N₂ atmosphere. The pH of the reaction mixture was adjusted to 4 by addition of aqueous HCl (2 M). The mixture was extracted with EtOAc (30 mL×3). The combined organic layers were washed with brine (300 mL), dried over Na₂SO₄, and filtered. The filtrate was concentrated under reduced pressure to give a residue. The residue was purified by flash silica gel chromatography (ISCO®; 40 g SepaFlash® Silica Flash Column, eluent of 0~10% ethyl acetate/petroleum ether gradient at 80 mL/min) to furnish 1-(2-trimethylsilylethoxymethyl)indazole-5-thiol.

Step 4. To a solution of 1-(2-trimethylsilylethoxymethyl)indazole-5-thiol (589 mg, 2.10 mmol, 1.1 eq), TEA (9.65 mg, 95.4 μmol, 0.05 eq) and distilled DCM (10 mL) at 0° C., sulfuryl chloride (309 mg, 2.29 mmol, 0.23 mL, 1.2 eq) was carefully added in portions. After stirring the mixture for 10 minutes, ethyl 1,5-dimethylpyrrole-2-carboxylate (319 mg, 1.91 mmol, 1 eq) was then added. The solution was stirred for another 45 min. The reaction mixture was concentrated under reduced pressure to remove solvent. The residue was purified by flash silica gel chromatography (ISCO®; 12 g SepaFlash® Silica Flash Column, eluent of 0-10% ethyl acetate/petroleum ether gradient at 60 mL/min) to furnish ethyl 1,5-dimethyl-4-[1-(2-trimethylsilylethoxymethyl)indazol-5-yl]sulfanyl-pyrrole-2-carboxylate.

Step 5. To a solution of ethyl 1,5-dimethyl-4-[1-(2-trimethylsilylethoxymethyl)indazol-5-yl]ulfanyl-pyrrole-2-carboxylate (520 mg, 1.17 mmol, 1 eq) in DCM (10 mL) was added m-CPBA (629 mg, 2.92 mmol, 80% purity, 2.5 eq) at 0° C. The mixture was stirred at 25° C. for 1 h. The reaction mixture was washed with Na₂SO₃ (40 mL), NaHCO₃ (20 ml), dried over Na₂SO₄, and filtered. The filtrate was concentrated under reduced pressure to give a residue. The residue was purified by flash silica gel chromatography (ISCO®; 12 g SepaFlash® Silica Flash Column, eluent of 0~25% ethyl acetate/petroleum ether gradient at 60 mL/min) to furnish ethyl 1,5-dimethyl-4-[1-(2-trimethylsilylethoxymethyl)indazol-5-yl]sulfonyl-pyrrole-2-carboxylate

Step 6. A mixture of ethyl 1,5-dimethyl-4-[1-(2-trimethylsilylethoxymethyl)indazol-5-yl]sulfonyl-pyrrole-2-carboxylate (125 mg, 0.261 mmol, 1 eq) and LiOH·H₂O (16 mg, 0.39 mmol, 1.5 eq) in EtOH (5 mL) and H₂O (1 mL) was degassed and purged with N₂ (3×). The mixture was stirred at 25° C. for 2 h under a N₂ atmosphere. The mixture was treated with aqueous HCl (2 M, 3 mL) to adjust the pH to 2. The mixture was extracted with ethyl acetate (10 mL×3). The combined organic phase was washed with brine (10 mL), dried with anhydrous Na₂SO₄, and filtered. The filtrate was concentrated to give 1,5-dimethyl-4-[1-(2-trimethylsilylethoxymethyl)indazol-5-yl]sulfonyl-pyrrole-2-carboxylic acid.

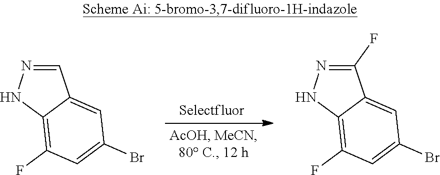 [SMILES: Fc1cc(Br)cc2cn[nH]c12 ; Fc1n[nH]c2c(F)cc(Br)cc12]

A mixture of 5-bromo-7-fluoro-1H-indazole (5.00 g, 23.3 mmol, 1 eq), Selectfluor (12.4 g, 34.9 mmol, 1.5 eq), in MeCN (160 mL) and AcOH (16 mL) was degassed and purged with N₂ for (3×). The mixture was stirred at 80° C. for 12 h under a N₂ atmosphere. The reaction mixture was partitioned between H₂O (20 mL), and the mixture was extracted with ethyl acetate (10 mL×3). The combined organic phase was washed with a brine solution (20 mL), dried over Na₂SO₄, and filtered. The filtrate was concentrated under reduced pressure to give a residue. The residue was purified by column chromatography (SiO₂, petroleum ether/ethyl acetate=1/0 to 5/1) to furnish 5-bromo-3,7-difluoro-1H-indazole.

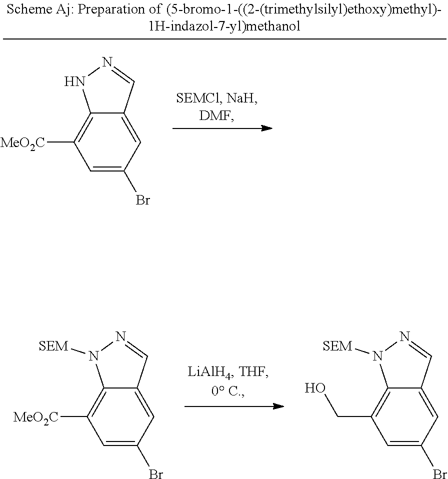 [SMILES: COC(=O)c1cc(Br)cc2cn[nH]c12 ; COC(=O)c1cc(Br)cc2cnn(COCC[Si](C)(C)C)c12 ; C[Si](C)(C)CCOCn1ncc2cc(Br)cc(CO)c21]

At 0° C., to a solution of methyl 5-bromo-1H-indazole-7-carboxylate (2.00 g, 7.84 mmol, 1 eq) in DMF (20 mL) was added NaH (627 mg, 15.7 mmol, 60 wt % dispersion in oil) carefully in portions. The solution was allowed to stir for 0.5 h. SEMCl (2.61 g, 15.7 mmol, 2.78 mL, 2 eq) was added, and the solution was stirred for another 2 h at 25° C. The reaction mixture was quenched with H₂O (100 mL). The mixture was extracted with EtOAc (50 mL×3). The combined organic layers were washed with brine (100 mL), dried over Na₂SO₄, and filtered. The filtrate was concentrated under reduced pressure. The residue was purified by flash silica gel chromatography (ISCO®; 12 g SepaFlash® silica flash column, eluent of 3-10% ethyl acetate/petroleum ether gradient at 50 mL/min) to furnish methyl 5-bromo-1-(2-trimethylsilylethoxymethyl) indazole-7-carboxylate.

To a solution of methyl 5-bromo-1-(2-trimethylsilylethoxymethyl)indazole-7-carboxylate (1.60 g, 4.15 mmol, 1 eq) in THE (20 mL) was added LiAlH₄ (2.5 M, 2.99 mL, 1.8 eq) at 0° C. in small portions. The reaction mixture was stirred at 0° C. for 0.5 h and then warmed to 20° C. for 0.5 h. The reaction mixture was quenched sequentially with H₂O (0.3 mL), 15% NaOH (0.3 mL) and H₂O (0.9 mL). The mixture was filtered and concentrated under reduced pressure to furnish [5-bromo-1-(2-trimethylsilylethoxymethyl)indazol-7-yl]methanol. The material was used without further purification.

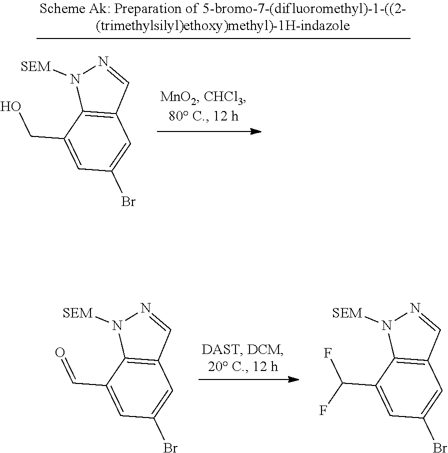 [SMILES: C[Si](C)(C)CCOCn1ncc2cc(Br)cc(CO)c21 ; C[Si](C)(C)CCOCn1ncc2cc(Br)cc(C=O)c21 ; C[Si](C)(C)CCOCn1ncc2cc(Br)cc(C(F)F)c21]

To a solution of [5-bromo-1-(2-trimethylsilylethoxymethyl)indazol-7-yl]methanol (3.10 g, 8.68 mmol, 1 eq) in CHCl₃ (90 mL) was added MnO₂ (7.54 g, 86.8 mmol, 10 eq). The mixture was stirred at 80° C. for 12 h. The reaction mixture was filtered and concentrated under reduced pressure to furnish 5-bromo-1-(2-trimethylsilylethoxymethyl)indazole-7-carbaldehyde. The material was used without further purification.

To a solution of 5-bromo-1-(2-trimethylsilylethoxymethyl)indazole-7-carbaldehyde (2.45 g, 6.90 mmol, 1 eq) in DCM (60 mL) was added DAST (4.45 g, 27.6 mmol, 3.64 mL, 4 eq). The mixture was stirred at 20° C. for 12 h. The reaction mixture was concentrated under reduced pressure to remove the solvent. The residue was purified by flash silica gel chromatography (ISCO®; 20 g SepaFlash® silica flash column, eluent of 0 to 100% ethyl acetate/petroleum ether gradient at 60 mL/min) to furnish 2-[[5-bromo-7-(difluoromethyl)indazol-1-yl]methoxy]ethyl-trimethyl-silane.

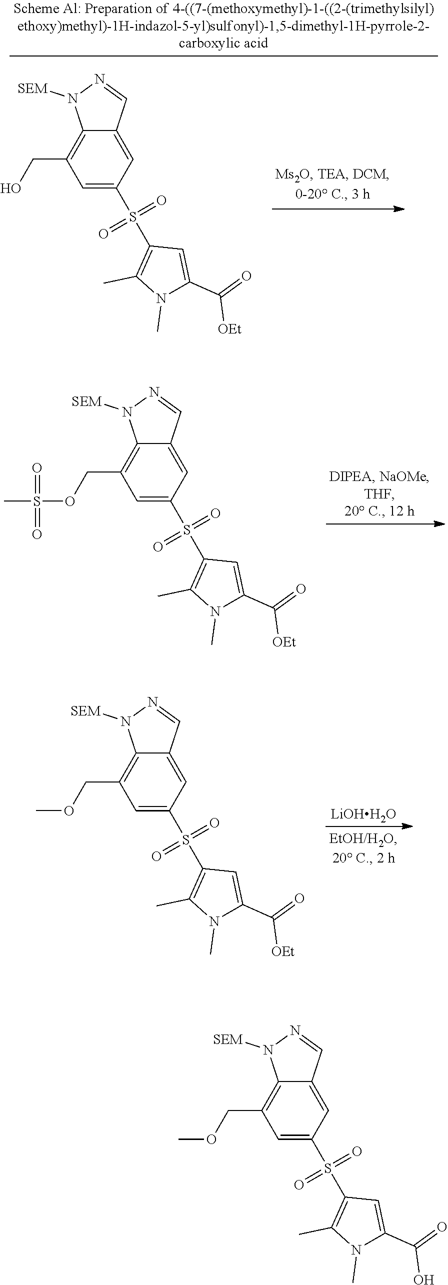 [SMILES: CCOC(=O)c1cc(S(=O)(=O)c2cc(CO)c3c(cnn3COCC[Si](C)(C)C)c2)c(C)n1C ; CCOC(=O)c1cc(S(=O)(=O)c2cc(COS(C)(=O)=O)c3c(cnn3COCC[Si](C)(C)C)c2)c(C)n1C ; CCOC(=O)c1cc(S(=O)(=O)c2cc(COC)c3c(cnn3COCC[Si](C)(C)C)c2)c(C)n1C ; "LiOH·H2O" ; COCc1cc(S(=O)(=O)c2cc(C(=O)O)n(C)c2C)cc2cnn(COCC[Si](C)(C)C)c12]

To a solution of ethyl 4-[7-(hydroxymethyl)-1-(2-trimethylsilylethoxymethyl)indazol-5-yl]sulfonyl-1,5-dimethyl-pyrrole-2-carboxylate (300 mg, 590 mol, 1 eq) in DCM (1 mL) was added methylsulfonyl methanesulfonate (123 mg, 709 mol, 1.2 eq) and TEA (90 mg, 890 mol, 123 μL, 1.5 eq) at 0° C. The mixture was stirred at 20° C. for 3 h. The reaction mixture was concentrated under reduced pressure to furnish ethyl 1,5-dimethyl-4-[7-(methylsulfonyloxymethyl)-1-(2-trimethylsilylethoxymethyl)indazol-5-yl]sulfonyl-pyrrole-2-carboxylate. The material was used without further purification.

To a solution of ethyl 1,5-dimethyl-4-[7-(methylsulfonyloxymethyl)-1-(2-trimethylsilylethoxymethyl)indazol-5-yl]sulfonyl-pyrrole-2-carboxylate (320 mg, 546 mol, 1 eq) in THF (5 mL) was added DIPEA (106 mg, 819 mol, 143 μL, 1.5 eq) and NaOMe (148 mg, 2.73 mmol, 5 eq). The mixture was stirred at 20° C. for 12 h. The mixture was poured into H₂O (20 mL). The mixture was extracted with DCM (20 ml×3). The combined organic layers were washed with brine (20 mL), dried over Na₂SO₄, and filtered. The filtrate was concentrated under reduced pressure. The residue was purified by column chromatography (SiO₂, petroleum ether/ethyl acetate=5/1) to furnish ethyl 4-[7-(methoxymethyl)-1-(2-trimethylsilylethoxymethyl) indazol-5-yl]sulfonyl-1,5-dimethyl-pyrrole-2-carboxylate.

To a solution of ethyl 4-[7-(methoxymethyl)-1-(2-trimethylsilylethoxymethyl) indazol-5-yl]sulfonyl-1,5-dimethyl-pyrrole-2-carboxylate (280 mg, 537 mol, 1 eq) in H₂O (1 mL) and EtOH (3 mL) was added LiOH·H₂O (34 mg, 0.81 mol, 1.5 eq). The mixture was stirred at 20° C. for 2 h. The reaction mixture was concentrated under reduced pressure. The mixture was poured into H₂O (4 mL), the pH of the mixture was adjusted to 5 by addition of aqueous HCl (2 N). The white precipitate was collected in a Buchner funnel and dried to furnish 4-[7-(methoxymethyl)-1-(2-trimethylsilylethoxymethyl) indazol-5-yl]sulfonyl-1,5-dimethyl-pyrrole-2-carboxylic acid.

Scheme Am: Preparation of 5-bromo-1-((2-(trimethylsilyl)ethoxy)methyl)-1H-indazole-7-carbonitrile

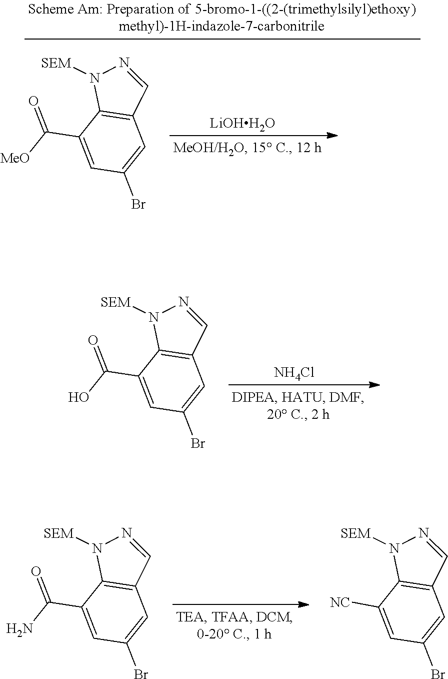 [SMILES: COC(=O)c1cc(Br)cc2cnn(COCC[Si](C)(C)C)c12 ; "LiOH·H2O" ; C[Si](C)(C)CCOCn1ncc2cc(Br)cc(C(=O)O)c21 ; "NH4Cl" ; C[Si](C)(C)CCOCn1ncc2cc(Br)cc(C(N)=O)c21 ; C[Si](C)(C)CCOCn1ncc2cc(Br)cc(C#N)c21]

A mixture of methyl 5-bromo-1-(2-trimethylsilylethoxymethyl)indazole-7-carboxylate (4.39 g, 11.4 mmol, 1 eq) and LiOH·H₂O (717 mg, 17.1 mmol, 1.5 eq) in MeOH (50 mL) and H₂O (10 mL) was degassed and purged with N₂ (3×). The mixture was stirred at 15° C. for 12 h under a N₂ atmosphere. The reaction mixture was concentrated under reduced pressure to remove solvent. The residue was diluted with H₂O (20 mL), and the pH was adjusted to 3 by addition of aqueous HCl (2M). The solid was collected by filtration and dried to furnish 5-bromo-1-(2-trimethylsilylethoxymethyl)indazole-7-carboxylic acid. The material was used without further purification.

A mixture of 5-bromo-1-(2-trimethylsilylethoxymethyl)indazole-7-carboxylic acid (4.40 g, 11.9 mmol, 1 eq), NH₄Cl (3.17 g, 59.3 mmol, 5 eq), HATU (5.41 g, 14.2 mmol, 1.2 eq) and DIPEA (7.66 g, 59.3 mmol, 10.3 mL, 5 eq) in DMF (80 mL) was degassed and purged with N₂ (3×). The mixture was stirred at 20° C. for 2 h under a N₂ atmosphere. The mixture was poured into water (150 mL) and extracted with ethyl acetate (100 mL×3). The combined organic phase was washed with brine (80 mL×2), dried with anhydrous Na₂SO₄, and filtered. The filtrate was concentrated under vacuum. The residue was purified by flash silica gel chromatography (ISCO®; 40 g SepaFlash® silica flash column, eluent of 0 to 40% ethyl acetate/petroleum ether gradient at 100 mL/min) to furnish 5-bromo-1-(2-trimethylsilylethoxymethyl)indazole-7-carboxamide.

To a solution of 5-bromo-1-(2-trimethylsilylethoxymethyl)indazole-7-carboxamide (4.30 g, 11.6 mmol, 1 eq) in DCM (50 mL) was added TFAA (6.10 g, 29.0 mmol, 4.04 mL, 2.5 eq) and TEA (2.94 g, 29.0 mmol, 4.04 mL, 2.5 eq) sequentially at 0° C. The mixture was stirred at 20° C. for 1 h. The reaction mixture was concentrated under reduced pressure to remove solvent. The residue was purified by flash silica gel chromatography (ISCO®; 40 g SepaFlash® silica flash column, eluent of 0 to 15% ethyl acetate/petroleum ether gradient at 100 mL/min) to furnish 5-bromo-1-(2-trimethylsilylethoxymethyl)indazole-7-carbonitrile.

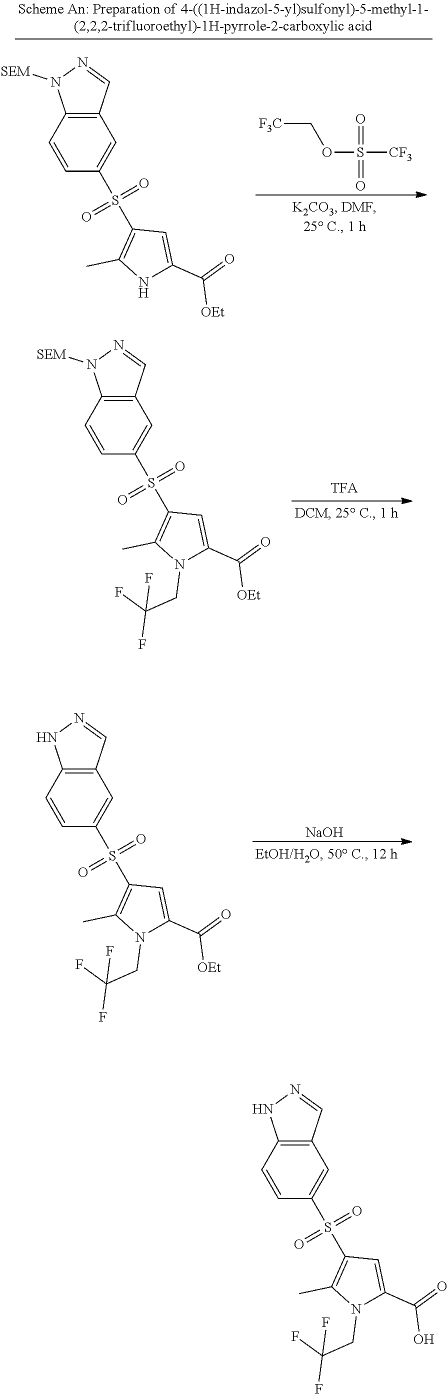 [SMILES: CCOC(=O)c1cc(S(=O)(=O)c2ccc3c(cnn3COCC[Si](C)(C)C)c2)c(C)[nH]1 ; O=S(=O)(OCC(F)(F)F)C(F)(F)F ; CCOC(=O)c1cc(S(=O)(=O)c2ccc3c(cnn3COCC[Si](C)(C)C)c2)c(C)n1CC(F)(F)F ; CCOC(=O)c1cc(S(=O)(=O)c2ccc3[nH]ncc3c2)c(C)n1CC(F)(F)F ; "NaOH" ; Cc1c(S(=O)(=O)c2ccc3[nH]ncc3c2)cc(C(=O)O)n1CC(F)(F)F]

A mixture of ethyl 5-methyl-4-[1-(2-trimethylsilylethoxymethyl)indazol-5-yl]sulfonyl-1H-pyrrole-2-carboxylate (250 mg, 539 mol, 1 eq), 2,2,2-trifluoroethyl trifluoromethanesulfonate (250 mg, 1.08 mmol, 2 eq), K₂CO₃ (149 mg, 1.08 mmol, 2 eq) in DMF (5 mL) was degassed and purged with N₂ (3×). The mixture was stirred at 25° C. for 1 h under a N₂ atmosphere. The mixture was poured into water (20 mL) and extracted with ethyl acetate (15 mL×3). The combined organic phase was washed with brine (15 mL×1), dried with anhydrous Na₂SO₄, and filtered. The filtrate was concentrated under vacuum. The residue was purified by flash silica gel chromatography (ISCO®; 4 g SepaFlash® silica flash column, eluent of 0-50% ethyl acetate/petroleum ether gradient at 40 mL/min) to furnish ethyl 5-methyl-1-(2,2,2-trifluoroethyl)-4-[1-(2-trimethylsilylethoxymethyl)indazol-5-yl]sulfonyl-pyrrole-2-carboxylate.

A mixture of ethyl 5-methyl-1-(2,2,2-trifluoroethyl)-4-[1-(2-trimethylsilylethoxymethyl) indazol-5-yl]sulfonyl-pyrrole-2-carboxylate (290 mg, 531 mol, 1 eq) in TFA (2 mL) and DCM (4 mL) was degassed and purged with N₂ (3×). The mixture was stirred at 25° C. for 1 h under a N₂ atmosphere. The reaction mixture was concentrated under reduced pressure to remove solvent to furnish ethyl 4-(1H-indazol-5-ylsulfonyl)-5-methyl-1-(2,2,2-trifluoroethyl)pyrrole-2-carboxylate.

A mixture of ethyl 4-(1H-indazol-5-ylsulfonyl)-5-methyl-1-(2,2,2-trifluoroethyl)pyrrole-2-carboxylate (300 mg, 722 mol, 1 eq) and NaOH (58 mg, 1.44 mmol, 2 eq) in EtOH (3 mL) and H₂O (1 mL) was degassed and purged with N₂ (3×). The mixture was stirred at 50° C. for 12 h under a N₂ atmosphere. The mixture was poured into water (20 mL) and treated with aqueous HCl(2M) to adjust the pH to 3. The mixture was extracted with ethyl acetate (15 mL×3). The combined organic phase was washed with brine (15 mL×1), dried with anhydrous Na₂SO₄, and filtered. The filtrate was concentrated to furnish 4-(1H-indazol-5-ylsulfonyl)-5-methyl-1-(2,2,2-trifluoroethyl)pyrrole-2-carboxylic acid.

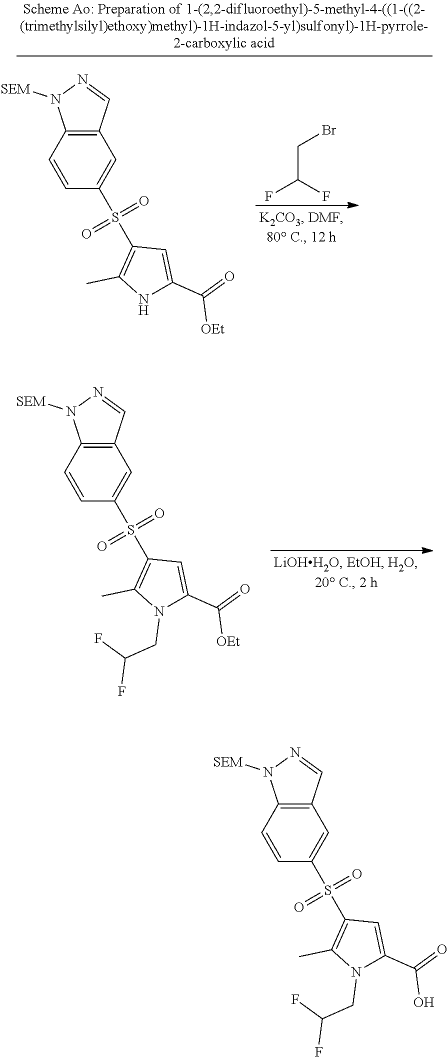 [SMILES: CCOC(=O)c1cc(S(=O)(=O)c2ccc3c(cnn3COCC[Si](C)(C)C)c2)c(C)[nH]1 ; FC(F)CBr ; CCOC(=O)c1cc(S(=O)(=O)c2ccc3c(cnn3COCC[Si](C)(C)C)c2)c(C)n1CC(F)F ; Cc1c(S(=O)(=O)c2ccc3c(cnn3COCC[Si](C)(C)C)c2)cc(C(=O)O)n1CC(F)F]

A mixture of ethyl 5-methyl-4-[1-(2-trimethylsilylethoxymethyl)indazol-5-yl]sulfonyl-1H-pyrrole-2-carboxylate (300 mg, 647 mol, 1 eq), 2-bromo-1,1-difluoro-ethane (469 mg, 3.24 mmol, 5 eq), K₂CO₃ (179 mg, 1.29 mmol, 2 eq) in DMF (5 mL) was degassed and purged with N₂ (3×). The mixture was stirred at 80° C. for 12 h under a N₂ atmosphere. The reaction mixture was diluted with H₂O (40 mL) and extracted with EtOAc (20 mL×3). The combined organic layers were washed with brine (40 mL), dried over Na₂SO₄, and filtered. The filtrate was concentrated under reduced pressure to give a residue. The residue was purified by flash silica gel chromatography (ISCO®; 4 g SepaFlash® silica flash column, eluent of 8 to 15% ethyl acetate/petroleum ether gradient at 40 mL/min) to furnish ethyl 1-(2,2-difluoroethyl)-5-methyl-4-[1-(2-trimethylsilylethoxymethyl)indazol-5-yl]sulfonyl-pyrrole-2-carboxylate.

To a solution of ethyl 1-(2,2-difluoroethyl)-5-methyl-4-[1-(2-trimethylsilylethoxymethyl) indazol-5-yl]sulfonyl-pyrrole-2-carboxylate (400 mg, 758 mol, 1 eq) in EtOH (3 mL) and H₂O (1 mL) was added LiOH·H₂O (64 mg, 1.5 mmol, 2 eq). The mixture was stirred at 20° C. for 2 h. The reaction mixture was concentrated under reduced pressure to remove solvent. The residue was diluted with H₂O (3 mL), and the pH was adjusted to 3 by addition of aqueous HCl (2M). The mixture was extracted with EtOAc (10 mL×3). The combined organic layers were washed with brine (20 mL), dried over Na₂SO₄, and filtered. The filtrate was concentrated under reduced pressure to furnish 1-(2,2-difluoroethyl)-5-methyl-4-[1-(2-trimethylsilylethoxymethyl) indazol-5-yl]sulfonyl-pyrrole-2-carboxylic acid.

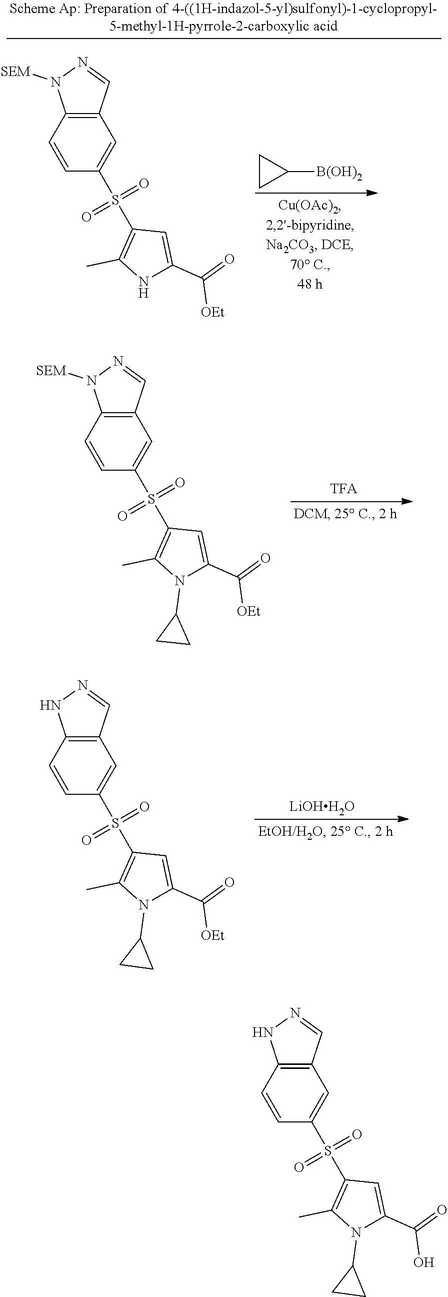 [SMILES: CCOC(=O)c1cc(S(=O)(=O)c2ccc3c(cnn3COCC[Si](C)(C)C)c2)c(C)[nH]1 ; OB(O)C1CC1 ; CCOC(=O)c1cc(S(=O)(=O)c2ccc3c(cnn3COCC[Si](C)(C)C)c2)c(C)n1C1CC1 ; CCOC(=O)c1cc(S(=O)(=O)c2ccc3[nH]ncc3c2)c(C)n1C1CC1 ; "LiOH·H2O" ; Cc1c(S(=O)(=O)c2ccc3[nH]ncc3c2)cc(C(=O)O)n1C1CC1]

A mixture of ethyl 5-methyl-4-[1-(2-trimethylsilylethoxymethyl)indazol-5-yl]sulfonyl-1H-pyrrole-2-carboxylate (290 mg, 626 mol, 1 eq), cyclopropylboronic acid (215 mg, 2.50 mmol, 4 eq), Na₂CO₃ (265 mg, 2.50 mmol, 4 eq), 2-(2-pyridyl)pyridine (195 mg, 1.25 mmol, 2 eq) and Cu(OAc)₂ (227 mg, 1.25 mmol, 2 eq) in DCE (5 mL) was degassed and purged with N₂ (3×). The mixture was stirred at 70° C. for 48 h under an O₂ atmosphere (15 Psi). The mixture was filtered, and the filtrate was concentrated under reduced pressure to give a residue. The residue was purified by flash silica gel chromatography (ISCO®; 4 g SepaFlash® silica flash column, eluent of 0-30% ethyl acetate/petroleum ether gradient at 40 mL/min) to furnish ethyl 1-cyclopropyl-5-methyl-4-[1-(2-trimethylsilylethoxymethyl)indazol-5-yl]sulfonyl-pyrrole-2-carboxylate.

A mixture of ethyl 1-cyclopropyl-5-methyl-4-[1-(2-trimethylsilylethoxymethyl)indazol-5-yl]sulfonyl-pyrrole-2-carboxylate (240 mg, 476 mol, 1 eq) in TFA (0.5 mL) and DCM (1 mL) was degassed and purged with N₂ (3×). The mixture was stirred at 25° C. for 2 h under a N₂ atmosphere. The mixture was poured extracted with ethyl acetate (10 mL). The combined organic phase was washed with 15% NaOH (15 mL×3), dried with anhydrous Na₂SO₄, and filtered. The filtrate was concentrated to furnish ethyl 1-cyclopropyl-4-(1H-indazol-5-ylsulfonyl)-5-methyl-pyrrole-2-carboxylate.

A mixture of ethyl 1-cyclopropyl-4-(1H-indazol-5-ylsulfonyl)-5-methyl-pyrrole-2-carboxylate (180 mg, 482 mol, 1 eq), LiOH·H₂O (61 mg, 1.45 mmol, 3 eq) in EtOH (1 mL) and H₂O (0.2 mL) was degassed and purged with N₂ (3×). The mixture was stirred at 25° C. for 2 h under a N₂ atmosphere. The reaction mixture was concentrated under reduced pressure to remove solvent. The residue was diluted with H₂O (2 mL). The pH of the aqueous phase was adjust to 1 by addition of aqueous HCl(2M). The solid was collected by filtration and dried under reduced pressure to furnish 1-cyclopropyl-4-(1H-indazol-5-ylsulfonyl)-5-methyl-pyrrole-2-carboxylic acid.

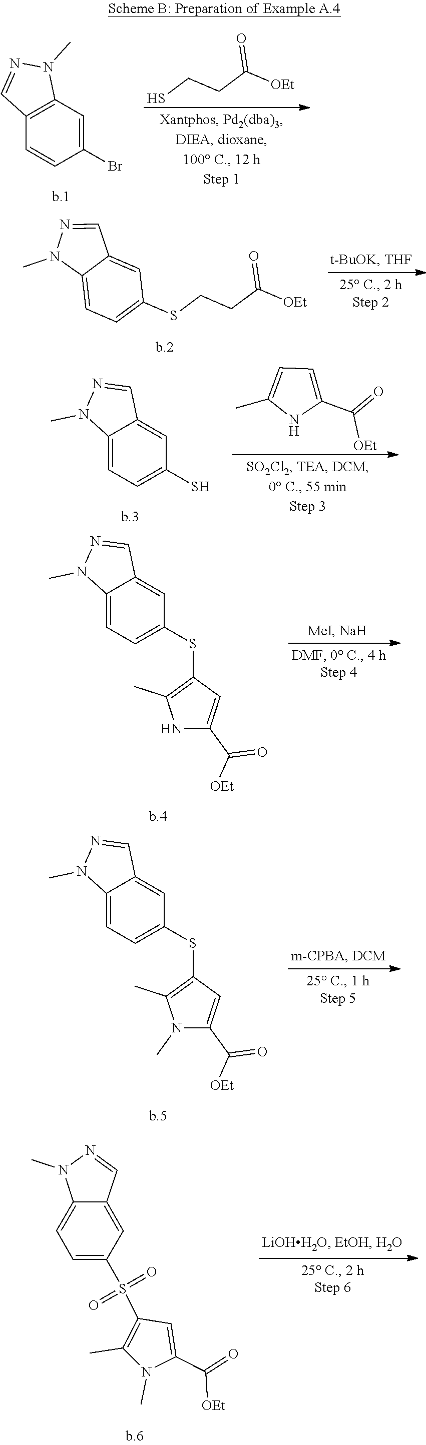

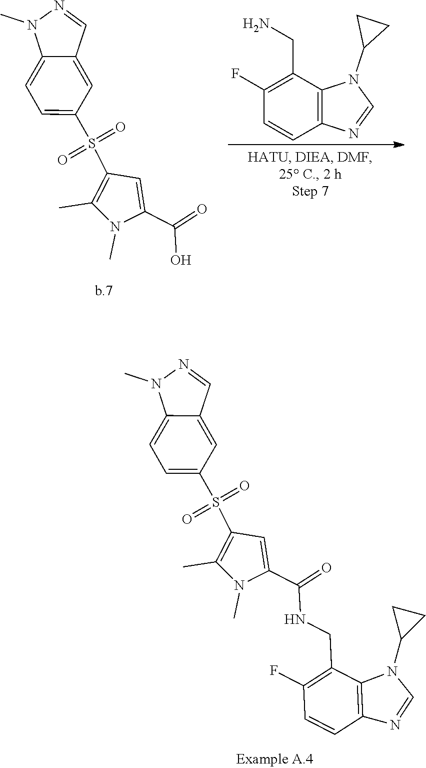 [SMILES: Cc1c(S(=O)(=O)c2ccc3c(cnn3C)c2)cc(C(=O)O)n1C ; NCc1c(F)ccc2ncn(C3CC3)c12 ; Cc1c(S(=O)(=O)c2ccc3c(cnn3C)c2)cc(C(=O)NCc2c(F)ccc3ncn(C4CC4)c23)n1C]

The acid b.7 was prepared in a similar fashion to that described previously using the appropriate reagents in Step 1 (halide b.1) and Step 2 (amine) (Scheme B).

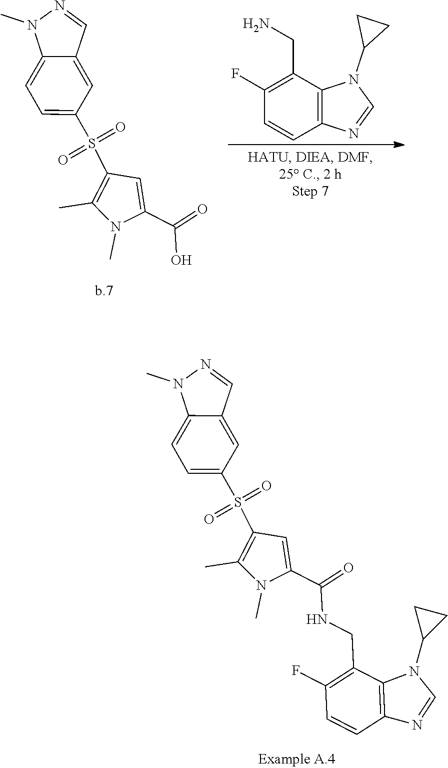 [SMILES: Cc1c(S(=O)(=O)c2ccc3c(cnn3C)c2)cc(C(=O)O)n1C ; NCc1c(F)ccc2ncn(C3CC3)c12 ; Cc1c(S(=O)(=O)c2ccc3c(cnn3C)c2)cc(C(=O)NCc2c(F)ccc3ncn(C4CC4)c23)n1C]

A mixture of 1,5-dimethyl-4-(1-methylindazol-5-yl)sulfonyl-pyrrole-2-carboxylic acid (70 mg, 0.21 mmol, 1 eq), (3-cyclopropyl-5-fluoro-benzimidazol-4-yl)methanamine (86 mg, 0.42 mmol, 2 eq), HATU (120 mg, 0.315 mmol, 1.5 eq) and DIPEA (54 mg, 0.42 mmol, 2 eq) in DMF (1 mL) was degassed and purged with N₂ (3×). The mixture was stirred at 20° C. for 12 hr. The mixture was poured into water (20 mL), and the mixture was extracted with ethyl acetate (15 mL×3). The combined organic phase was washed with brine (15 mL), dried with anhydrous Na₂SO₄, and filtered. The filtrate was concentrated. The residue was purified by preparative-HPLC (column: Waters Xbridge Prep OBD C18 150*40 mm*10 um;mobile phase: [water(NH₄HCO₃)-ACN];B %: 25%-45%,8 min) to furnish N-[(3-cyclopropyl-5-fluoro-benzimidazol-4-yl)methyl]-1,5-dimethyl-4-(1-methylindazol-5-yl)sulfonyl-pyrrole-2-carboxamide. LCMS: (M+H⁺): 521.0; ¹H NMR: (400 MHz, DMSO-d6) δ 8.58 (t, J=4.3 Hz, 1H), 8.37 (s, 1H), 8.28-8.17 (m, 2H), 7.81-7.74 (m, 1H), 7.73-7.67 (m, 1H), 7.62 (dd, J=4.8, 8.8 Hz, 1H), 7.28-7.19 (m, 1H), 7.10 (dd, J=8.9, 10.6 Hz, 1H), 4.96 (br d, J=3.3 Hz, 2H), 4.06 (s, 3H), 3.79-3.72 (m, 4H), 2.37 (s, 3H), 1.26-1.17 (m, 2H), 1.10-1.01 (m, 2H).

The following examples can be prepared in a similar fashion to that described above in Step 7 of Scheme B using the appropriate acid and amine for the amide coupling step.

TABLE C

| | | | | ¹H NMR |
|---|---|---|---|---|
| Ex. | Acid | Amine | Structure | LCMS |
| A.18 | 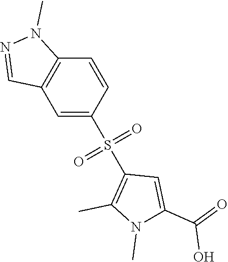 | 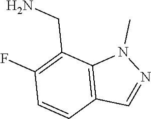 | 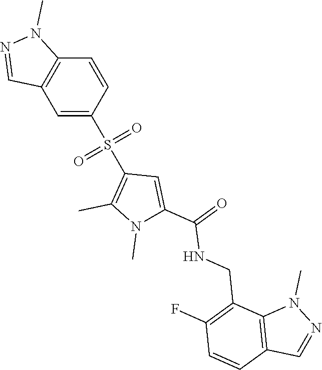 [SMILES: Cc1c(S(=O)(=O)c2ccc3c(cnn3C)c2)cc(C(=O)NCc2c(F)ccc3cnn(C)c23)n1C] | LCMS (M + H⁺): 495.0; 1H NMR (400 MHz, METHANOL- d4) δ 8.52- 8.37 (m, 1H), 8.22-8.14 (m, 1H), 8.06-8.01 (m, 1H), 7.85- 7.78 (m, 1H), 7.67 (d, J = 8.9 Hz, 1H), 7.30 (dd, J = 4.9, 7.9 Hz, 1H), 7.19 (s, 1H), 6.78 (dd, J = 8.0, 9.6 Hz, 1H), 4.93 (s, 2H), 4.29 (s, 3H), 4.11-4.07 (m, 3H), 3.83-3.78 (m, 3H), 2.49-2.43 (m, 3H) |
| A.19 | 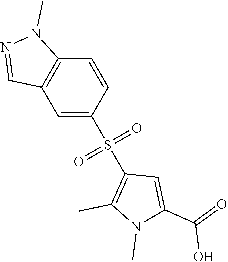 | 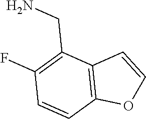 [SMILES: NCc1c(F)ccc2occc12] | 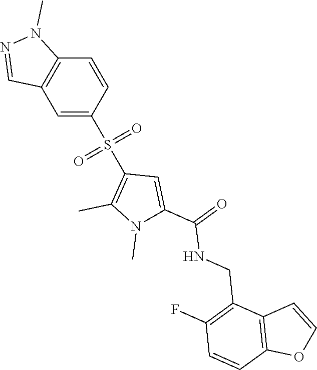 [SMILES: Cc1c(S(=O)(=O)c2ccc3c(cnn3C)c2)cc(C(=O)NCc2c(F)ccc3occc23)n1C] | LCMS (M + H⁺): 481.1; 1H NMR (400 MHz, DMSO- d6) δ 8.86-8.79 (m, 1H), 8.35 (d, J = 0.7 Hz, 1H), 8.26 (s, 1H), 8.03 (d, J = 2.2 Hz, 1H), 7.83-7.77 (m, 1H), 7.74-7.67 (m, 1H), 7.54 (dd, J = 3.5, 8.8 Hz, 1H), 7.23 (s, 1H), 7.16 (d, J = 10.1 Hz, 1H), 7.13-7.10 (m, 1H), 4.62 (d, J = 5.5 Hz, 2H), 4.06 (s, 3H), 3.74 (s, 3H), 2.37 (s, 3H) |
| A.20 | 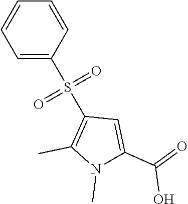 | 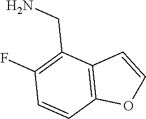 [SMILES: NCc1c(F)ccc2occc12] | 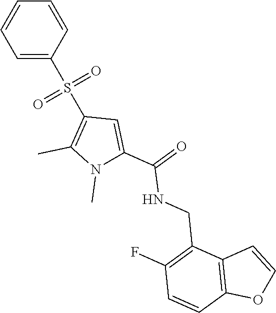 [SMILES: Cc1c(S(=O)(=O)c2ccccc2)cc(C(=O)NCc2c(F)ccc3occc23)n1C] | LCMS (M + H⁺): 427.0; 1H NMR (400 MHz, DMSO-d6) δ 8.83 (t, J = 5.6 Hz, 1H), 8.03 (d, J = 2.3 Hz, 1H), 7.86- 7.75 (m, 2H), 7.68-7.49 (m, 4H), 7.25- 7.06 (m, 3H), 4.62 (d, J = 5.6 Hz, 2H), 3.76 (s, 3H), 2.38 (s, 3H) |
| A.21 | 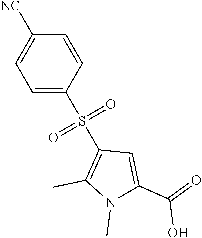 (prepared by Thiol route - Scheme D) | 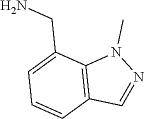 | 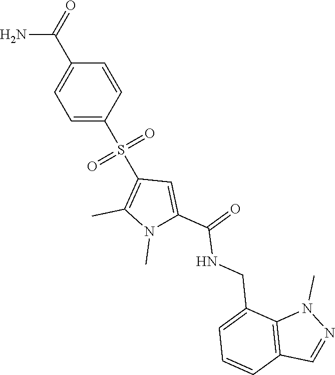 [SMILES: Cc1c(S(=O)(=O)c2ccc(C(N)=O)cc2)cc(C(=O)NCc2cccc3cnn(C)c23)n1C] | LCMS (M + H⁺): 466.0; 1H NMR (400 MHz, METHANOL- d4) δ 8.07- 7.86 (m, 5H), 7.72-7.65 (m, 1H), 7.34 (br d, J = 6.9 Hz, 1H), 7.22-7.18 (m, 1H), 7.15-7.06 (m, 1H), 4.99 (s, 2H), 4.30 (s, 3H), 3.82 (s, 3H), 2.46 (s, 3H) |
| | | | (cyano group | |
| | | | hydrolyzed during | |
| | | | amide coupling step) | |
| A22 | 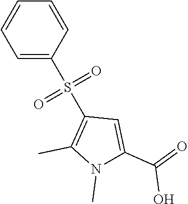 | 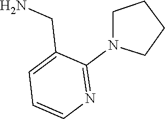 [SMILES: NCc1cccnc1N1CCCC1] | 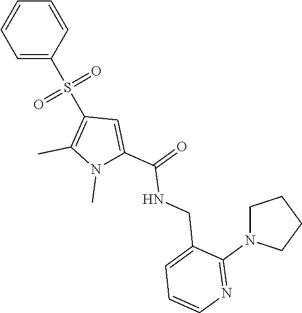 [SMILES: Cc1c(S(=O)(=O)c2ccccc2)cc(C(=O)NCc2cccnc2N2CCCC2)n1C] | LCMS (M + H⁺): 439.1; 1H NMR (400 MHz, DMSO-d6) δ 8.70 (br t, J = 5.5 Hz, 1H), 8.00 (dd, J = 1.6, 4.8 Hz, 1H), 7.88-7.79 (m, 2H), 7.67-7.55 (m, 3H), 7.41 (dd, J = 1.6, 7.3 Hz, 1H), 7.27 (s, 1H), 6.70 (dd, J = 4.8, 7.4 Hz, 1H), 4.38 (br d, J = 5.5 Hz, 2H), 3.75 (s, 3H), 3.47-3.43 (m, |
| | | | | 4H), 2.43-2.37 |
| | | | | (m, 3H), 1.85 (br |
| | | | | t, J = 6.6 Hz, |
| | | | | 4H) |
| A.23 | 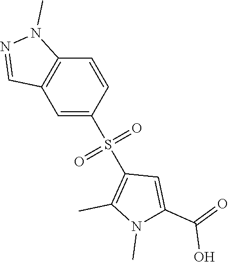 | 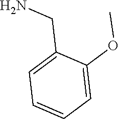 | 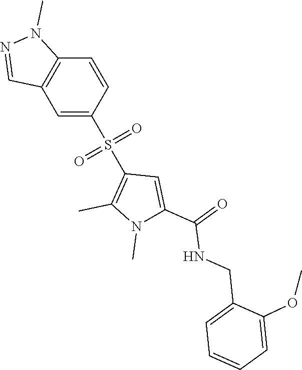 [SMILES: COc1ccccc1CNC(=O)c1cc(S(=O)(=O)c2ccc3c(cnn3C)c2)c(C)n1C] | LCMS (M + H⁺): 453.2; 1H NMR (400 MHz, DMSO- d6) δ 8.66 (t, J = 5.9 Hz, 1H), 8.38 (s, 1H), 8.28 (s, 1H), 7.84-7.80 (m, 1H), 7.78- 7.74 (m, 1H), 7.30 (s, 1H), 7.26-7.20 (m, 1H), 7.15 (d, J = 7.0 Hz, 1H), 6.98 (d, J = 7.9 Hz, 1H), 6.93- 6.87 (m, 1H), 4.33 (d, J = 5.9 Hz, 2H), 4.08 (s, 3H),3.81 (s, 3H), 3.74 (s, 3H), 2.40 (s, 3H) |
| A.25 | 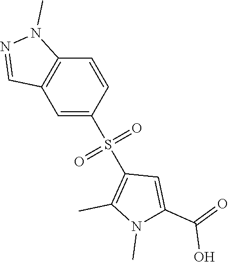 | 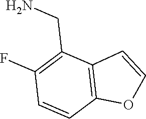 [SMILES: NCc1c(F)ccc2occc12] | 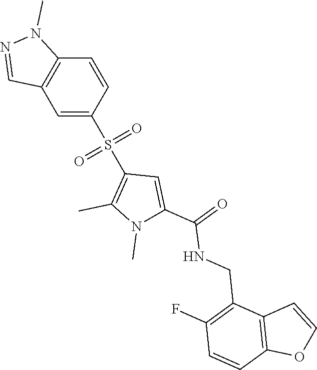 | LCMS (M + H⁺): 467.0; 1H NMR (400 MHz, DMSO-d6) δ 12.24 (br s, 1H), 8.74 (br t, J = 5.69 Hz, 1H), 8.33 (s, 1H), 8.26 (s, 1H), 8.02 (d, J = 1.75 Hz, 1H), 7.82-7.67 (m, 2H), 7.54 (dd, J = 8.76, 3.63 Hz, 1H), 7.22- 7.11 (m, 2H), 7.08 (d, J = 1.13 Hz, 1H), 4.64 (br d, J = 5.50 Hz, 2H), 4.06 (s, 3H), 2.34 (s, 3H) |
| A.26 | 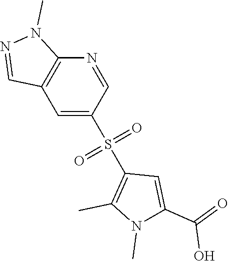 (prepared using route like that shown in Scheme B) | 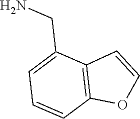 [SMILES: NCc1cccc2occc12] | 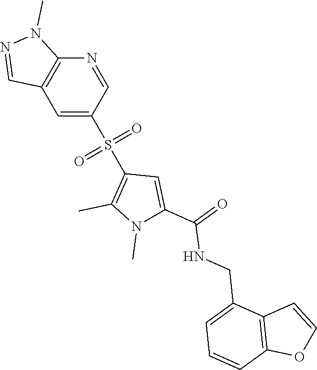 | LCMS (M + H⁺): 482.0; 1H NMR (400 MHz, DMSO- d6) δ 8.91 (d, J = 2.1 Hz, 1H), 8.82 (br t, J = 5.5 Hz, 1H), 8.76 (d, J = 2.1 Hz, 1H), 8.33 (s, 1H), 8.03 (d, J = 2.0 Hz, 1H), 7.54 (dd, J = 3.6, 8.9 Hz, 1H), 7.24 (s, 1H), 7.20-7.08 (m, 2H), 4.62 (d, J = 5.5 Hz, 2H), 4.07 (s, 3H), 3.75 (s, 3H), 2.41 (s, 3H) |
| A.27 | 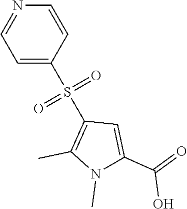 (prepared using route like that shown in Scheme C) | 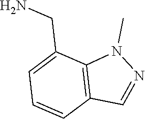 | 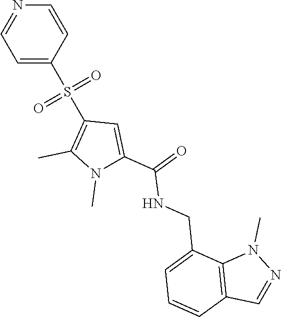 [SMILES: Cc1c(S(=O)(=O)c2ccncc2)cc(C(=O)NCc2cccc3cnn(C)c23)n1C] | LCMS (M + H⁺): 424.0; 1H NMR (400 MHz, DMSO-d6) δ 8.89-8.78 (m, 3H), 8.03 (s, 1H), 7.79-7.72 (m, 2H), 7.68 (d, J = 7.6 Hz, 1H), 7.35 (s, 1H), 7.29-7.23 (m, 1H), 7.13-7.04 (m, 1H), 4.89 (d, J = 5.1 Hz, 2H), 4.28-4.19 (m, 3H), 3.82- 3.78 (m, 3H), 2.42 (s, 3H) |
| A.28 | 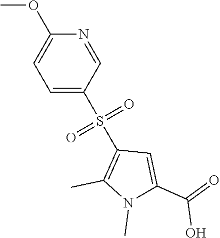 (prepared using route like that shown in Scheme B) | 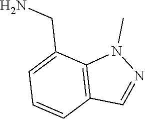 | 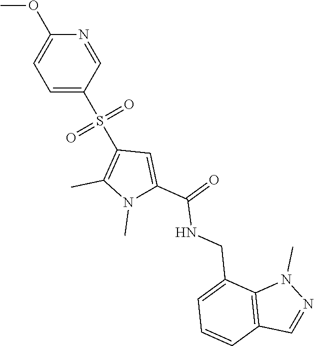 [SMILES: COc1ccc(S(=O)(=O)c2cc(C(=O)NCc3cccc4cnn(C)c34)n(C)c2C)cn1] | LCMS (M + H⁺): 454.0; 1H NMR (400 MHz, DMSO-d6) δ 8.83 (t, J = 5.3 Hz, 1H), 8.64 (d, J = 2.6 Hz, 1H), 8.06-7.99 (m, 2H), 7.67 (d, J = 8.1 Hz, 1H), 7.33-7.24 (m, 2H), 7.08 (t, J = 7.7 Hz, 1H), 6.98 (d, J = 8.8 Hz, 1H), 4.89 (d, J = 5.3 Hz, 2H), 4.25 (s, 3H), 3.91 (s, 3H), 3.78 (s, 3H), 2.41 (s, 3H) |
| A.29 | 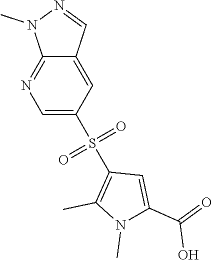 | 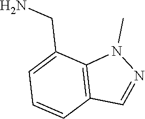 | 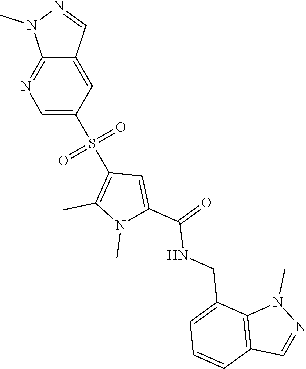 [SMILES: Cc1c(S(=O)(=O)c2cnc3c(cnn3C)c2)cc(C(=O)NCc2cccc3cnn(C)c23)n1C] | LCMS (M + H⁺): 478.1; 1H NMR (400 MHz, DMSO- d6) δ 8.94 (d, J = 2.0 Hz, 1H), 8.83 (br t, J = 5.1 Hz, 1H), 8.78 (d, J = 2.1 Hz, 1H), 8.34 (s, 1H), 8.03 (s, 1H), 7.67 (d, J = 8.0 Hz, 1H), 7.35 (s, 1H), 7.27 (d, J = 7.1 Hz, 1H), 7.08 (t, J = 7.6 Hz, 1H), 4.88 (d, J = 5.1 Hz, 2H), 4.25 (s, 3H), 4.08 (s, 3H), 3.78-3.75 (m, 3H), 2.43 (s, 3H) |
| A.30 | 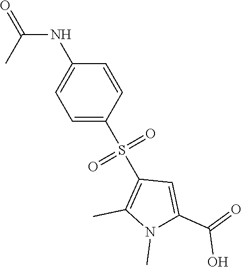 (prepared using route like that shown in Scheme B) | 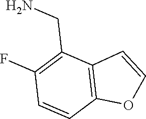 [SMILES: NCc1c(F)ccc2occc12] | 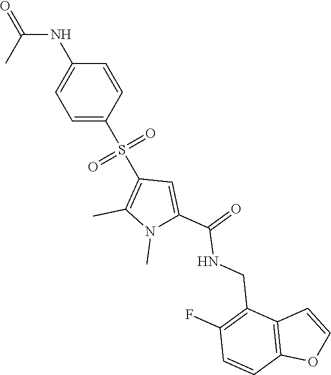 [SMILES: CC(=O)Nc1ccc(S(=O)(=O)c2cc(C(=O)NCc3c(F)ccc4occc34)n(C)c2C)cc1] | LCMS (M + H⁺): 484.1; 1H NMR (400 MHz, METHANOL- d4) δ 7.78 (d, J = 9.0 Hz, 3H), 7.74-7.69 (m, 2H), 7.42 (dd, J = 3.6, 8.9 Hz, 1H), 7.11-6.99 (m, 3H), 4.75 (s, 2H), 3.79 (s, 3H), 2.42 (s, 3H), 2.13 (s, 3H) |

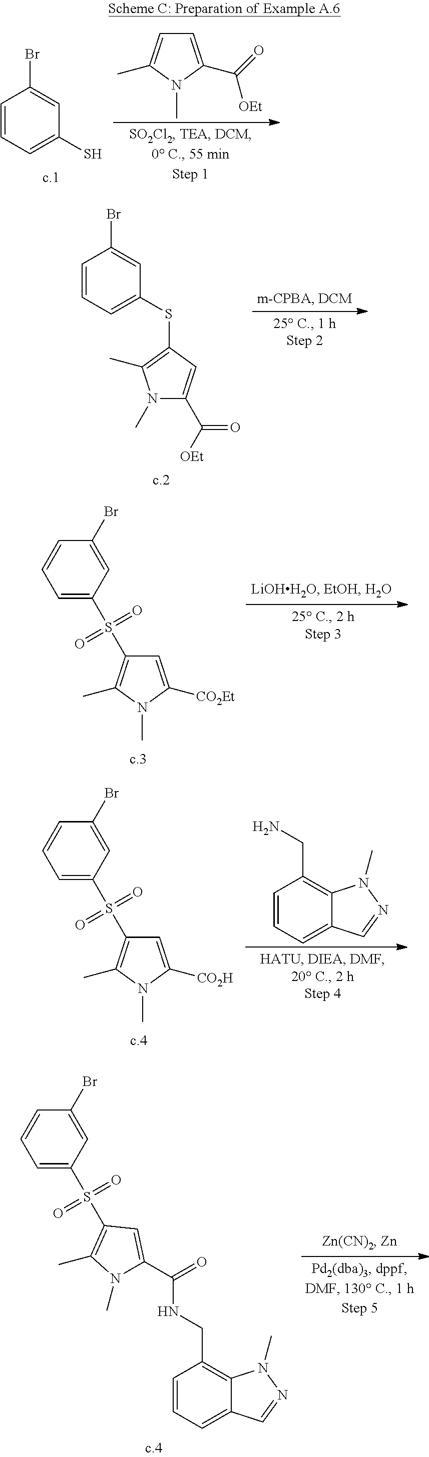

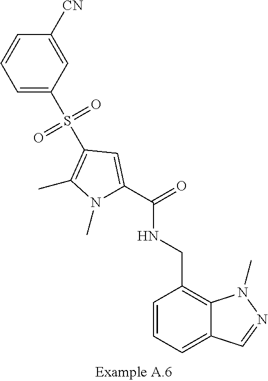 [SMILES: Cc1c(S(=O)(=O)c2cccc(C#N)c2)cc(C(=O)NCc2cccc3cnn(C)c23)n1C]

Step-by-step preparation of Example A.6

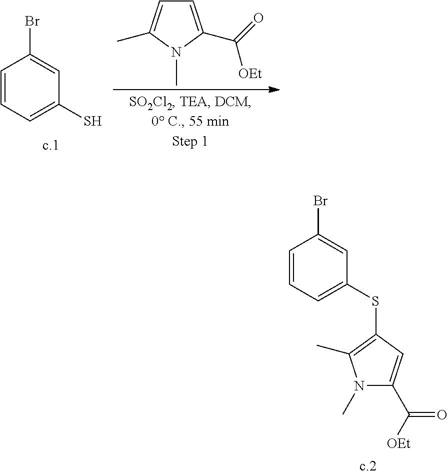 [SMILES: Sc1cccc(Br)c1 ; CCOC(=O)c1ccc(C)n1C ; CCOC(=O)c1cc(Sc2cccc(Br)c2)c(C)n1C]

To a solution of 3-bromobenzenethiol (2.00 g, 10.6 mmol, 1.09 mL, 1 eq) in DCM (40 mL) was added TEA (54 mg, 0.53 mmol, 0.05 eq) and sulfuryl chloride (1.43 g, 10.6 mmol, 1.06 mL, 1 eq) at 0° C. After stirring the mixture at 0° C. for 0.5 h, ethyl 1,5-dimethylpyrrole-2-carboxylate (2.12 g, 12.7 mmol, 1.2 eq) was added, and the mixture was stirred at 0° C. for 1 h. The reaction was partitioned between H₂O (20 mL), and the mixture was extracted with ethyl acetate (25 mL×3). The organic phase was separated, washed with brine solution (20 mL), dried over Na₂SO₄, and filtered. The filtrate was concentrated under reduced pressure to furnish ethyl 4-(3-bromophenyl)sulfanyl-1,5-dimethyl-pyrrole-2-carboxylate. LCMS: (M+H⁺): 354.0.

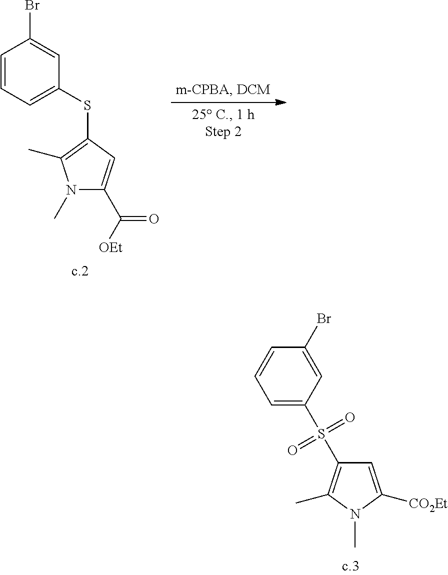 [SMILES: CCOC(=O)c1cc(Sc2cccc(Br)c2)c(C)n1C ; CCOC(=O)c1cc(S(=O)(=O)c2cccc(Br)c2)c(C)n1C]

To a solution of ethyl 4-(3-bromophenyl)sulfanyl-1,5-dimethyl-pyrrole-2-carboxylate (1.28 g, 3.61 mmol, 1 eq) in DCM (30 mL) was degassed and purged with N₂ (3×). m-CPBA (1.83 g, 9.03 mmol, 85% purity, 2.5 eq) was added at 0° C. The mixture was stirred at 25° C. for 1 h. The mixture was poured into sat. aqueous Na₂SO₃ (50 mL), and the mixture was extracted with ethyl acetate (50 mL×3). The combined organic phase was washed with brine (50 mL×2), dried with anhydrous Na₂SO₄, and filtered. The filtrate was concentrated. The residue was purified by column chromatography (SiO₂, petroleum ether/ethyl acetate=0/1 to 1/1) to furnish ethyl 4-(3-bromophenyl)sulfonyl-1,5-dimethyl-pyrrole-2-carboxylate. LCMS: (M+H⁺): 276.0.

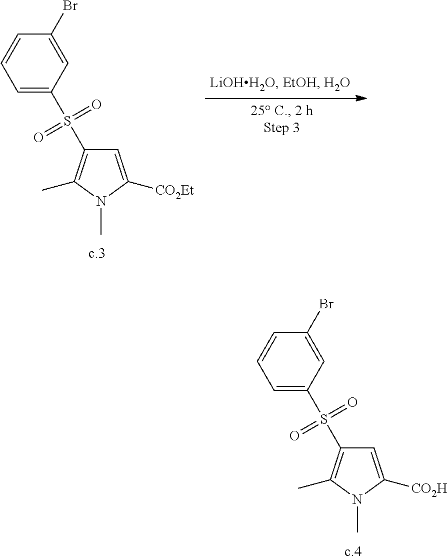 [SMILES: CCOC(=O)c1cc(S(=O)(=O)c2cccc(Br)c2)c(C)n1C ; Cc1c(S(=O)(=O)c2cccc(Br)c2)cc(C(=O)O)n1C]

A mixture of ethyl 4-(3-bromophenyl)sulfonyl-1,5-dimethyl-pyrrole-2-carboxylate (400 mg, 1.04 mmol, 1 eq), LiOH·H₂O (65 mg, 1.55 mmol, 1.5 eq) in EtOH (5 mL) and H₂O (1 mL) was degassed and purged with N₂ (3×). The mixture was stirred at 25° C. for 12 h under a N₂ atmosphere. The reaction mixture was concentrated under reduced pressure. The residue was diluted with H₂O (10 mL), and the pH was adjusted to 1-2 by addition of aq. HCl (2 M). The mixture was filtered to furnish 4-(3-bromophenyl)sulfonyl-1,5-dimethyl-pyrrole-2-carboxylic acid. LCMS: (M+H⁺): 421.0 & 423.0.

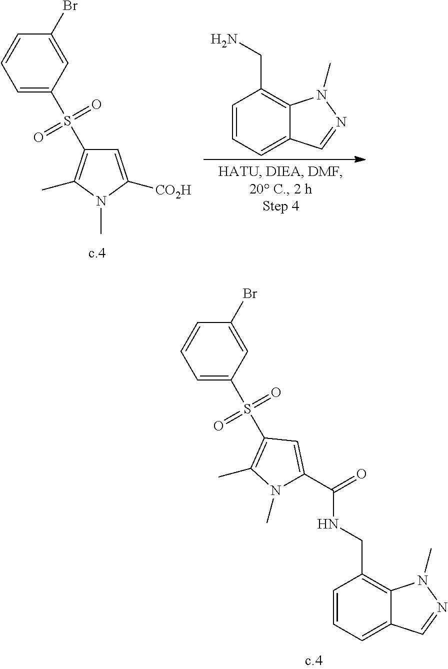 [SMILES: Cc1c(S(=O)(=O)c2cccc(Br)c2)cc(C(=O)O)n1C ; Cn1ncc2cccc(CN)c21 ; Cc1c(S(=O)(=O)c2cccc(Br)c2)cc(C(=O)NCc2cccc3cnn(C)c23)n1C]

A mixture of 4-(3-bromophenyl)sulfonyl-1,5-dimethyl-pyrrole-2-carboxylic acid (200 mg, 0.558 mmol, 1 eq), HATU (318 mg, 0.837 mmol, 1.5 eq) in DMF (1 mL) was added (1-methylindazol-7-yl)methanamine (90 mg, 0.558 mmol, 1 eq) and DIPEA (216 mg, 1.68 mmol, 3 eq) in DMF (1 mL). The mixture was stirred at 25° C. for 2 h under a N₂ atmosphere. The reaction mixture was diluted with H₂O (10 mL), and the mixture was extracted with ethyl acetate (10 mL×3). The organic phase was separated, washed with brine solution (20 mL), dried over Na₂SO₄, and filtered. The filtrate was concentrated under reduced pressure to give a residue. The residue was purified by column chromatography (SiO₂, petroleum ether/ethyl acetate=0/1 to 1/1) to furnish 4-(3-bromophenyl)sulfonyl-1,5-dimethyl-N-[(1-methylindazol-7-yl)methyl]pyrrole-2-carboxamide. LCMS: (M+H⁺): 501.1.

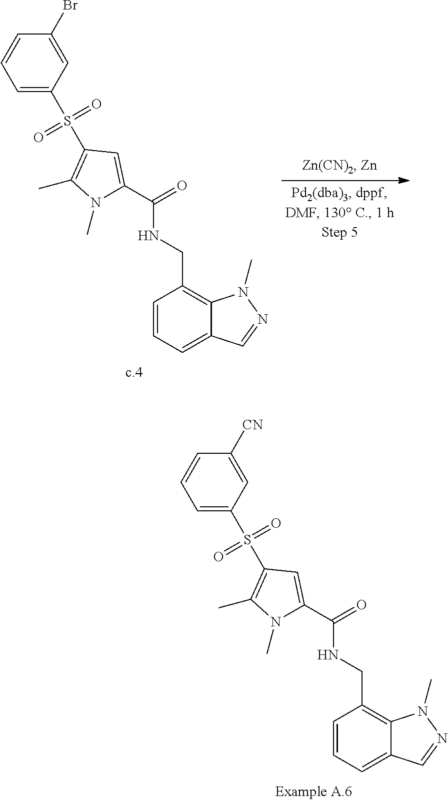 [SMILES: Cc1c(S(=O)(=O)c2cccc(Br)c2)cc(C(=O)NCc2cccc3cnn(C)c23)n1C ; Cc1c(S(=O)(=O)c2cccc(C#N)c2)cc(C(=O)NCc2cccc3cnn(C)c23)n1C]

4-(3-bromophenyl)sulfonyl-1,5-dimethyl-N-[(1-methylindazol-7-yl)methyl]pyrrole-2-carboxamide (136 mg, 0.271 mmol, 1 eq), Zn(CN)₂ (96 mg, 0.81 mmol, 3 eq) and DPPF (30 mg, 0.054 mmol, 0.2 eq), Pd₂(dba)₃ (25 mg, 0.027 mmol, 0.1 eq), Zn (35 mg, 0.54 mmol, 2 eq) were taken up into a microwave tube in DMF (2 mL). The sealed tube was heated at 150° C. for 60 min under microwave irradiation. The reaction mixture was poured into H₂O (5 mL). The mixture was extracted with EtOAc (5 mL×3). The combined organic layers were washed with brine (10 mL), dried over Na₂SO₄, and filtered. The filtrate was concentrated under reduced pressure. The residue was purified by preparative-HPLC (column: Waters Xbridge Prep OBD C18 150*40 mm*10 um; mobile phase: [water(NH₄HCO₃)-ACN];B %: 30%-60%,8 min) to furnish 4-(3-cyanophenyl)sulfonyl-1,5-dimethyl-N-[(1-methylindazol-7-yl)methyl]pyrrole-2-carboxamide. LCMS: (M+H⁺): 448.0; ¹H NMR: (400 MHz, DMSO-d₆) δ 8.84 (br t, J=5.2 Hz, 1H), 8.26 (s, 1H), 8.13 (br t, J=8.1 Hz, 2H), 8.03 (s, 1H), 7.80 (t, J=7.9 Hz, 1H), 7.68 (d, J=7.9 Hz, 1H), 7.33 (s, 1H), 7.28 (d, J=6.9 Hz, 1H), 7.12-7.05 (m, 1H), 4.89 (br d, J=5.0 Hz, 2H), 4.25 (s, 3H), 3.79 (s, 3H), 2.42 (s, 3H).

The following examples can be prepared in a similar fashion to that described above in Scheme C using the appropriate thiol and amine.

TABLE A

| | Thiol | Amine | | ¹H NMR |
|---|---|---|---|---|
| Ex. | (Step 1) | (Step 4) | Structure | LCMS |
| A.24 | 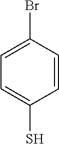 | 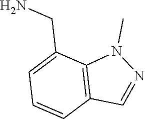 | 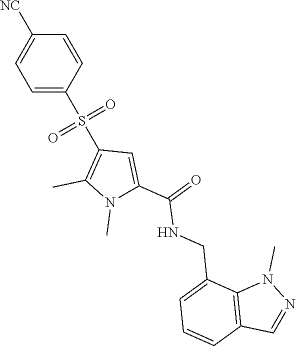 [SMILES: Cc1c(S(=O)(=O)c2ccc(C#N)cc2)cc(C(=O)NCc2cccc3cnn(C)c23)n1C] | LCMS (M + H⁺): 448.0; 1H NMR (400 MHz, DMSO-d6) δ 8.85 (t, J = 5.1 Hz, 1H), 8.09-8.03 (m, 2H), 8.02-7.92 (m, 2H), 7.67 (d, J = 8.0 Hz, 1H), 7.33 (s, 1H), 7.27 (d, J = 7.0 Hz, 1H), 7.08 (t, J = 7.6 Hz, 1H), 4.88 (d, J = 5.3 Hz, 2H), 4.24 (s, 3H), 3.79 (s, 3H), 2.42-2.39 (m, 3H) |

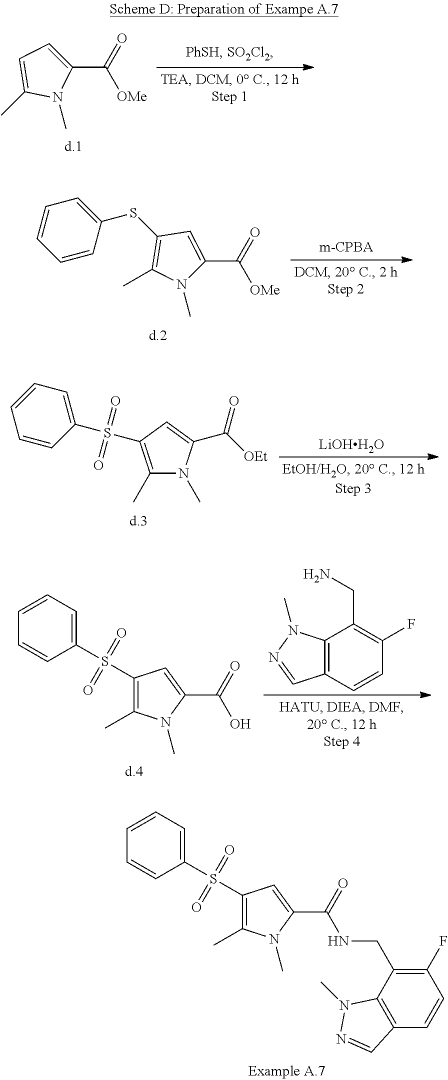

### Step-by-Step Preparation of Example A.7

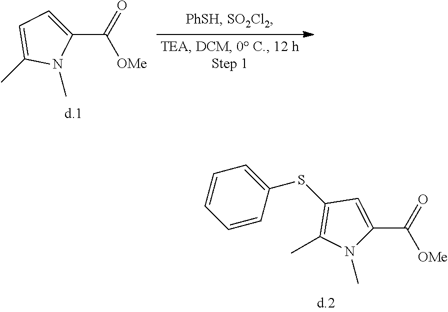 [SMILES: COC(=O)c1ccc(C)n1C ; COC(=O)c1cc(Sc2ccccc2)c(C)n1C]

A mixture of benzenethiol (2.00 g, 18.2 mmol, 1.85 mL, 1 eq), ethyl 1,5-dimethyl-1H-pyrrole-2-carboxylate (3.64 g, 21.8 mmol, 1.2 eq), sulfuryl chloride (2.45 g, 18.2 mmol, 1.81 mL, 1 eq), TEA (92 mg, 0.91 mmol, 0.05 eq) in DCM (20 mL) was degassed and purged with N₂ (3×). The mixture was stirred at 0° C. for 12 h under N₂ atmosphere. The reaction mixture was poured into H₂O (30 mL). The mixture was extracted with DCM (10 mL*3). The combined organic layers were washed with brine (50 mL), dried over Na₂SO₄, and filtered. The filtrate was concentrated under reduced pressure. The residue was purified by column chromatography (SiO₂, petroleum ether/ethyl acetate=1/0 to 1/0) to furnish ethyl 1,5-dimethyl-4-(phenylthio)-1H-pyrrole-2-carboxylate.

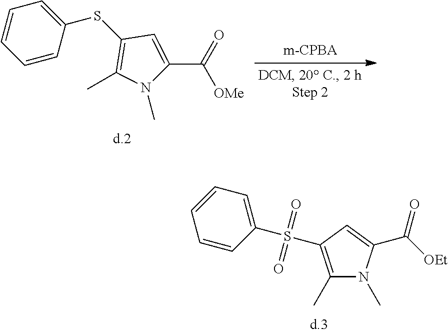 [SMILES: COC(=O)c1cc(Sc2ccccc2)c(C)n1C ; CCOC(=O)c1cc(S(=O)(=O)c2ccccc2)c(C)n1C]

A mixture of ethyl 1,5-dimethyl-4-(phenylthio)-1H-pyrrole-2-carboxylate (0.500 g, 1.82 mmol, 1 eq), m-CPBA (799 mg, 4.54 mmol, 98% purity, 2.5 eq) in DCM (10 mL) was degassed and purged with N₂ (3×). The mixture was stirred at 20° C. for 2 h under a N₂ atmosphere. The mixture was diluted with H₂O (10 mL), and the mixture was extracted with EtOAc (15 mL×3). The combined organic layers were washed with brine (20 mL), dried over Na₂SO₄, and filtered. The filtrate was concentrated under reduced pressure to give a residue. The residue was purified by column chromatography (SiO₂, petroleum ether/ethyl acetate=1/0 to 3/1) to furnish ethyl 1,5-dimethyl-4-(phenylsulfonyl)-1H-pyrrole-2-carboxylate.

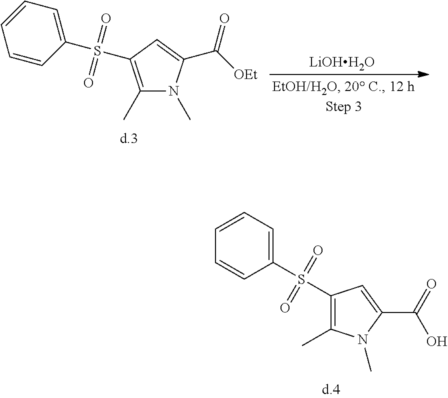 [SMILES: CCOC(=O)c1cc(S(=O)(=O)c2ccccc2)c(C)n1C ; "LiOH·H2O" ; Cc1c(S(=O)(=O)c2ccccc2)cc(C(=O)O)n1C]

A mixture of ethyl 1,5-dimethyl-4-(phenylsulfonyl)-1H-pyrrole-2-carboxylate (0.100 g, 0.325 mmol, 1 eq), NaOH (26 mg, 0.65 mmol, 2 eq) in EtOH (2 mL) and H₂O (0.2 mL) was degassed and purged with N₂ (3×). The mixture was stirred at 20° C. for 12 h under N₂ atmosphere. The mixture was concentrated under reduced pressure to remove EtOH. The pH of the mixture was adjusted to 3 by addition with aq. HCl (2 N). The solid was collected and dried to furnish 1,5-dimethyl-4-(phenylsulfonyl)-1H-pyrrole-2-carboxylic acid. LCMS: (M−H⁺): 277.9.

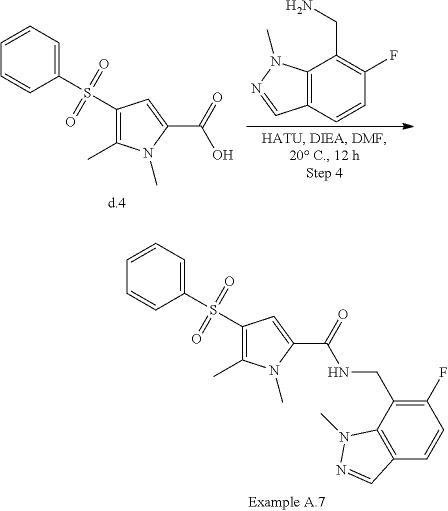

A mixture of 1,5-dimethyl-4-(phenylsulfonyl)-1H-pyrrole-2-carboxylic acid (50 mg, 0.18 mmol, 1 eq), (6-fluoro-8-methyl-indazol-7-yl)methanamine (32 mg, 0.18 mmol, 1 eq), HATU (82 mg, 0.21 mmol, 1.2 eq), DTPEA (116 mg, 0.895 mmol, 5 eq) in DMF (0.5 mL) was degassed and purged with N₂ (3×). The mixture was stirred at 20° C. for 12 h under a N₂ atmosphere. The mixture was filtered and concentrated under reduced pressure to give a residue. The residue was purified by preparative-LPLC (column: Waters Xbridge BEH C18 100*30 mm*10 um;mobile phase: [water(NH₃H₂O+NH₄HCO₃)-ACN];B %: 350%-65%,8 min). to furnish N-((6-fluoro-1-methyl-1H-indazol-7-yl)methyl)-1,5-dimethyl-4-(phenylsulfonyl)-1H-pyrrole-2-carboxamide. LCMS: (M+H⁺): 441.0, ¹H NMR: (400 MHz, DMSO-d₆) δ 8.50 (br s, 1H), 8.02 (s, 1H), 7.80 (br d, J=7.0 Hz, 2H), 7.74 (dd, J=5.2, 8.8 Hz, 1H), 7.63-7.53 (m, 3H), 7.23 (s, 1H), 7.06-6.97 (m, 1H), 4.82 (br d, J=3.1 Hz, 2H), 4.24-4.19 (5, 3H), 3.80 (s, 3H), 2.39 (s, 3H).

The following examples in Table 2 can be prepared in a similar fashion to that described above in Scheme D using the appropriate reagents (thiol in Step 1 and amine in Step 4 of Scheme D).

TABLE 2

| | Thiol | Amine | | ¹H NMR |
|---|---|---|---|---|
| Ex. | (Step 1) | (Step 4) | Structure | LCMS |
| A.8 | 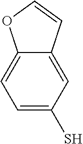 | 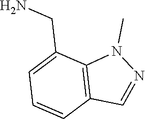 | 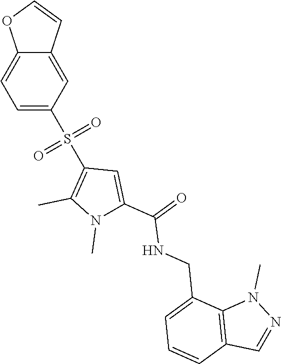 [SMILES: Cc1c(S(=O)(=O)c2ccc3occc3c2)cc(C(=O)NCc2cccc3cnn(C)c23)n1C] | LCMS (M + H⁺): 463.0 ¹H NMR: (400 MHz, DMSO-d6) δ 8.83 (br t, J = 5.0 Hz, 1H), 8.26- 8.13 (m, 2H), 8.03 (s, 1H), 7.83- 7.72 (m, 2H), 7.67 (d, J = 8.0 Hz, 1H), 7.33 (s, 1H), 7.26 (d, J = 7.0 Hz, 1H), 7.18-7.02 (m, 2H), 4.88 (d, J = 5.1 Hz, 2H), 4.25 (s, 3H), 3.77 (s, 3H), 2.41 (s, 3H) |
| A.9 | 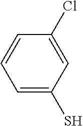 | 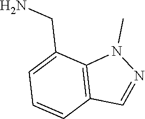 | 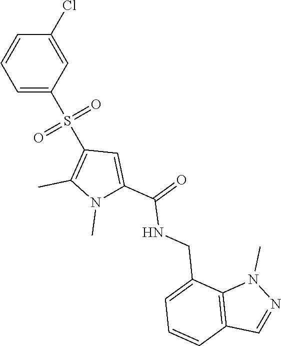 [SMILES: Cc1c(S(=O)(=O)c2cccc(Cl)c2)cc(C(=O)NCc2cccc3cnn(C)c23)n1C] | LCMS (M + H⁺): 457.0 ¹H NMR: (400 MHz, DMSO-d6) δ 8.84 (t, J = 5.3 Hz, 1H), 8.03 (s, 1H), 7.82-7.78 (m, 2H), 7.75-7.71 (m, 1H), 7.69-7.64 (m, 1H), 7.64- 7.60 (m, 1H), 7.33 (s, 1H), 7.27 (d, J = 7.0 Hz, 1H), 7.12-7.05 (m, 1H), 4.89 (d, J = 5.1 Hz, 2H), 4.25 (s, 3H), 3.79 (s, 3H), 2.42 (s, 3H) |
| A.10 | 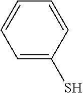 | 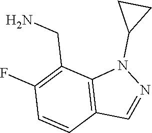 [SMILES: NCc1c(F)ccc2cnn(C3CC3)c12] | 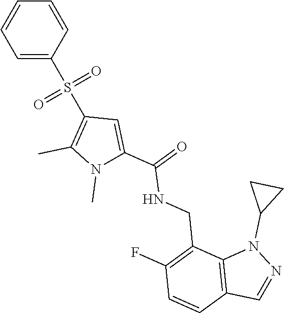 [SMILES: Cc1c(S(=O)(=O)c2ccccc2)cc(C(=O)NCc2c(F)ccc3cnn(C4CC4)c23)n1C] | LCMS (M + H⁺): 467.0 ¹H NMR: (400 MHz, DMSO-d6) δ δ 8.63 (br t, J = 4.2 Hz, 1H), 8.03 (s, 1H), 7.81-7.74 (m, 3H), 7.62- 7.54 (m, 3H), 7.24 (s, 1H), 7.07 (dd, J = 8.9, 10.1 Hz, 1H), 5.00 (br d, J = 2.8 Hz, 2H), 3.95 (td, J = 3.5, 7.1 Hz, 1H), 3.78 (s, 3H), 2.38 (s, 3H), 1.32-1.23 (m, 2H), 1.12- 1.03 (m, 2H) |

TABLE B

| | Thiol | Amine | | ¹H NMR |
|---|---|---|---|---|
| Ex. | (Step 1) | (Step 4) | Structure | LCMS |
| A.11 | 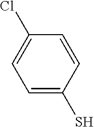 | 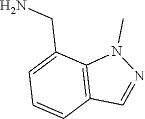 | 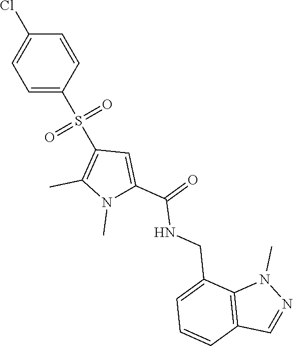 [SMILES: Cc1c(S(=O)(=O)c2ccc(Cl)cc2)cc(C(=O)NCc2cccc3cnn(C)c23)n1C] | LCMS (M + H⁺): 457.0; ¹H NMR: (400 MHz, DMSO-d6) δ 8.84 (t, J = 5.25 Hz, 1H), 8.03 (s, 1H), 7.88-7.78 (m, 2H), 7.71-7.61 (m, 3H), 7.32 (s, 1H), 7.27 (d, J = 7.00 Hz, 1H), 7.08 (t, J = 7.14 Hz, 1H), 4.89 (d, J = 5.13 Hz, 2H), 4.25 (s, 3H), 3.79 (s, 3H), 2.40 (s, 3H) |
| A.12 | 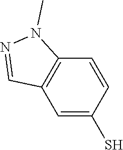 | 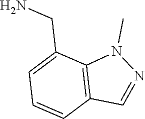 | 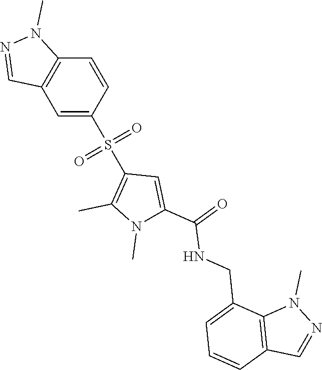 [SMILES: Cc1c(S(=O)(=O)c2ccc3c(cnn3C)c2)cc(C(=O)NCc2cccc3cnn(C)c23)n1C] | LCMS (M + H⁺): 477.1; ¹H NMR (400 MHz, DMSO-d6) δ 8.83 (t, J = 5.3 Hz, 1H), 8.37 (d, J = 1.0 Hz, 1H), 8.27 (d, J = 0.6 Hz, 1H), 8.03 (s, 1H), 7.83-7.78 (m, 1H), 7.76-7.72 (m, 1H), 7.67 (d, J = 7.6 Hz, 1H), 7.33 (s, 1H), 7.27 (d, J = 6.9 Hz, 1H), 7.12-7.05 (m, 1H), 4.88 (d, J = 5.1 Hz, 2H), 4.25 (s, 3H), 4.07 (s, 3H), 3.77 (s, 3H), 2.40 (s, 3H) |
| A.14 | 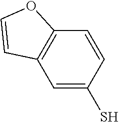 | 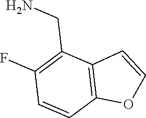 [SMILES: NCc1c(F)ccc2occc12] | 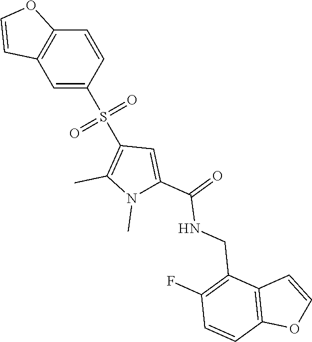 [SMILES: Cc1c(S(=O)(=O)c2ccc3occc3c2)cc(C(=O)NCc2c(F)ccc3occc23)n1C] | LCMS (M + H⁺): 467.0; ¹H NMR (400 MHz, DMSO-d6) δ 8.84 (t, J = 5.6 Hz, 1H), 8.19 (dd, J = 1.9, 6.8 Hz, 2H), 8.04 (d, J = 2.1 Hz, 1H), 7.83- 7.71 (m, 2H), 7.55 (dd, J = 3.6, 8.9 Hz, 1H), 7.23 (s, 1H), 7.16 (d, J = 10.1 Hz, 1H), 7.12 (dd, J = 2.0, 4.9 Hz, 2H), 4.62 (d, J = 5.5 Hz, 2H), 3.76 (s, 3H), 2.40 (s, 3H) |
| A.16 | 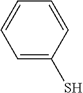 | 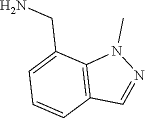 | 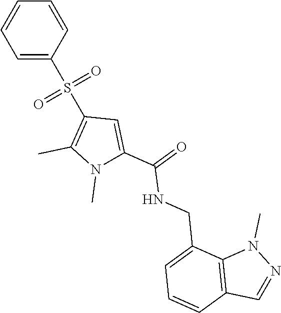 [SMILES: Cc1c(S(=O)(=O)c2ccccc2)cc(C(=O)NCc2cccc3cnn(C)c23)n1C] | LCMS (M + H⁺): 423.0; 1H NMR (400 MHz, DMSO-d6) δ 8.84 (t, J = 5.1Hz, 1H), 8.03 (s, 1H), 7.86- 7.78 (m, 2H), 7.71-7.53 (m, 4H), 7.32 (s, 1H), 7.27 (d, J = 7.0 Hz, 1H), 7.08 (t, J = 7.5 Hz, 1H), 4.88 (d, J = 5.0 Hz, 2H), 4.25 (s, 3H), 3.79 (s, 3H), 2.42-2.38 (m, 3H) |
| A.17 | 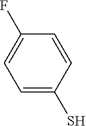 | 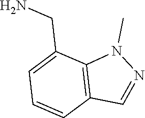 | 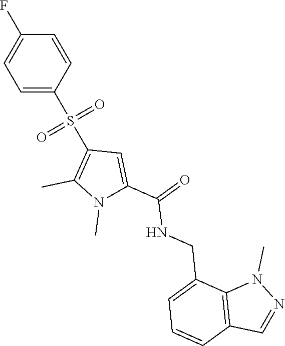 [SMILES: Cc1c(S(=O)(=O)c2ccc(F)cc2)cc(C(=O)NCc2cccc3cnn(C)c23)n1C] | LCMS (M + H⁺): 441.0; 1H NMR (400 MHz, DMSO-d6) δ 8.83 (br t, J = 4.7 Hz, 1H), 8.03 (s, 1H), 7.93-7.85 (m, 2H), 7.67 (d, J = 8.0 Hz, 1H), 7.42 (br t, J = 8.8 Hz, 2H), 7.31 (s, 1H), 7.27 (br d, J = 6.9 Hz, 1H), 7.12-7.05 (m, 1H), 4.88 (br d, J = 5.0 Hz, 2H), 4.25 (s, 3H), 3.79 (s, 3H), 2.40 (s, 3H) |

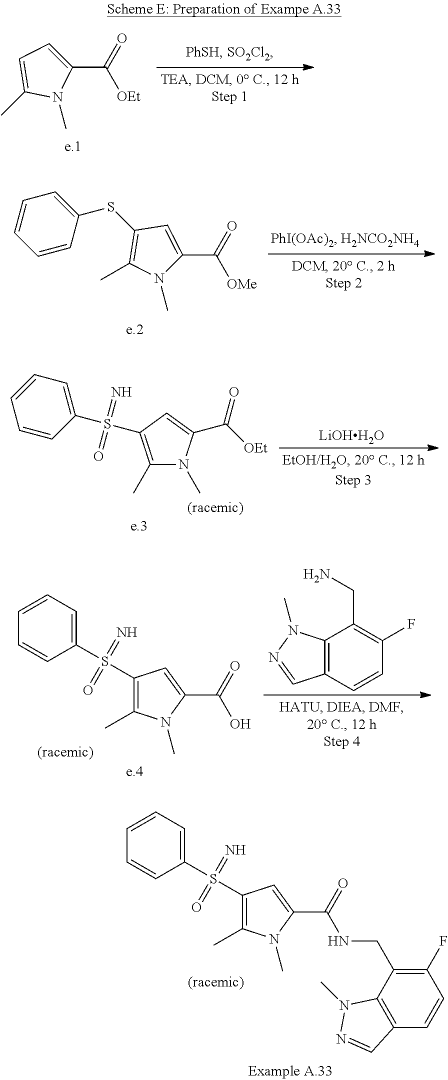

Example A.33 can be prepared in a similar fashion to the steps described in Scheme Ac using the appropriate reagents depicted in Scheme E. Example A.33: ¹H NMR (400 MHz, DMSO-d6) δ 9.01-8.82 (m, 1H), 8.10-7.88 (m, 3H), 7.82-7.57 (m, 4H), 7.54-7.40 (m, 1H), 7.33-7.21 (m, 1H), 7.15-7.02 (m, 1H), 5.00-4.77 (m, 2H), 4.31-4.20 (m, 3H), 3.82 (br s, 3H), 2.45 (br s, 3H); LCMS (M+H⁺): 422.1.

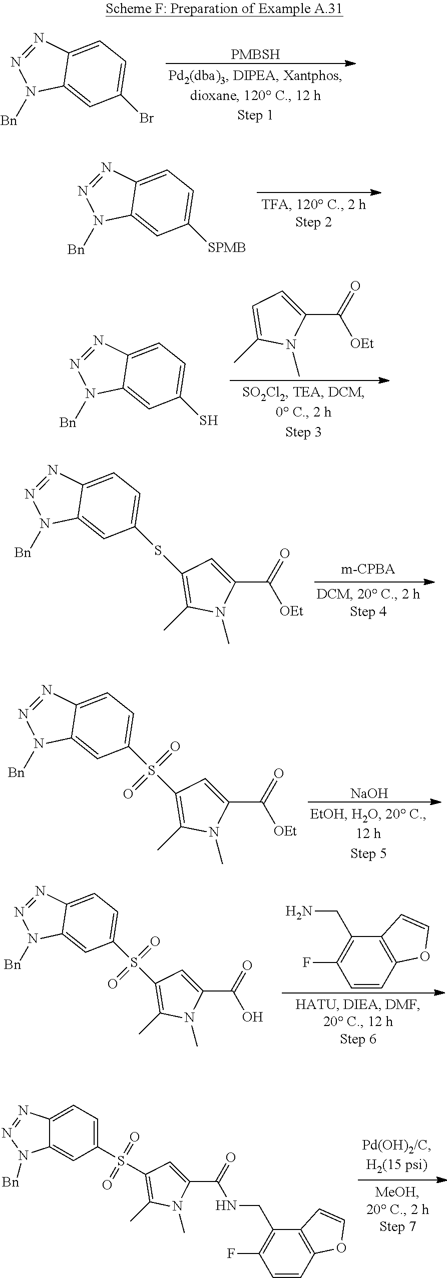

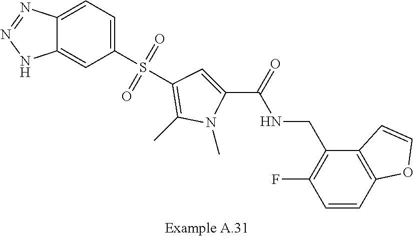 [SMILES: Cc1c(S(=O)(=O)c2ccc3nn[nH]c3c2)cc(C(=O)NCc2c(F)ccc3occc23)n1C]

Step 1. A mixture of 1-benzyl-6-bromo-1H-benzo[d][1,2,3]triazole (9.00 g, 31.2 mmol, 1 eq), (4-methoxyphenyl)methanethiol (4.82 g, 31.2 mmol, 4.34 mL, 1 eq), Pd₂(dba)₃ (2.86 g, 3.12 mmol, 0.1 eq), Xantphos (2.71 g, 4.69 mmol, 0.15 eq) and DIPEA (12.1 g, 93.7 mmol, 16.3 mL, 3 eq) in dioxane (90 mL) was degassed and purged with N₂ (3×). The mixture was stirred at 120° C. for 12 h under a N₂ atmosphere. The mixture was diluted with H₂O (10 mL), and the mixture was extracted with EtOAc (15 mL×3). The combined organic layers were washed with brine (20 mL), dried over Na₂SO₄, and filtered. The filtrate was concentrated under reduced pressure. The residue was purified by column chromatography (SiO₂, petroleum ether/ethyl acetate=10/1 to 5/1) to furnish 1-benzyl-6-((4-methoxybenzyl)thio)-1H-benzo[d][1,2,3]triazole.

Step 2. A mixture of 1-benzyl-6-((4-methoxybenzyl)thio)-1H-benzo[d][1,2,3]triazole (5 g, 13.83 mmol, 1 eq) in TFA (50 mL) was degassed and purged with N₂ (3×). The mixture was stirred at 120° C. for 2 h under a N₂ atmosphere. The reaction was diluted with H₂O (10 mL), and the mixture was extracted with EtOAc (15 mL×3). The combined organic layers were washed with brine (20 mL), dried over Na₂SO₄, and filtered. The filtrate was concentrated under reduced pressure. The residue was purified by column chromatography (SiO₂, petroleum ether/ethyl acetate=10/1 to 5/1) to furnish 1-benzyl-1H-benzo[d][1,2,3]triazole-6-thiol.

Steps 3-6. 1-benzyl-1H-benzo[d][1,2,3]triazole-6-thiol was converted into 4-((1-benzyl-1H-benzo[d][1,2,3]triazol-6-yl)sulfonyl)-N-((5-fluorobenzofuran-4-yl)methyl)-1,5-dimethyl-1H-pyrrole-2-carboxamide using conditions like those outlined above in Scheme D.

Step 7. To a solution of 4-((1-benzyl-1H-benzo[d][1,2,3]triazol-6-yl)sulfonyl)-N-((5-fluorobenzofuran-4-yl)methyl)-1,5-dimethyl-1H-pyrrole-2-carboxamide (0.1 g, 0.179 mmol, 1 eq) in MeOH (2 mL) was added Pd(OH)₂/C (20 wt %, 0.1 g) under a N₂ atmosphere. The suspension was degassed and purged with H₂ (3×). The mixture was stirred under H₂ (15 Psi) at 20° C. for 2 h. The mixture was filtered and concentrated under reduced pressure. The residue was purified by preparative-HPLC (FA conditions, Column: Phenomenex Luna C18 75×30 mm×3 μm; mobile phase: [water(FA)-ACN]; B %: 30%-65%, 8 min) to furnish 4-((1H-benzo[d][1,2,3]triazol-6-yl)sulfonyl)-N-((5-fluorobenzofuran-4-yl)methyl)-1,5-dimethyl-1H-pyrrole-2-carboxamide Example A.31. LCMS: (M+H⁺): 468.1; ¹H NMR: (400 MHz, DMSO-d₆) δ 8.88-8.80 (m, 1H), 8.44 (d, J=0.7 Hz, 1H), 8.06-7.99 (m, 2H), 7.80-7.72 (m, 1H), 7.58-7.51 (m, 1H), 7.36-7.33 (m, 1H), 7.26 (s, 1H), 7.16 (d, J=9.9 Hz, 1H), 7.14-7.09 (m, 1H), 4.62 (d, J=5.3 Hz, 2H), 3.75 (s, 3H), 2.40 (s, 3H).

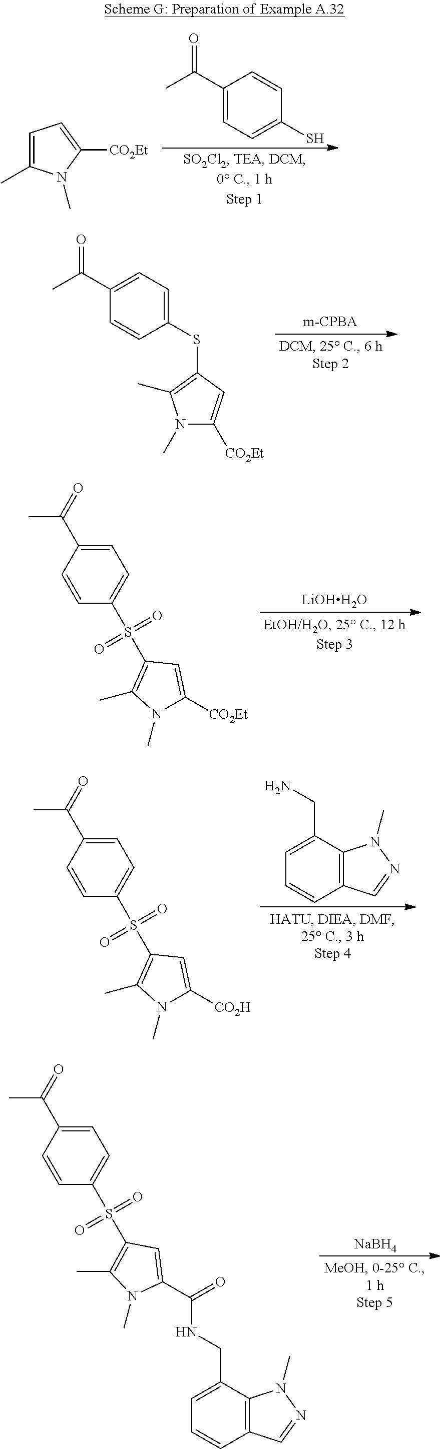

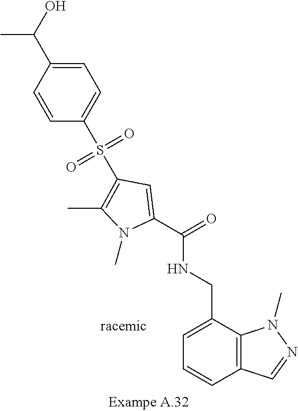 [SMILES: Cc1c(S(=O)(=O)c2ccc(C(C)O)cc2)cc(C(=O)NCc2cccc3cnn(C)c23)n1C]

4-(4-acetylphenyl)sulfonyl-1,5-dimethyl-N-[(1-methylindazol-7-yl)methyl]pyrrole-2-carboxamide was prepared in a like manner to that shown in Scheme D using the appropriate reagents.

Step 5. To a solution of 4-(4-acetylphenyl)sulfonyl-1,5-dimethyl-N-[(1-methylindazol-7-yl)methyl]pyrrole-2-carboxamide (0.200 g, 0.431 mmol, 1 eq) in MeOH (5 mL) was added NaBH₄ (49 mg, 1.29 mmol, 3 eq) at 0° C. The mixture was stirred at 25° C. for 1 h. The reaction was diluted with water (30 mL), and the mixture was extracted with ethyl acetate (20 mL×2). The combined organic phase was washed with brine (10 mL×2), dried over Na₂SO₄, and filtered. The filtrate was concentrated. The residue was purified by preparative-HPLC (neutral condition: column: Waters Xbridge Prep OBD C18 150×40 mm×10 μm; mobile phase: [water (NH₄HCO₃)-ACN]; B %: 25%-65%, 8 min) to furnish 4-[4-(1-hydroxyethyl)phenyl]sulfonyl-1,5-dimethyl-N-[(1-methylindazol-7-yl)methyl]pyrrole-2-carboxamide Example A.32. LCMS: (M+H⁺):467.1; ¹H NMR: (400 MHz, METHANOL-d4) δ 7.98 (d, J=2.13 Hz, 1H), 7.83 (d, J=8.38 Hz, 2H), 7.68 (br d, J=7.88 Hz, 1H), 7.53 (br d, J=8.25 Hz, 2H), 7.34 (br d, J=7.00 Hz, 1H), 7.18 (s, 1H), 7.11 (td, J=7.57, 2.63 Hz, 1H), 4.98 (br d, J=2.13 Hz, 2H), 4.89-4.87 (m, 1H), 4.29 (d, J=2.75 Hz, 3H), 3.81 (d, J=3.13 Hz, 3H), 2.45 (s, 3H), 1.40 (d, J=6.50 Hz, 3H).

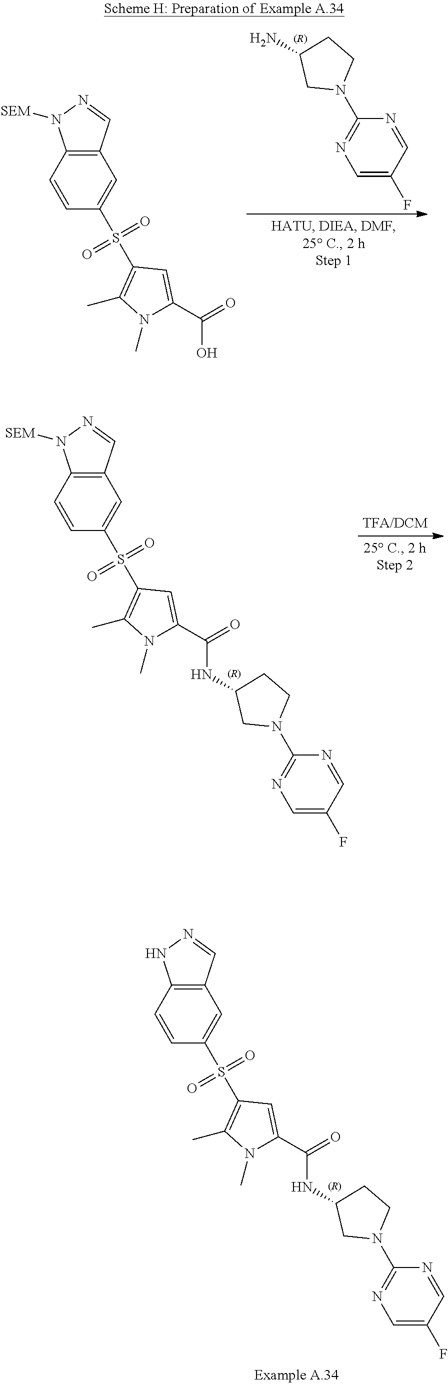

Step 1. A mixture of 1,5-dimethyl-4-[1-(2-trimethylsilylethoxymethyl)indazol-5-yl]sulfonyl-pyrrole-2-carboxylic acid (1.00 g, 2.22 mmol, 1 eq), (3R)-1-(5-fluoropyrimidin-2-yl)pyrrolidin-3-amine (486 mg, 2.22 mmol, 1 eq, HCl), HATU (1.01 g, 2.67 mmol, 1.2 eq), DIPEA (862 mg, 6.67 mmol, 1.16 mL, 3 eq) in DMF (10 mL) was degassed and purged with N₂ (3×). The mixture was stirred at 25° C. for 2 h under a N₂ atmosphere. The reaction was poured into water (30 mL), and the mixture was extracted with ethyl acetate (15 mL×3). The combined organic phase was washed with brine (15 mL), dried with anhydrous Na₂SO₄, and filtered. The filtrate was concentrated. The residue was purified by flash silica gel chromatography (ISCO®; 12 g SepaFlash® Silica Flash Column, gradient elution of 0~50% ethyl acetate/petroleum at 40 mL/min) to furnish N-[(3R)-1-(5-fluoropyrimidin-2-yl)pyrrolidin-3-yl]-1,5-dimethyl-4-[1-(2-trimethylsilylethoxymethyl)indazol-5-yl]sulfonyl-pyrrole-2-carboxamide.

Step 2. A mixture of N-[(3R)-1-(5-fluoropyrimidin-2-yl)pyrrolidin-3-yl]-1,5-dimethyl-4-[1-(2-trimethylsilylethoxymethyl)indazol-5-yl]sulfonyl-pyrrole-2-carboxamide (0.98 g, 2.1 mmol, 1 eq) in DCM (10 mL) and TFA (3 mL) was degassed and purged with N₂ (3×). The mixture was stirred at 25° C. for 2 h under a N₂ atmosphere. The mixture was poured into water (20 mL), and the mixture was extracted with ethyl acetate (15 mL×3). The combined organic phase was washed with brine (15 mL), dried with anhydrous Na₂SO₄, and filtered. The filtrate was concentrated. The residue was purified by preparative-HPLC (neutral condition, column: Waters Xbridge Prep OBD C18 150×40 mm×10 μm; mobile phase: [H₂O(10 mM NH₄HCO₃)-ACN]; gradient: 30%-60% B over 8.0 min) to furnish N-[(3R)-1-(5-fluoropyrimidin-2-yl)pyrrolidin-3-yl]-4-(1H-indazol-5-ylsulfonyl)-1,5-dimethyl-pyrrole-2-carboxamide Example A.34. LCMS: (M+H⁺): 484.1; ¹H NMR: (400 MHz, METHANOL-d4) δ 8.43 (d, J=1.0 Hz, 1H), 8.26 (s, 2H), 8.21 (s, 1H), 7.78 (dd, J=1.6, 8.9 Hz, 1H), 7.63 (d, J=8.9 Hz, 1H), 7.20 (s, 1H), 4.57 (quin, J=6.1 Hz, 1H), 3.84 (dd, J=6.6, 11.4 Hz, 1H), 3.77 (s, 3H), 3.70 (td, J=7.1, 10.7 Hz, 1H), 3.57 (td, J=7.1, 11.0 Hz, 1H), 3.47 (dd, J=5.3, 11.4 Hz, 1H), 2.44 (s, 3H), 2.35-2.25 (m, 1H), 2.13-2.03 (i, 1H).

The following examples can be made in a similar fashion to that described above in Scheme H using the appropriate reagents shown in Table D.

TABLE D

| | | | | ¹H NMR |
|---|---|---|---|---|
| Ex. | Amine | Acid | Structure | LCMS |
| A.35 | 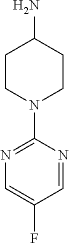 [SMILES: NC1CCN(c2ncc(F)cn2)CC1] | 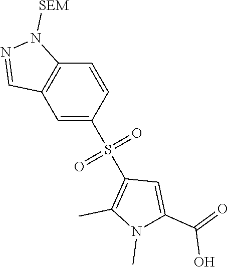 | 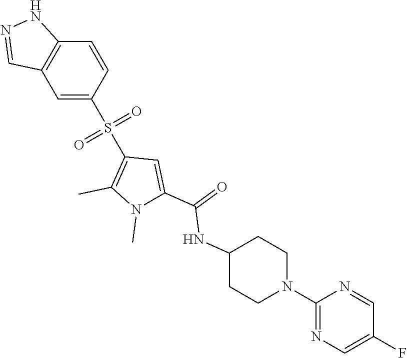 [SMILES: Cc1c(S(=O)(=O)c2ccc3[nH]ncc3c2)cc(C(=O)NC2CCN(c3ncc(F)cn3)CC2)n1C] | LCMS (M + H⁺): 498.1; 1H NMR (400 MHz, METHANOL-d4) δ 8.44 (s, 1H), 8.28-8.21 (m, 3H), 7.80 (dd, J = 1.6, 8.4 Hz, 1H), 7.65 (d, J = 8.8 Hz, 1H), 7.14 (s, 1H), 4.70 (br d, J = 13.6 Hz, 2H), 4.13-3.98 (m, 1H), 3.78 (s, 3H), 3.06-2.97 (m, 2H), 2.45 (s, 3H), 1.97-1.91 (m, 2H), 1.52 (dt, J = 8.1, 12.1 Hz, 2H) |
| A.36 | 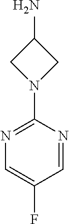 [SMILES: NC1CN(c2ncc(F)cn2)C1] | 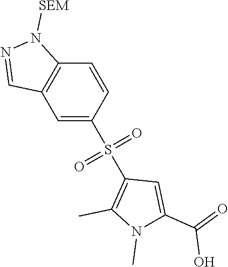 | 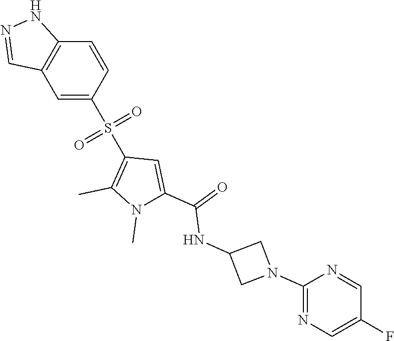 [SMILES: Cc1c(S(=O)(=O)c2ccc3[nH]ncc3c2)cc(C(=O)NC2CN(c3ncc(F)cn3)C2)n1C] | LCMS (M + H⁺): 470.0; 1H NMR (400 MHz, DMSO-d6) δ 8.82 (br d, J = 7.1 Hz, 1H), 8.46 (s, 2H), 8.38 (s, 1H), 8.30 (s, 1H), 7.70 (s, 2H), 7.30 (s, 1H), 4.78-4.67 (m, 1H), 4.29 (t, J = 8.2 Hz, 2H), 3.99 (br dd, J = 5.6, 8.8 Hz, 2H), 3.74 (s, 3H), 2.40 (s, 3H) |
| A.37 | 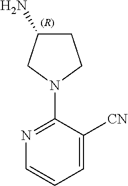 | 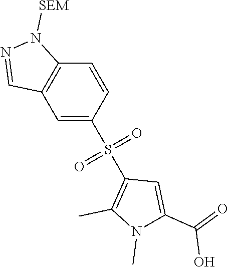 | 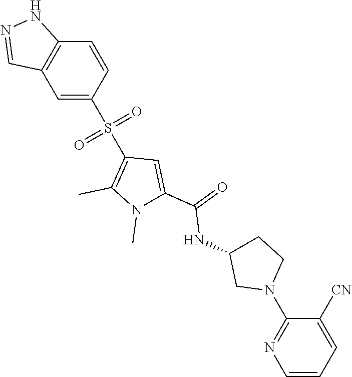 [SMILES: Cc1c(S(=O)(=O)c2ccc3[nH]ncc3c2)cc(C(=O)N[C@@H]2CCN(c3ncccc3C#N)C2)n1C] | LCMS (M + H⁺): 490.0; 1H NMR (400 MHz, METHANOL-d4) δ 8.45 (d, J = 0.9 Hz, 1H), 8.30- 8.20 (m, 2H), 7.90-7.76 (m, 2H), 7.65 (d, J = 8.9 Hz, 1H), 7.21 (s, 1H), 6.70 (dd, J = 4.8, 7.7 Hz, 1H), 4.60-4.56 (m, 1H), 4.06 (dd, J = 6.6, 11.2 Hz, 1H), 3.95 (td, J = 7.1, 10.6 Hz, 1H), 3.90- 3.82 (m, 1H), 3.79 (s, 3H), 3.73 (dd, J = 5.0, 11.1 Hz, 1H), 2.45 (s, 3H), 2.36-2.25 (m, 1H), 2.12 (qd, J = 6.5, 13.0 Hz, 1H) |
| A.38 | 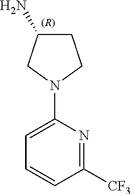 | 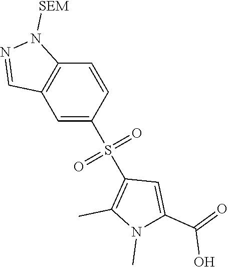 | 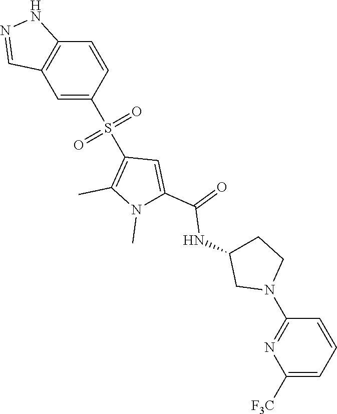 [SMILES: Cc1c(S(=O)(=O)c2ccc3[nH]ncc3c2)cc(C(=O)N[C@@H]2CCN(c3cccc(C(F)(F)F)n3)C2)n1C] | LCMS (M + H⁺): 533.1; 1H NMR (400 MHz, METHANOL-d4) δ8.45 (s, 1H), 8.23 (s, 1H), 7.80 (dd, J = 1.4, 8.8 Hz, 1H), 7.68- 7.60 (m, 2H), 7.20 (s, 1H), 6.91 (d, J = 7.3 Hz, 1H), 6.67 (d, J = 8.8 Hz, 1H), 4.60 (quin, J = 6.2 Hz, 1H), 3.87-3.81 (m, 1H), 3.79 (s, 3H), 3.71-3.63 (m, 1H), 3.55 (td, J = 7.1, 10.6 Hz, 1H), 3.43 (dd, J = 5.3, 10.8 Hz, 1H), 2.45 (s, 3H), 2.40-2.30 (m, 1H), 2.17- 2.06 (m, 1H) |
| A.39 | 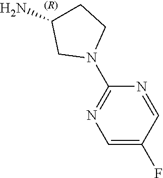 | 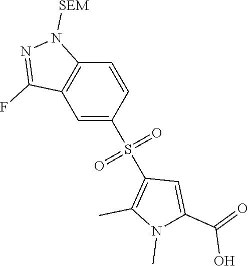 [SMILES: Cc1c(S(=O)(=O)c2ccc3c(c2)c(F)nn3COCC[Si](C)(C)C)cc(C(=O)O)n1C] | 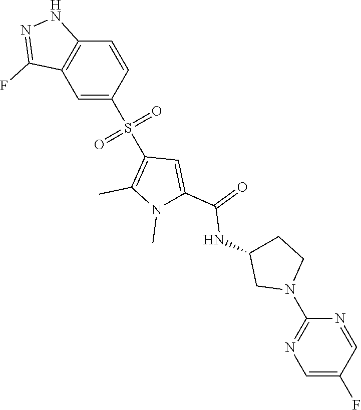 [SMILES: Cc1c(S(=O)(=O)c2ccc3[nH]nc(F)c3c2)cc(C(=O)N[C@@H]2CCN(c3ncc(F)cn3)C2)n1C] | LCMS (M + H⁺): 502.1; 1H NMR (400 MHz, DMSO-d6) δ 8.47- 8.39 (m, 3H), 8.24 (d, J = 0.8 Hz, 1H), 7.76 (dd, J = 1.6, 9.0 Hz, 1H), 7.66 (dd, J = 1.6, 9.0 Hz, 1H), 7.29 (s, 1H), 4.52-4.42 (m, 1H), 3.75 (s, 3H), 3.72 (d, J = 6.6 Hz, 1H), 3.65-3.58 (m, 1H), 3.55-3.51 (m, 1H), 3.45 (br d, J = 4.5 Hz, 1H), 2.40 (s, 3H), 2.19 (br dd, J = 6.5, 13.0 Hz, 1H), 2.07-1.95 (m, 1H) |
| A.40 | 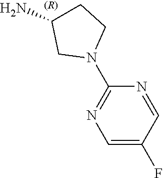 | 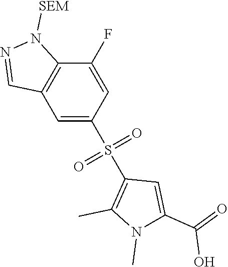 [SMILES: Cc1c(S(=O)(=O)c2cc(F)c3c(cnn3COCC[Si](C)(C)C)c2)cc(C(=O)O)n1C] | 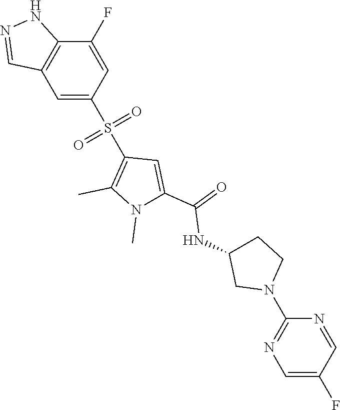 [SMILES: Cc1c(S(=O)(=O)c2cc(F)c3[nH]ncc3c2)cc(C(=O)N[C@@H]2CCN(c3ncc(F)cn3)C2)n1C] | LCMS (M + H⁺): 502.0; 1H NMR (400 MHz, DMSO-d6) δ 8.48- 8.38 (m, 4H), 8.24 (d, J = 1.3 Hz, 1H), 7.48 (d, J = 10.4 Hz, 1H), 7.31-7.25 (m, 1H), 4.53-4.39 (m, 1H), 3.75 (s, 3H), 3.72 (br d, J = 6.6 Hz, 1H), 3.65-3.58 (m, 1H), 3.57-3.51 (m, 1H), 3.46-3.42 (m, 1H), 2.40 (s, 3H), 2.25-2.14 (m, 1H), 2.05-1.95 (m, 1H) |
| A.41 | 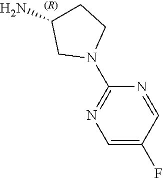 | 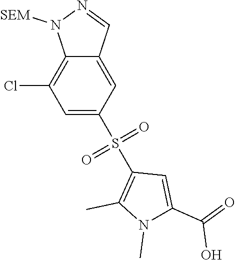 [SMILES: Cc1c(S(=O)(=O)c2cc(Cl)c3c(cnn3COCC[Si](C)(C)C)c2)cc(C(=O)O)n1C] | 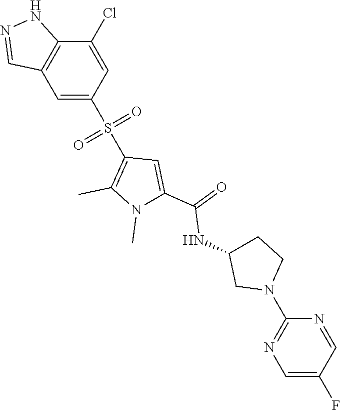 [SMILES: Cc1c(S(=O)(=O)c2cc(Cl)c3[nH]ncc3c2)cc(C(=O)N[C@@H]2CCN(c3ncc(F)cn3)C2)n1C] | LCMS (M + H⁺): 518.0; 1H NMR (400 MHz, DMSO-d6) δ 14.21- 14.02 (m, 1H), 8.50-8.40 (m, 4H), 8.38 (d, J = 1.4 Hz, 1H), 7.72 (s, 1H), 7.32-7.26 (m, 1H), 4.56-4.42 (m, 1H), 3.75 (s, 4H), 3.66-3.58 (m, 1H), 3.57-3.50 (m, 1H), 3.44 (dd, J = 4.5, 11.4 Hz, 1H), 2.40 (s, 3H), 2.20 (br dd, J = 6.3, 13.0 Hz, 1H), 2.08-1.93 (m, 1H) |
| A.42 | 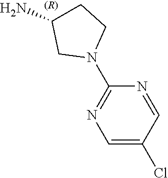 | 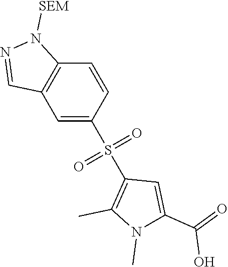 | 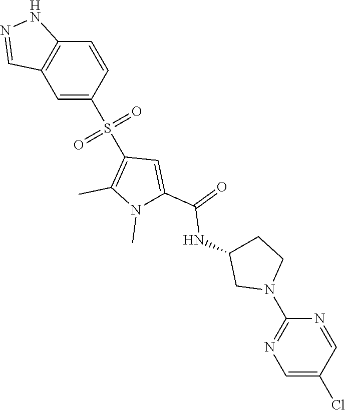 [SMILES: Cc1c(S(=O)(=O)c2ccc3[nH]ncc3c2)cc(C(=O)N[C@@H]2CCN(c3ncc(Cl)cn3)C2)n1C] | LCMS (M + H⁺): 500.0; 1H NMR (400 MHz, DMSO-d6) δ 8.47- 8.36 (m, 4H), 8.30 (s, 1H), 7.70 (d, J = 0.9 Hz, 2H), 7.28 (s, 1H), 4.52-4.42 (m, 1H), 3.76-3.73 (m, 3H), 3.72 (br d, J = 6.5 Hz, 1H), 3.65-3.59 (m, 1H), 3.52 (br s, 1H), 3.46 (br d, J = 4.5 Hz, 1H), 2.39 (s, 3H), 2.24-2.15 (m, 1H), 2.06-1.96 (m, 1H) |
| A.43 | 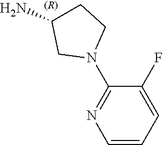 | 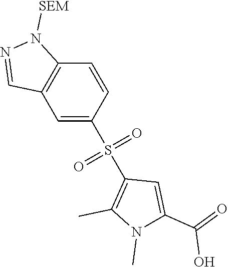 | 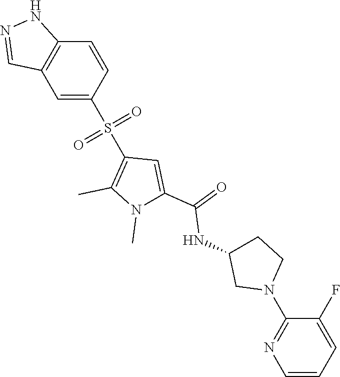 [SMILES: Cc1c(S(=O)(=O)c2ccc3[nH]ncc3c2)cc(C(=O)N[C@@H]2CCN(c3ncccc3F)C2)n1C] | LCMS (M + H⁺): 483.1; 1H NMR (400 MHz, METHANOL-d4) δ 8.45 (d, J = 0.9 Hz, 1H), 8.23 (s, 1H), 7.92-7.75 (m, 2H), 7.65 (d, J = 8.9 Hz, 1H), 7.33-7.24 (m, 1H), 7.20 (s, 1H), 6.60 (ddd, J = 3.1, 4.9, 7.8 Hz, 1H), 4.60-4.52 (m, 1H), 4.00-3.88 (m, 1H), 3.84-3.76 (m, 4H), 3.74-3.65 (m, 1H), 3.58 (ddd, J = 2.6, 5.2, 10.9 Hz, 1H), 2.45 (s, 3H), 2.35-2.21 (m, 1H), 2.14-1.98 (m, 1H) |
| A.44 | 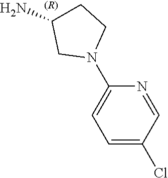 | 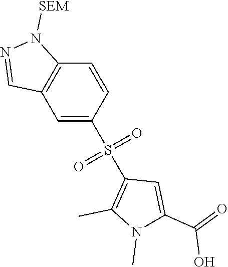 | 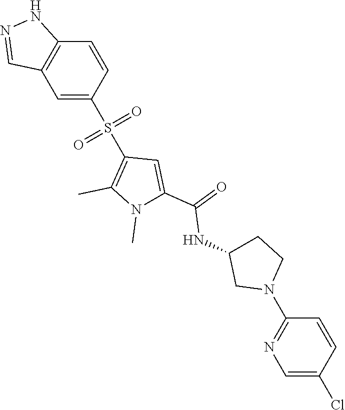 [SMILES: Cc1c(S(=O)(=O)c2ccc3[nH]ncc3c2)cc(C(=O)N[C@@H]2CCN(c3ccc(Cl)cn3)C2)n1C] | LCMS (M + H⁺): 499.0; 1H NMR (400 MHz, METHANOL-d4) δ 8.44 (d, J = 1.0 Hz, 1H), 8.23 (d, J = 0.9 Hz, 1H), 8.01 (d, J = 2.1 Hz, 1H), 7.95 (dd, J = 2.5, 9.6 Hz, 1H), 7.79 (dd, J = 1.8, 8.9 Hz, 1H), 7.66 (d, J = 8.9 Hz, 1H), 7.21 (s, 1H), 7.08 (d, J = 9.6 Hz, 1H), 4.72-4.67 (m, 1H), 3.98-3.87 (m, 1H), 3.82-3.75 (m, 4H), 3.73-3.66 (m, 1H), 3.56 (dd, J = 4.6, 11.1 Hz, 1H), 2.48- 2.40 (m, 4H), 2.31-2.23 (m, 1H) |
| A.45 | 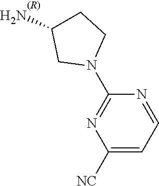 | 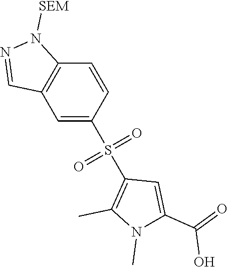 | 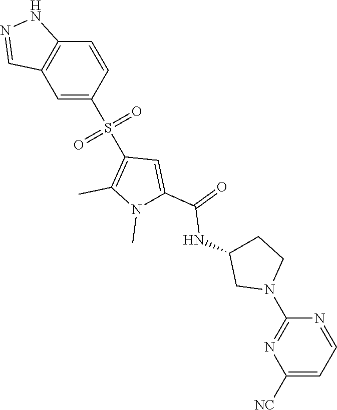 [SMILES: Cc1c(S(=O)(=O)c2ccc3[nH]ncc3c2)cc(C(=O)N[C@@H]2CCN(c3nccc(C#N)n3)C2)n1C] | LCMS (M + H⁺): 491.0; 1H NMR (400 MHz, METHANOL-d4) δ 8.52 (d, J = 4.8 Hz, 1H), 8.45 (s, 1H), 8.23 (s, 1H), 7.80 (dd, J = 1.6, 8.9 Hz, 1H), 7.65 (d, J = 9.0 Hz, 1H), 7.20 (s, 1H), 6.93 (d, J = 4.8 Hz, 1H), 4.58 (br s, 1H), 3.94-3.86 (m, 1H), 3.79 (s, 3H), 3.77-3.70 (m, 1H), 3.69-3.61 (m, 1H), 3.56-3.49 (m, 1H), 2.45 (s, 3H), 2.38-2.28 (m, 1H), 2.17- 2.06 (m, 1H) |
| A.46 | 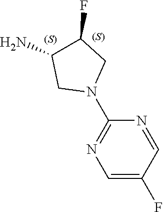 | 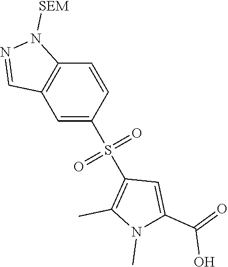 | 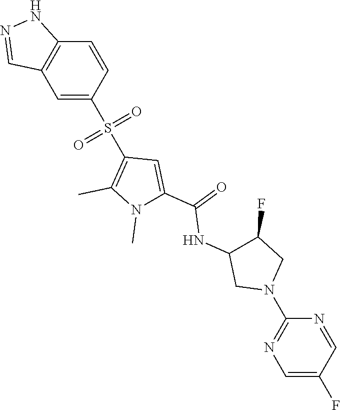 [SMILES: Cc1c(S(=O)(=O)c2ccc3[nH]ncc3c2)cc(C(=O)NC2CN(c3ncc(F)cn3)C[C@@H]2F)n1C] | LCMS (M + H⁺): 502.0; 1H NMR (400 MHz, DMSO-d6) δ 8.54- 8.48 (m, 3H), 8.37 (s, 1H), 8.29 (s, 1H), 7.69 (s, 2H), 7.29 (s, 1H), 5.27-5.09 (m, 1H), 4.60- 4.50 (m, 1H), 3.86-3.78 (m, 2H), 3.75-3.67 (m, 5H), 2.39 (s, 3H) |
| A.47 | 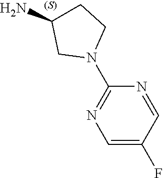 | 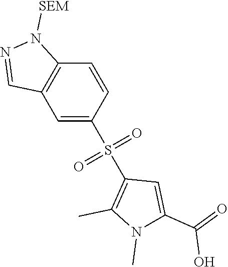 [SMILES: Cc1c(S(=O)(=O)c2ccc3c(cnn3COCC[Si](C)(C)C)c2)cc(C(=O)O)n1C] | 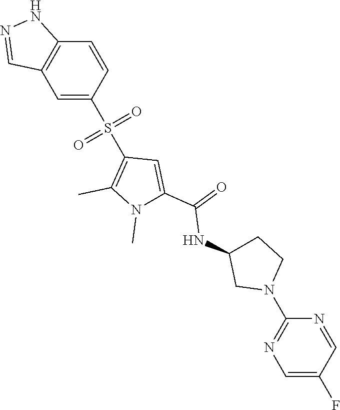 [SMILES: Cc1c(S(=O)(=O)c2ccc3[nH]ncc3c2)cc(C(=O)N[C@H]2CCN(c3ncc(F)cn3)C2)n1C] | LCMS (M + H⁺): 484.1; 1H NMR (400 MHz, METHANOL-d4) δ 8.49-8.40 (m, 1H), 8.31-8.18 (m, 3H), 7.79 (br d, J = 8.9 Hz, 1H), 7.69-7.61 (m, 1H), 7.19 (s, 1H), 4.57 (quin, J = 5.8 Hz, 1H), 3.90- 3.83 (m, 1H), 3.78 (d, J = 1.3 Hz, 3H), 3.75-3.66 (m, 1H), 3.64- 3.55 (m, 1H), 3.52-3.42 (m, 1H), 2.45 (s, 3H), 2.31 (qd, J = 6.1, 12.3 Hz, 1H), 2.09 (qd, J = 6.6, 13.0 Hz, 1H) |
| A.48 | 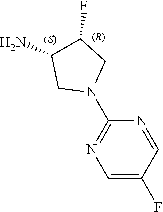 | 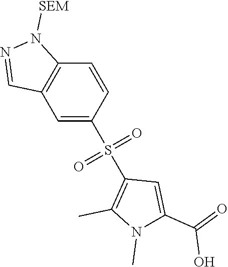 | 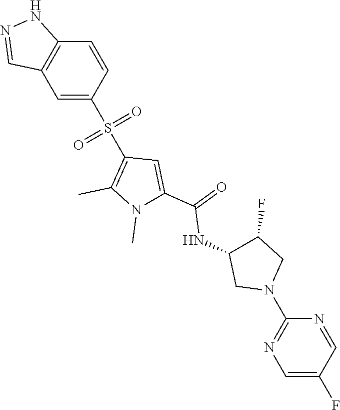 [SMILES: Cc1c(S(=O)(=O)c2ccc3[nH]ncc3c2)cc(C(=O)N[C@H]2CN(c3ncc(F)cn3)C[C@H]2F)n1C] | LCMS (M + H⁺): 502.1; 1H NMR (400 MHz, METHANOL-d4) δ 8.47 (d, J = 1.0 Hz, 1H), 8.32 (s, 2H), 8.25 (s, 1H), 7.83 (dd, J = 1.7, 8.8 Hz, 1H), 7.67 (d, J = 8.9 Hz, 1H), 7.29 (s, 1H), 5.35-5.18 (m, 1H), 4.06 (dd, J = 8.8, 10.3 Hz, 1H), 4.01-3.91 (m, 1H), 3.91-3.85 (m, 1H), 3.85-3.73 (m, 4H), 3.53 (t, J = 10.5 Hz, 1H), 2.47 (s, 3H) |
| A.49 | 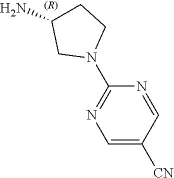 | 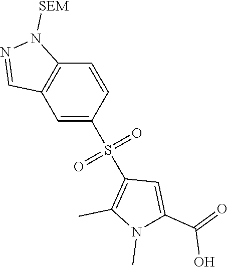 | 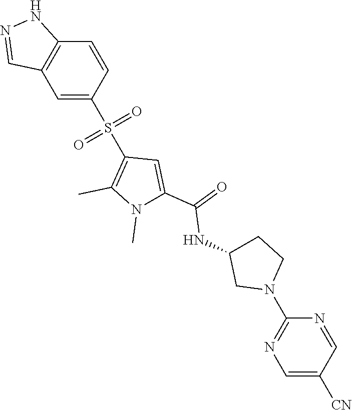 [SMILES: Cc1c(S(=O)(=O)c2ccc3[nH]ncc3c2)cc(C(=O)N[C@@H]2CCN(c3ncc(C#N)cn3)C2)n1C] | LCMS (M + H⁺): 491.1; 1H NMR (400 MHz, METHANOL-d4) δ 8.65-8.56 (m, 2H), 8.44 (d, J = 1.00 Hz, 1H), 8.23 (s, 1H), 7.79 (dd, J = 8.88, 1.75 Hz, 1H), 7.65 (d, J = 9.01 Hz, 1H), 7.20 (s, 1H), 4.60 (br t, J = 5.69 Hz, 1H), 3.94 (dd, J = 12.32, 6.57 Hz, 1H), 3.85-3.76 (m, 4H), 3.75- 3.66 (m, 1H), 3.64-3.55 (m, 1H), 2.45 (s, 3H), 2.39-2.28 (m, 1H), 2.18-2.07 (m, 1H) |
| A.50 | 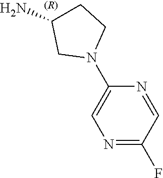 | 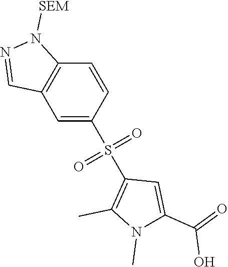 [SMILES: Cc1c(S(=O)(=O)c2ccc3c(cnn3COCC[Si](C)(C)C)c2)cc(C(=O)O)n1C] | 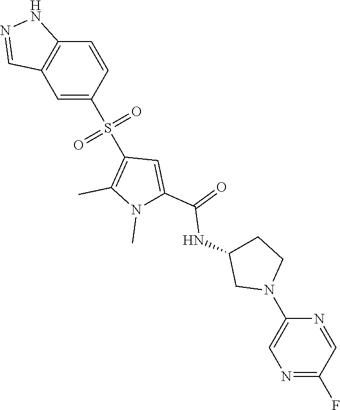 [SMILES: Cc1c(S(=O)(=O)c2ccc3[nH]ncc3c2)cc(C(=O)N[C@@H]2CCN(c3cnc(F)cn3)C2)n1C] | LCMS (M + H⁺): 484.0; 1H NMR (400 MHz, DMSO-d6) δ 8.43 (d, J = 6.75 Hz, 1H), 8.37 (s, 1H), 8.29 (s, 1H), 8.11 (dd, J = 8.25, 1.25 Hz, 1H), 7.69 (s, 2H), 7.54 (d, J = 1.38 Hz, 1H), 7.27 (s, 1H), 4.58-4.45 (m, 1 H), 3.75 (s, 3H), 3.69 (dd, J = 10.69, 6.57 Hz, 1H), 3.58-3.53 (m, 1H), 3.51-3.46 (m, 2H), 2.39 (s, 3H), 2.22 (dq, J = 13.16, 6.62 Hz, 1H), 2.09-1.99 (m, 1H) |
| A.51 | 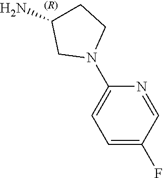 | 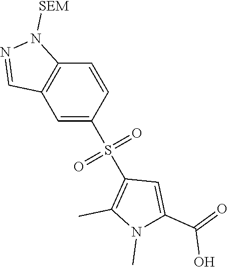 | 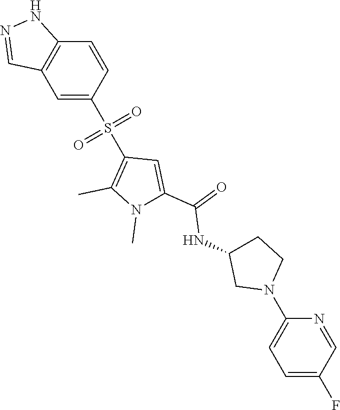 [SMILES: Cc1c(S(=O)(=O)c2ccc3[nH]ncc3c2)cc(C(=O)N[C@@H]2CCN(c3ccc(F)cn3)C2)n1C] | LCMS (M + H⁺): 483.1; 1H NMR (METHANOL-d4, 400 MHz): δ 8.44 (s, 1H), 8.23 (s, 1H), 7.91 (d, J = 2.6 Hz, 1H), 7.80 (dd, J = 8.8, 1.6 Hz, 1H), 7.65 (d, J = 8.9 Hz, 1H), 7.39 (td, J = 8.7, 3.0 Hz, 1H), 7.19 (s, 1H), 6.50 (dd, J = 9.3, 3.4 Hz, 1H), 4.61 (br s, 1H), 3.78 (s, 3H), 3.75 (br d, J = 6.8 Hz, 1H), 3.60 (ddd, J = 9.8, 7.8, 6.1 Hz, 1H), 3.44-3.51 (m, 1H), 3.36 (dd, J = 10.4, 5.3 Hz, 1H), 2.45 (s, 3H), 2.34 (dd, J = 13.6, 6.7 Hz, 1H), 2.11 (dd, J = 13.1, 6.9 Hz, 1H) |
| A.52 | 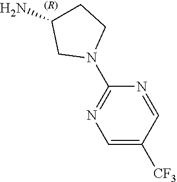 | 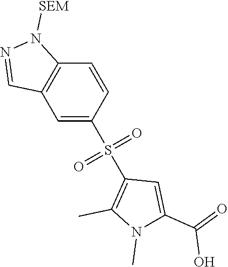 | 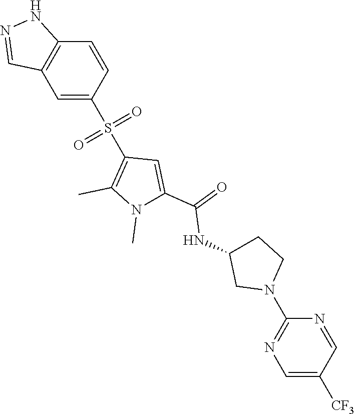 [SMILES: Cc1c(S(=O)(=O)c2ccc3[nH]ncc3c2)cc(C(=O)N[C@@H]2CCN(c3ncc(C(F)(F)F)cn3)C2)n1C] | LCMS (M + H⁺): 534.1; 1H NMR (400 MHz, DMSO-d6) δ 13.51 (br s, 1H), 8.70 (d, J = 3.0 Hz, 2H), 8.45 (d, J = 6.5 Hz, 1H), 8.38 (s, 1H), 8.30 (s, 1H), 7.69 (s, 2H), 7.27 (s, 1H), 4.55-4.43 (m, 1H), 3.81 (dd, J = 6.5, 11.9 Hz, 1H), 3.75 (s, 3H), 3.73-3.68 (m, 1H), 3.65 (dd, J = 6.2, 7.8 Hz, 1H), 3.55 (dd, J = 4.3, 11.9 Hz, 1H), 2.39 (s, 3H), 2.27-2.16 (m, 1H), 2.09-1.99 (m, 1H) |
| A.53 | 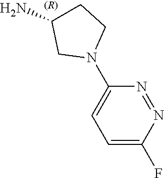 | 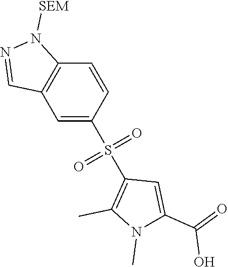 [SMILES: Cc1c(S(=O)(=O)c2ccc3c(cnn3COCC[Si](C)(C)C)c2)cc(C(=O)O)n1C] | 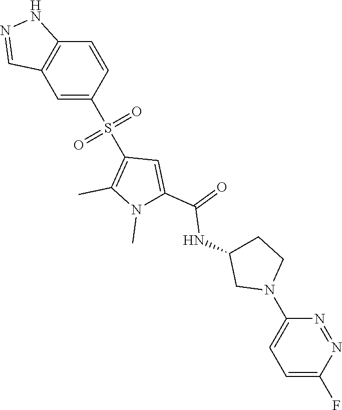 [SMILES: Cc1c(S(=O)(=O)c2ccc3[nH]ncc3c2)cc(C(=O)N[C@@H]2CCN(c3ccc(F)nn3)C2)n1C] | LCMS (M + H⁺): 484.1; 1H NMR (400 MHz, DMSO-d6) δ 13.53 (br s, 1H), 8.45 (d, J = 6.6 Hz, 1H), 8.38 (s, 1H), 8.30 (s, 1H), 7.70 (s, 2H), 7.36 (dd, J = 1.9, 9.6 Hz, 1H), 7.28 (s, 1H), 7.16 (dd, J = 6.8, 9.6 Hz, 1H), 4.60- 4.42 (m, 1H), 3.75 (s, 3H), 3.73- 3.69 (m, 1H), 3.58 (s, 1H), 3.54- 3.48 (m, 1H), 3.41 (br s, 1H), 2.39 (s, 3H), 2.21 (s, 1H), 2.11- 1.98 (m, 1H) |
| A.54 | 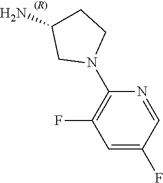 | 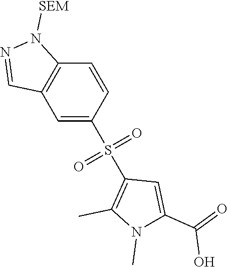 | 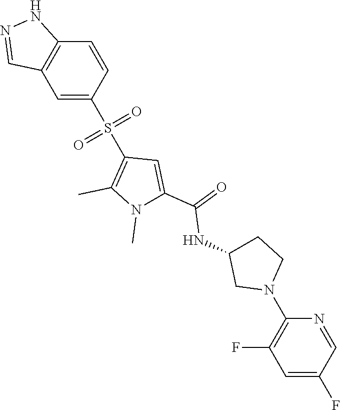 [SMILES: Cc1c(S(=O)(=O)c2ccc3[nH]ncc3c2)cc(C(=O)N[C@@H]2CCN(c3ncc(F)cc3F)C2)n1C] | LCMS (M + H⁺): 501.1; 1H NMR (400 MHz, DMSO-d6) δ 8.40 (d, J = 6.6 Hz, 1H), 8.38 (t, J = 1.2 Hz, 1H), 8.30 (s, 1H), 7.99 (d, J = 2.5 Hz, 1H), 7.71-7.65 (m, 3H), 7.29 (s, 1H), 4.46-4.40 (m, 1H), 3.78 (dt, J = 3.4, 7.1 Hz, 1H), 3.75-3.73 (m, 3H), 3.70- 3.64 (m, 1H), 3.60-3.54 (m, 1H), 3.51-3.46 (m, 1H), 2.40-2.37 (m, 3H), 2.19-2.09 (m, 1H), 1.97 (td, J = 6.4, 12.7 Hz, 1H) |
| A.55 | 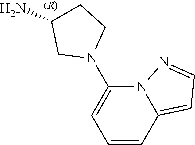 | 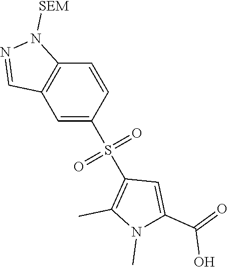 | 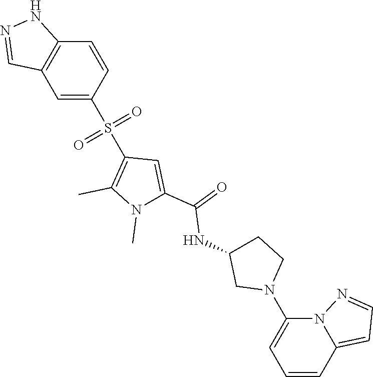 [SMILES: Cc1c(S(=O)(=O)c2ccc3[nH]ncc3c2)cc(C(=O)N[C@@H]2CCN(c3cccc4ccnn34)C2)n1C] | LCMS (M + H⁺): 504.1; 1H NMR (400 MHz, DMSO-d6) δ 8.46 (br d, J = 6.4 Hz, 1H), 8.38 (s, 1H), 8.30 (s, 1H), 7.91 (s, 1H), 7.70 (s, 2H), 7.31 (s, 1H), 7.19-7.04 (m, 2H), 6.48 (br d, J = 1.1 Hz, 1H), 6.01 (br d, J = 4.8 Hz, 1H), 4.51-4.45 (m, 1H), 4.00 (br dd, J = 7.4, 10.1 Hz, 2H), 3.74 (s, 3H), 3.71 (br dd, J = 4.6, 10.4 Hz, 2H), 2.39 (s, 3H), 2.30-1.99 (m, 2H) |
| A.56 | 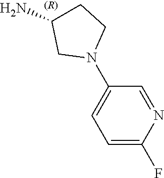 | 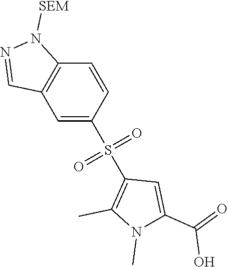 | 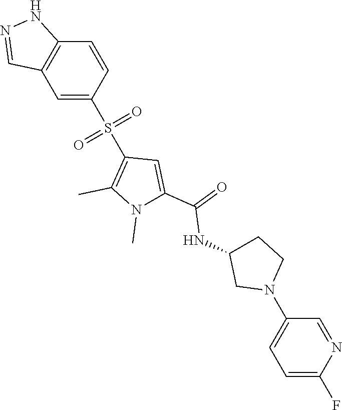 [SMILES: Cc1c(S(=O)(=O)c2ccc3[nH]ncc3c2)cc(C(=O)N[C@@H]2CCN(c3ccc(F)nc3)C2)n1C] | LCMS (M + H⁺): 483.1; 1H NMR (400 MHz, DMSO-d6) δ 13.67- 13.36 (m, 1H), 8.44 (br d, J = 6.9 Hz, 1H), 8.38 (s, 1H), 8.33-8.26 (m, 1H), 7.70 (s, 2H), 7.44 (br s, 1H), 7.28 (s, 1H), 7.19-7.10 (m, 1H), 6.98 (dd, J = 3.2, 8.8 Hz, 1H), 4.53 (qd, J = 6.2, 11.9 Hz, 1H), 3.75 (s, 3H), 3.54 (br dd, J = 7.0, 9.6 Hz, 2H), 3.45-3.40 (m, 1H), 3.31-3.25 (m, 1H), 3.17 (br dd, J = 4.7, 9.7 Hz, 1H), 2.39 (s, 3H), 2.22 (qd, J = 6.7, 13.0 Hz, 1H), 2.12-1.95 (m, 1H) |
| A.57 | 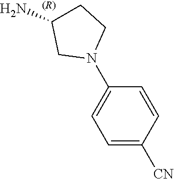 | 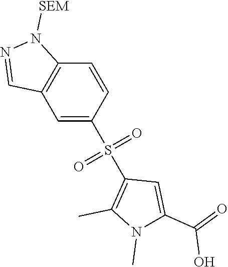 | 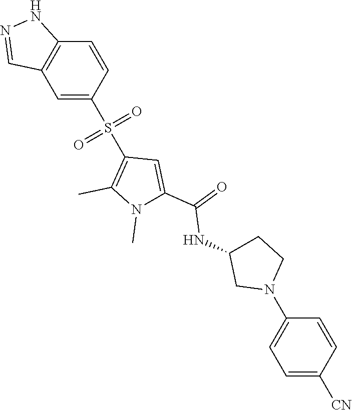 [SMILES: Cc1c(S(=O)(=O)c2ccc3[nH]ncc3c2)cc(C(=O)N[C@@H]2CCN(c3ccc(C#N)cc3)C2)n1C] | LCMS (M + H⁺): 489.2; 1H NMR (400 MHz, METHANOL-d4) δ 8.44 (d, J = 0.9 Hz, 1H), 8.23 (s, 1H), 7.79 (dd, J = 1.7, 8.8 Hz, 1H), 7.65 (d, J = 8.9 Hz, 1H), 7.51-7.45 (m, 2H), 7.19 (s, 1H), 6.64 (d, J = 8.9 Hz, 2H), 4.66- 4.59 (m, 1H), 3.79 (s, 3H), 3.70 (dd, J = 6.7, 10.2 Hz, 1H), 3.59- 3.51 (m, 1H), 3.49-3.40 (m, 1H), 3.30-3.26 (m, 1H), 2.45 (s, 3H), 2.36 (dt, J = 6.8, 13.6 Hz, 1H), 2.19-2.09 (m, 1H) |
| A.58 | 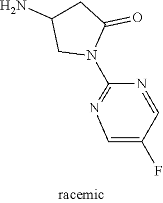 [SMILES: NC1CC(=O)N(c2ncc(F)cn2)C1] | 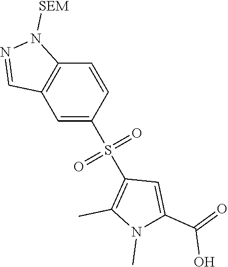 | 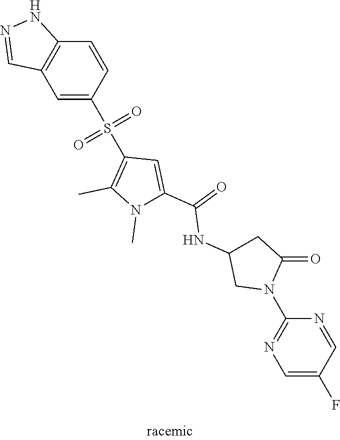 | LCMS (M + H⁺): 491.2; 1H NMR (400 MHz, DMSO-d6) δ 13.52 (br s, 1H), 8.83 (s, 2H), 8.70 (d, J = 6.4 Hz, 1H), 8.37 (s, 1H), 8.31-8.28 (m, 1H), 7.69 (s, 2H), 7.30 (s, 1H), 4.55-4.46 (m, 1H), 4.23 (dd, J = 6.7, 11.2 Hz, 1H), 3.92 (dd, J = 3.1, 11.2 Hz, 1H), 3.75 (s, 3H), 2.99 (dd, J = 8.2, 17.4 Hz, 1H), 2.58 (dd, J = 3.6, 17.4 Hz, 1H), 2.39 (s, 3H) |
| A.59 | 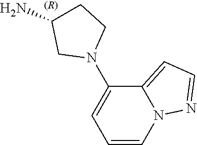 | 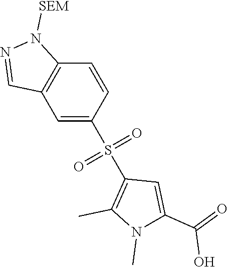 | 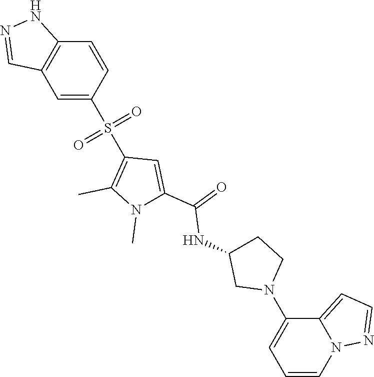 [SMILES: Cc1c(S(=O)(=O)c2ccc3[nH]ncc3c2)cc(C(=O)N[C@@H]2CCN(c3cccn4nccc34)C2)n1C] | LCMS (M + H⁺): 504.1; 1H NMR (400 MHz, DMSO-d6) δ 8.44 (d, J = 5.9 Hz, 1H), 8.36 (s, 1H), 8.28 (s, 1H), 8.02 (d, J = 6.8 Hz, 1H), 7.84 (d, J = 2.5 Hz, 1H), 7.68 (s, 2H), 7.29 (s, 1H), 6.82 (d, J = 2.0 Hz, 1H), 6.69 (t, J = 7.2 Hz, 1H), 6.01 (d, J = 7.5 Hz, 1H), 4.55-4.48 (m, 1H), 3.88- 3.82 (m, 1H), 3.75 (s, 3H), 3.70- 3.65 (m, 1H), 3.59 (br d, J = 7.3 Hz, 1H), 3.53 (br dd, J = 4.9, 9.9 Hz, 1H), 2.39 (s, 3H), 2.23 (dd, J = 6.8, 11.5 Hz, 1H), 2.10-2.01 (m, 1H) |
| A.60 | 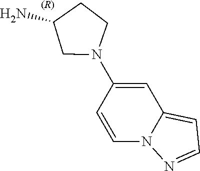 | 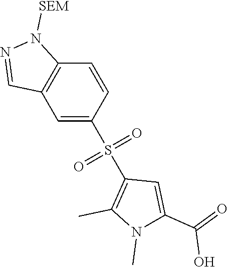 | 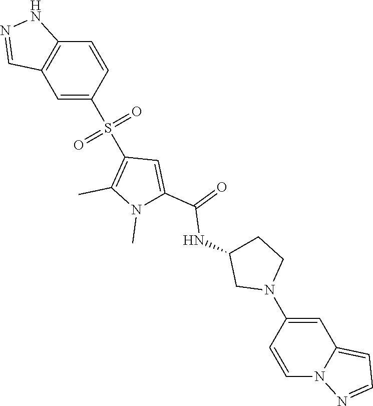 [SMILES: Cc1c(S(=O)(=O)c2ccc3[nH]ncc3c2)cc(C(=O)N[C@@H]2CCN(c3ccn4nccc4c3)C2)n1C] | LCMS (M + H⁺): 504.1; 1H NMR (400 MHz, METHANOL-d4) δ 8.44 (s, 1H), 8.29-8.18 (m, 2H), 7.84-7.75 (m, 1H), 7.72 (d, J = 2.3 Hz, 1H), 7.64 (d, J = 8.9 Hz, 1H), 7.21 (s, 1H), 6.52 (dd, J = 2.5, 7.6 Hz, 1H), 6.40 (d, J = 2.4 Hz, 1H), 6.19-6.13 (m, 1H), 4.64 (br d, J = 6.3 Hz, 1H), 4.61 (br s, 1H), 3.79 (s, 3H), 3.71 (dd, J = 6.8, 9.9 Hz, 1H), 3.61-3.53 (m, 1H), 3.48-3.40 (m, 1H), 2.46 (s, 3H), 2.37 (qd, J = 6.6, 13.3 Hz, 1H), 2.14 (qd, J = 6.7, 13.1 Hz, 1H) |
| A.61 | 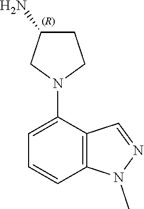 | 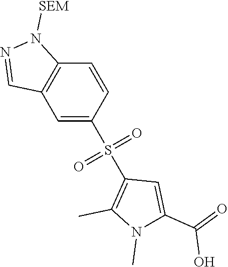 | 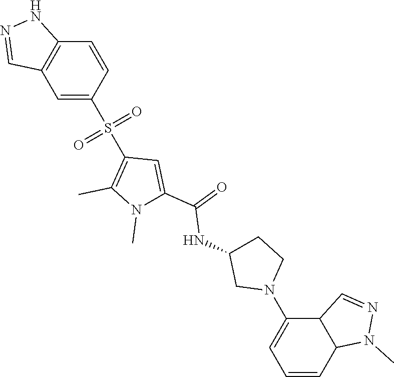 [SMILES: Cc1c(S(=O)(=O)c2ccc3[nH]ncc3c2)cc(C(=O)N[C@@H]2CCN(C3=CC=CC4C3C=NN4C)C2)n1C] | LCMS (M + H⁺): 518.2; 1H NMR (400 MHz, DMSO-d6) δ 8.48 (d, J = 6.6 Hz, 1H), 8.36 (s, 1H), 8.28 (s, 1H), 8.11 (s, 1H), 7.70- 7.66 (m, 2H), 7.29 (s, 1H), 7.15 (t, J = 7.9 Hz, 1H), 6.74 (d, J = 8.3 Hz, 1H), 6.01 (d, J = 7.5 Hz, 1H), 4.59-4.52 (m, 1H), 3.92 (s, 3H), 3.85 (dd, J = 6.7, 9.6 Hz, 1H), 3.75 (s, 3H), 3.74-3.68 (m, 1H), 3.66-3.59 (m, 1H), 3.55- 3.49 (m, 1H), 2.39 (s, 3H), 2.26 (dd, J = 6.6, 12.9 Hz, 1H), 2.08 (s, 1H) |
| A.62 | 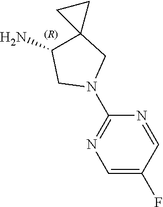 | 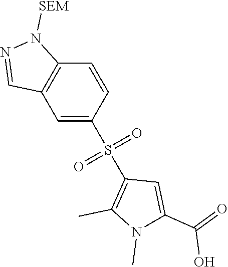 | 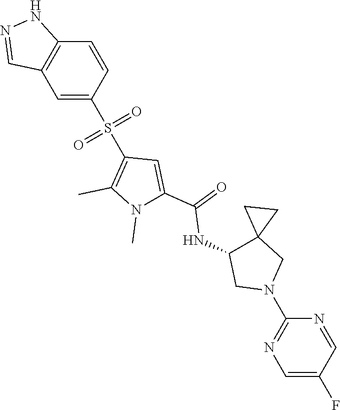 [SMILES: Cc1c(S(=O)(=O)c2ccc3[nH]ncc3c2)cc(C(=O)N[C@H]2CN(c3ncc(F)cn3)CC23CC3)n1C] | LCMS (M + H⁺): 510.1; 1H NMR (400 MHz, METHANOL-d4) δ 8.46-8.40 (m, 1H), 8.26 (d, J = 0.9 Hz, 2H), 8.21 (s, 1H), 7.79 (dd, J = 1.8, 8.9 Hz, 1H), 7.63 (d, J = 8.9 Hz, 1H), 7.20 (s, 1H), 4.25 (dd, J = 3.2, 6.1 Hz, 1H), 3.93 (dd, J = 6.2, 11.6 Hz, 1H), 3.81 (d, J = 11.0 Hz, 1H), 3.77- 3.72 (m, 4H), 3.38 (d, J = 10.9 Hz, 1H), 2.44 (s, 3H), 0.95-0.89 (m, 1H), 0.83-0.77 (m, 1H), 0.73-0.64 (m, 2H) |
| A.63 | 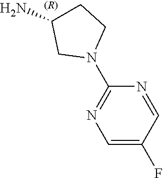 | 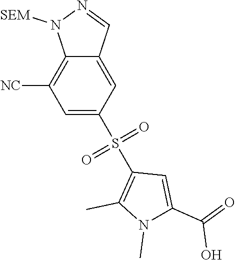 [SMILES: Cc1c(S(=O)(=O)c2cc(C#N)c3c(cnn3COCC[Si](C)(C)C)c2)cc(C(=O)O)n1C] | 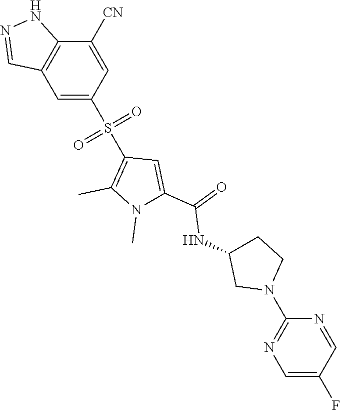 [SMILES: Cc1c(S(=O)(=O)c2cc(C#N)c3[nH]ncc3c2)cc(C(=O)N[C@@H]2CCN(c3ncc(F)cn3)C2)n1C] | LCMS (M + H⁺): 509.1; 1H NMR (400 MHz, METHANOL-d4) δ 8.72 (d, J = 1.38 Hz, 1H), 8.41 (br s, 1H), 8.29 (s, 2H), 8.24 (d, J = 1.13 Hz, 1H), 7.20 (s, 1H), 4.60-4.56 (m, 1H), 3.91-3.83 (m, 1H), 3.79 (s, 3H), 3.76-3.68 (m, 1H), 3.65-3.57 (m, 1H), 3.49 (dd, J = 11.38, 5.00 Hz, 1H), 2.47 (s, 3H), 2.38-2.27 (m, 1H), 2.16-2.04 (m, 1H) |
| A.64 | 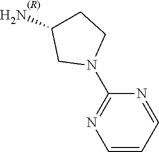 | 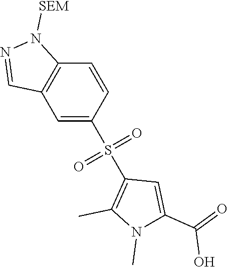 | 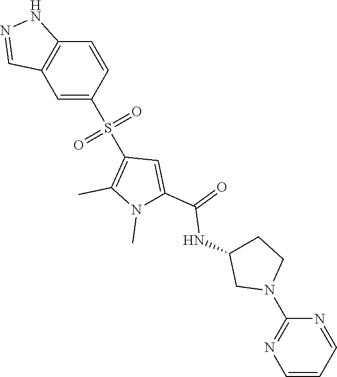 [SMILES: Cc1c(S(=O)(=O)c2ccc3[nH]ncc3c2)cc(C(=O)N[C@@H]2CCN(c3ncccn3)C2)n1C] | LCMS (M + H⁺): 466.1; 1H NMR (400 MHz, METHANOL-d4) δ 8.46-8.42 (m, 1H), 8.32 (d, J = 4.8 Hz, 2H), 8.23 (s, 1H), 7.80 (dd, J = 1.8, 8.9 Hz, 1H), 7.65 (d, J = 8.9 Hz, 1H), 7.19 (s, 1H), 6.62 (t, J = 4.8 Hz, 1H), 4.59 (quin, J = 6.2 Hz, 1H), 3.88 (dd, J = 6.6, 11.4 Hz, 1H), 3.78 (s, 3H), 3.76-3.69 (m, 1H), 3.66- 3.57 (m, 1H), 3.50 (dd, J = 5.3, 11.4 Hz, 1H), 2.45 (s, 3H), 2.33 (qd, J = 6.3, 13.9 Hz, 1H), 2.04 (s, 1H) |
| A.65 | 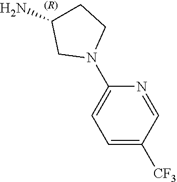 | 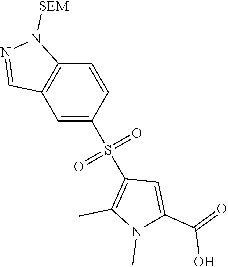 | 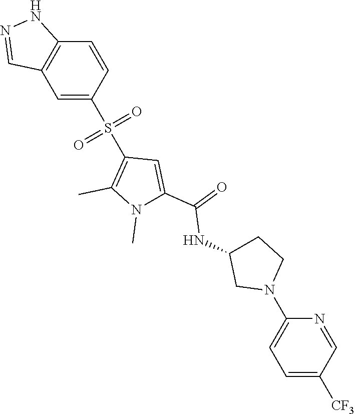 [SMILES: Cc1c(S(=O)(=O)c2ccc3[nH]ncc3c2)cc(C(=O)N[C@@H]2CCN(c3ccc(C(F)(F)F)cn3)C2)n1C] | LCMS (M + H⁺): 533.1; 1H NMR (400 MHz, METHANOL-d4) δ 8.48-8.41 (m, 1H), 8.32-8.26 (m, 1H), 8.22 (s, 1H), 7.79 (dd, J = 1.7, 8.9 Hz, 1H), 7.70 (dd, J = 2.4, 9.0 Hz, 1H), 7.64 (d, J = 8.9 Hz, 1H), 7.20 (s, 1H), 6.59 (d, J = 9.0 Hz, 1H), 4.62 (quin, J = 6.1 Hz, 1H), 3.89-3.81 (m, 1H), 3.78 (s, 3H), 3.73-3.64 (m, 1H), 3.62- 3.52 (m, 1H), 3.45 (dd, J = 5.1, 10.9 Hz, 1H), 2.45 (s, 3H), 2.41- 2.29 (m, 1H), 2.20-2.09 (m, 1H) |
| A.66 | 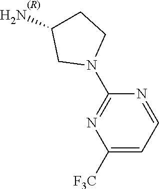 | 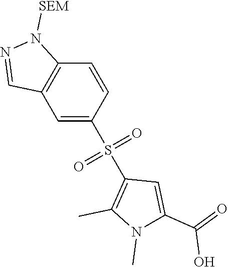 | 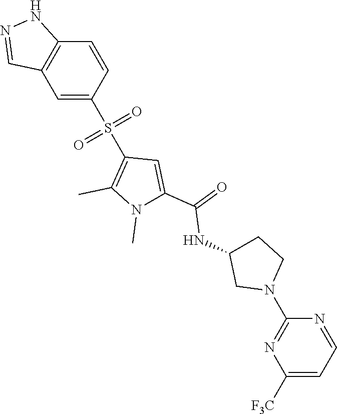 [SMILES: Cc1c(S(=O)(=O)c2ccc3[nH]ncc3c2)cc(C(=O)N[C@@H]2CCN(c3nccc(C(F)(F)F)n3)C2)n1C] | LCMS (M + H⁺): 534.1; 1H NMR (400 MHz, METHANOL-d4) δ 8.54 (d, J = 4.9 Hz, 1H), 8.44 (d, J = 1.0 Hz, 1H), 8.22 (s, 1H), 7.79 (dd, J = 1.6, 8.9 Hz, 1H), 7.64 (d, J = 8.9 Hz, 1H), 7.20 (s, 1H), 6.88 (d, J = 4.9 Hz, 1H), 4.59 (quin, J = 6.0 Hz, 1H), 3.96- 3.87 (m, 1H), 3.78 (s, 3H), 3.77- 3.73 (m, 1H), 3.70-3.61 (m, 1H), 3.54 (dd, J = 5.1, 11.8 Hz, 1H), 2.45 (s, 3H), 2.38-2.28 (m, 1H), 2.11 (qd, J = 6.6, 13.1 Hz, 1H) |
| A.67 | 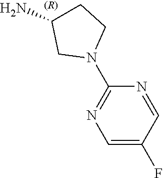 | 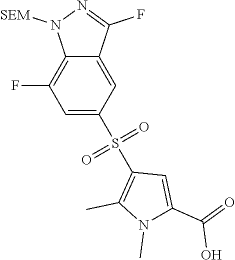 [SMILES: Cc1c(S(=O)(=O)c2cc(F)c3c(c2)c(F)nn3COCC[Si](C)(C)C)cc(C(=O)O)n1C] | 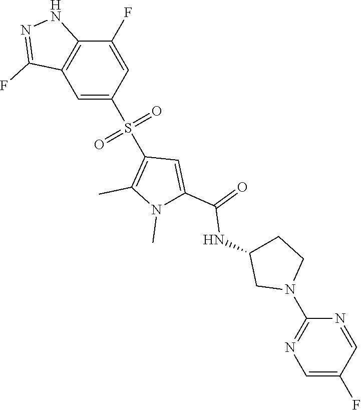 [SMILES: Cc1c(S(=O)(=O)c2cc(F)c3[nH]nc(F)c3c2)cc(C(=O)N[C@@H]2CCN(c3ncc(F)cn3)C2)n1C] | LCMS (M + H⁺): 520.0; 1H NMR (METHANOL-d4, 400 MHz) δ 8.29 (s, 2H), 8.15 (d, J = 1.0 Hz, 1H), 7.58 (dd, J = 10.4, 1.0 Hz, 1H), 7.19 (s, 1H), 4.59 (quin, J = 6.1 Hz, 1H), 3.88 (dd, J = 11.4, 6.6 Hz, 1H), 3.80 (s, 3H), 3.73 (dt, J = 10.7, 7.1 Hz, 1H), 3.57- 3.65 (m, 1H), 3.46-3.52 (m, 1H), 2.47 (s, 3H), 2.28-2.37 (m, 1H), 2.04-2.15 (m, 1H) |
| A.68 | 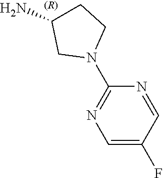 | 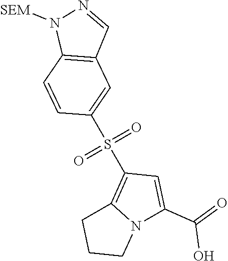 [SMILES: C[Si](C)(C)CCOCn1ncc2cc(S(=O)(=O)c3cc(C(=O)O)n4c3CCC4)ccc21] | 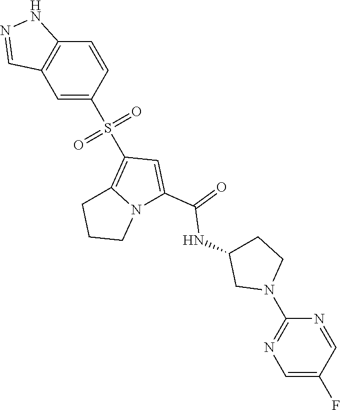 [SMILES: O=C(N[C@@H]1CCN(c2ncc(F)cn2)C1)c1cc(S(=O)(=O)c2ccc3[nH]ncc3c2)c2n1CCC2] | LCMS (M + H⁺): 496.1; 1H NMR (400 MHz, CHLOROFORM-d) δ 8.41 (d, J = 1.0 Hz, 1H), 8.23 (s, 2H), 8.18 (s, 1H), 7.85 (dd, J = 1.6, 8.8 Hz, 1H), 7.56 (d, J = 8.9 Hz, 1H), 6.96 (s, 1H), 6.18 (br d, J = 7.4 Hz, 1H), 4.72-4.63 (m, 1H), 4.32 (t, J = 7.3 Hz, 2H), 3.85 (dd, J = 6.1, 11.6 Hz, 1H), 3.72-3.64 (m, 2H), 3.53 (dd, J = 3.9, 11.8 Hz, 1H), 3.06 (t, J = 7.6 Hz, 2H), 2.53 (quin, J = 7.4 Hz, 2H), 2.38-2.27 (m, 1H), 2.06 (br dd, J = 6.6, 12.5 Hz, 1H) |
| A.69 | 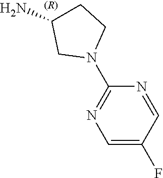 | 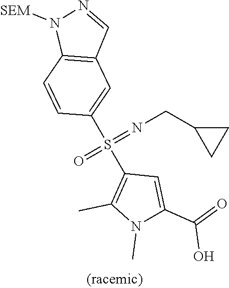 [SMILES: Cc1c(S(=O)(=NCC2CC2)c2ccc3c(cnn3COCC[Si](C)(C)C)c2)cc(C(=O)O)n1C] | 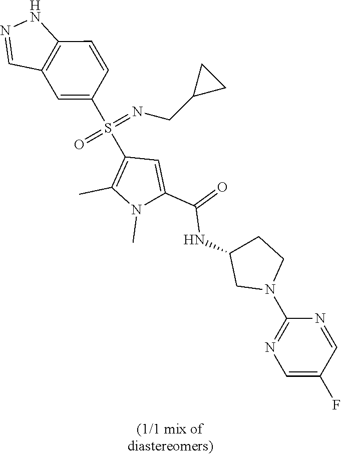 [SMILES: Cc1c(S(=O)(=NCC2CC2)c2ccc3[nH]ncc3c2)cc(C(=O)N[C@@H]2CCN(c3ncc(F)cn3)C2)n1C] | LCMS (M + H⁺): 537.2; 1H NMR (400 MHz, DMSO-d6) δ 8.47- 8.40 (m, 3H), 8.37 (s, 1H), 8.27 (s, 1H), 7.74-7.70 (m, 1H), 7.68- 7.63 (m, 1H), 7.23 (s, 1H), 4.51- 4.43 (m, 1H), 3.76-3.70 (m, 4H), 3.65-3.59 (m, 1H), 3.56-3.50 (m, 1H), 3.45-3.41 (m, 1H), 2.81 (d, J = 6.1 Hz, 2H), 2.34 (s, 3H), 2.24-2.14 (m, 1H), 2.06-1.97 (m, 1H), 1.02-0.93 (m, 1H), 0.41- 0.34 (m, 2H), 0.12 (q, J = 5.0 Hz, 2H) |
| A.70 | 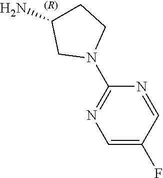 | 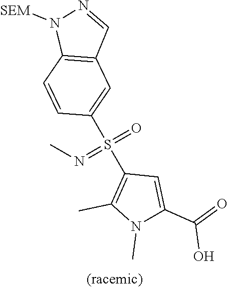 [SMILES: CN=S(=O)(c1ccc2c(cnn2COCC[Si](C)(C)C)c1)c1cc(C(=O)O)n(C)c1C] | 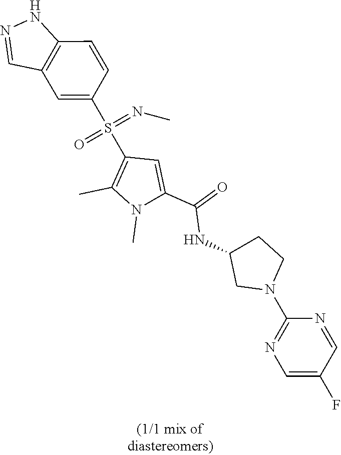 [SMILES: CN=S(=O)(c1ccc2[nH]ncc2c1)c1cc(C(=O)N[C@@H]2CCN(c3ncc(F)cn3)C2)n(C)c1C] | LCMS (M + H⁺): 497.1; ¹H NMR (400 MHz, METHANOL-d4) δ 8.46 (d, J = 1.3 Hz, 1H), 8.28 (d, J = 0.9 Hz, 2H), 8.22 (s, 1H), 7.83 (td, J = 1.8, 8.9 Hz, 1H), 7.67-7.62 (m, 1H), 7.14 (s, 1H), 4.58 (quin, J = 6.1 Hz, 1H), 3.86 (dd, J = 6.6, 11.4 Hz, 1H), 3.78 (s, 3H), 3.76-3.68 (m, 1H), 3.60 (td, J = 7.0, 11.1 Hz, 1H), 3.49 (dd, J = 5.2, 11.3 Hz, 1H), 2.75 (s, 3H), 2.45 (s, 3H), 2.37-2.26 (m, 1H), 2.10 (qd, J = 6.6, 13.2 Hz, 1H) |
| A.71 | 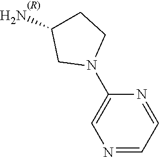 | 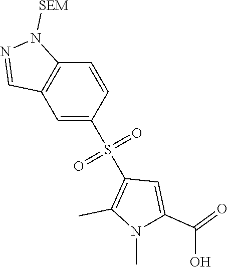 | 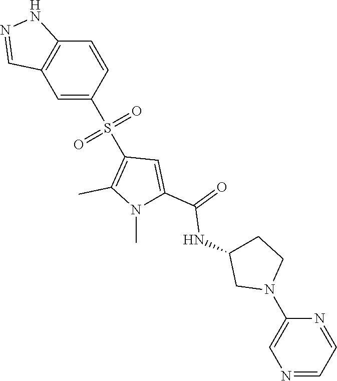 [SMILES: Cc1c(S(=O)(=O)c2ccc3[nH]ncc3c2)cc(C(=O)N[C@@H]2CCN(c3cnccn3)C2)n1C] | LCMS (M + H⁺): 466.1; ¹H NMR (400 MHz, DMSO-d6) δ 8.34 (s, 1H), 8.25 (s, 1H), 8.23 (br s, 1H), 8.05-8.00 (m, 1H), 7.94 (s, 1H), 7.76 (d, J = 2.6 Hz, 1H), 7.67 (s, 2H), 7.24 (s, 1H), 4.59- 4.49 (m, 1H), 3.78-3.76 (m, 3H), 3.76-3.72 (m, 1H), 3.65-3.61 (m, 1H), 3.53-3.49 (m, 1H), 3.41 (br d, J = 6.1 Hz, 1H), 2.40 (s, 3H), 2.29-2.22 (m, 1H), 2.07 (td, J = 6.5, 12.9 Hz, 1H) |
| A.72 | 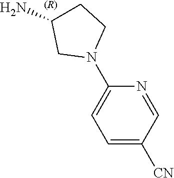 | 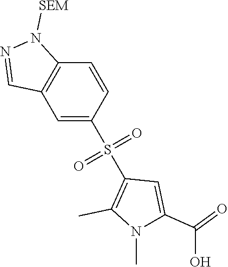 | 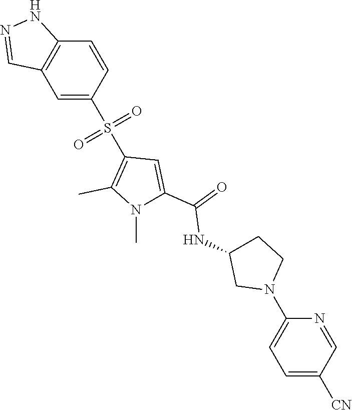 [SMILES: Cc1c(S(=O)(=O)c2ccc3[nH]ncc3c2)cc(C(=O)N[C@@H]2CCN(c3ccc(C#N)cn3)C2)n1C] | LCMS (M + H⁺): 490.1; 1H NMR (400 MHz, METHANOL-d4) δ 8.44 (dd, J = 0.8, 1.6 Hz, 1H), 8.39-8.34 (m, 1H), 8.23 (d, J = 0.6 Hz, 1H), 7.79 (dd, J = 1.8, 8.9 Hz, 1H), 7.71 (dd, J = 2.3, 8.9 Hz, 1H), 7.65 (d, J = 8.9 Hz, 1H), 7.20 (s, 1H), 6.56 (d, J = 9.0 Hz, 1H), 4.62 (td, J = 6.1, 11.7 Hz, 1H), 3.89-3.81 (m, 1H), 3.78 (s, 3H), 3.73-3.43 (m, 3H), 2.45 (s, 3H), 2.41-2.30 (m, 1H), 2.14 (qd, J = 6.5, 13.3 Hz, 1H) |
| A.73 | 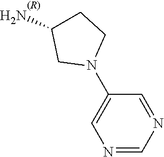 | 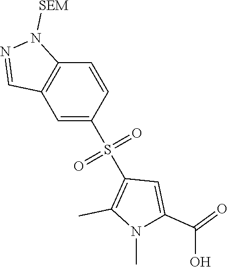 | 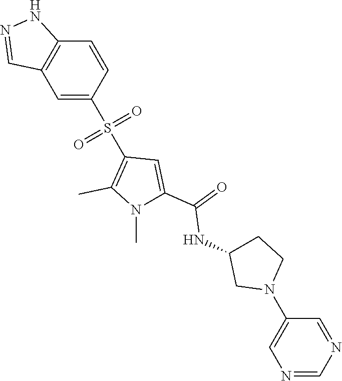 [SMILES: Cc1c(S(=O)(=O)c2ccc3[nH]ncc3c2)cc(C(=O)N[C@@H]2CCN(c3cncnc3)C2)n1C] | LCMS (M + H⁺): 466.1; 1H NMR (400 MHz, DMSO-d6) δ 8.47- 8.42 (m, 2H), 8.39-8.36 (m, 1H), 8.30 (s, 1H), 8.12 (s, 2H), 7.71- 7.68 (m, 2H), 7.27 (s, 1H), 4.60- 4.50 (m, 1H), 3.76-3.74 (m, 3H), 3.59 (dd, J = 6.7, 10.1 Hz, 1H), 3.47-3.44 (m, 1H), 3.37 (br s, 1H), 3.24-3.21 (m, 1H), 2.39 (s, 3H), 2.22 (dd, J = 6.6, 13.1 Hz, 1H), 2.09-1.99 (m, 1H) |
| A.74 | 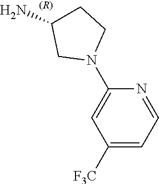 | 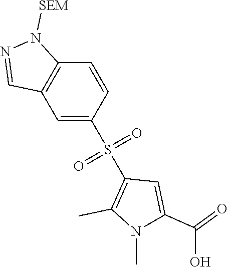 | 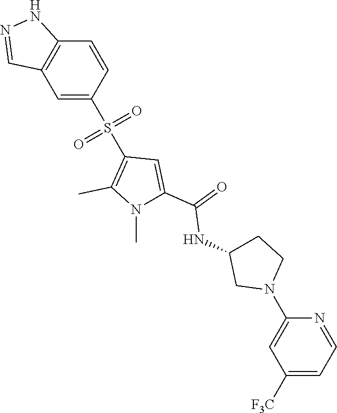 [SMILES: Cc1c(S(=O)(=O)c2ccc3[nH]ncc3c2)cc(C(=O)N[C@@H]2CCN(c3cc(C(F)(F)F)ccn3)C2)n1C] | LCMS (M + H⁺): 533.1; 1H NMR (400 MHz, METHANOL-d4) δ 8.45 (s, 1H), 8.25-8.20 (m, 2H), 7.80 (dd, J = 1.5, 8.9 Hz, 1H), 7.65 (d, J = 8.9 Hz, 1H), 7.20 (s, 1H), 6.79 (d, J = 5.4 Hz, 1H), 6.70 (s, 1H), 4.64 (br d, J = 6.1 Hz, 1H), 3.87-3.81 (m, 1H), 3.79 (s, 3H), 3.70-3.53 (m, 2H), 3.45 (dd, J = 5.1, 10.7 Hz, 1H), 2.45 (s, 3H), 2.37 (qd, J = 6.6, 13.2 Hz, 1H), 2.20-2.11 (m, 1H) |
| A.75 | 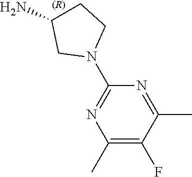 | 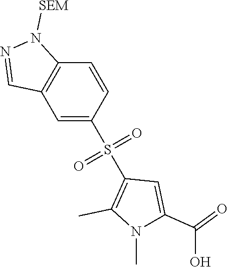 | 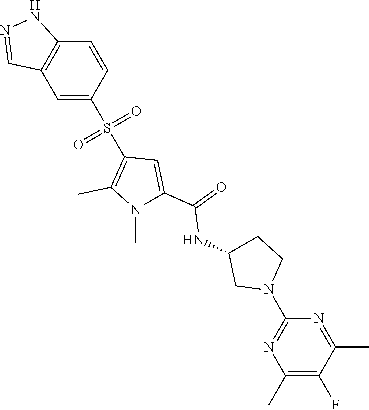 [SMILES: Cc1nc(N2CC[C@@H](NC(=O)c3cc(S(=O)(=O)c4ccc5[nH]ncc5c4)c(C)n3C)C2)nc(C)c1F] | LCMS (M + H⁺): 512.1; 1H NMR (400 MHz, METHANOL-d4) δ 8.44 (d, J = 1.0 Hz, 1H), 8.22 (d, J = 0.6 Hz, 1H), 7.79 (dd, J = 1.6, 8.9 Hz, 1H), 7.64 (d, J = 8.9 Hz, 1H), 7.18 (s, 1H), 4.53 (quin, J = 6.1 Hz, 1H), 3.84 (dd, J = 6.6, 11.4 Hz, 1H), 3.78 (s, 3H), 3.73-3.65 (m, 1H), 3.57 (td, J = 7.2, 10.8 Hz, 1H), 3.45 (dd, J = 5.3, 11.4 Hz, 1H), 2.45 (s, 3H), 2.30 (d, J = 2.8 Hz, 6H), 2.29- 2.23 (m, 1H), 2.12-1.99 (m, 1H) |
| A.76 | 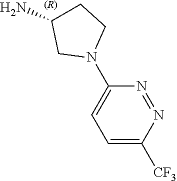 | 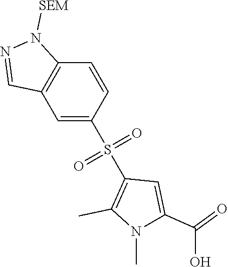 | 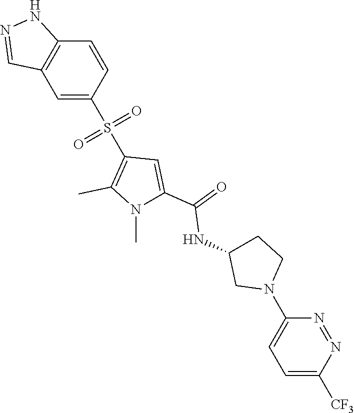 [SMILES: Cc1c(S(=O)(=O)c2ccc3[nH]ncc3c2)cc(C(=O)N[C@@H]2CCN(c3ccc(C(F)(F)F)nn3)C2)n1C] | LCMS (M + H⁺): 534.1; 1H NMR (400 MHz, DMSO-d6) δ 13.68- 13.39 (m, 1H), 8.47 (d, J = 6.5 Hz, 1H), 8.38 (s, 1H), 8.30 (s, 1H), 7.77 (d, J = 9.5 Hz, 1H), 7.70 (s, 2H), 7.27 (s, 1H), 7.04 (d, J = 9.4 Hz, 1H), 4.56 (br d, J = 4.8 Hz, 1H), 3.81 (br dd, J = 6.1, 10.0 Hz, 1H), 3.75 (s, 3H), 3.72-3.45 (m, 3H), 2.39 (s, 3H), 2.32-2.20 (m, 1H), 2.13-2.03 (m, 1H) |
| A.77 | 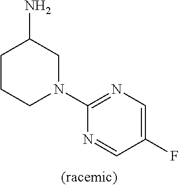 [SMILES: NC1CCCN(c2ncc(F)cn2)C1] | 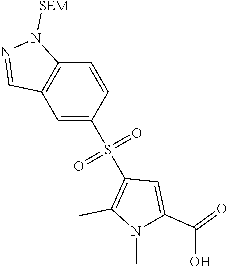 | 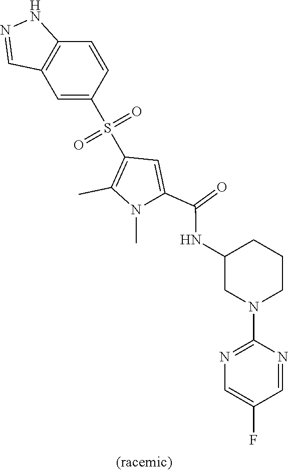 [SMILES: Cc1c(S(=O)(=O)c2ccc3[nH]ncc3c2)cc(C(=O)NC2CCCN(c3ncc(F)cn3)C2)n1C] | LCMS (M + H⁺): 498.2; 1H NMR (400 MHz, DMSO-d6) δ 13.91- 13.07 (m, 1H), 8.43 (s, 2H), 8.38 (s, 1H), 8.30 (s, 1H), 8.17 (d, J = 8.0 Hz, 1H), 7.70 (s, 2H), 7.24 (s, 1H), 4.55 (br dd, J = 4.1, 12.3 Hz, 1H), 4.43 (br d, J = 13.4 Hz, 1H), 3.74 (s, 4H), 2.94-2.80 (m, 2H), 2.40 (s, 3H), 1.93-1.86 (m, 1H), 1.82-1.74 (m, 1H), 1.63- 1.52 (m, 1H), 1.51-1.38 (m, 1H) |
| A.78 | 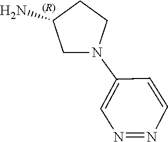 | 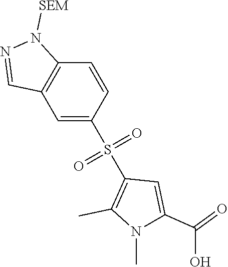 | 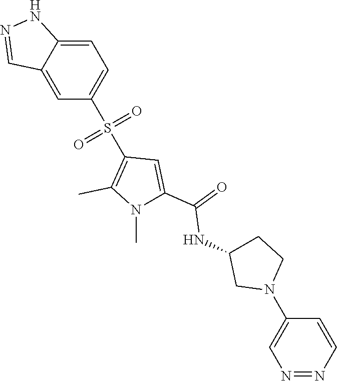 [SMILES: Cc1c(S(=O)(=O)c2ccc3[nH]ncc3c2)cc(C(=O)N[C@@H]2CCN(c3ccnnc3)C2)n1C] | LCMS (M + H⁺): 466.1; 1H NMR (400 MHz, METHANOL-d4) δ 8.55 (d, J = 2.8 Hz, 1H), 8.52 (d, J = 6.4 Hz, 1H), 8.46-8.40 (m, 1H), 8.25-8.19 (m, 1H), 7.82- 7.75 (m, 1H), 7.68-7.60 (m, 1H), 7.20 (s, 1H), 6.67 (dd, J = 3.1, 6.4 Hz, 1H), 4.70-4.61 (m, 1H), 3.80-3.76 (m, 3H), 3.76-3.69 (m, 1H), 3.65-3.45 (m, 2H), 3.38 (br dd, J = 4.8, 10.5 Hz, 1H), 2.45 (s, 3H), 2.42-2.31 (m, 1H), 2.23- 2.11 (m, 1H) |
| A.79 | 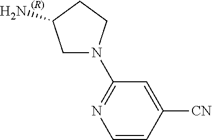 | 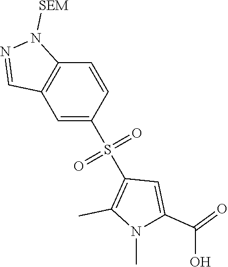 | 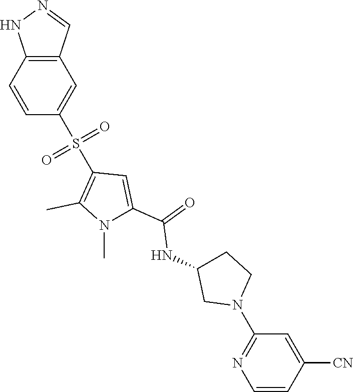 [SMILES: Cc1c(S(=O)(=O)c2ccc3[nH]ncc3c2)cc(C(=O)N[C@@H]2CCN(c3cc(C#N)ccn3)C2)n1C] | LCMS (M + H⁺): 490.1; 1H NMR (400 MHz, DMSO-d6) δ 8.52 (d, J = 6.6 Hz, 1H), 8.46 (s, 1H), 8.40-8.33 (m, 2H), 7.78 (s, 2H), 7.36 (s, 1H), 7.03-6.93 (m, 2H), 4.66-4.53 (m, 1H), 3.84 (s, 3H), 3.78 (br dd, J = 6.7, 11.1 Hz, 1H), 3.69-3.56 (m, 3H), 2.48 (s, 3H), 2.37-2.22 (m, 1H), 2.19- 2.03 (m, 1H) |
| A.80 | 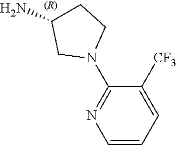 | 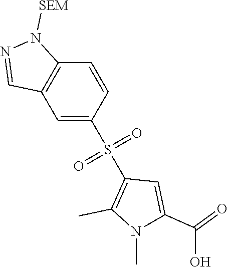 | 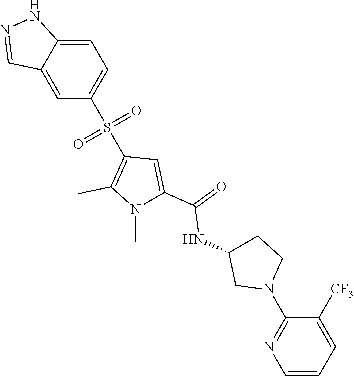 [SMILES: Cc1c(S(=O)(=O)c2ccc3[nH]ncc3c2)cc(C(=O)N[C@@H]2CCN(c3ncccc3C(F)(F)F)C2)n1C] | LCMS (M + H⁺): 533.1; 1H NMR (400 MHz, DMSO-d6) δ 8.42 (d, J = 6.4 Hz, 1H), 8.37 (s, 1H), 8.36-8.33 (m, 1H), 8.29 (s, 1H), 7.93 (dd, J = 1.6, 7.8 Hz, 1H), 7.72-7.66 (m, 2H), 7.27 (s, 1H), 6.81 (dd, J = 4.6, 7.8 Hz, 1H), 4.45-4.37 (m, 1H), 3.76-3.72 (m, 4H), 3.70-3.65 (m, 1H), 3.63- 3.58 (m, 1H), 3.52-3.48 (m, 1H), 2.40-2.37 (m, 3H), 2.15 (qd, J = 6.4, 12.4 Hz, 1H), 1.98 (qd, J = 6.5, 12.6 Hz, 1H) |
| A.81 | 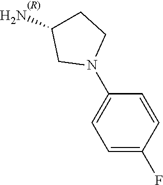 | 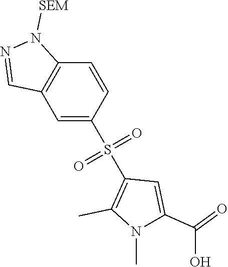 | 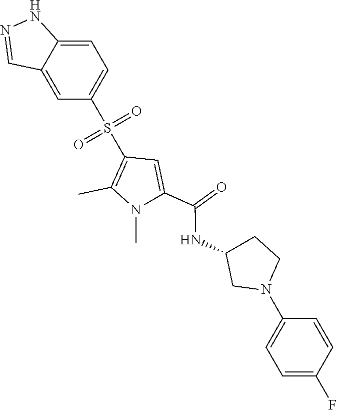 [SMILES: Cc1c(S(=O)(=O)c2ccc3[nH]ncc3c2)cc(C(=O)N[C@@H]2CCN(c3ccc(F)cc3)C2)n1C] | LCMS (M + H⁺): 482.1; 1H NMR (400 MHz, DMSO-d6) δ 13.95- 13.03 (m, 1H), 8.42 (d, J = 6.8 Hz, 1H), 8.37 (s, 1H), 8.30 (s, 1H), 7.69 (s, 2H), 7.28 (s, 1H), 7.01 (t, J = 8.9 Hz, 2H), 6.56- 6.47 (m, 2H), 4.55-4.46 (m, 1H), 3.75 (s, 3H), 3.50 (dd, J = 6.9, 9.6 Hz, 1H), 3.36 (br d, J = 8.1 Hz, 1H), 3.24 (br d, J = 6.8 Hz, 1H), 3.13 (dd, J = 4.8, 9.6 Hz, 1H), 2.39 (s, 3H), 2.27-2.17 (m, 1H), 2.01 (qd, J = 6.4, 12.8 Hz, 1H) |
| A.82 | 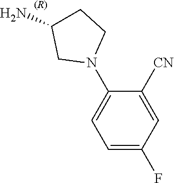 | 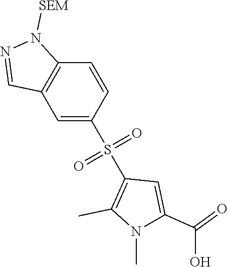 | 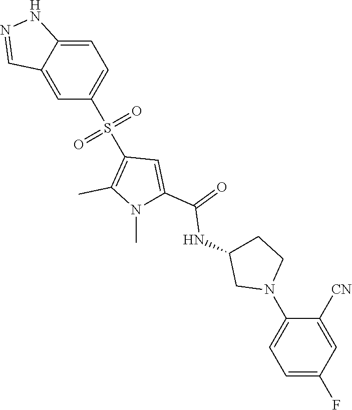 [SMILES: Cc1c(S(=O)(=O)c2ccc3[nH]ncc3c2)cc(C(=O)N[C@@H]2CCN(c3ccc(F)cc3C#N)C2)n1C] | LCMS (M + H⁺): 507.2; 1H NMR (400 MHz, DMSO-d6) δ 13.73- 13.22 (m, 1H), 8.44 (d, J = 6.4 Hz, 1H), 8.38 (s, 1H), 8.30 (s, 1H), 7.70 (s, 2H), 7.47 (dd, J = 3.1, 8.7 Hz, 1H), 7.39-7.32 (m, 1H), 7.28 (s, 1H), 6.81 (dd, J = 4.6, 9.6 Hz, 1H), 4.52-4.45 (m, 1H), 3.78 (br d, J = 3.5 Hz, 1H), 3.74 (s, 3H), 3.66 (br d, J = 9.0 Hz, 1H), 3.54 (br d, J = 7.4 Hz, 1H), 3.48 (br d, J = 5.6 Hz, 1H), 2.39 (s, 3H), 2.22-2.15 (m, 1H), 2.07-1.99 (m, 1H) |
| A.83 | 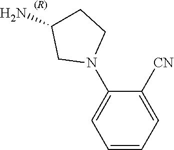 | 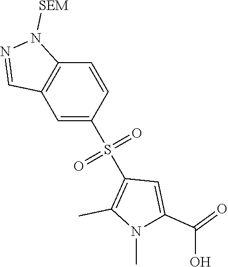 | 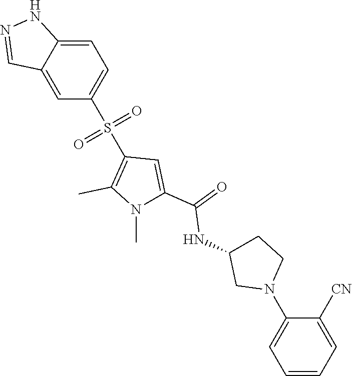 [SMILES: Cc1c(S(=O)(=O)c2ccc3[nH]ncc3c2)cc(C(=O)N[C@@H]2CCN(c3ccccc3C#N)C2)n1C] | LCMS (M + H⁺): 489.1; 1H NMR (400 MHz, DMSO-d6) δ 13.69- 13.32 (m, 1H), 8.45 (d, J = 6.5 Hz, 1H), 8.38 (s, 1H), 8.30 (s, 1H), 7.70 (s, 2H), 7.49 (dd, J = 1.6, 7.8 Hz, 1H), 7.42 (ddd, J = 1.7, 7.2, 8.7 Hz, 1H), 7.29 (s, 1H), 6.78 (d, J = 8.8 Hz, 1H), 6.72 (t, J = 7.4 Hz, 1H), 4.54- 4.44 (m, 1H), 3.81 (dd, J = 6.4, 10.1 Hz, 1H), 3.75 (s, 3H), 3.72- 3.67 (m, 1H), 3.60-3.56 (m, 1H), 3.52 (br d, J = 4.6 Hz, 1H), 2.39 (s, 3H), 2.19 (br dd, J = 6.1, 12.7 Hz, 1H), 2.08-2.00 (m, 1H) |
| A.84 | 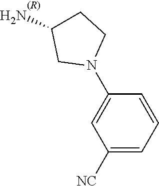 | 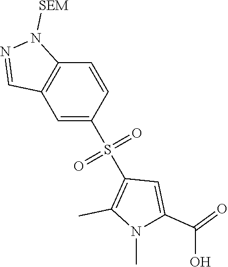 | 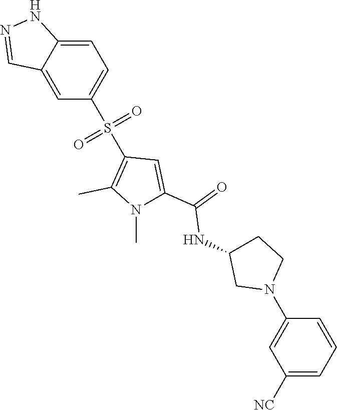 [SMILES: Cc1c(S(=O)(=O)c2ccc3[nH]ncc3c2)cc(C(=O)N[C@@H]2CCN(c3cccc(C#N)c3)C2)n1C] | LCMS (M + H⁺): 489.1; 1H NMR (400 MHz, DMSO-d6) δ 13.52 (br s, 1H), 8.44 (d, J = 6.8 Hz, 1H), 8.39 (s, 1H), 8.31 (s, 1H), 7.70 (s, 2H), 7.35 (t, J = 7.9 Hz, 1H), 7.28 (s, 1H), 6.99 (d, J = 7.5 Hz, 1H), 6.90 (s, 1H), 6.86 (dd, J = 2.1, 8.6 Hz, 1H), 4.59-4.48 (m, 1H), 3.80-3.73 (m, 3H), 3.62- 3.53 (m, 1H), 3.40-3.39 (m, 1H), 3.48-3.37 (m, 1H), 3.21 (dd, J = 4.6, 10.3 Hz, 1H), 2.40 (s, 3H), 2.23 (qd, J = 6.7, 13.0 Hz, 1H), 2.10-1.98 (m, 1H) |
| A.85 | 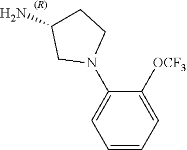 | 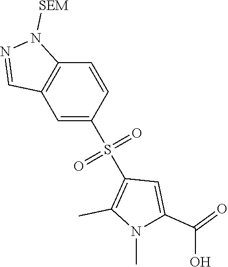 | 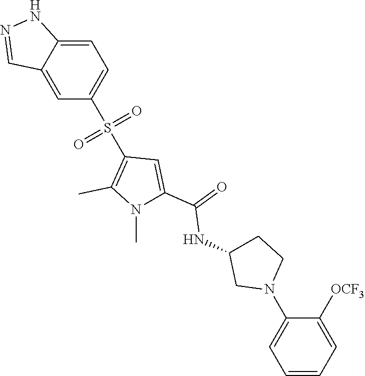 [SMILES: Cc1c(S(=O)(=O)c2ccc3[nH]ncc3c2)cc(C(=O)N[C@@H]2CCN(c3ccccc3OC(F)(F)F)C2)n1C] | LCMS (M + H⁺): 548.1; 1H NMR (400 MHz, DMSO-d6) δ 13.98- 13.06 (m, 1H), 8.39 (d, J = 6.6 Hz, 1H), 8.37 (s, 1H), 8.29 (s, 1H), 7.69 (s, 2H), 7.27 (s, 1H), 7.19 (d, J = 7.9 Hz, 2H), 6.85 (d, J = 7.1 Hz, 1H), 6.78-6.73 (m, 1H), 4.46-4.39 (m, 1H), 3.74 (s, 3H), 3.61 (dd, J = 6.8, 9.8 Hz, 1H), 3.47-3.43 (m, 1H), 3.38 (br s, 2H), 2.39 (s, 3H), 2.19-2.11 (m, 1H), 2.03-1.95 (m, 1H) |
| A.86 | 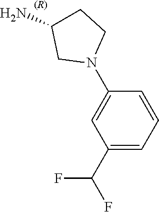 | 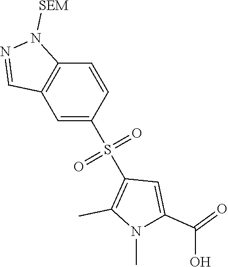 | 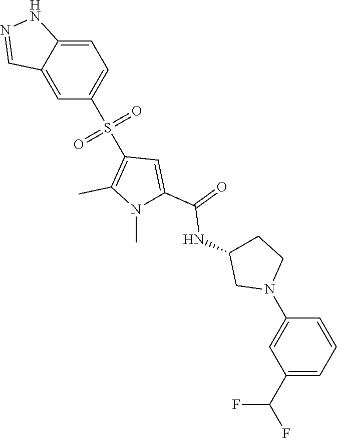 [SMILES: Cc1c(S(=O)(=O)c2ccc3[nH]ncc3c2)cc(C(=O)N[C@@H]2CCN(c3cccc(C(F)F)c3)C2)n1C] | LCMS (M + H⁺): 514.1; 1H NMR (400 MHz, DMSO-d6) δ 13.51 (br s, 1H), 8.44 (d, J = 6.8 Hz, 1H), 8.38 (s, 1H), 8.30 (s, 1H), 7.69 (s, 2H), 7.32-7.25 (m, 2H), 7.07-6.88 (m, 1H), 6.78 (d, J = 9.0 Hz, 1H), 6.72-6.63 (m, 2H), 4.58-4.47 (m, 1H), 3.75 (s, 3H), 3.56 (dd, J = 6.9, 9.8 Hz, 1H), 3.46-3.38 (m, 2H), 3.20 (dd, J = 4.7, 9.8 Hz, 1H), 2.39 (s, 3H), 2.30-2.18 (m, 1H), 2.10-1.99 (m, 1H) |
| A.87 | 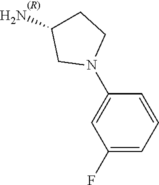 | 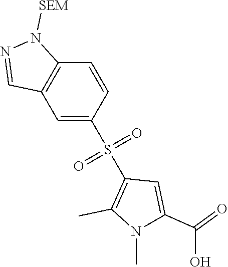 | 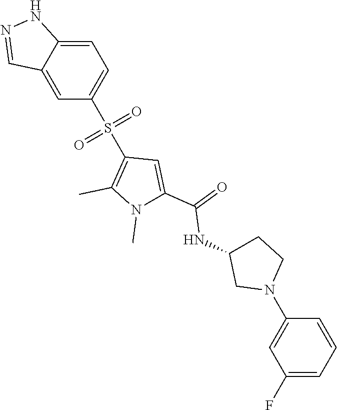 [SMILES: Cc1c(S(=O)(=O)c2ccc3[nH]ncc3c2)cc(C(=O)N[C@@H]2CCN(c3cccc(F)c3)C2)n1C] | LCMS (M + H⁺): 482.1; 1H NMR (400 MHz, DMSO-d6) δ 8.35 (d, J = 0.6 Hz, 1H), 8.27 (s, 1H), 8.24 (br d, J = 6.6 Hz, 1H), 7.74- 7.66 (m, 2H), 7.25 (s, 1H), 7.16 (q, J = 8.1 Hz, 1H), 6.39-6.33 (m, 2H), 6.30 (dd, J = 2.3, 12.6 Hz, 1H), 4.53 (br d, J = 5.5 Hz, 1H), 3.77 (s, 3H), 3.55 (br s, 1H), 3.41 (br s, 1H), 3.32-3.28 (m, 1H), 3.19 (dd, J = 5.0, 9.9 Hz, 1H), 2.41 (s, 3H), 2.24 (td, J = 6.5, 13.4 Hz, 1H), 2.12-1.99 (m, 1H) |
| A.88 | 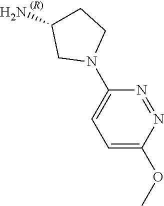 | 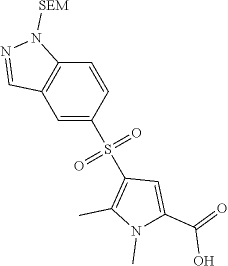 | 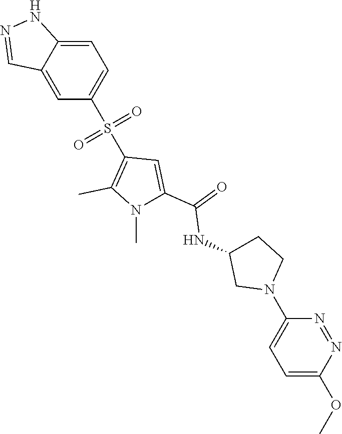 [SMILES: COc1ccc(N2CC[C@@H](NC(=O)c3cc(S(=O)(=O)c4ccc5[nH]ncc5c4)c(C)n3C)C2)nn1] | LCMS (M + H⁺): 496.1; 1H NMR (400 MHz, DMSO-d6) δ 13.55 (br d, J = 3.1 Hz, 1H), 8.49- 8.29 (m, 3H), 7.70 (s, 2H), 7.28 (s, 1H), 7.00 (s, 2H), 4.57-4.45 (m, 1H), 3.89-3.86 (m, 3H), 3.76-3.74 (m, 3H), 3.69 (dd, J = 6.7, 10.6 Hz, 1H), 3.59-3.52 (m, 1H), 3.50-3.44 (m, 2H), 2.39 (s, 3H), 2.21 (br dd, J = 6.6, 12.9 Hz, 1H), 2.08-1.96 (m, 1H) |
| A.89 | 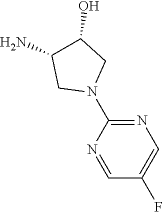 [SMILES: N[C@H]1CN(c2ncc(F)cn2)C[C@H]1O] | 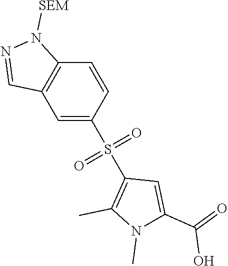 | 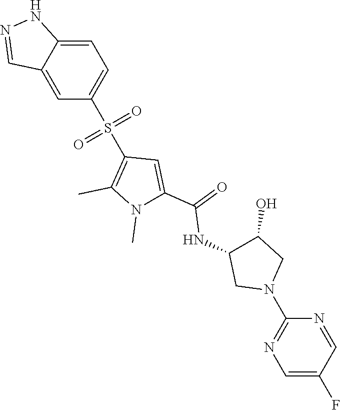 [SMILES: Cc1c(S(=O)(=O)c2ccc3[nH]ncc3c2)cc(C(=O)N[C@H]2CN(c3ncc(F)cn3)C[C@H]2O)n1C] | LCMS (M + H⁺): 500.1; 1H NMR (400 MHz, METHANOL-d4) δ 8.47 (d, J = 0.9 Hz, 1H), 8.29 (d, J = 0.8 Hz, 2H), 8.24 (d, J = 0.6 Hz, 1H), 7.82 (dd, J = 1.8, 8.9 Hz, 1H), 7.66 (d, J = 8.9 Hz, 1H), 7.28 (s, 1H), 4.57-4.53 (m, 1H), 4.45 (dt, J = 1.7, 3.8 Hz, 1H), 3.94 (dd, J = 8.1, 10.5 Hz, 1H), 3.79 (s, 3H), 3.75-3.66 (m, 2H), 3.51 (t, J = 10.1 Hz, 1H), 2.47 (s, 3H) |
| A.90 | 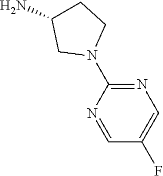 [SMILES: N[C@@H]1CCN(c2ncc(F)cn2)C1] | 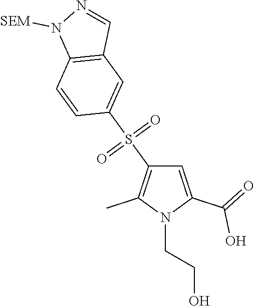 [SMILES: Cc1c(S(=O)(=O)c2ccc3c(cnn3COCC[Si](C)(C)C)c2)cc(C(=O)O)n1CCO] | 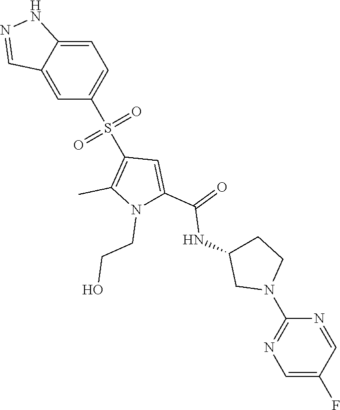 [SMILES: Cc1c(S(=O)(=O)c2ccc3[nH]ncc3c2)cc(C(=O)N[C@@H]2CCN(c3ncc(F)cn3)C2)n1CCO] | LCMS (M + H⁺): 514.1; 1H NMR (400 MHz, METHANOL-d4) δ 8.45 (s, 1H), 8.28 (s, 2H), 8.24 (s, 1H), 7.81 (br d, J = 8.9 Hz, 1H), 7.66 (d, J = 9.0 Hz, 1H), 7.26-7.22 (m, 1H), 4.62-4.53 (m, 1H), 4.41 (br t, J = 4.9 Hz, 2H), 3.91-3.83 (m, 1H), 3.79-3.67 (m, 3H), 3.65-3.56 (m, 1H), 3.49 (br dd, J = 5.2, 11.3 Hz, 1H), 2.52 (s, 3H), 2.31 (qd, J = 6.3, 12.6 Hz, 1H), 2.10 (qd, J = 6.6, 12.7 Hz, 1H) |
| A.91 | 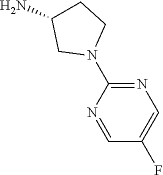 [SMILES: N[C@@H]1CCN(c2ncc(F)cn2)C1] | 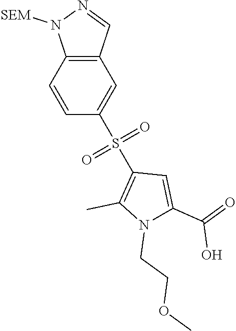 [SMILES: COCCn1c(C(=O)O)cc(S(=O)(=O)c2ccc3c(cnn3COCC[Si](C)(C)C)c2)c1C] | 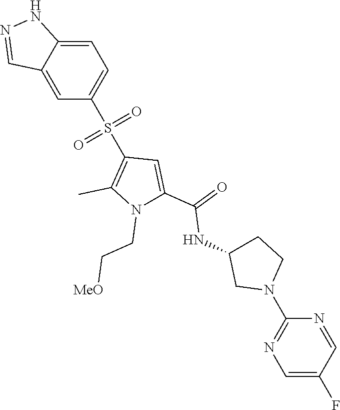 [SMILES: COCCn1c(C(=O)N[C@@H]2CCN(c3ncc(F)cn3)C2)cc(S(=O)(=O)c2ccc3[nH]ncc3c2)c1C] | LCMS (M + H⁺): 528.1; 1H NMR (METHANOL-d4, 400 MHz): δ 8.44 (s, 1H), 8.28 (s, 2H), 8.23 (s, 1H), 7.77-7.84 (m, 1H), 7.66 (d, J = 8.6 Hz, 1H), 7.21 (s, 1H), 4.57 (br d, J = 6.1 Hz, 1H), 4.48 (br d, J = 4.8 Hz, 2H), 3.86 (dd, J = 11.4, 6.6 Hz, 1H), 3.72 (br dd, J = 7.0, 4.4 Hz, 1H), 3.56-3.64 (m, 3H), 3.48-3.52 (m, 1H), 3.18 (s, 3H), 2.48 (s, 3H), 2.32 (br d, J = 6.3 Hz, 1H), 2.05-2.16 (m, 1H) |
| A.92 | 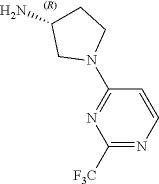 | 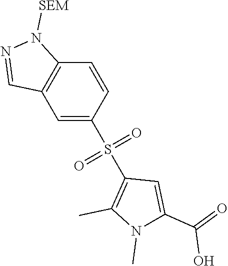 | 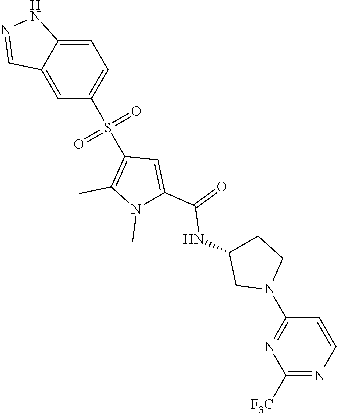 [SMILES: Cc1c(S(=O)(=O)c2ccc3[nH]ncc3c2)cc(C(=O)N[C@@H]2CCN(c3ccnc(C(F)(F)F)n3)C2)n1C] | LCMS (M + H⁺): 534.1; 1H NMR (400 MHz, DMSO-d6) δ 8.34 (s, 1H), 8.28 (d, J = 6.1 Hz, 1H), 8.26 (s, 1H), 7.75-7.63 (m, 2H), 7.23 (s, 1H), 6.69 (d, J = 6.1 Hz, 1H), 4.58-4.48 (m, 1H), 3.81- 3.73 (m, 4H), 3.68-3.62 (m, 1H), 3.55 (br s, 1H), 3.47-3.43 (m, 1H), 2.40 (s, 3H), 2.25 (br dd, J = 6.4, 12.7 Hz, 1H), 2.11-2.03 (m, 1H) |
| A.93 | 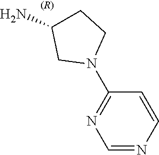 | 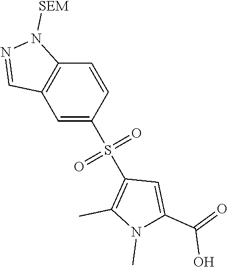 | 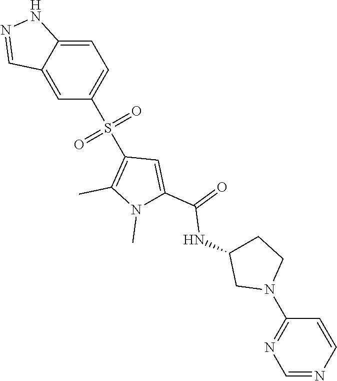 [SMILES: Cc1c(S(=O)(=O)c2ccc3[nH]ncc3c2)cc(C(=O)N[C@@H]2CCN(c3ccncn3)C2)n1C] | LCMS (M + H⁺): 466.1; 1H NMR (400 MHz, METHANOL-d4) δ 8.69 (d, J = 6.1 Hz, 1H), 8.45- 8.42 (m, 1H), 8.23 (s, 1H), 8.17 (ddd, J = 1.4, 7.6, 9.3 Hz, 1H), 7.79 (dd, J = 1.6, 8.9 Hz, 1H), 7.65 (d, J = 8.8 Hz, 1H), 7.21 (d, J = 1.6 Hz, 1H), 6.90-6.80 (m, 1H), 4.74-4.59 (m, 1H), 4.12- 3.87 (m, 2H), 3.83 (br d, J = 5.0 Hz, 4H), 3.76-3.54 (m, 1H), 2.45 (d, J = 1.6 Hz, 4H), 2.30-2.14 (m, 1H) |
| A.94 | 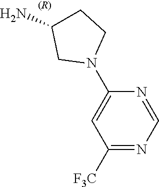 | 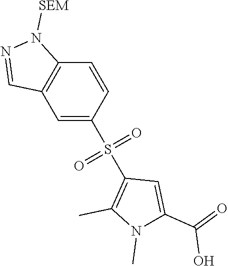 | 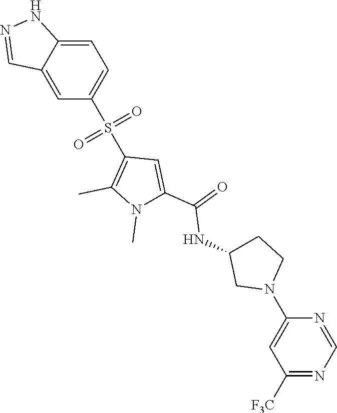 [SMILES: Cc1c(S(=O)(=O)c2ccc3[nH]ncc3c2)cc(C(=O)N[C@@H]2CCN(c3cc(C(F)(F)F)ncn3)C2)n1C] | LCMS (M + H⁺): 534.1; 1H NMR (400 MHz, METHANOL-d4) δ 8.55 (s, 1H), 8.45 (d, J = 1.0 Hz, 1H), 8.23 (s, 1H), 7.80 (dd, J = 1.6, 8.9 Hz, 1H), 7.65 (d, J = 8.8 Hz, 1H), 7.20 (s, 1H), 6.86 (br d, J = 13.3 Hz, 1H), 4.70-4.65 (m, 1H), 4.01-3.74 (m, 5H), 3.71- 3.61 (m, 1H), 3.60-3.44 (m, 1H), 2.46 (s, 3H), 2.41-2.29 (m, 1H), 2.25-2.08 (m, 1H) |
| A.95 | 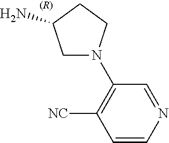 | 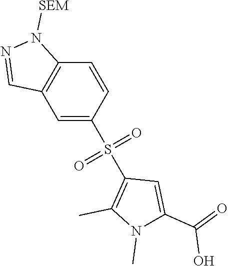 | 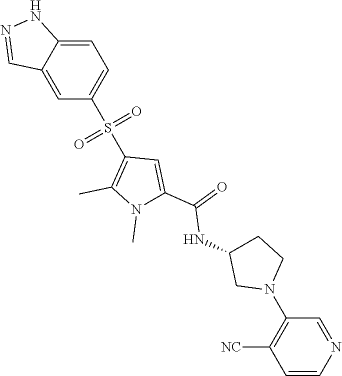 [SMILES: Cc1c(S(=O)(=O)c2ccc3[nH]ncc3c2)cc(C(=O)N[C@@H]2CCN(c3cnccc3C#N)C2)n1C] | LCMS (M + H⁺): 490.2; 1H NMR (400 MHz, DMSO-d6) δ 13.51 (br s, 1H), 8.44 (d, J = 6.8 Hz, 1H), 8.38 (t, J = 1.2 Hz, 1H), 8.30 (s, 1H), 8.24 (s, 1H), 7.89 (d, J = 5.1 Hz, 1H), 7.70 (s, 2H), 7.45 (d, J = 5.4 Hz, 1H), 7.27 (s, 1H), 4.57-4.49 (m, 1H), 3.89 (dd, J = 6.6, 10.3 Hz, 1H), 3.81- 3.76 (m, 1H), 3.75 (s, 3H), 3.70- 3.64 (m, 1H), 3.58 (dd, J = 4.3, 10.4 Hz, 1H), 2.39 (s, 3H), 2.25- 2.16 (m, 1H), 2.09-2.03 (m, 1H) |
| A.96 | 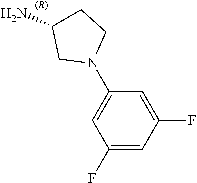 | 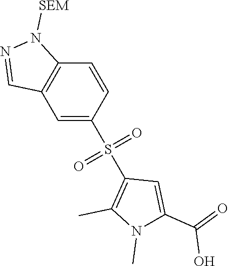 | 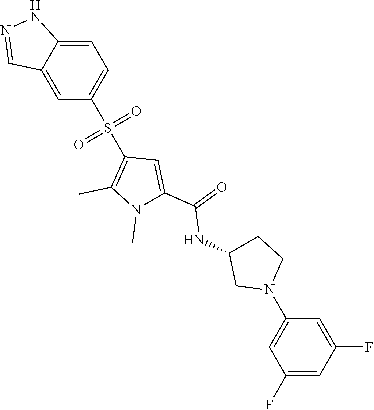 [SMILES: Cc1c(S(=O)(=O)c2ccc3[nH]ncc3c2)cc(C(=O)N[C@@H]2CCN(c3cc(F)cc(F)c3)C2)n1C] | LCMS (M + H⁺): 500.2; 1H NMR (400 MHz, METHANOL-d4) δ 8.44 (dd, J = 0.7, 1.7 Hz, 1H), 8.23 (d, J = 0.9 Hz, 1H), 7.80 (dd, J = 1.8, 8.9 Hz, 1H), 7.65 (d, J = 8.9 Hz, 1H), 7.20 (s, 1H), 6.17-6.05 (m, 3H), 4.60 (dd, J = 5.4, 6.5 Hz, 1H), 3.78 (s, 3H), 3.60 (dd, J = 6.8, 9.9 Hz, 1H), 3.51-3.40 (m, 1H), 3.37-3.33 (m, 1H), 3.23-3.15 (m, 1H), 2.45 (s, 3H), 2.34 (br d, J = 7.4 Hz, 1H), 2.11 (s, 1H) |
| A.97 | 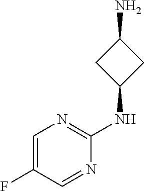 [SMILES: N[C@H]1C[C@@H](Nc2ncc(F)cn2)C1] | 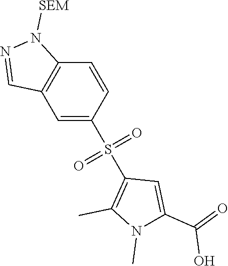 [SMILES: Cc1c(S(=O)(=O)c2ccc3c(cnn3COCC[Si](C)(C)C)c2)cc(C(=O)O)n1C] | 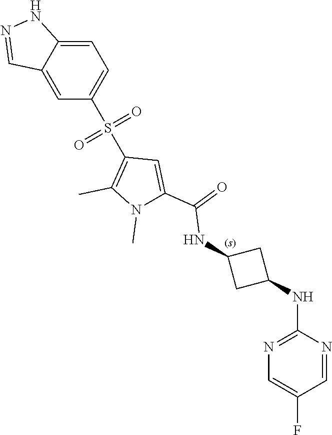 [SMILES: Cc1c(S(=O)(=O)c2ccc3[nH]ncc3c2)cc(C(=O)N[C@H]2C[C@@H](Nc3ncc(F)cn3)C2)n1C] | LCMS (M + H⁺): 484.1; 1H NMR (400 MHz, CHLOROFORM-d) δ 8.47 (s, 1H), 8.28 (s, 2H), 8.20 (s, 1H), 7.87 (d, J = 8.4 Hz, 1H), 7.59 (d, J = 8.3 Hz, 1H), 7.21 (s, 1H), 6.66-6.58 (m, 1H), 4.32- 4.21 (m, 2H), 3.80 (s, 3H), 2.96- 2.91 (m, 2H), 2.46 (s, 3H), 2.06- 2.02 (m, 2H) |
| A.98 | 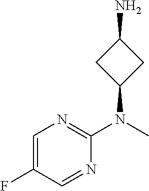 | 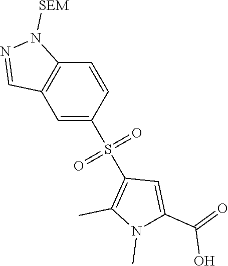 | 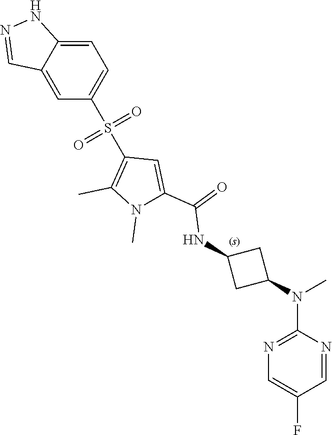 [SMILES: Cc1c(S(=O)(=O)c2ccc3[nH]ncc3c2)cc(C(=O)N[C@H]2C[C@@H](N(C)c3ncc(F)cn3)C2)n1C] | LCMS (M + H⁺): 498.1; 1H NMR (400 MHz, DMSO-d6) δ 13.50 (br s, 1H), 8.46-8.41 (m, 3H), 8.39 (s, 1H), 8.30 (s, 1H), 7.71 (s, 2H), 7.24 (s, 1H), 4.83-4.75 (m, 1H), 4.10 (br dd, J = 8.0, 15.4 Hz, 2H), 3.73 (s, 3H), 3.08 (s, 3H), 2.39 (s, 3H), 2.33 (br d, J = 1.8 Hz, 1H), 2.30-2.21 (m, 2H) |
| A.99 | 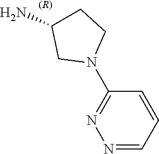 | 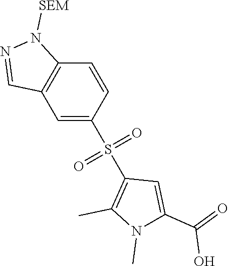 | 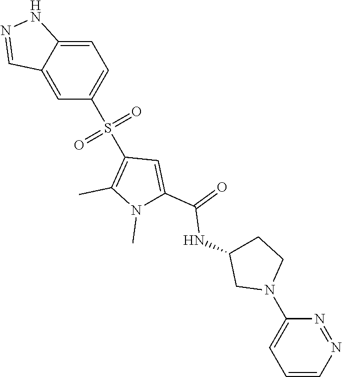 [SMILES: Cc1c(S(=O)(=O)c2ccc3[nH]ncc3c2)cc(C(=O)N[C@@H]2CCN(c3cccnn3)C2)n1C] | LCMS (M + H⁺): 466.1; 1H NMR (400 MHz, DMSO-d6) δ 13.53 (br d, J = 1.0 Hz, 1H), 8.55 (d, J = 3.9 Hz, 1H), 8.45 (d, J = 6.5 Hz, 1H), 8.38 (s, 1H), 8.30 (s, 1H), 7.70 (s, 3H), 7.53-7.42 (m, 1H), 7.26 (s, 1H), 4.57 (br d, J = 4.8 Hz, 1H), 3.81 (br dd, J = 6.1, 11.4 Hz, 1H), 3.75 (s, 3H), 3.71- 3.60 (m, 2H), 3.53 (br d, J = 8.8 Hz, 1H), 2.40 (s, 3H), 2.26 (br dd, J = 6.0, 13.5 Hz, 1H), 2.16- 2.07 (m, 1H) |
| A.100 | 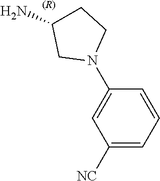 | 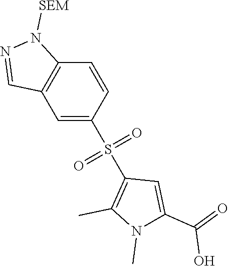 | 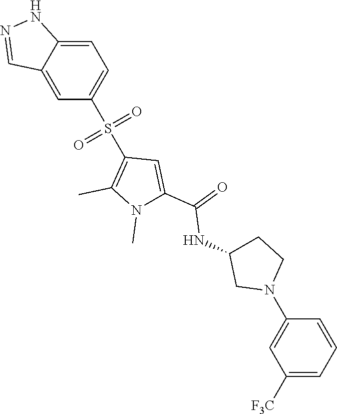 [SMILES: Cc1c(S(=O)(=O)c2ccc3[nH]ncc3c2)cc(C(=O)N[C@@H]2CCN(c3cccc(C(F)(F)F)c3)C2)n1C] | LCMS (M + H⁺): 532.1; 1H NMR (400 MHz, DMSO-d6) δ 8.44 (d, J = 6.8 Hz, 1H), 8.38 (s, 1H), 8.30 (s, 1H), 7.69 (d, J = 0.9 Hz, 2H), 7.37 (t, J = 8.0 Hz, 1H), 7.28 (s, 1H), 6.89 (d, J = 7.5 Hz, 1H), 6.80 (br d, J = 8.3 Hz, 1H), 6.72 (s, 1H), 4.58-4.48 (m, 1H), 3.75 (s, 3H), 3.60-3.57 (m, 1H), 3.45 (br d, J = 8.6 Hz, 1H), 3.37- 3.31 (m, 1H), 3.22 (dd, J = 4.7, 9.9 Hz, 1H), 2.39 (s, 3H), 2.23 (br dd, J = 6.9, 12.9 Hz, 1H), 2.09-2.00 (m, 1H) |
| A.101 | 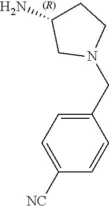 | 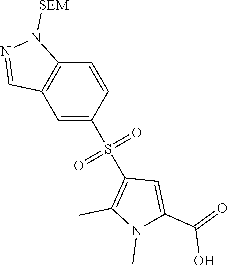 | 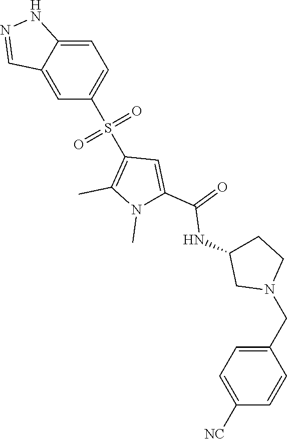 [SMILES: Cc1c(S(=O)(=O)c2ccc3[nH]ncc3c2)cc(C(=O)N[C@@H]2CCN(Cc3ccc(C#N)cc3)C2)n1C] | LCMS (M + H⁺): 503.1; 1H NMR (400 MHz, METHANOL-d4) δ 8.46 (d, J = 0.9 Hz, 1H), 8.25 (s, 1H), 7.82 (dd, J = 1.6, 8.9 Hz, 1H), 7.73-7.64 (m, 3H), 7.56 (d, J = 8.3 Hz, 2H), 7.17 (s, 1H), 4.45 (tdd, J = 4.8, 7.0, 9.1 Hz, 1H), 3.76 (s, 3H), 3.74 (s, 2H), 2.86 (dd, J = 7.1, 9.8 Hz, 1H), 2.79 (dt, J = 5.9, 8.6 Hz, 1H), 2.60-2.53 (m, 2H), 2.45 (s, 3H), 2.35-2.24 (m, 1H), 1.86-1.75 (m, 1H) |
| A.102 | 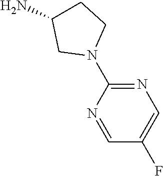 [SMILES: N[C@@H]1CCN(c2ncc(F)cn2)C1] | 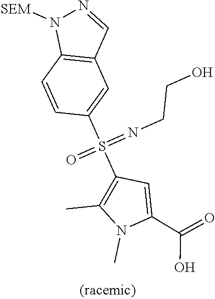 [SMILES: Cc1c(S(=O)(=NCCO)c2ccc3c(cnn3COCC[Si](C)(C)C)c2)cc(C(=O)O)n1C] | 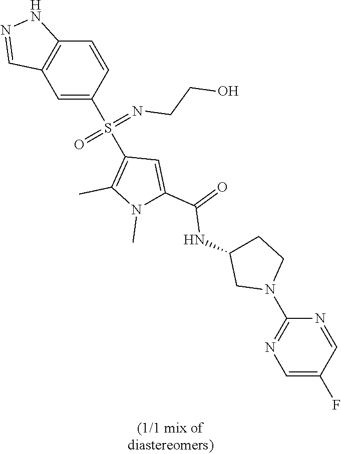 [SMILES: Cc1c(S(=O)(=NCCO)c2ccc3[nH]ncc3c2)cc(C(=O)N[C@@H]2CCN(c3ncc(F)cn3)C2)n1C] | LCMS (M + H⁺): 527.2; 1H NMR (400 MHz, METHANOL-d4) δ 8.51 (s, 1H), 8.29 (s, 2H), 8.22 (s, 1H), 7.93-7.84 (m, 1H), 7.70- 7.60 (m, 1H), 7.18 (s, 1H), 4.68- 4.54 (m, 2H), 3.87 (br dd, J = 6.8, 11.5 Hz, 2H), 3.78 (s, 3H), 3.75-3.67 (m, 3H), 3.65-3.57 (m, 1H), 3.49 (br dd, J = 5.0, 11.8 Hz, 1H), 3.25-3.09 (m, 2H), 2.45 (s, 3H), 2.41-2.27 (m, 1H), 2.20- 2.04 (m, 1H) |
| A.103 | 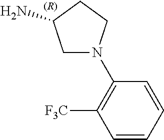 | 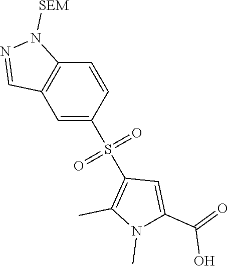 | 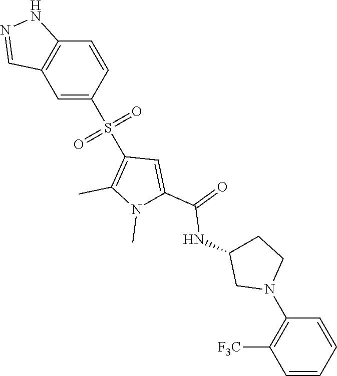 [SMILES: Cc1c(S(=O)(=O)c2ccc3[nH]ncc3c2)cc(C(=O)N[C@@H]2CCN(c3ccccc3C(F)(F)F)C2)n1C] | LCMS (M + H⁺): 532.2; 1H NMR (400 MHz, DMSO-d6) δ 13.55 (br s, 1H), 8.42-8.37 (m, 2H), 8.30 (s, 1H), 7.70 (s, 2H), 7.58 (d, J = 7.4 Hz, 1H), 7.49 (t, J = 7.6 Hz, 1H), 7.28 (s, 1H), 7.12 (d, J = 8.4 Hz, 1H), 6.95 (t, J = 7.5 Hz, 1H), 4.43 (sxt, J = 6.4 Hz, 1H), 3.74 (s, 3H), 3.55-3.49 (m, 1H), 3.40 (br d, J = 7.3 Hz, 1H), 3.28-3.21 (m, 2H), 2.39 (s, 3H), 2.18 (qd, J = 6.5, 12.5 Hz, 1H), 2.04-1.95 (m, 1H) |
| A.104 | 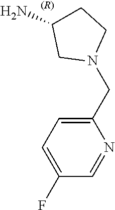 | 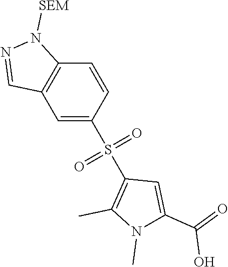 | 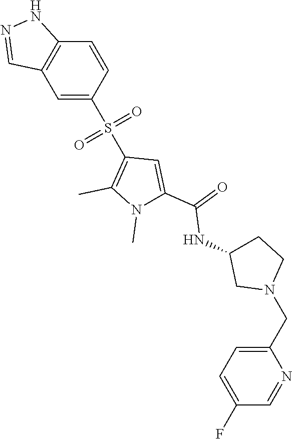 [SMILES: Cc1c(S(=O)(=O)c2ccc3[nH]ncc3c2)cc(C(=O)N[C@@H]2CCN(Cc3ccc(F)cn3)C2)n1C] | LCMS (M + H⁺): 497.1; 1H NMR (400 MHz, DMSO-d6) δ 13.65- 13.35 (m, 1H), 8.46 (d, J = 2.8 Hz, 1H), 8.38 (s, 1H), 8.32-8.28 (m, 2H), 7.73-7.65 (m, 3H), 7.49 (dd, J = 4.5, 8.6 Hz, 1H), 7.27 (s, 1H), 4.33-4.24 (m, 1H), 3.71 (s, 3H), 3.70 (s, 2H), 2.83-2.77 (m, 1H), 2.68-2.60 (m, 2H), 2.43- 2.40 (m, 1H), 2.38 (s, 3H), 2.16- 2.04 (m, 1H), 1.79-1.67 (m, 1H) |
| A.105 | 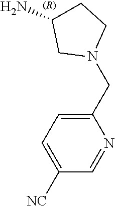 | 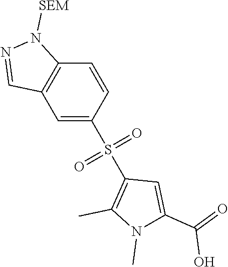 | 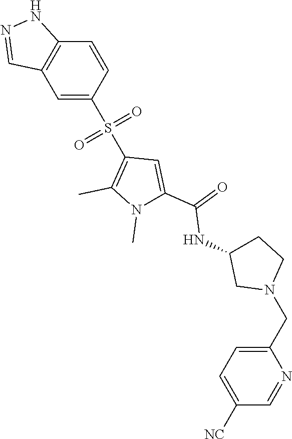 [SMILES: Cc1c(S(=O)(=O)c2ccc3[nH]ncc3c2)cc(C(=O)N[C@@H]2CCN(Cc3ccc(C#N)cn3)C2)n1C] | LCMS (M + H⁺): 504.1; 1H NMR (400 MHz, DMSO-d6) δ 13.80- 13.32 (m, 1H), 10.99 (br d, J = 3.5 Hz, 1H), 9.13 (d, J = 1.8 Hz, 1H), 8.63-8.49 (m, 1H), 8.45 (dd, J = 2.1, 8.1 Hz, 1H), 8.39 (s, 1H), 8.31 (s, 1H), 7.81 (br d, J = 8.5 Hz, 1H), 7.71 (s, 2H), 7.36- 7.27 (m, 1H), 4.81-4.68 (m, 2H), 4.61-4.38 (m, 1H), 3.94-3.79 (m, 1H), 3.72 (s, 3H), 3.69-3.58 (m, 1H), 3.33-3.05 (m, 2H), 2.40 (s, 3H), 2.35-2.18 (m, 1H), 2.17- 1.96 (m, 1H) |
| A.106 | 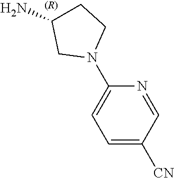 | 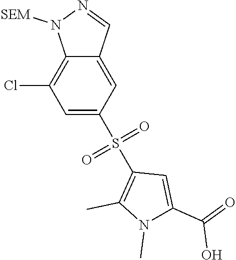 | 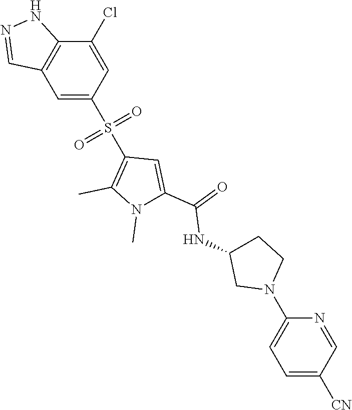 [SMILES: Cc1c(S(=O)(=O)c2cc(Cl)c3[nH]ncc3c2)cc(C(=O)N[C@@H]2CCN(c3ccc(C#N)cn3)C2)n1C] | LCMS (M + H⁺): 523.9; 1H NMR (400 MHz, METHANOL-d4) δ 8.46 (d, J = 1.8 Hz, 1H), 8.39 (d, J = 1.5 Hz, 1H), 8.32 (s, 1H), 7.93 (dd, J = 2.1, 9.4 Hz, 1H), 7.77 (d, J = 1.5 Hz, 1H), 7.21 (s, 1H), 6.92 (d, J = 9.4 Hz, 1H), 4.67 (quin, J = 5.7 Hz, 1H), 3.94 (dd, J = 6.3, 11.5 Hz, 1H), 3.84- 3.76 (m, 4H), 3.74-3.66 (m, 1H), 3.58 (br dd, J = 4.6, 11.1 Hz, 1H), 2.46 (s, 3H), 2.44-2.37 (m, 1H), 2.24 (td, J = 6.5, 13.0 Hz, 1H) |
| A.107 | 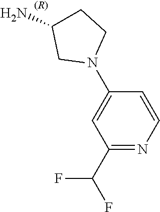 | 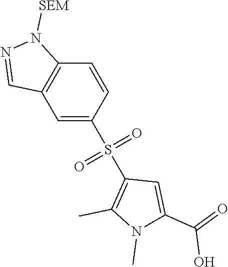 | 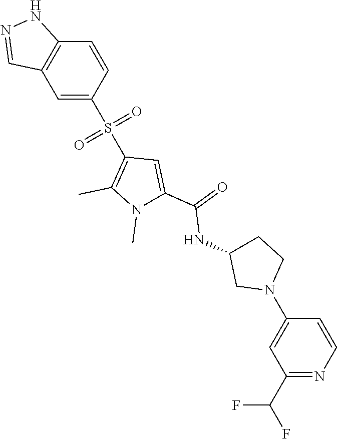 [SMILES: Cc1c(S(=O)(=O)c2ccc3[nH]ncc3c2)cc(C(=O)N[C@@H]2CCN(c3ccnc(C(F)F)c3)C2)n1C] | LCMS (M + H⁺): 515.1; 1H NMR (400 MHz, METHANOL-d4) δ 8.44 (d, J = 0.88 Hz, 1H), 8.26- 8.15 (m, 2H), 7.79 (dd, J = 8.88, 1.75 Hz, 1H), 7.66 (d, J = 9.01 Hz, 1H), 7.24-7.10 (m, 2H), 7.02-6.83 (m, 2H), 4.70 (quin, J = 5.72 Hz, 1H), 4.02-3.91 (m, 1H), 3.89-3.67 (m, 5H), 3.67- 3.55 (m, 1H), 2.51-2.38 (m, 4H), 2.32-2.21 (m, 1H) |
| A.108 | 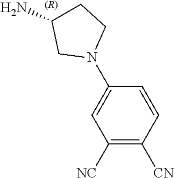 | 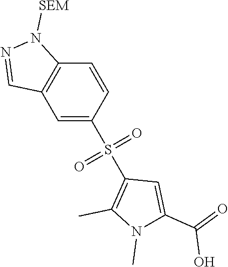 | 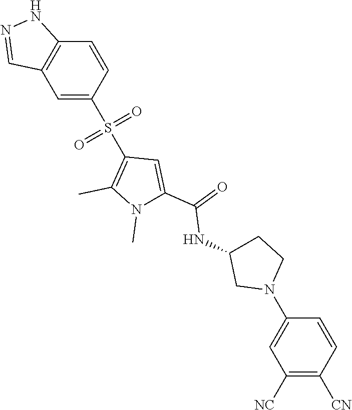 [SMILES: Cc1c(S(=O)(=O)c2ccc3[nH]ncc3c2)cc(C(=O)N[C@@H]2CCN(c3ccc(C#N)c(C#N)c3)C2)n1C] | LCMS (M + H⁺): 514.1; 1H NMR (400 MHz, DMSO-d6) δ 13.70- 13.35 (m, 1H), 8.44 (d, J = 6.75 Hz, 1H), 8.38 (s, 1H), 8.30 (s, 1H), 7.77 (d, J = 9.01 Hz, 1H), 7.69 (s, 2H), 7.26 (s, 1H), 7.19 (d, J = 2.38 Hz, 1H), 6.89 (dd, J = 8.94, 2.44 Hz, 1H), 4.60-4.48 (m, 1H), 3.75 (s, 3 H), 3.65 (dd, J = 10.88, 6.50 Hz, 1H), 3.55- 3.46 (m, 2H), 3.32 (br dd, J = 10.82, 3.94 Hz, 1H), 2.39 (s, 3H), 2.27-2.17 (m, 1H), 2.11- 2.01 (m, 1H) |
| A.109 | 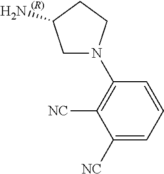 | 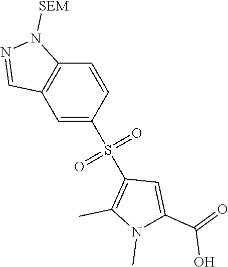 | 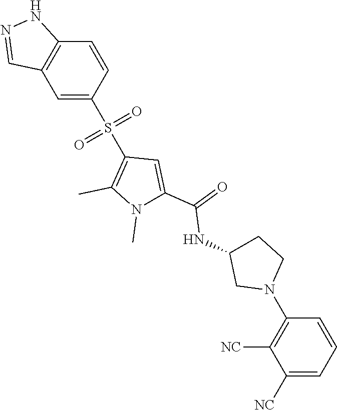 [SMILES: Cc1c(S(=O)(=O)c2ccc3[nH]ncc3c2)cc(C(=O)N[C@@H]2CCN(c3cccc(C#N)c3C#N)C2)n1C] | LCMS (M + H⁺): 514.1; 1H NMR (400 MHz, DMSO-d6) δ 8.44 (d, J = 6.5 Hz, 1H), 8.38 (t, J = 1.1 Hz, 1H), 8.30 (s, 1H), 7.70 (d, J = 0.9 Hz, 2H), 7.56 (dd, J = 7.3, 8.9 Hz, 1H), 7.31-7.23 (m, 2H), 7.14 (d, J = 8.9 Hz, 1H), 4.51 (qd, J = 5.5, 10.7 Hz, 1H), 3.88 (dd, J = 6.3, 10.3 Hz, 1H), 3.81- 3.72 (m, 4H), 3.70-3.62 (m, 1H), 3.61-3.54 (m, 1H), 2.39 (s, 3H), 2.26-2.14 (m, 1H), 2.12-2.00 (m, 1H) |
| A.110 | 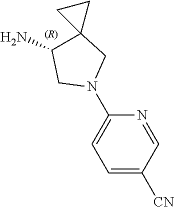 | 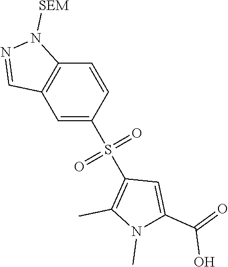 | 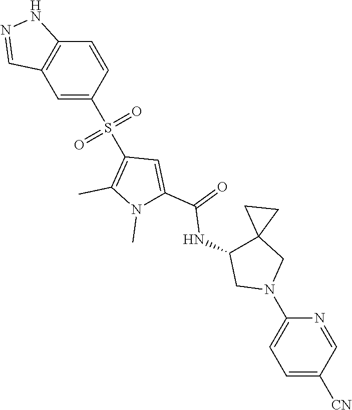 [SMILES: Cc1c(S(=O)(=O)c2ccc3[nH]ncc3c2)cc(C(=O)N[C@H]2CN(c3ccc(C#N)cn3)CC23CC3)n1C] | LCMS (M + H⁺): 516.3; 1H NMR (400 MHz, DMSO-d6) δ 13.37- 13.70 (m, 1H), 8.45-8.54 (m, 2H), 8.37 (s, 1H), 8.30 (s, 1H), 7.83 (br d, J = 9.01 Hz, 1H), 7.69 (s, 2H), 7.29 (s, 1H), 6.49-6.61 (m, 1H), 4.20-4.32 (m, 1H), 3.72 (s, 7H), 2.38 (s, 3H), 0.80-0.89 (m, 1H), 0.57-0.77 (m, 3H) |
| A.111 | 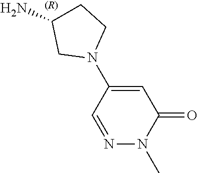 | 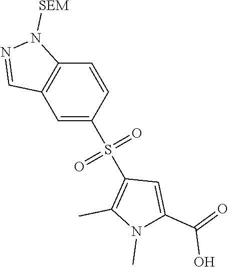 | 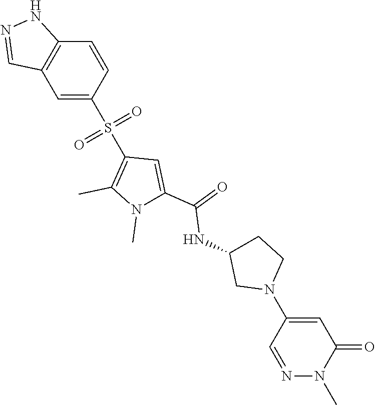 [SMILES: Cc1c(S(=O)(=O)c2ccc3[nH]ncc3c2)cc(C(=O)N[C@@H]2CCN(c3cnn(C)c(=O)c3)C2)n1C] | LCMS (M + H⁺): 496.1; 1H NMR (400 MHz, DMSO-d6) δ 13.51 (br s, 1H), 8.42 (d, J = 6.8 Hz, 1H), 8.38 (s, 1H), 8.30 (s, 1H), 7.70 (s, 2H), 7.67 (d, J = 2.5 Hz, 1H), 7.26 (s, 1H), 5.46 (d, J = 2.4 Hz, 1H), 4.54-4.46 (m, 1H), 3.74 (s, 3H), 3.64-3.57 (m, 1H), 3.51 (s, 3H), 3.46 (br d, J = 1.6 Hz, 2H), 3.26 (br s, 1H), 2.39 (s, 3H), 2.22-2.13 (m, 1H), 2.05- 1.96 (m, 1H) |
| A.112 | 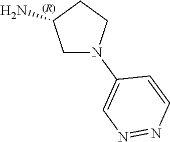 | 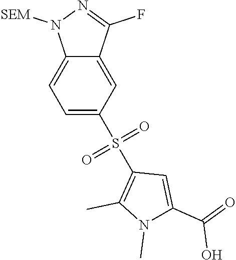 | 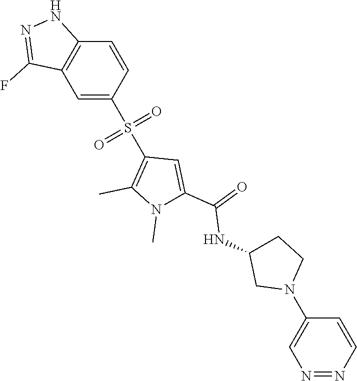 [SMILES: Cc1c(S(=O)(=O)c2ccc3[nH]nc(F)c3c2)cc(C(=O)N[C@@H]2CCN(c3ccnnc3)C2)n1C] | LCMS (M + H⁺): 484.1; 1H NMR (400 MHz, DMSO-d6) δ 8.63 (d, J = 3.0 Hz, 1H), 8.56 (d, J = 6.1 Hz, 1H), 8.43 (d, J = 6.8 Hz, 1H), 8.20 (s, 1H), 7.72-7.65 (m, 1H), 7.64-7.58 (m, 1H), 7.26 (s, 1H), 6.59 (dd, J = 3.1, 6.3 Hz, 1H), 4.54 (qd, J = 5.8, 11.3 Hz, 1H), 3.75 (s, 3H), 3.63 (br dd, J = 6.7, 10.3 Hz, 1H), 3.52-3.42 (m, 3H), 2.40 (s, 3H), 2.27-2.17 (m, 1H), 2.09-2.00 (m, 1H) |
| A.113 | 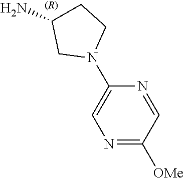 | 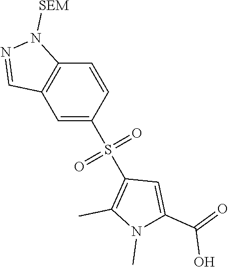 | 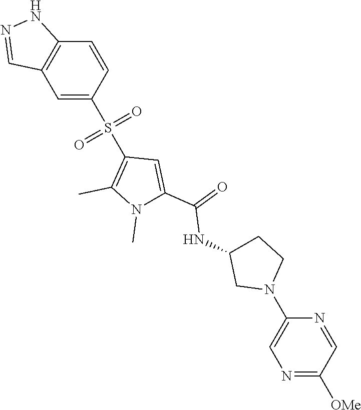 [SMILES: COc1cnc(N2CC[C@@H](NC(=O)c3cc(S(=O)(=O)c4ccc5[nH]ncc5c4)c(C)n3C)C2)cn1] | LCMS (M + H⁺): 496.0; 1H NMR (400 MHz, METHANOL-d4) δ 8.44 (d, J = 1.0 Hz, 1H), 8.23 (d, J = 0.8 Hz, 1H), 7.80 (dd, J = 1.6, 8.9 Hz, 1H), 7.76 (d, J = 1.4 Hz, 1H), 7.65 (d, J = 8.8 Hz, 1H), 7.48 (d, J = 1.4 Hz, 1H), 7.19 (s, 1H), 4.61 (quin, J = 6.3 Hz, 1H), 3.86 (s, 3H), 3.83-3.73 (m, 4H), 3.64-3.58 (m, 1H), 3.51-3.46 (m, 1H), 3.37 (br dd, J = 5.2, 10.3 Hz, 1H), 2.45 (s, 3H), 2.35 (dt, J = 6.3, 13.3 Hz, 1H), 2.17-2.07 (m, 1H) |
| A.118 | 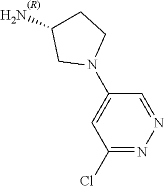 | 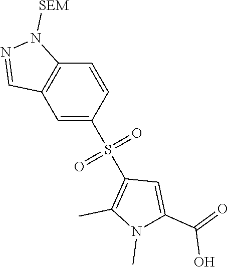 | 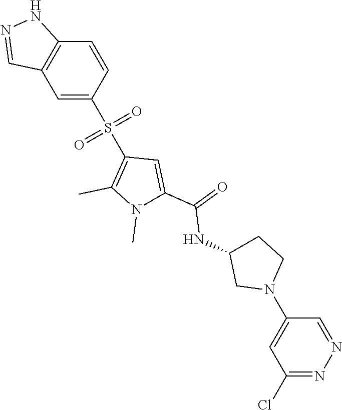 [SMILES: Cc1c(S(=O)(=O)c2ccc3[nH]ncc3c2)cc(C(=O)N[C@@H]2CCN(c3cnnc(Cl)c3)C2)n1C] | LCMS (M + H⁺): 499.9; 1H NMR (400 MHz, DMSO-d6) δ 8.63 (d, J = 1.5 Hz, 1H), 8.41 (d, J = 6.9 Hz, 1H), 8.35 (s, 1H), 8.32-8.26 (m, 1H), 7.73-7.61 (m, 2H), 7.26-7.22 (m, 1H), 6.74 (d, J = 2.4 Hz, 1H), 4.56-4.52 (m, 1H), 3.74 (s, 3H), 3.69-3.51 (m, 2H), 3.50-3.38 (m, 2H), 2.39 (s, 3H), 2.24-2.16 (m, 1H), 2.08-2.00 (m, 1H) |
| A.119 | 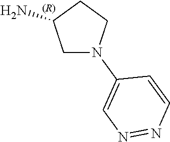 | 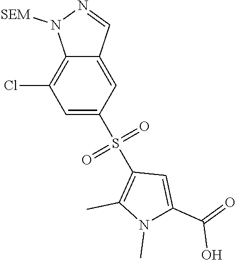 | 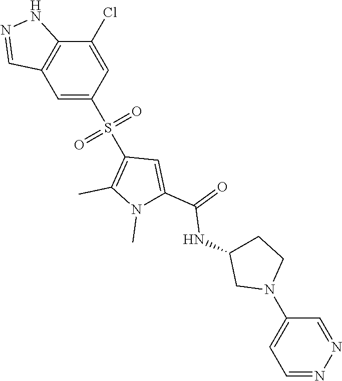 [SMILES: Cc1c(S(=O)(=O)c2cc(Cl)c3[nH]ncc3c2)cc(C(=O)N[C@@H]2CCN(c3ccnnc3)C2)n1C] | LCMS (M + H⁺): 500.1; 1H NMR (400 MHz, METHANOL-d4) δ 8.56 (d, J = 2.89 Hz, 1H), 8.53 (d, J = 6.27 Hz, 1H), 8.41-8.36 (m, 1H), 8.33-8.27 (m, 1H), 7.76 (d, J = 1.25 Hz, 1H), 7.21 (s, 1H), 6.69 (dd, J = 6.40, 3.14 Hz, 1H), 4.7-4.61 (m, 1H), 3.83-3.72 (m, 4H), 3.66-3.46 (m, 2H), 3.39 (br dd, J = 10.92, 4.14 Hz, 1H), 2.46 (s, 3H), 2.43-2.32 (m, 1H), 2.24-2.12 (m, 1H) |
| A.120 | 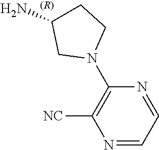 | 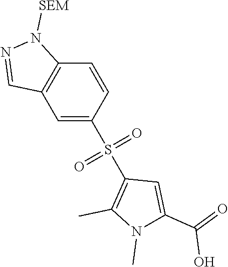 | 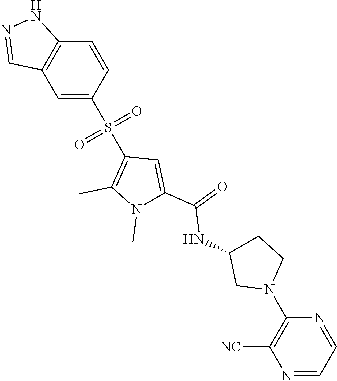 [SMILES: Cc1c(S(=O)(=O)c2ccc3[nH]ncc3c2)cc(C(=O)N[C@@H]2CCN(c3nccnc3C#N)C2)n1C] | LCMS (M + H⁺): 491.1; 1H NMR (400 MHz, DMSO-d6) δ 13.51 (s, 1H), 8.43 (br d, J = 6.5 Hz, 1H), 8.41-8.37 (m, 2H), 8.30 (s, 1H), 7.99 (d, J = 2.1 Hz, 1H), 7.70 (s, 2H), 7.27 (s, 1H), 4.57- 4.46 (m, 1H), 3.98-3.84 (m, 2H), 3.81-3.77 (m, 1H), 3.74 (s, 3H), 3.69 (br dd, J = 3.8, 11.4 Hz, 1H), 2.39 (s, 3H), 2.20 (br dd, J = 5.7, 12.7 Hz, 1H), 2.06 (td, J = 6.2, 12.4 Hz, 1H) |
| A.121 | 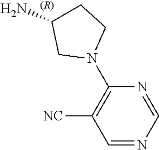 | 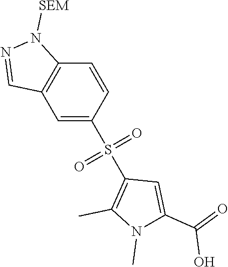 | 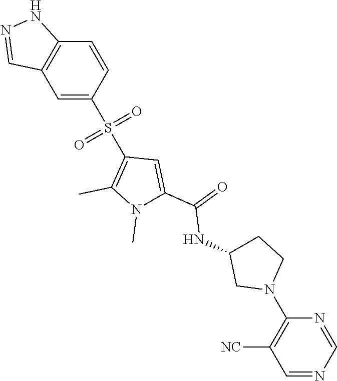 [SMILES: Cc1c(S(=O)(=O)c2ccc3[nH]ncc3c2)cc(C(=O)N[C@@H]2CCN(c3ncncc3C#N)C2)n1C] | LCMS (M + H⁺): 491.1; 1H NMR (400 MHz, DMSO-d6) δ 13.52 (br s, 1H), 8.65 (d, J = 5.9 Hz, 2H), 8.44 (br d, J = 5.9 Hz, 1H), 8.38 (s, 1H), 8.30 (s, 1H), 7.70 (s, 2H), 7.27 (s, 1H), 4.54-4.43 (m, 1H), 4.18-3.62 (m, 7H), 2.39 (s, 3H), 2.26-2.12 (m, 1H), 2.12- 1.98 (m, 1H) |
| A.122 | 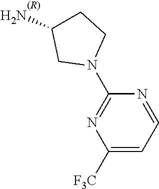 | 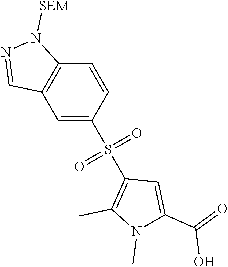 | 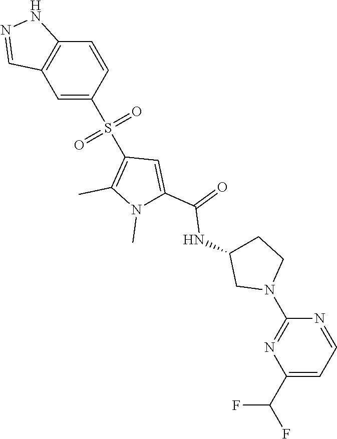 [SMILES: Cc1c(S(=O)(=O)c2ccc3[nH]ncc3c2)cc(C(=O)N[C@@H]2CCN(c3nccc(C(F)F)n3)C2)n1C] | LCMS (M + H⁺): 516.2; 1H NMR (400 MHz, DMSO-d6) δ 13.50 (br s, 1H), 8.56 (d, J = 4.8 Hz, 1H), 8.45 (d, J = 6.5 Hz, 1H), 8.38 (s, 1H), 8.29 (s, 1H), 7.69 (s, 2H), 7.28 (s, 1H), 6.89-6.58 (m, 2H), 4.54-4.42 (m, 1H), 3.82-3.76 (m, 1H), 3.75 (s, 3H), 3.70-3.62 (m, 1H), 3.61-3.55 (m, 1H), 3.48 (br d, J = 4.1 Hz, 1H), 2.39 (s, 3H), 2.25-2.16 (m, 1H), 2.06-1.97 (m, 1H) |
| A.123 | 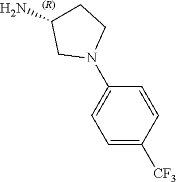 | 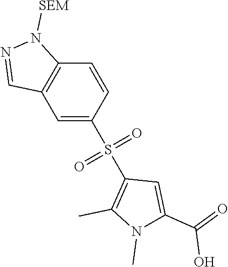 | 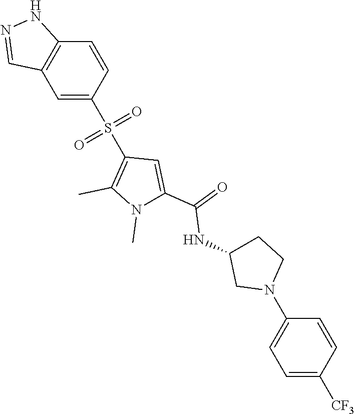 [SMILES: Cc1c(S(=O)(=O)c2ccc3[nH]ncc3c2)cc(C(=O)N[C@@H]2CCN(c3ccc(C(F)(F)F)cc3)C2)n1C] | LCMS (M + H⁺): 532.1; 1H NMR (400 MHz, DMSO-d6) δ 13.91- 13.09 (m, 1H), 8.45 (d, J = 6.8 Hz, 1H), 8.37 (s, 1H), 8.29 (s, 1H), 7.72-7.64 (m, 2H), 7.46 (d, J = 8.8 Hz, 2H), 7.27 (s, 1H), 6.64 (d, J = 8.6 Hz, 2H), 4.57- 4.49 (m, 1H), 3.75 (s, 3H), 3.60 (dd, J = 6.8, 10.1 Hz, 1H), 3.50- 3.43 (m, 1H), 3.39 (br s, 1H), 3.23 (dd, J = 4.6, 10.0 Hz, 1H), 2.39 (s, 3H), 2.27-2.18 (m, 1H), 2.10-2.00 (m, 1H) |
| A.124 | 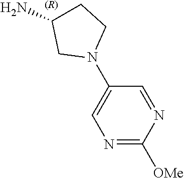 | 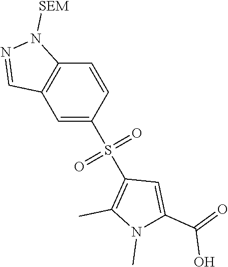 | 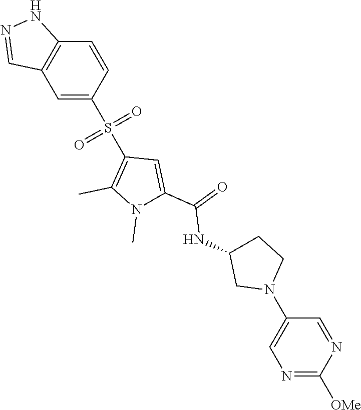 [SMILES: COc1ncc(N2CC[C@@H](NC(=O)c3cc(S(=O)(=O)c4ccc5[nH]ncc5c4)c(C)n3C)C2)cn1] | LCMS (M + H⁺): 496.0; 1H NMR (400 MHz, DMSO-d6) δ 8.43 (d, J = 7.0 Hz, 1H), 8.36 (d, J = 0.9 Hz, 1H), 8.28 (s, 1H), 8.00 (s, 2H), 7.77-7.71 (m, 1H), 7.70- 7.65 (m, 1H), 7.27 (s, 1H), 4.58- 4.48 (m, 1H), 3.81 (s, 3H), 3.75 (s, 3H), 3.52 (dd, J = 6.9, 9.7 Hz, 1H), 3.42-3.39 (m, 1H), 3.27- 3.23 (m, 1H), 3.15 (dd, J = 4.8, 9.6 Hz, 1H), 2.39 (s, 3H), 2.22 (br d, J = 7.1 Hz, 1H), 2.01 (br d, J = 5.5 Hz, 1H) |

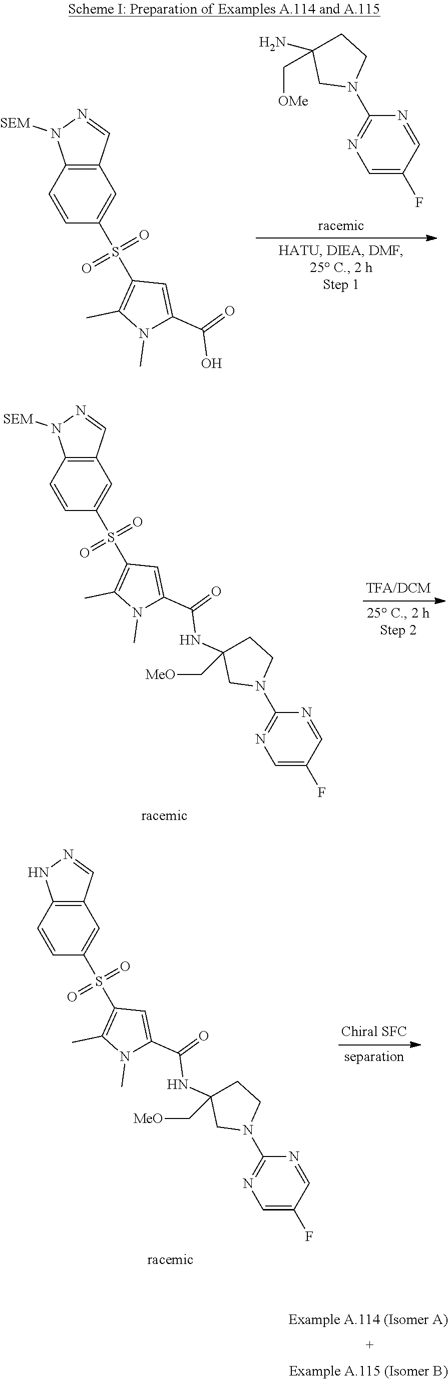 [SMILES: Cc1c(S(=O)(=O)c2ccc3c(cnn3COCC[Si](C)(C)C)c2)cc(C(=O)O)n1C ; COCC1(N)CCN(c2ncc(F)cn2)C1 ; COCC1(NC(=O)c2cc(S(=O)(=O)c3ccc4c(cnn4COCC[Si](C)(C)C)c3)c(C)n2C)CCN(c2ncc(F)cn2)C1 ; COCC1(NC(=O)c2cc(S(=O)(=O)c3ccc4[nH]ncc4c3)c(C)n2C)CCN(c2ncc(F)cn2)C1]

Racemic-4-((1H-indazol-5-yl)sulfonyl)-N-(1-(5-fluoropyrimidin-2-yl)-3-(methoxymethyl)pyrrolidin-3-yl)-1,5-dimethyl-1H-pyrrole-2-carboxamide was prepared using conditions outlined in Scheme I. ¹H NMR (400 MHz, METHANOL-d4) δ 8.46 (s, 1H), 8.29 (s, 2H), 8.25-8.24 (m, 1H), 7.82 (dd, J=1.5, 9.1 Hz, 1H), 7.67 (d, J=8.6 Hz, 1H), 7.16 (s, 1H), 3.97 (d, J=11.8 Hz, 1H), 3.81-3.77 (m, 2H), 3.75 (s, 3H), 3.73 (s, 1H), 3.69-3.63 (m, 2H), 3.39 (s, 3H), 2.51-2.47 (m, 1H), 2.46 (s, 3H), 2.37-2.29 (m, 1H); LCMS (M+H)+=528.1. The racemic material was purified via chiral SFC chromatography to furnish the resolved enantiomers. Chiral SFC conditions: Column: Chiralcel OD-3, 150×4.6 mm, I.D., 3 uM; Mobile phase: A: CO2, B: IPA (0.2% NH3(7 M in MeOH); Gradient: A:B=50:50; Flow rate: 2.5 ml/min; Column temp: 35° C.; ABPR: 2000 psi. Example A.114 (Faster eluting isomer. Example A.115 (Slower eluting isomer).

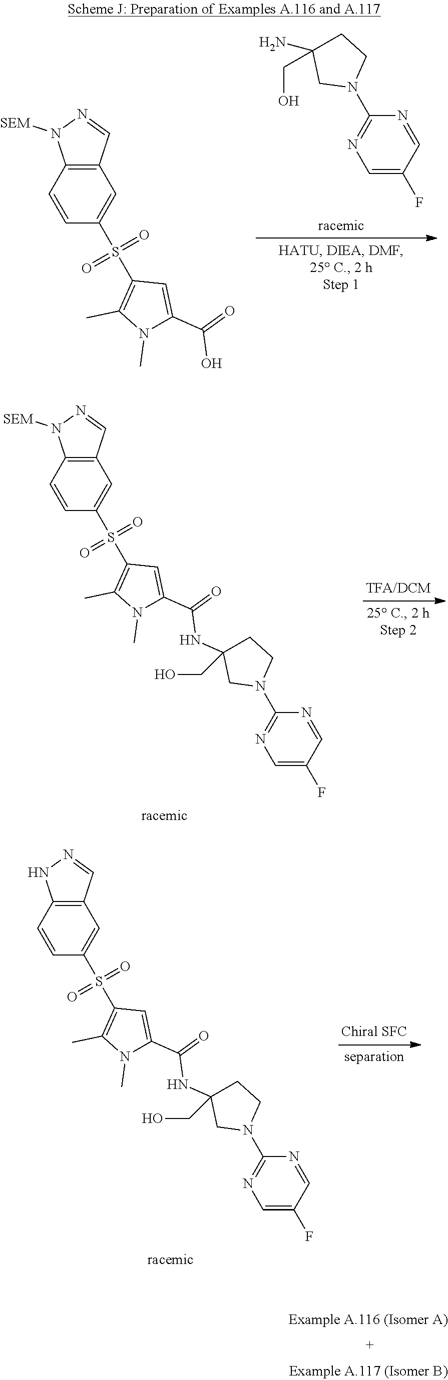 [SMILES: Cc1c(S(=O)(=O)c2ccc3c(cnn3COCC[Si](C)(C)C)c2)cc(C(=O)O)n1C ; NC1(CO)CCN(c2ncc(F)cn2)C1 ; Cc1c(S(=O)(=O)c2ccc3c(cnn3COCC[Si](C)(C)C)c2)cc(C(=O)NC2(CO)CCN(c3ncc(F)cn3)C2)n1C ; Cc1c(S(=O)(=O)c2ccc3[nH]ncc3c2)cc(C(=O)NC2(CO)CCN(c3ncc(F)cn3)C2)n1C]

Racemic-4-((1H-indazol-5-yl)sulfonyl)-N-(1-(5-fluoropyrimidin-2-yl)-3-(hydroxymethyl)pyrrolidin-3-yl)-1,5-dimethyl-1H-pyrrole-2-carboxamide was prepared using conditions outlined in Scheme J. ¹H NMR (400 MHz, METHANOL-d4) δ 8.44 (s, 1H), 8.27 (s, 2H), 8.23 (s, 1H), 7.83-7.76 (m, 1H), 7.64 (d, J=8.9 Hz, 1H), 7.17 (s, 1H), 3.96-3.87 (m, 2H), 3.84-3.77 (m, 2H), 3.74 (s, 3H), 3.70-3.58 (m, 2H), 2.44 (s, 3H), 2.42-2.27 (m, 2H)

LCMS (M+H)+=514.1. The racemic material was purified via chiral SFC chromatography to furnish the resolved enantiomers. Chiral SFC conditions: Column: Chiralcel OD-3, 50×4.6 mm, I.D., 3 uM; Mobile phase: A: C₀₂, B: IPA (0.2% NH3(7 M in MeOH); Gradient: A:B=50:50; Flow rate: 4 ml/min; Column temp: 35° C.; ABPR: 1800 psi. Example A.116 (Faster eluting isomer. Example A.117 (Slower eluting isomer).

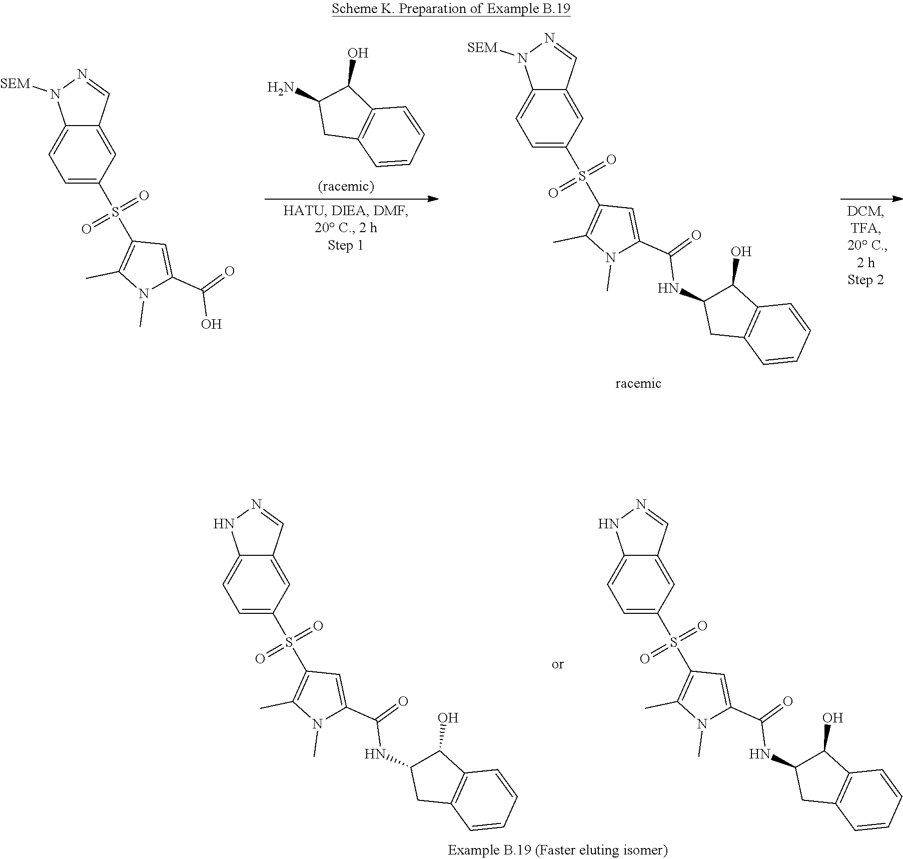 [SMILES: Cc1c(S(=O)(=O)c2ccc3c(cnn3COCC[Si](C)(C)C)c2)cc(C(=O)O)n1C ; N[C@@H]1Cc2ccccc2[C@@H]1O ; Cc1c(S(=O)(=O)c2ccc3c(cnn3COCC[Si](C)(C)C)c2)cc(C(=O)N[C@@H]2Cc3ccccc3[C@@H]2O)n1C ; Cc1c(S(=O)(=O)c2ccc3[nH]ncc3c2)cc(C(=O)N[C@H]2Cc3ccccc3[C@H]2O)n1C ; Cc1c(S(=O)(=O)c2ccc3[nH]ncc3c2)cc(C(=O)N[C@@H]2Cc3ccccc3[C@@H]2O)n1C]

The racemic indazole-amide analog was prepared using the appropriate SEM-protected acid and amine following conditions outlined. The racemic material was resolved via chiral SFC to furnish Example B.19 (faster eluting isomer). SFC conditions (column: DAICEL CHIRALPAK AD(250 mm×30 mm, 10 um);mobile phase: [CO₂-IPA(0.1% NH₃H₂O)];B %:45%, isocratic elution).

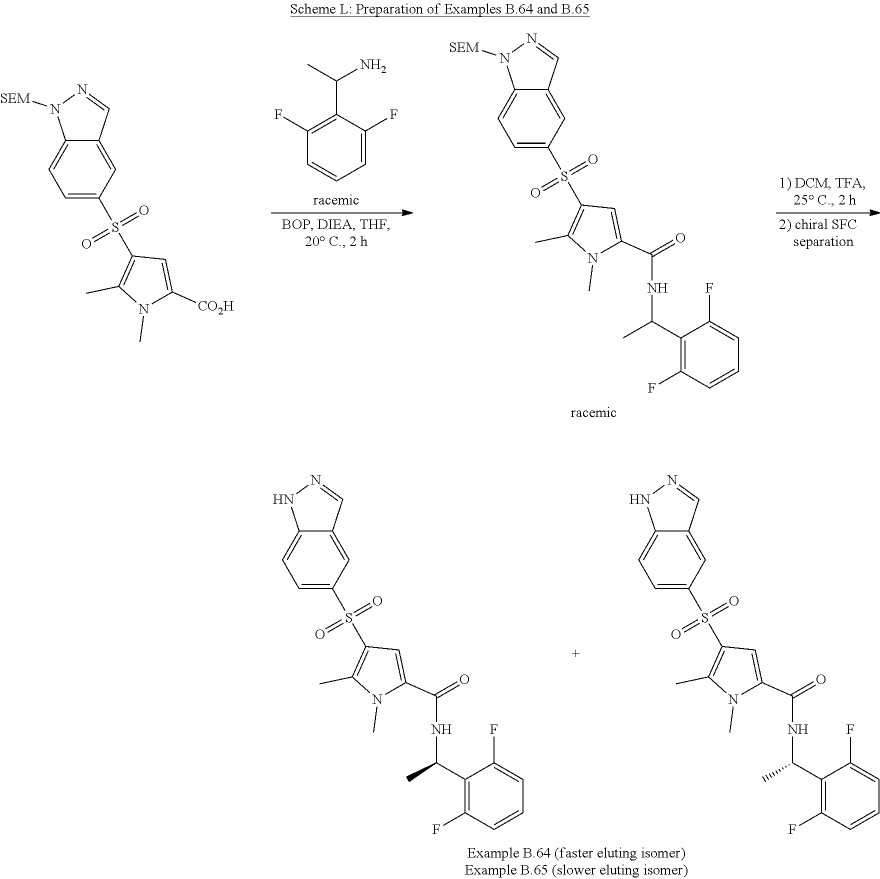 [SMILES: Cc1c(S(=O)(=O)c2ccc3c(cnn3COCC[Si](C)(C)C)c2)cc(C(=O)O)n1C ; CC(N)c1c(F)cccc1F ; Cc1c(S(=O)(=O)c2ccc3c(cnn3COCC[Si](C)(C)C)c2)cc(C(=O)NC(C)c2c(F)cccc2F)n1C ; Cc1c(S(=O)(=O)c2ccc3[nH]ncc3c2)cc(C(=O)N[C@H](C)c2c(F)cccc2F)n1C ; Cc1c(S(=O)(=O)c2ccc3[nH]ncc3c2)cc(C(=O)N[C@@H](C)c2c(F)cccc2F)n1C]

The racemic indazole-amide analog was prepared using the appropriate SEM-protected acid and amine following conditions outlined. The racemic material was resolved via chiral SFC to furnish Example B.64 (faster eluting isomer) and Example B.65 (slower eluting isomer). SFC conditions: Column: Chiralpak AD-3, 50×4.6 mm I.D., 3 um; Mobile phase: A: CO₂ B: EtOH[0.2% NH₃(7M in MeOH), v/v]; Gradient: Time, A %, B %; 0.0, 95, 5; 0.2, 95, 5; 1.2, 50, 50; 2.2, 50, 50; 2.6, 95, 5; 3.0, 95, 5; Flow rate: 3.4 mL/min; Column temp.:35° C.; ABPR:1800 psi.

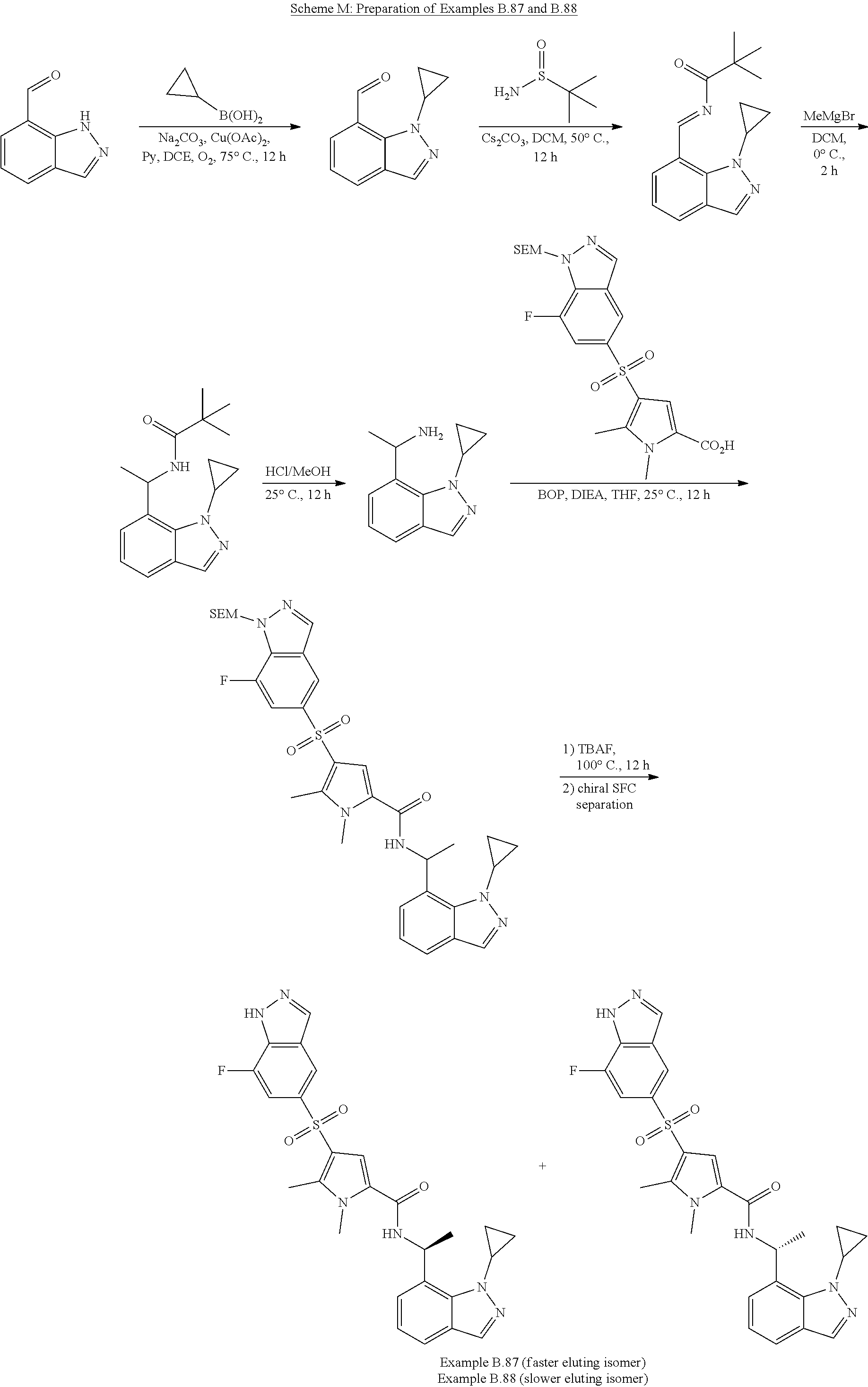

The racemic indazole-amide analog was prepared using the appropriate SEM-protected acid and amine following conditions outlined in Scheme M. The racemic material was resolved via chiral SFC to furnish Example B.87 (faster eluting isomer) and Example B.88 (slower eluting isomer). SFC conditions (column: DAICEL CHIRALPAK IG (250 mm×30 mm, 10 um);mobile phase: [CO₂-EtOH (0.1% NH₃H₂O)]; B %:40%, isocratic elution).

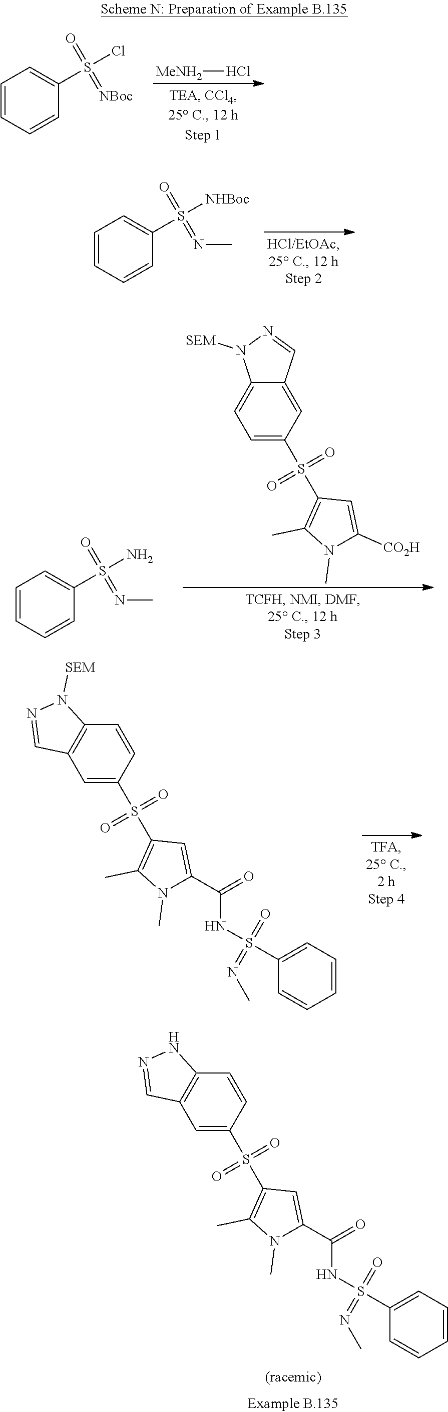

Step 1. To a solution of tert-butyl N-(chloro-oxo-phenyl-sulfanyl)carbamate (115 mg, 414 μmol, 1 eq) in CCl₄ (5 mL) was added methylamine HCl (70 mg, 1.04 mmol, 2.5 eq) and TEA (293 mg, 2.90 mmol, 403 μL, 7 eq). The mixture was stirred at 25° C. for 12 h. The reaction mixture was poured into H₂O (15 mL) and extracted with EtOAc (5 mL×3). The combined organic layers were washed with brine(15 mL), dried over Na₂SO₄, and filtered. The filtrate was concentrated under reduced pressure to give tert-butyl N—(N-methyl-S-phenyl-sulfonimidoyl)carbamate.

Step 2. To a solution of tert-butyl N—(N-methyl-S-phenyl-sulfonimidoyl)carbamate (180 mg, 665 mol, 1 eq) was added HCl/EtOAc (2 mL).The mixture was stirred at 25° C. for 12 h. The mixture was filtered and concentrated under reduced pressure to give (S-amino-N-methyl-sulfonimidoyl)benzene.

Step 3. To a solution of (S-amino-N-methyl-sulfonimidoyl)benzene (70 mg, 411 mol, 1 eq) and 1,5-dimethyl-4-[1-(2-trimethylsilylethoxymethyl)indazol-5-yl]sulfonyl-pyrrole-2-carboxylic acid (185 mg, 411 mol, 1 eq) in DMF (2 mL) was added TCFH ([chloro(dimethylamino)methylidene]-dimethylazanium;hexafluorophosphate) (231 mg, 822 mol, 2 eq) and NMI (N-methylimidazole) (169 mg, 2.06 mmol, 164 μL, 5 eq). The mixture was stirred at 25° C. for 12 h. The reaction mixture was poured into H₂O (15 mL), and the mixture was extracted with EtOAc (5 mL×3). The combined organic layers were washed with brine (15 mL), dried over Na₂SO₄, and filtered. The filtrate was concentrated under reduced pressure to furnish 1,5-dimethyl-N—(N-methyl-S-phenyl-sulfonimidoyl)-4-[1-(2-trimethylsilylethoxymethyl)indazol-5-yl]sulfonyl-pyrrole-2-carboxamide.

Step 4. To a solution of 1,5-dimethyl-N—(N-methyl-S-phenyl-sulfonimidoyl)-4-[1-(2-trimethylsilylethoxymethyl)indazol-5-yl]sulfonyl-pyrrole-2-carboxamide (50 mg, 83 mol, 1 eq) in TFA (1 mL). The mixture was stirred at 25° C. for 2 h and then concentrated. The residue was purified by prep-HPLC (formic acid condition, column: Phenomenex Luna C18 100×30 mm×3 um;mobile phase: [H₂O(0.2% FA)-ACN];gradient:25%-55% B over 8.0 min) to furnish 4-(1H-indazol-5-ylsulfonyl)-1,5-dimethyl-N—(N-methyl-S-phenyl-sulfonimidoyl)pyrrole-2-carboxamide Example B.135.

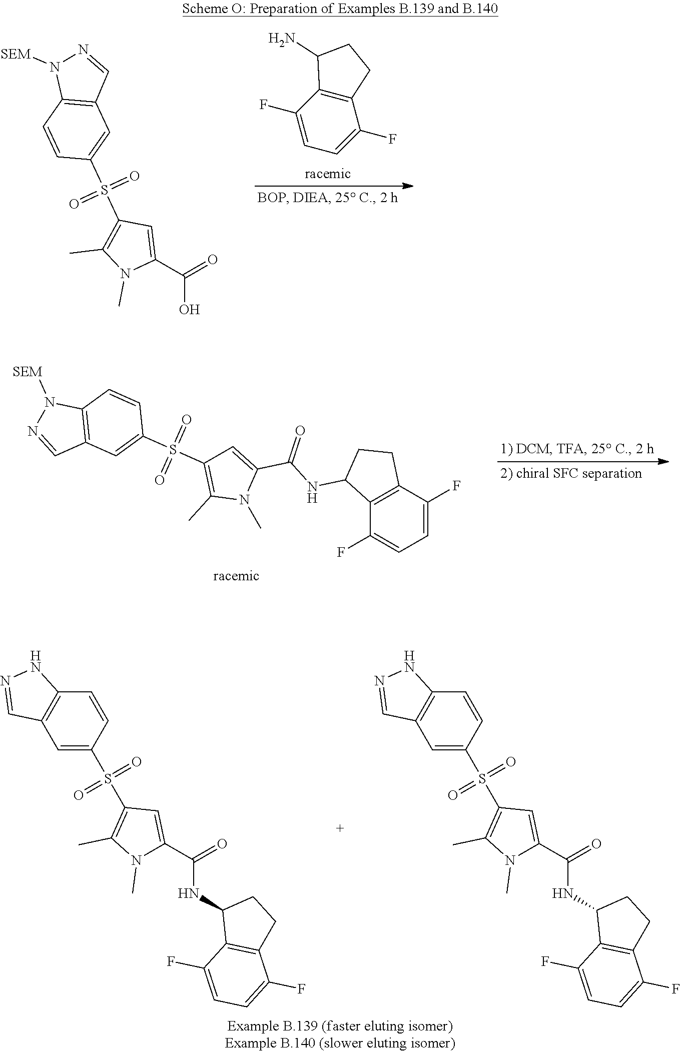 [SMILES: Cc1c(S(=O)(=O)c2ccc3c(cnn3COCC[Si](C)(C)C)c2)cc(C(=O)O)n1C ; NC1CCc2c(F)ccc(F)c21 ; Cc1c(S(=O)(=O)c2ccc3c(cnn3COCC[Si](C)(C)C)c2)cc(C(=O)NC2CCc3c(F)ccc(F)c32)n1C ; Cc1c(S(=O)(=O)c2ccc3[nH]ncc3c2)cc(C(=O)N[C@H]2CCc3c(F)ccc(F)c32)n1C ; Cc1c(S(=O)(=O)c2ccc3[nH]ncc3c2)cc(C(=O)N[C@@H]2CCc3c(F)ccc(F)c32)n1C]

The racemic indazole-amide analog was prepared using the appropriate SEM-protected acid and amine following conditions outlined in Scheme O. The racemic material was resolved via chiral SFC to furnish Example B.139 (faster eluting isomer) and Example B.140 (slower eluting isomer). SFC conditions (column: DAICEL CHIRALPAK AD(250 mm×30 mm, 10 um);mobile phase: [CO₂-MeOH(0.1% NH₃H₂O)]; B %:50%, isocratic elution).

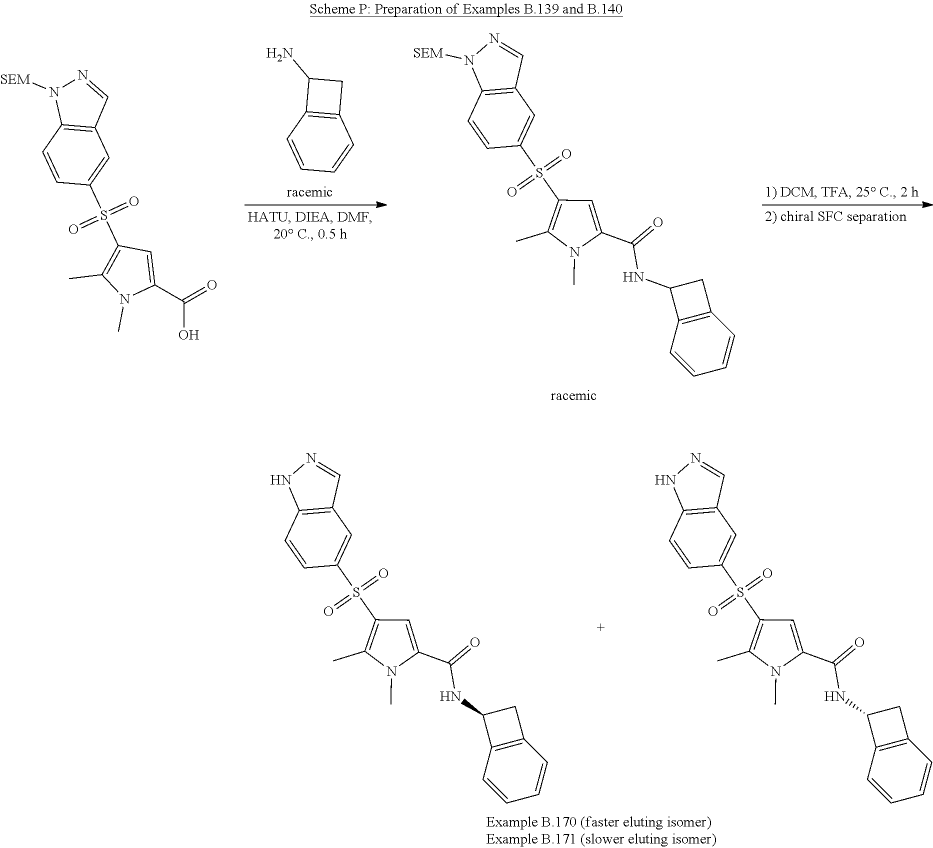 [SMILES: Cc1c(S(=O)(=O)c2ccc3c(cnn3COCC[Si](C)(C)C)c2)cc(C(=O)O)n1C ; NC1Cc2ccccc21 ; Cc1c(S(=O)(=O)c2ccc3c(cnn3COCC[Si](C)(C)C)c2)cc(C(=O)NC2Cc3ccccc32)n1C ; Cc1c(S(=O)(=O)c2ccc3[nH]ncc3c2)cc(C(=O)N[C@H]2Cc3ccccc32)n1C ; Cc1c(S(=O)(=O)c2ccc3[nH]ncc3c2)cc(C(=O)N[C@@H]2Cc3ccccc32)n1C]

The racemic indazole-amide analog was prepared using the appropriate SEM-protected acid and amine following conditions outlined in Scheme P. The racemic material was resolved via chiral SFC to furnish Example B.170 (faster eluting isomer) and Example B.171 (slower eluting isomer). SFC conditions (column: DAICEL CHIRALCEL OZ 250×25 mm I.D. 10 μm;mobile phase: [CO₂-EtOH(0.1% NH₃H₂O)]; B %:50%, isocratic).

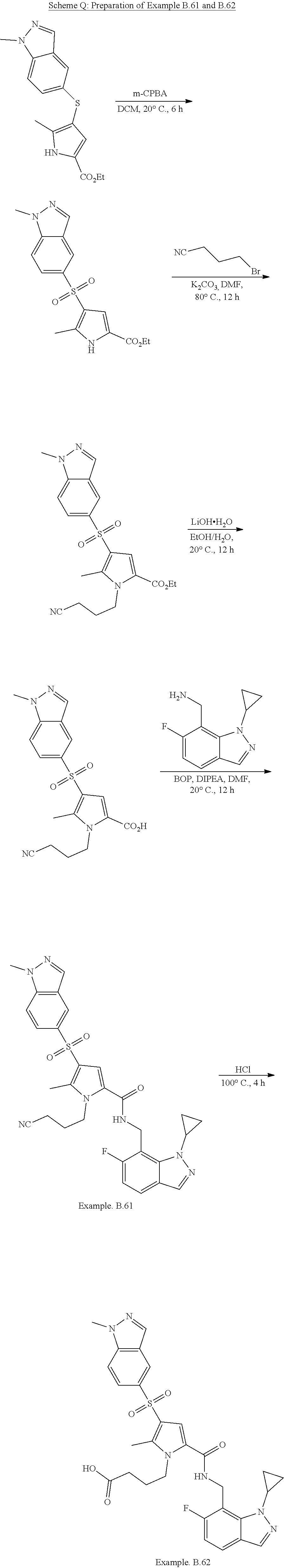

A mixture of ethyl 5-methyl-4-(1-methylindazol-5-yl)sulfanyl-1H-pyrrole-2-carboxylate (0.900 g, 2.85 mmol, 1 eq), m-CPBA (1.45 g, 7.13 mmol, 85% purity, 2.5 eq) in DCM (3 mL) was degassed and purged with N₂ (3×). The mixture was stirred at 20° C. for 6 h under a N₂ atmosphere. The reaction mixture was quenched with saturated Na₂SO₃ (aq, 50 mL), and the mixture was stirred at 20° C. for 0.5 h. The mixture was extracted with DCM (10 mL×3). The combined organic layers were washed with saturated NaHCO₃ (20 mL×3), brine (50 mL), dried over Na₂SO₄, and filtered. The filtrate was concentrated under reduced pressure. The residue was purified by column chromatography (SiO₂, petroleum ether/ethyl acetate=1/0 to 1/1) to furnish ethyl 5-methyl-4-((1-methyl-1H-indazol-5-yl)sulfonyl)-1H-pyrrole-2-carboxylate.

A mixture of ethyl 5-methyl-4-(1-methylindazol-5-yl)sulfonyl-1H-pyrrole-2-carboxylate (0.900 g, 2.59 mmol, 1 eq), 4-bromobutanenitrile (460 mg, 3.11 mmol, 309 μL, 1.2 eq), K₂CO₃ (716 mg, 5.18 mmol, 2 eq) in DMF (10 mL) was degassed and purged with N₂ (3×). The mixture was stirred at 80° C. for 12 h under a N₂ atmosphere. Water was added (10 mL), and the mixture was extracted with EtOAc (15 mL×3). The combined organic layers were washed with brine (20 mL), dried over Na₂SO₄, and filtered. The filtrate was concentrated under reduced pressure to give a residue. The residue was purified by column chromatography (SiO₂, petroleum ether/ethyl acetate=1/0 to 3/1) to furnish ethyl 1-(3-cyanopropyl)-5-methyl-4-((1-methyl-1H-indazol-5-yl)sulfonyl)-1H-pyrrole-2-carboxylate Example B.61.

A mixture of ethyl 1-(3-cyanopropyl)-5-methyl-4-(1-methylindazol-5-yl)sulfonyl-pyrrole-2-carboxylate (0.900 g, 2.17 mmol, 1 eq), LiOH·H₂O (182 mg, 4.34 mmol, 2 eq) in EtOH (9 mL) and H₂O (0.9 mL) was degassed and purged with N₂ (3×). The mixture was stirred at 20° C. for 12 h under a N₂ atmosphere. The mixture was concentrated under reduced pressure to remove the MeOH and THF. The pH of the mixture was adjusted to 3 by addition of aqueous HCl (2 N). The formed solid was collected and dried to furnish 1-(3-cyanopropyl)-5-methyl-4-((1-methyl-1H-indazol-5-yl)sulfonyl)-1H-pyrrole-2-carboxylic acid Example B.62.

### NSP14 MTase Inhibition Assay

NSP14 MTase assay at 37° C. (0.3 nM NSP14 as final enzyme concentration)

The NSP14 MTase assay at 37° C. is carried out in 384-well flat bottom polystyrene microplates (Greiner, 781075). Per well, 10 μl of 1×NSP14 reaction buffer (50 mM Tris-HCl, pH 7.5, 6 mM KCl, 1.25 mM MgCl₂, 1 mM DTT, and 0.01% Tween-20) are dispensed in columns 1-23 using a Thermo Multidrop Combi dispenser (Thermo Scientific). 0.1 μl of serially diluted compounds in DMSO are dispensed into columns 1-22 of the assay microplates with a Janus 384 MDT NanoHead (PerkinElmer). In wells of column 23, which serve as negative controls, no compounds are added. In wells of column 24, which serve as positive controls, 10 μl 1.6× Sinefungin (80 μM in 1×NSP14 reaction buffer) are added.

For the NSP14 enzyme reaction, 2 μl of 8×NSP14 solution (2.4 nM in 1×NSP14 reaction buffer) are added to all wells followed by incubation for 5 minutes at RT. The NSP14 reaction is started by adding 4 μl of a 4× substrate solution containing S-adenosyl methionine (SAM, 10 μM) and GpppA RNA (10 μM) prepared in 1×NSP14 reaction buffer. Any addition of reagents is followed by a 30 second centrifugation at 500×g to ensure that all liquid was collected at the bottom of the well. The final concentrations of NSP14, SAM, and GpppA RNA are 0.3 nM, 2.5 μM, and 2.5 μM, respectively. The final reaction volume is 16 μl. Plates are sealed and incubated for 45 min at 37° C.

For SAH detection, 1 μl 5× MTase-Glo™ Reagent (Promega) is added to each well, plates are mixed for 1-2 minutes and incubated at room temperature for 30 minutes. 5 μl of room-temperature MTase-Glo™ Detection Solution (Promega) is added to each well, plates are mixed for 1-2 minutes and incubated at room temperature for 30 minutes. Luminescence signals of each well are recorded in relative light units (RLUs) using a Biotek Synergy Neo plate reader (BioTek, Winooski, VT). The SAH production is normalized against the positive control (PK, column 24) and negative control (NK, column 23) as follows: normalized % inhibition=100×(RLUsample-RLUaverageNK)/(RLUaveragePK-RLUaverageNK). Dose response curves are fitted with the four parameter Hill Equation and half-maximal inhibitory constants (IC₅₀) are calculated using GraphPad Prism (Version 9.4.1) from three replicate experiments.

Nsp14 Mtase inhibition data (IC₅₀, nM) for representative examples is shown in Table 3.

TABLE 3
(Nsp14 Mtase inhibition assay)

| | Nsp14 Mtase |
|---|---|
| | Inh. IC₅₀ |
| Example | (nM) |
| A.1 | 0.20 |
| A.2 | 0.32 |
| A.3 | 0.35 |
| A.4 | 0.50 |
| A.5 | 1.5 |
| A.6 | 1.7 |
| A.7 | 1.7 |
| A.8 | 1.8 |
| A.9 | 2.1 |
| A.10 | 4.9 |
| A.11 | 5.3 |
| A.12 | 5.6 |
| A.13 | 7.2 |
| A.14 | 7.7 |
| A.15 | 13.4 |
| A.16 | 15 |
| A.17 | 16 |
| A.18 | 18 |
| A.19 | 21 |
| A.20 | 22 |
| A.21 | 53 |
| A.22 | 67 |
| A.23 | 68 |
| A.24 | 89 |
| A.25 | 90 |
| A.26 | 98 |
| A.27 | 102 |
| A.28 | 119 |
| A.29 | 160 |
| A.30 | 243 |
| A.31 | 315 |
| A.32 | 389 |
| A.33 | 257 |
| A.34 | 0.17 |
| A.35 | 0.56 |
| A.36 | 2.2 |
| A.37 | <0.15 |
| A.38 | 0.80 |
| A.39 | <0.15 |
| A.40 | <0.15 |
| A.41 | <0.15 |
| A.42 | 0.42 |
| A.43 | 0.42 |
| A.44 | <0.15 |
| A.45 | 0.37 |
| A.46 | 0.77 |
| A.47 | 2.2 |
| A.48 | 33 |
| A.49 | 0.63 |
| A.50 | <0.15 |
| A.51 | <0.15 |
| A.52 | 0.30 |
| A.53 | 0.36 |
| A.54 | <0.15 |
| A.55 | 0.90 |
| A.56 | 0.20 |
| A.57 | 0.57 |
| A.58 | 3.4 |
| A.59 | 0.18 |
| A.60 | 0.28 |
| A.61 | 0.39 |
| A.62 | 0.41 |
| A.63 | 0.46 |
| A.64 | 0.78 |
| A.65 | 1.0 |
| A.66 | 0.81 |
| A.67 | 0.38 |
| A.68 | 0.36 |
| A.69 | 8.7 |
| A.70 | 3.9 |
| A.71 | 0.55 |
| A.72 | 0.29 |
| A.73 | 0.41 |
| A.74 | 0.57 |
| A.75 | 0.51 |
| A.76 | 0.44 |
| A.77 | 7.9 |
| A.78 | 0.53 |
| A.79 | 0.60 |
| A.80 | 1.5 |
| A.81 | 0.58 |
| A.82 | 0.45 |
| A.83 | 0.84 |
| A.84 | <0.15 |
| A.85 | 27 |
| A.86 | 2.5 |
| A.87 | 0.75 |
| A.88 | <0.15 |
| A.89 | 14 |
| A.90 | <0.15 |
| A.91 | 0.17 |
| A.92 | <0.15 |
| A.93 | 0.20 |
| A.94 | 0.15 |
| A.95 | <0.15 |
| A.96 | 0.85 |
| A.97 | 13 |
| A.98 | 0.39 |
| A.99 | 0.38 |
| A.100 | 2.9 |
| A.101 | 26 |
| A.102 | 0.46 |
| A.103 | 5.1 |
| A.104 | 4.3 |
| A.105 | 14 |
| A.106 | <0.15 |
| A.107 | 0.17 |
| A.108 | <0.15 |
| A.109 | <0.15 |
| A.110 | 0.70 |
| A.111 | <0.15 |
| A.112 | 0.53 |
| A.113 | <0.15 |
| A.114 | 0.87 |
| A.115 | 37 |
| A.116 | 44 |
| A.117 | 2.8 |
| A.118 | <0.15 |
| A.119 | 0.37 |
| A.120 | 0.26 |
| A.121 | 0.30 |
| A.122 | 0.30 |
| A.123 | 8.6 |
| A.124 | 0.23 |

Examples B.1 through B.198 were prepared according to similar procedures as described above.

| | | Nsp14 | | |
|---|---|---|---|---|
| | | Mtase Inh. | | LCMS |
| Ex | Structure | IC₅₀ (nM) | ¹H NMR(400 Mhz) | [M + H]+ |
| B.1 | 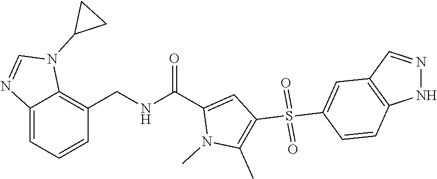 [SMILES: Cc1c(S(=O)(=O)c2ccc3[nH]ncc3c2)cc(C(=O)NCc2cccc3ncn(C4CC4)c23)n1C] | 0.38 | A δ 13.79-13.27 (m, 1H), 9.14-9.03 (m, 1H), 8.92 (br t, J = 5.1 Hz, 1H), 8.40 (s, 1H), 8.31 (s, 1H), 7.76-7.59 (m, 3H), 7.47-7.30 (m, 3H), 5.10 (br d, J = 5.6 Hz, 2H), 4.07- 3.98 (m, 1H), 3.75 (s, 3H), 2.41 (s, 3H), 1.39-1.29 (m, 2H), 1.22 (q, J = 5.8 Hz, 2H) | 489.1 |
| B.2 | 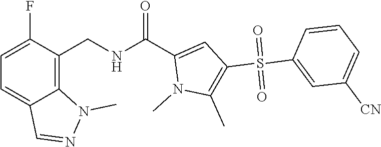 [SMILES: Cc1c(S(=O)(=O)c2cccc(C#N)c2)cc(C(=O)NCc2c(F)ccc3cnn(C)c23)n1C] | 0.61 | A δ 8.71 (br t, J = 4.2 Hz, 1H), 8.23 (s, 1H), 8.10 (dd, J = 1.6, 7.9 Hz, 2H), 8.06 (s, 1H), 7.81-7.73 (m, 2H), 7.26 (s, 1H), 7.05 (t, J = 9.4 Hz, 1H), 4.80 (br d, J = 3.0 Hz, 2H), 4.19 (s, 3H), 3.79 (s, 3H), 2.41 (s, 3H) | 466.0 |
| B.3 | 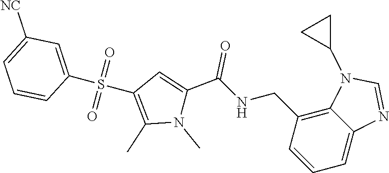 [SMILES: Cc1c(S(=O)(=O)c2cccc(C#N)c2)cc(C(=O)NCc2cccc3ncn(C4CC4)c23)n1C] | 5.3 | A δ 9.46 (br s, 1H), 9.00 (br t, J = 5.8 Hz, 1H), 8.28 (s, 1H), 8.19-8.11 (m, 2H), 7.82 (t, J = 7.9 Hz, 1H), 7.71 (d, J = 7.8 Hz, 1H), 7.56-7.49 (m, 1H), 7.48-7.44 (m, 1H), 7.37 (s, 1H), 5.13 (br d, J = 5.4 Hz, 2H), 4.16-4.08 (m, 1H), 3.77 (s, 3H), 2.43 (s, 3H), 1.43-1.36 (m, 2H), 1.29-1.22 (m, 2H) | 474.1 |
| B.4 | 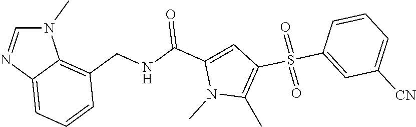 [SMILES: Cc1c(S(=O)(=O)c2cccc(C#N)c2)cc(C(=O)NCc2cccc3ncn(C)c23)n1C] | 4.9 | A 9.37-9.23 (m, 1H), 8.96 (br t, J = 4.8 Hz, 1H), 8.27 (s, 1H), 8.14 (br t, J = 7.8 Hz, 2H), 7.81 (br t, J = 7.9 Hz, 1H), 7.74 (br d, J = 8.0 Hz, 1H), 7.55-7.41 (m, 2H), 7.34 (s, 1H), 4.99-4.90 (m, 2H), 4.26 (s, 3H), 3.77 (s, 3H), 2.42 (s, 3H) | 448.0 |
| B.5 | 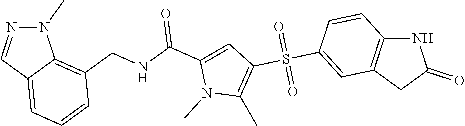 [SMILES: Cc1c(S(=O)(=O)c2ccc3c(c2)CC(=O)N3)cc(C(=O)NCc2cccc3cnn(C)c23)n1C] | 8.7 | A 10.77 (s, 1H), 8.82 (br t, J = 5.4 Hz, 1H), 8.03 (s, 1H), 7.71-7.65 (m, 2H), 7.59 (s, 1H), 7.29-7.24 (m, 2H), 7.12- 7.05 (m, 1H), 6.94 (d, J = 8.4 Hz, 1H), 6.91-6.87 (m, 1H), 4.88 (d, J = 5.0 Hz, 2H), 4.25 (s, 3H), 3.78 (s, 3H), 3.55 (s, 2H), 2.39 (s, 3H) | 478.2 |
| B.6 | 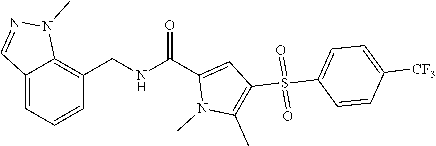 [SMILES: Cc1c(S(=O)(=O)c2ccc(C(F)(F)F)cc2)cc(C(=O)NCc2cccc3cnn(C)c23)n1C] | 35 | A δ 8.86 (t, J = 5.2 Hz, 1H), 8.06-8.01 (m, 3H), 7.99-7.95 (m, 2H), 7.68 (d, J = 7.9 Hz, 1H), 7.36 (s, 1H), 7.27 (d, J = 7.0 Hz, 1H), 7.08 (t, J = 7.6 Hz, 1H), 4.89 (d, J = 5.1 Hz, 2H), 4.25 (s, 3H), 3.79 (s, 3H), 2.42 (s, 3H) | 491.2 |
| B.7 | 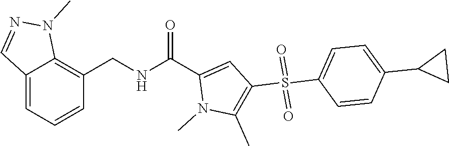 [SMILES: Cc1c(S(=O)(=O)c2ccc(C3CC3)cc2)cc(C(=O)NCc2cccc3cnn(C)c23)n1C] | 114 | A δ 8.82 (t, J = 5.2 Hz, 1H), 8.03 (s, 1H), 7.67 (d, J = 8.3 Hz, 3H), 7.29 (s, 1H), 7.28- 7.23 (m, 3H), 7.11-7.06 (m, 1H), 4.88 (d, J = 5.3 Hz, 2H), 4.25 (s, 3H), 3.78 (s, 3H), 2.38 (s, 3H), 2.03-1.94 (m, 1H), 1.07-0.98 (m, 2H), 0.77-0.69 (m, 2H) | 463.1 |
| B.8 | 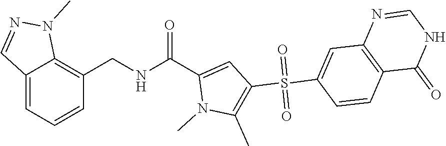 [SMILES: Cc1c(S(=O)(=O)c2ccc3c(=O)[nH]cnc3c2)cc(C(=O)NCc2cccc3cnn(C)c23)n1C] | 71 | A δ 8.88 (t, J = 5.19 Hz, 1H), 8.28 (d, J = 8.25 Hz, 1H), 8.24 (s, 1H), 8.05-8.00 (m, 2H), 7.87 (dd, J = 8.38, 1.88 Hz, 1H), 7.67 (d, J = 7.50 Hz, 1H), 7.39 (s, 1H), 7.27 (d, J = 7.00 Hz, 1H), 7.08 (dd, J = 7.82, 7.19 Hz, 1H), 4.89 (d, J = 5.13 Hz, 2H), 4.25 (s, 3H), 3.79 (s, 3H), 2.43 (s, 3H) | 491.0 |
| B.9 | 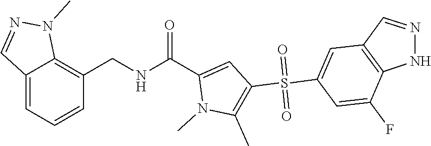 [SMILES: Cc1c(S(=O)(=O)c2cc(F)c3[nH]ncc3c2)cc(C(=O)NCc2cccc3cnn(C)c23)n1C] | 0.53 | B δ 8.33-8.24 (m, 2H), 8.00 (s, 1H), 7.69 (d, J = 8.13 Hz, 1H), 7.51 (d, J = 10.38 Hz, 1H), 7.35 (d, J = 7.00 Hz, 1H), 7.21 (s, 1H), 7.13 (t, J = 7.57 Hz, 1H), 4.99 (s, 2H), 4.30 (s, 3H), 3.81 (s, 3H), 2.47 (s, 3H) | 481.1 |
| B.10 | 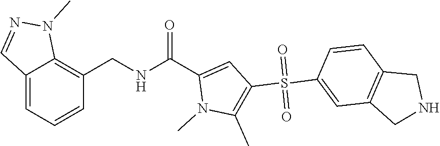 [SMILES: Cc1c(S(=O)(=O)c2ccc3c(c2)CNC3)cc(C(=O)NCc2cccc3cnn(C)c23)n1C] | 113 | A δ 8.82 (br s, 1H), 8.03 (s, 1H), 7.81-7.62 (m, 3H), 7.43 (br d, J = 7.3 Hz, 1H), 7.33- 7.23 (m, 2H), 7.13-7.03 (m, 1H), 4.89 (br s, 2H), 4.25 (s, 3H), 4.06 (br s, 4H), 3.78 (br s, 3H), 2.39 (br s, 3H) | 464.1 |
| B.11 | 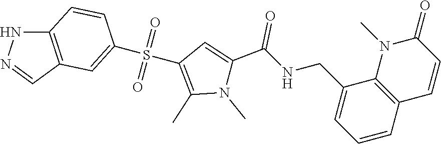 [SMILES: Cc1c(S(=O)(=O)c2ccc3[nH]ncc3c2)cc(C(=O)NCc2cccc3ccc(=O)n(C)c23)n1C] | 2.1 | A δ 13.51 (br s, 1H), 8.81 (br t, J = 5.3 Hz, 1H), 8.38 (s, 1H), 8.30 (s, 1H), 7.89 (d, J = 9.4 Hz, 1H), 7.70 (s, 2H), 7.62 (br d, J = 7.1 Hz, 1H), 7.57 (br d, J = 7.1 Hz, 1H), 7.29 (s, 1H), 7.28-7.20 (m, 1H), 6.61 (d, J = 9.3 Hz, 1H), 4.78 (br d, J = 5.0 Hz, 2H), 3.75 (s, 3H), 3.73 (s, 3H), 2.40 (s, 3H) | 490.0 |
| B.12 | 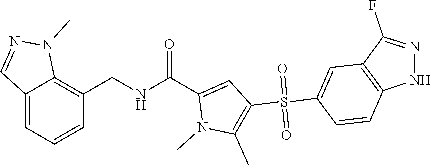 [SMILES: Cc1c(S(=O)(=O)c2ccc3[nH]nc(F)c3c2)cc(C(=O)NCc2cccc3cnn(C)c23)n1C] | 0.63 | A δ 8.82 (br t, J = 5.1 Hz, 1H), 8.24 (s, 1H), 8.03 (s, 1H), 7.76 (dd, J = 1.4, 8.9 Hz, 1H), 7.70- 7.63 (m, 2H), 7.34 (s, 1H), 7.27 (d, J = 7.0 Hz, 1H), 7.08 (t, J = 7.5 Hz, 1H), 4.89 (d, J = 5.3 Hz, 2H), 4.25 (s, 3H), 3.78 (s, 3H), 2.42 (s, 3H) | 481.0 |
| B.13 | 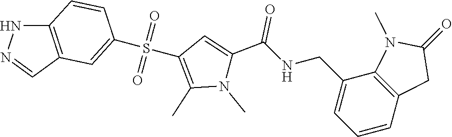 [SMILES: Cc1c(S(=O)(=O)c2ccc3[nH]ncc3c2)cc(C(=O)NCc2cccc3c2N(C)C(=O)C3)n1C] | 0.6 | A δ 13.63-13.39 (m, 1H), 8.73 (t, J = 5.2 Hz, 1H), 8.38 (s, 1H), 8.30 (s, 1H), 7.69 (s, 2H), 7.30 (s, 1H), 7.18 (dd, J = 2.0, 7.5 Hz, 2H), 7.00-6.93 (m, 1H), 4.64 (d, J = 5.3 Hz, 2H), 3.75 (s, 3H), 3.55 (s, 2H), 3.42 (s, 3H), 2.39 (s, 3H) | 500.0 |
| B.14 | 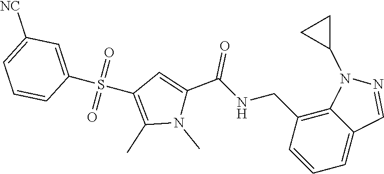 [SMILES: Cc1c(S(=O)(=O)c2cccc(C#N)c2)cc(C(=O)NCc2cccc3cnn(C4CC4)c23)n1C] | 23.1 | A δ 8.87 (br t, J = 5.1 Hz, 1H), 8.27 (s, 1H), 8.14 (br t, J = 9.1 Hz, 2H), 7.99 (s, 1H), 7.81 (br t, J = 7.8 Hz, 1H), 7.65 (br d, J = 8.0 Hz, 1H), 7.35 (s, 1H), 7.28 (br d, J = 7.0 Hz, 1H), 7.10 (t, J = 7.5 Hz, 1H), 5.12 (br d, J = 5.3 Hz, 2H), 4.03 (td, J = 3.3, 6.7 Hz, 1H), 3.78 (s, 3H), 2.45-2.40 (m, 3H), 1.31-1.24 (m, 2H), 1.17-1.10 (m, 2H) | 474.1 |
| B.15 | 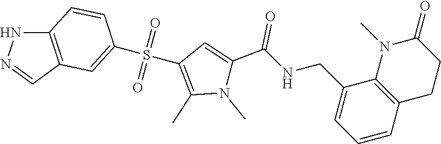 [SMILES: Cc1c(S(=O)(=O)c2ccc3[nH]ncc3c2)cc(C(=O)NCc2cccc3c2N(C)C(=O)CC3)n1C] | 14 | A δ 13.51 (br s, 1H), 8.75 (br t, J = 4.8 Hz, 1H), 8.39 (s, 1H), 8.30 (s, 1H), 7.70 (s, 2H), 7.26 (s, 1H), 7.22-7.11 (m, 2H), 7.07-7.01 (m, 1H), 4.43 (br d, J = 5.3 Hz, 2H), 3.74 (s, 3H), 3.26 (s, 3H), 2.82-2.78 (m, 2H), 2.44 (br d, J = 2.1 Hz, 2H), 2.40 (s, 3H) | 492.1 |
| B.16 | 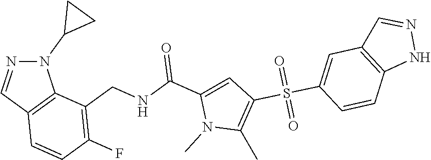 [SMILES: Cc1c(S(=O)(=O)c2ccc3[nH]ncc3c2)cc(C(=O)NCc2c(F)ccc3cnn(C4CC4)c23)n1C] | 0.38 | A δ 13.79-13.19 (m, 1H), 8.61 (br t, J = 4.1 Hz, 1H), 8.35 (s, 1H), 8.28 (s, 1H), 8.03 (s, 1H), 7.76 (dd, J = 5.2, 8.7 Hz, 1H), 7.71-7.63 (m, 2H), 7.25 (s, 1H), 7.07 (t, J = 9.4 Hz, 1H), 5.00 (br d, J = 2.9 Hz, 2H), 3.76 (s, 3H), 2.38 (s, 3H), 1.31-1.23 (m, 2H), 1.11-1.03 (m, 2H) | 507.1 |
| B.17 | 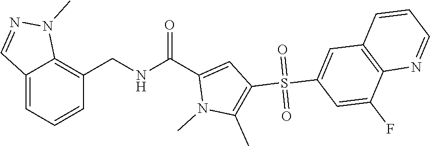 [SMILES: Cc1c(S(=O)(=O)c2cc(F)c3ncccc3c2)cc(C(=O)NCc2cccc3cnn(C)c23)n1C] | 41.8 | A δ 9.10 (dd, J = 1.6, 4.3 Hz, 1H), 8.87 (t, J = 5.3 Hz, 1H), 8.74 (d, J = 8.4 Hz, 1H), 8.52 (d, J = 1.5 Hz, 1H), 8.03 (s, 1H), 7.86-7.75 (m, 2H), 7.67 (d, J = 7.5 Hz, 1H), 7.37 (s, 1H), 7.32-7.23 (m, 1H), 7.08 (dd, J = 7.2, 7.9 Hz, 1H), 4.89 (d, J = 5.1 Hz, 2H), 4.25 (s, | 492.1 |
| | | | 3H), 3.78 (s, 3H), 2.45 (s, 3H) | |
| B.18 | 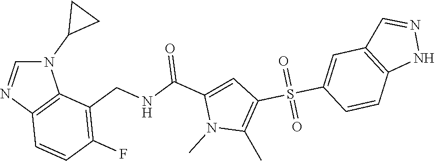 [SMILES: Cc1c(S(=O)(=O)c2ccc3[nH]ncc3c2)cc(C(=O)NCc2c(F)ccc3ncn(C4CC4)c23)n1C] | <0.15 | A δ 13.50 (br s, 1H), 8.58 (s, 1H), 8.35 (s, 1H), 8.28 (s, 1H), 8.25 (s, 1H), 7.68 (s, 2H), 7.63 (dd, J = 4.7, 8.8 Hz, 1H), 7.25 (s, 1H), 7.15-7.07 (m, 1H), 4.97 (br d, J = 3.1 Hz, 2H), 3.76 (s, 4H), 2.39 (s, 3H), 1.28-1.19 (m, 2H), 1.07 (br d, J = 5.5 Hz, 2H) | 507.0 |
| B.19 | 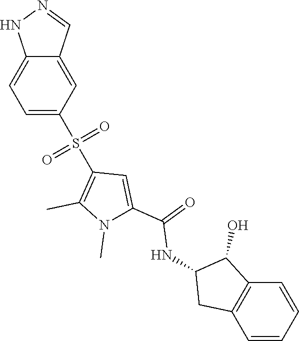 [SMILES: Cc1c(S(=O)(=O)c2ccc3[nH]ncc3c2)cc(C(=O)N[C@H]2Cc3ccccc3[C@H]2O)n1C] | 191 | C δ 10.30 (br s, 1H), 8.41 (d, J = 0.9 Hz, 1H), 8.19 (d, J = 0.9 Hz, 1H), 7.83 (dd, J = 1.7, 8.8 Hz, 1H), 7.56-7.48 (m, 2H), 7.36-7.27 (m, 4H), 7.14 (s, 1H), 5.91 (d, J = 7.8 Hz, 1H), 5.06 (dt, J = 1.8, 7.9 Hz, 1H), 3.83 (s, 3H), 3.51-3.41 (m, 1H), 3.22 (d, J = 17.0 Hz, 1H), 2.45 (s, 3H) | 433.0 |
| | or | | | |
| | 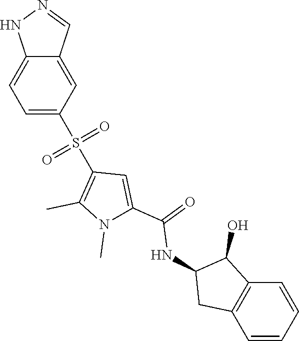 [SMILES: Cc1c(S(=O)(=O)c2ccc3[nH]ncc3c2)cc(C(=O)N[C@@H]2Cc3ccccc3[C@@H]2O)n1C] | | | |
| B.20 | 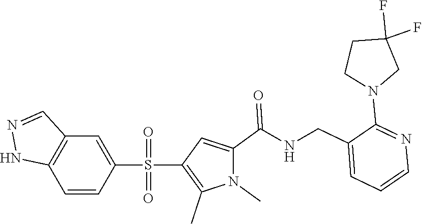 [SMILES: Cc1c(S(=O)(=O)c2ccc3[nH]ncc3c2)cc(C(=O)NCc2cccnc2N2CCC(F)(F)C2)n1C] | 0.75 | B δ 8.46 (d, J = 0.9 Hz, 1H), 8.24 (d, J = 0.6 Hz, 1H), 8.12- 8.08 (m, 1H), 7.97 (dd, J = 1.4, 6.2 Hz, 1H), 7.81 (dd, J = 1.6, 8.9 Hz, 1H), 7.67 (d, J = 8.9 Hz, 1H), 7.26 (s, 1H), 7.14 (dd, J = 6.5, 7.3 Hz, 1H), 4.67 (s, 2H), 4.28 (t, J = 12.3 Hz, 2H), 4.05 (t, J = 7.3 Hz, 2H), 3.79 (s, 3H), 2.69-2.60 (m, 2H), 2.47 (s, 3H) | 515.1 |
| B.21 | 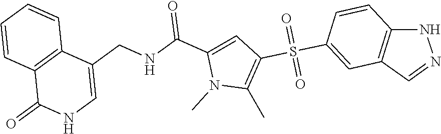 [SMILES: Cc1c(S(=O)(=O)c2ccc3[nH]ncc3c2)cc(C(=O)NCc2c[nH]c(=O)c3ccccc23)n1C] | 0.54 | A δ 13.79-13.15 (m, 1H), 11.23 (br d, J = 1.9 Hz, 1H), 8.59 (br t, J = 5.2 Hz, 1H), 8.35 (s, 1H), 8.28 (s, 1H), 8.23 (d, J = 8.0 Hz, 1H), 7.83-7.77 (m, 1H), 7.76-7.70 (m, 1H), 7.67 (s, 2H), 7.51 (t, J = 7.4 Hz, 1H), 7.19 (s, 1H), 7.14 (s, | 476.0 |
| | | | 1H), 4.45 (br d, J = 5.3 Hz, | |
| | | | 2H), 3.77 (s, 3H), 2.39 (s, 3H) | |
| B.22 | 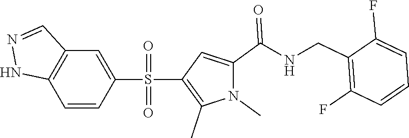 [SMILES: Cc1c(S(=O)(=O)c2ccc3[nH]ncc3c2)cc(C(=O)NCc2c(F)cccc2F)n1C] | 0.23 | A δ 13.52 (br s, 1H), 8.66 (t, J = 5.1 Hz, 1H), 8.37 (s, 1H), 8.30 (s, 1H), 7.69 (s, 2H), 7.45-7.34 (m, 1H), 7.23 (s, 1H), 7.14-7.02 (m, 2H), 4.41 (d, J = 5.1 Hz, 2H), 3.73 (s, 3H), 2.38 (s, 3H) | 445.0 |
| B.23 | 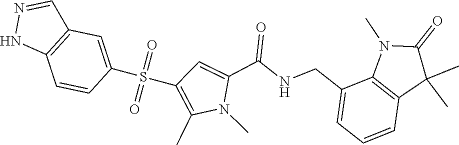 [SMILES: Cc1c(S(=O)(=O)c2ccc3[nH]ncc3c2)cc(C(=O)NCc2cccc3c2N(C)C(=O)C3(C)C)n1C] | 1.4 | A δ 13.52 (br s, 1H), 8.76 (t, J = 5.3 Hz, 1H), 8.39 (s, 1H), 8.30 (s, 1H), 7.70 (s, 2H), 7.31 (s, 1H), 7.27 (d, J = 6.5 Hz, 1H), 7.18 (d, J = 7.6 Hz, 1H), 7.04 - 6.99 (m, 1H), 4.67 (d, J = 5.3 Hz, 2H), 3.76 (s, 3H), 3.43 (s, 3H), 2.40 (s, 3H), 1.26 (s, 6H) | 504.1 |
| B.24 | 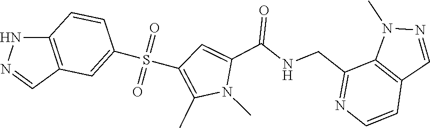 [SMILES: Cc1c(S(=O)(=O)c2ccc3[nH]ncc3c2)cc(C(=O)NCc2nccc3cnn(C)c23)n1C] | 0.62 | B δ 8.50-8.46 (m, 2H), 8.28- 8.24 (m, 2H), 8.22-8.18 (m, 1H), 7.82 (dd, J = 8.88, 1.75 Hz, 1H), 7.68 (d, J = 8.75 Hz, 1H), 7.40 (s, 1H), 5.38 (s, 2H), 4.57 (s, 3H), 3.80 (s, 3H), 2.48 (s, 3H) | 464.1 |
| B.25 | 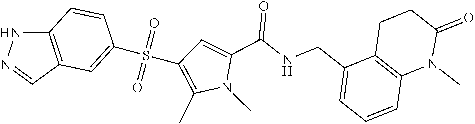 [SMILES: Cc1c(S(=O)(=O)c2ccc3[nH]ncc3c2)cc(C(=O)NCc2cccc3c2CCC(=O)N3C)n1C] | <0.15 | B δ 8.45 (d, J = 0.88 Hz, 1H), 8.25-8.25 (m, 1H), 7.80 (dd, J = 8.88, 1.63 Hz, 1H), 7.65 (d, J = 9.01 Hz, 1H), 7.29-7.23 (m, 1H), 7.15 (s, 1H), 7.09 (dd, J = 7.88, 2.50 Hz, 2H), 4.51 (s, 2H), 3.78 (s, 3H), 3.36 (s, 3H), 2.99-2.91 (m, 2H), | 492.1 |
| | | | 2.64-2.56 (m, 2H), 2.45 (s, | |
| | | | 3H) | |
| B.26 | 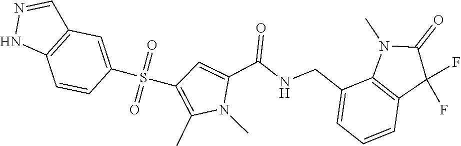 [SMILES: Cc1c(S(=O)(=O)c2ccc3[nH]ncc3c2)cc(C(=O)NCc2cccc3c2N(C)C(=O)C3(F)F)n1C] | 0.49 | B δ 8.45 (d, J = 0.9 Hz, 1H), 8.23 (s, 1H), 7.80 (dd, J = 1.6, 8.9 Hz, 1H), 7.65 (d, J = 8.9 Hz, 1H), 7.55-7.48 (m, 2H), 7.24-7.17 (m, 2H), 4.75 (s, 2H), 3.79 (s, 3H), 3.54 (s, 3H), 2.45 (s, 3H) | 514.1 |
| B.27 | 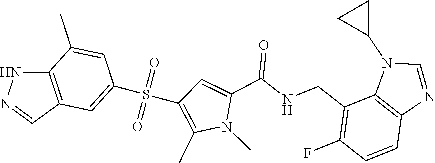 [SMILES: Cc1cc(S(=O)(=O)c2cc(C(=O)NCc3c(F)ccc4ncn(C5CC5)c34)n(C)c2C)cc2cn[nH]c12] | 0.21 | A δ 13.58 (br s, 1H), 8.61 (t, J = 4.3 Hz, 1H), 8.26 (s, 1H), 8.17 (s, 1H), 8.02 (s, 1H), 7.76 (dd, J = 5.1, 8.8 Hz, 1H), 7.43 (s, 1H), 7.22 (s, 1H), 7.07 (dd, J = 8.9, 10.1 Hz, 1H), 4.99 (br d, J = 2.9 Hz, 2H), 3.96 (tt, J = 3.6, 7.1 Hz, 1H), 3.76 (s, 3H), 2.53 (s, 3H), 2.38 (s, 3H), 1.30-1.24 (m, 2H), 1.11-1.02 (m, 2H) | 521.1 |
| B.28 | 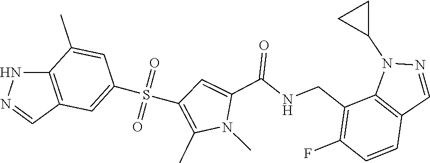 [SMILES: Cc1cc(S(=O)(=O)c2cc(C(=O)NCc3c(F)ccc4cnn(C5CC5)c34)n(C)c2C)cc2cn[nH]c12] | 0.24 | A δ 13.58 (br s, 1H), 8.61 (t, J = 4.3 Hz, 1H), 8.26 (s, 1H), 8.17 (s, 1H), 8.02 (s, 1H), 7.76 (dd, J = 5.1, 8.8 Hz, 1H), 7.43 (s, 1H), 7.22 (s, 1H), 7.07 (dd, J = 8.9, 10.1 Hz, 1H), 4.99 (br d, J = 2.9 Hz, 2H), 3.96 (tt, J = 3.6, 7.1 Hz, 1H), 3.76 (s, 3H), 2.53 (s, 3H), 2.38 (s, 3H), 1.30-1.24 (m, 2H), 1.11-1.02 (m, 2H) | 521.0 |
| B.29 | 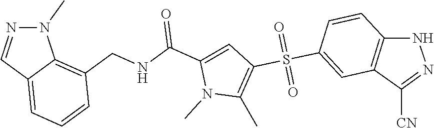 [SMILES: Cc1c(S(=O)(=O)c2ccc3[nH]nc(C#N)c3c2)cc(C(=O)NCc2cccc3cnn(C)c23)n1C] | 138 | A δ 14.99-14.53 (m, 1H), 8.83 (br s, 1H), 8.37 (br s, 1H), 8.03 (s, 1H), 7.98-7.91 (m, 1H), 7.91-7.84 (m, 1H), 7.68 (br d, J = 7.8 Hz, 1H), 7.38 (br s, 1H), 7.27 (br d, J = 6.6 Hz, 1H), 7.08 (br t, J = 7.4 Hz, 1H), 4.88 (br s, 2H), 4.25 (br | 488.1 |
| | | | s, 3H), 3.78 (br s, 3H), 2.43 | |
| | | | (br s, 3H) | |
| B.30 | 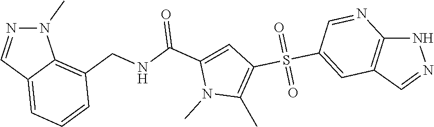 [SMILES: Cc1c(S(=O)(=O)c2cnc3[nH]ncc3c2)cc(C(=O)NCc2cccc3cnn(C)c23)n1C] | 4.6 | A δ 8.90 (d, J = 2.1 Hz, 1H), 8.84 (br t, J = 5.1 Hz, 1H), 8.79 (d, J = 2.1 Hz, 1H), 8.34 (s, 1H), 8.03 (s, 1H), 7.67 (d, J = 8.0 Hz, 1H), 7.35 (s, 1H), 7.27 (d, J = 7.0 Hz, 1H), 7.08 (t, J = 7.6 Hz, 1H), 4.89 (d, J = 5.1 Hz, 2H), 4.25 (s, 3H), 3.77 (s, 3H), 2.44 (s, 3H) | 464.1 |
| B.31 | 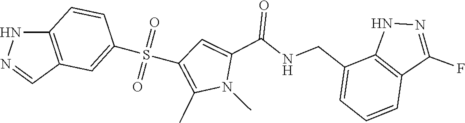 [SMILES: Cc1c(S(=O)(=O)c2ccc3[nH]ncc3c2)cc(C(=O)NCc2cccc3c(F)n[nH]c23)n1C] | 5.4 | A δ 13.77-13.25 (m, 1H), 12.81-12.40 (m, 1H), 8.89 (t, J = 5.8 Hz, 1H), 8.40 (s, 1H), 8.31 (s, 1H), 7.71 (s, 2H), 7.60 (d, J = 8.1 Hz, 1H), 7.32-7.27 (m, 2H), 7.18-7.12 (m, 1H), 4.63 (d, J = 5.6 Hz, 2H), 3.76 (s, 3H), 2.41 (s, 3H) | 467.0 |
| B.32 | 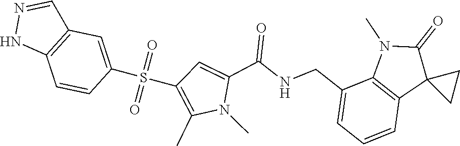 [SMILES: Cc1c(S(=O)(=O)c2ccc3[nH]ncc3c2)cc(C(=O)NCc2cccc3c2N(C)C(=O)C32CC2)n1C] | 0.18 | A δ 13.59-13.44 (m, 1H), 8.75 (t, J = 5.1 Hz, 1H), 8.38 (s, 1H), 8.30 (s, 1H), 7.70 (s, 2H), 7.31 (s, 1H), 7.16 (dd, J = 1.2, 7.6 Hz, 1H), 6.99-6.90 (m, 2H), 4.68 (d, J = 5.1 Hz, 2H), 3.76 (s, 3H), 3.50 (s, 3H), 2.39 (s, 3H), 1.60-1.51 (m, 4H) | 504.1 |
| B.33 | 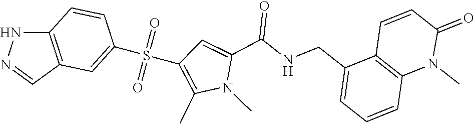 [SMILES: Cc1c(S(=O)(=O)c2ccc3[nH]ncc3c2)cc(C(=O)NCc2cccc3c2ccc(=O)n3C)n1C] | 0.35 | A δ 13.60-13.43 (m, 1H), 8.87 (t, J = 5.8 Hz, 1H), 8.38 (t, J = 1.1 Hz, 1H), 8.30 (s, 1H), 8.19 (d, J = 9.8 Hz, 1H), 7.69 (d, J = 0.8 Hz, 2H), 7.62-7.55 (m, 1H), 7.48 (d, J = 8.5 Hz, 1H), 7.29-7.20 (m, 2H), 6.66 (d, J = 9.9 Hz, 1H), 4.69 (d, J = 5.6 | 490.1 |
| | | | Hz, 2H), 3.75 (s, 3H), 3.63 (s, | |
| | | | 3H), 2.39 (s, 3H) | |
| B.34 | 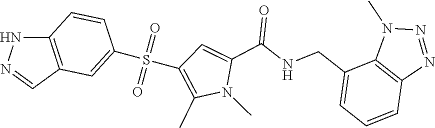 [SMILES: Cc1c(S(=O)(=O)c2ccc3[nH]ncc3c2)cc(C(=O)NCc2cccc3nnn(C)c23)n1C] | 0.2 | B δ 8.46-8.41 (m, 1H), 8.25- 8.21 (m, 1H), 7.89 (d, J = 8.4 Hz, 1H), 7.80 (dd, J = 1.6, 8.9 Hz, 1H), 7.65 (d, J = 8.9 Hz, 1H), 7.51-7.46 (m, 1H), 7.38 (dd, J = 7.3, 8.3 Hz, 1H), 7.23 (s, 1H), 5.00 (s, 2H), 4.56 (s, 3H), 3.80 (s, 3H), 2.46 (s, 3H) | 464.0 |
| B.35 | 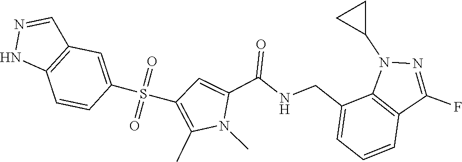 [SMILES: Cc1c(S(=O)(=O)c2ccc3[nH]ncc3c2)cc(C(=O)NCc2cccc3c(F)nn(C4CC4)c23)n1C] | 3.3 | A δ 13.54 (br s, 1H), 8.97-8.79 (m, 1H), 8.40 (s, 1H), 8.31 (s, 1H), 7.71 (s, 2H), 7.59 (d, J = 8.1 Hz, 1H), 7.39-7.31 (m, 2H), 7.21-7.14 (m, 1H), 5.06 (d, J = 5.4 Hz, 2H), 3.98-3.88 (m, 1H), 3.76 (s, 3H), 2.41 (s, 3H), 1.23 (br d, J = 3.0 Hz, 2H), 1.16-1.06 (m, 2H) | 507.1 |
| B.36 | 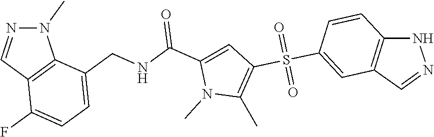 [SMILES: Cc1c(S(=O)(=O)c2ccc3[nH]ncc3c2)cc(C(=O)NCc2ccc(F)c3cnn(C)c23)n1C] | 0.41 | A δ 13.55-13.46 (m, 1H), 8.82- 8.76 (m, 1H), 8.38 (s, 1H), 8.29 (s, 1H), 8.14 (s, 1H), 7.69 (s, 2H), 7.31 (s, 1H), 7.26 (dd, J = 5.3, 7.8 Hz, 1H), 6.87 (dd, J = 7.9, 9.9 Hz, 1H), 4.83 (d, J = 5.0 Hz, 2H), 4.26 (s, 3H), 3.77 (s, 3H), 2.40 (s, 3H) | 481.1 |
| B.37 | 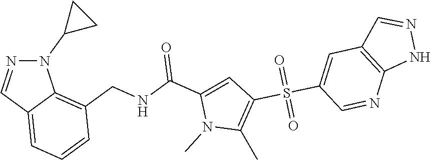 [SMILES: Cc1c(S(=O)(=O)c2cnc3[nH]ncc3c2)cc(C(=O)NCc2cccc3cnn(C4CC4)c23)n1C] | 10 | B δ 8.95 (d, J = 1.8 Hz, 1H), 8.80 (d, J = 1.8 Hz, 1H), 8.27 (s, 1H), 7.94 (s, 1H), 7.65 (d, J = 8.0 Hz, 1H), 7.36 (d, J = 7.1 Hz, 1H), 7.26 (s, 1H), 7.13 (t, J = 7.6 Hz, 1H), 5.23 (s, 2H), 3.99-3.92 (m, 1H), 3.82 (s, 3H), 2.50 (s, 3H), 1.38-1.30 (m, 2H), 1.26-1.18 (m, 2H) | 490.1 |
| B.38 | 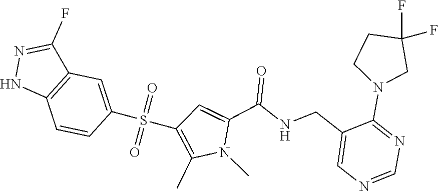 [SMILES: Cc1c(S(=O)(=O)c2ccc3[nH]nc(F)c3c2)cc(C(=O)NCc2cncnc2N2CCC(F)(F)C2)n1C] | 0.56 | B δ 8.71 (d, J = 0.9 Hz, 1H), 8.32-8.26 (m, 1H), 8.23 (s, 1H), 7.84 (dd, J = 1.6, 9.0 Hz, 1H), 7.58 (dd, J = 1.8, 8.9 Hz, 1H), 7.25 (d, J = 4.6 Hz, 1H), 4.66 (s, 2H), 4.37 (br t, J = 12.1 Hz, 2H), 4.26 (br t, J = 7.3 Hz, 2H), 3.80 (s, 3H), 2.64-2.53 (m, 2H), 2.50-2.41 (m, 3H) | :534.1 |
| B.39 | 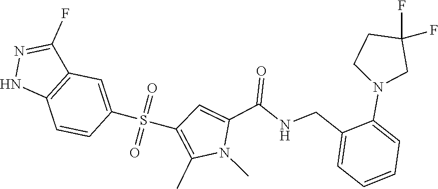 [SMILES: Cc1c(S(=O)(=O)c2ccc3[nH]nc(F)c3c2)cc(C(=O)NCc2ccccc2N2CCC(F)(F)C2)n1C] | 5.2 | B δ 8.32 (s, 1H), 7.84 (dd, J = 1.8, 9.0 Hz, 1H), 7.57 (dd, J = 1.7, 8.9 Hz, 1H), 7.39-7.29 (m, 3H), 7.24-7.17 (m, 2H), 4.57 (s, 2H), 3.80 (s, 3H), 3.69 (br t, J = 12.5 Hz, 2H), 3.54 (br t, J = 6.9 Hz, 2H), 2.57 (dt, J = 7.2, 14.4 Hz, 2H), 2.47 (s, 3H) | 532.1 |
| B.40 | 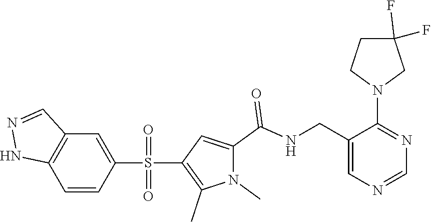 [SMILES: Cc1c(S(=O)(=O)c2ccc3[nH]ncc3c2)cc(C(=O)NCc2cncnc2N2CCC(F)(F)C2)n1C] | 0.22 | B δ 8.49-8.41 (m, 2H), 8.23 (s, 1H), 8.16 (s, 1H), 7.79 (dd, J = 1.6, 8.9 Hz, 1H), 7.65 (d, J = 8.9 Hz, 1H), 7.18 (s, 1H), 4.55 (s, 2H), 4.09 (t, J = 13.0 Hz, 2H), 3.98 (t, J = 7.4 Hz, 2H), 3.79 (s, 3H), 2.52-2.41 (m, 5H) | 516.1 |
| B.41 | 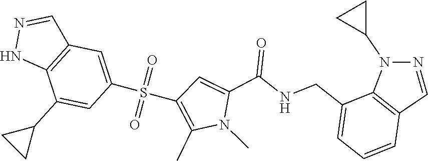 [SMILES: Cc1c(S(=O)(=O)c2cc(C3CC3)c3[nH]ncc3c2)cc(C(=O)NCc2cccc3cnn(C4CC4)c23)n1C] | 50 | A δ 8.85 (t, J = 5.4 Hz, 1H), 8.30 (s, 1H), 8.18 (d, J = 1.5 Hz, 1H), 7.99 (s, 1H), 7.64 (d, J = 7.8 Hz, 1H), 7.35 (s, 1H), 7.25 (d, J = 7.1 Hz, 1H), 7.19 (d, J = 0.8 Hz, 1H), 7.10 (t, J = 7.6 Hz, 1H), 5.11 (d, J = 5.4 Hz, 2H), 4.03 (tt, J = 3.5, 7.0 Hz, 1H), 3.76 (s, 3H), 2.41 (s, 3H), 2.37-2.28 (m, 1H), 1.31- | 529.1 |
| | | | 1.24 (m, 2H), 1.16-1.07 (m, | |
| | | | 4H), 0.83-0.76 (m, 2H) | |
| B.42 | 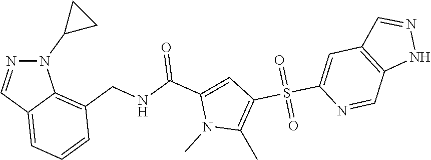 [SMILES: Cc1c(S(=O)(=O)c2cc3cn[nH]c3cn2)cc(C(=O)NCc2cccc3cnn(C4CC4)c23)n1C] | 9.3 | B δ 9.02 (s, 1H), 8.62 (d, J = 1.0 Hz, 1H), 8.38 (s, 1H), 7.95 (s, 1H), 7.65 (d, J = 8.0 Hz, 1H), 7.35 (d, J = 7.3 Hz, 1H), 7.24 (s, 1H), 7.13 (t, J = 7.6 Hz, 1H), 5.22 (s, 2H), 3.96 (tt, J = 3.6, 7.0 Hz, 1H), 3.82 (s, 3H), 2.54 (s, 3H), 1.38-1.27 (m, 2H), 1.26-1.18 (m, 2H) | 490.1 |
| B.43 | 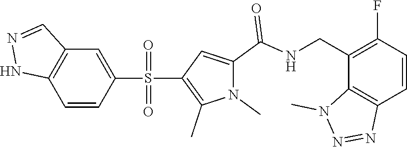 [SMILES: Cc1c(S(=O)(=O)c2ccc3[nH]ncc3c2)cc(C(=O)NCc2c(F)ccc3nnn(C)c23)n1C] | <0.15 | A δ 8.72 (t, J = 4.6 Hz, 1H), 8.36 (s, 1H), 8.32-8.25 (m, 1H), 8.06 (dd, J = 4.6, 9.1 Hz, 1H), 7.67 (s, 2H), 7.33 (t, J = 9.6 Hz, 1H), 7.27-7.21 (m, 1H), 4.79 (br d, J = 3.6 Hz, 2H), 4.45 (s, 3H), 3.75 (s, 3H), 2.38 (s, 3H) | :482.2 |
| B.44 | 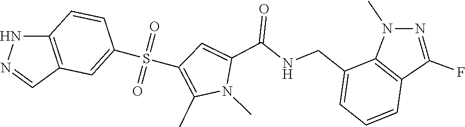 [SMILES: Cc1c(S(=O)(=O)c2ccc3[nH]ncc3c2)cc(C(=O)NCc2cccc3c(F)nn(C)c23)n1C] | 0.24 | A δ 13.67-13.35 (m, 1H), 8.85 (t, J = 5.2 Hz, 1H), 8.39 (s, 1H), 8.30 (s, 1H), 7.70 (s, 2H), 7.62 (d, J = 8.1 Hz, 1H), 7.38- 7.32 (m, 2H), 7.15 (t, J = 7.6 Hz, 1H), 4.84 (d, J = 5.3 Hz, 2H), 4.10 (s, 3H), 3.76 (s, 3H), 2.41 (s, 3H) | 481.1 |
| B.45 | 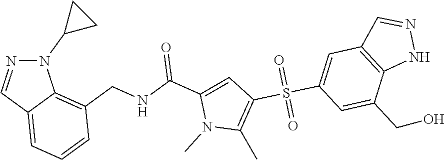 [SMILES: Cc1c(S(=O)(=O)c2cc(CO)c3[nH]ncc3c2)cc(C(=O)NCc2cccc3cnn(C4CC4)c23)n1C] | 4 | A δ 13.79-13.28 (m, 1H), 8.88 (t, J = 5.4 Hz, 1H), 8.33-8.26 (m, 2H), 7.98 (s, 1H), 7.72 (s, 1H), 7.64 (d, J = 7.9 Hz, 1H), 7.34 (s, 1H), 7.26 (d, J = 7.1 Hz, 1H), 7.10 (t, J = 7.6 Hz, 1H), 5.11 (d, J = 5.4 Hz, 2H), 4.83 (s, 2H), 4.03 (tt, J = 3.6, 7.0 Hz, 1H), 3.77 (s, 3H), 2.41 (s, 3H), 1.31-1.24 (m, 2H), 1.17-1.10 (m, 2H) | 519.1 |
| B.46 | 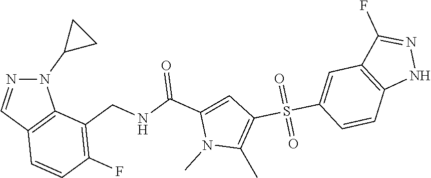 [SMILES: Cc1c(S(=O)(=O)c2ccc3[nH]nc(F)c3c2)cc(C(=O)NCc2c(F)ccc3cnn(C4CC4)c23)n1C] | 0.53 | A δ 13.07 (s, 1H), 8.64-8.56 (m, 1H), 8.21 (s, 1H), 8.03 (s, 1H), 7.80-7.69 (m, 2H), 7.67- 7.61 (m, 1H), 7.25 (s, 1H), 7.07 (t, J = 9.5 Hz, 1H), 5.00 (br d, J = 2.8 Hz, 2H), 3.96 (tt, J = 3.7, 7.0 Hz, 1H), 3.77 (s, 3H), 2.39 (s, 3H), 1.31-1.23 (m, 2H), 1.07 (dd, J = 1.8, 6.9 Hz, 2H) | 525.1 |
| B.47 | 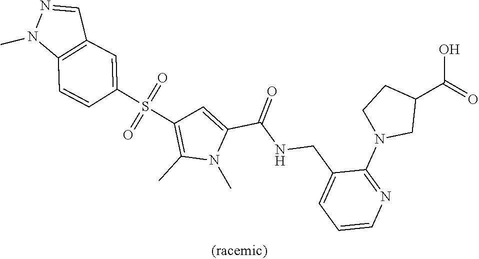 [SMILES: Cc1c(S(=O)(=O)c2ccc3c(cnn3C)c2)cc(C(=O)NCc2cccnc2N2CCC(C(=O)O)C2)n1C] | 1.7 | A δ 8.82 (br t, J = 5.0 Hz, 1H), 8.39 (d, J = 1.0 Hz, 1H), 8.29 (s, 1H), 7.97 (d, J = 4.8 Hz, 1H), 7.89-7.79 (m, 2H), 7.79- 7.73 (m, 1H), 7.32 (s, 1H), 7.01-6.92 (m, 1H), 4.54 (br d, J = 4.5 Hz, 2H), 4.09 (s, 3H), 3.92-3.84 (m, 2H), 3.77 (br s, 2H), 3.73 (s, 3H), 3.29-3.19 (m, 1H), 2.41 (s, 3H), 2.29- 2.10 (m, 2H) | 537.2 |
| B.48 | 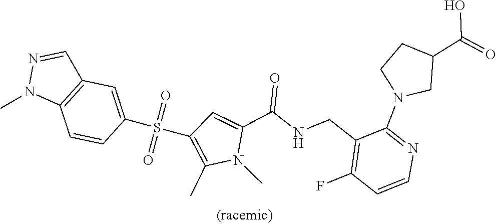 [SMILES: Cc1c(S(=O)(=O)c2ccc3c(cnn3C)c2)cc(C(=O)NCc2c(F)ccnc2N2CCC(C(=O)O)C2)n1C] | 0.15 | A δ 8.52 (br s, 1H), 8.36 (s, 1H), 8.27 (s, 1H), 8.12 (t, J = 6.9 Hz, 1H), 7.85-7.78 (m, 1H), 7.77-7.69 (m, 1H), 7.24 (s, 1H), 6.93-6.79 (m, 1H), 4.49-4.38 (m, 2H), 4.07 (s, 3H), 3.83 (br d, J = 7.0 Hz, 2H), 3.74 (s, 3H), 3.71 (br s, 2H), 3.22-3.13 (m, 1H), 2.39 (s, 3H), 2.23-2.04 (m, 2H) | 555.1 |
| B.49 | 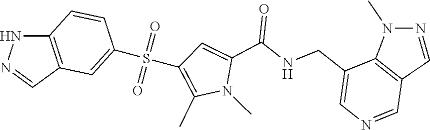 [SMILES: Cc1c(S(=O)(=O)c2ccc3[nH]ncc3c2)cc(C(=O)NCc2cncc3cnn(C)c23)n1C] | 0.49 | A & 13.69-13.26 (m, 1H), 9.02 (s, 1H), 8.80 (t, J = 5.1 Hz, 1H), 8.37 (s, 1H), 8.32-8.24 (m, 3H), 7.69 (d, J = 0.8 Hz, 2H), 7.33-7.26 (m, 1H), 4.85 (d, J = 4.9 Hz, 2H), 4.22 (s, 3H), 3.77 (s, 3H), 2.40 (s, 3H) | 464.0 |
| B.50 | 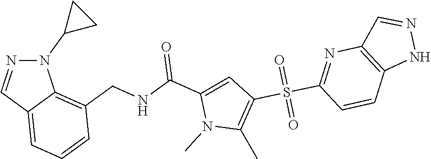 [SMILES: Cc1c(S(=O)(=O)c2ccc3[nH]ncc3n2)cc(C(=O)NCc2cccc3cnn(C4CC4)c23)n1C] | 51 | B δ 8.34 (s, 1H), 8.23-8.17 (m, 1H), 8.14-8.08 (m, 1H), 7.93 (s, 1H), 7.65 (d, J = 8.0 Hz, 1H), 7.35 (d, J = 7.4 Hz, 1H), 7.25 (s, 1H), 7.16-7.10 (m, 1H), 5.23 (s, 2H), 3.96 (td, J = 3.4, 7.0 Hz, 1H), 3.83 (s, 3H), 2.56 (s, 3H), 1.37-1.30 (m, 2H), 1.26-1.17 (m, 2H) | 490.1 |
| B.51 | 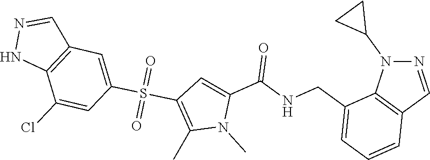 [SMILES: Cc1c(S(=O)(=O)c2cc(Cl)c3[nH]ncc3c2)cc(C(=O)NCc2cccc3cnn(C4CC4)c23)n1C] | 0.22 | A δ 8.86 (t, J = 5.4 Hz, 1H), 8.44 (br s, 1H), 8.40 (d, J = 1.3 Hz, 1H), 7.99 (s, 1H), 7.73 (s, 1H), 7.64 (d, J = 7.8 Hz, 1H), 7.36 (s, 1H), 7.27 (d, J = 7.1 Hz, 1H), 7.10 (t, J = 7.6 Hz, 1H), 5.11 (d, J = 5.4 Hz, 2H), 4.03 (td, J = 3.5, 7.0 Hz, 1H), 3.77 (s, 3H), 2.42 (s, 3H), 1.31-1.24 (m, 2H), 1.17-1.09 (m, 2H) | 523.0 |
| B.52 | 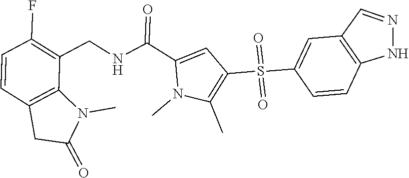 [SMILES: Cc1c(S(=O)(=O)c2ccc3[nH]ncc3c2)cc(C(=O)NCc2c(F)ccc3c2N(C)C(=O)C3)n1C] | 0.15 | A δ 13.55-13.48 (m, 1H), 8.58 (t, J = 4.00 Hz, 1H), 8.36 (s, 1H), 8.31-8.24 (m, 1H), 7.68 (s, 2H), 7.29-7.19 (m, 2H), 6.90-6.81 (m, 1H), 4.64-4.55 (m, 2H), 3.76 (s, 3H), 3.56 (s, 2H), 3.39 (s, 3H), 2.38 (s, 3H) | 496.0 |
| B.53 | 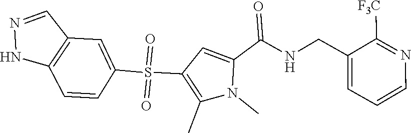 [SMILES: Cc1c(S(=O)(=O)c2ccc3[nH]ncc3c2)cc(C(=O)NCc2cccnc2C(F)(F)F)n1C] | 7 | A δ 8.94 (t, J = 5.8 Hz, 1H), 8.62 (d, J = 4.4 Hz, 1H), 8.41 (s, 1H), 8.31 (s, 1H), 7.96 (d, J = 7.8 Hz, 1H), 7.74-7.68 (m, 3H), 7.33 (s, 1H), 4.57 (br d, J = 5.3 Hz, 2H), 3.74 (s, 3H), 2.41 (s, 3H) | 478.0 |
| B.54 | 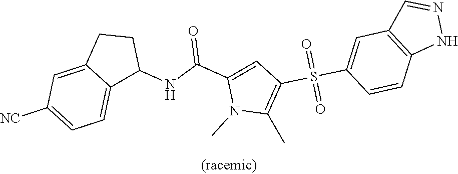 [SMILES: Cc1c(S(=O)(=O)c2ccc3[nH]ncc3c2)cc(C(=O)NC2CCc3cc(C#N)ccc32)n1C] | 2.3 | B δ 8.44 (s, 1H), 8.23 (s, 1H), 7.83-7.76 (m, 1H), 7.67-7.59 (m, 2H), 7.55 (br d, J = 7.88 Hz, 1H), 7.42 (d, J = 7.75 Hz, 1H), 7.20 (s, 1H), 5.57 (t, J = 8.13 Hz, 1H), 3.83 (s, 3H), 3.14-3.03 (m, 1H), 2.94 (dt, J = 16.42, 8.36 Hz, 1H), 2.63- 2.52 (m, 1H), 2.47 (s, 3H), 2.04 (dq, J = 12.60, 8.97 Hz, 1H) | 460.0 |
| B.55 | 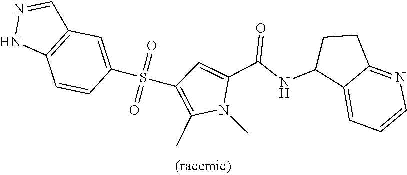 [SMILES: Cc1c(S(=O)(=O)c2ccc3[nH]ncc3c2)cc(C(=O)NC2CCc3ncccc32)n1C] | 5.6 | B δ 8.48-8.40 (m, 1H), 8.39- 8.31 (m, 1H), 8.23 (s, 1H), 7.80 (dd, J = 8.88, 1.50 Hz, 1H), 7.74 (d, J = 7.63 Hz, 1H), 7.65 (d, J = 8.88 Hz, 1H), 7.25 (dd, J = 7.57, 5.07 Hz, 1H), 7.19 (s, 1H), 5.57 (t, J = 7.69 Hz, 1H), 3.83 (s, 3H), 3.18- 3.08 (m, 1H), 3.06-2.94 (m, 1H), 2.68-2.54 (m, 1H), 2.47 (s, 3H), 2.07 (dq, J = 13.09, 8.40 Hz, 1H) | 436.1 |
| B.56 | 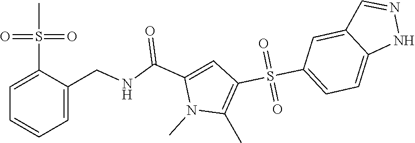 [SMILES: Cc1c(S(=O)(=O)c2ccc3[nH]ncc3c2)cc(C(=O)NCc2ccccc2S(C)(=O)=O)n1C] | 2.9 | A δ 13.54 (br s, 1H), 9.02 (t, J = 6.0 Hz, 1H), 8.42 (s, 1H), 8.32 (s, 1H), 7.93 (dd, J = 1.3, 7.9 Hz, 1H), 7.80-7.65 (m, 3H), 7.63-7.48 (m, 2H), 7.36 (s, 1H), 4.78 (d, J = 5.9 Hz, 2H), 3.75 (s, 3H), 3.36 (s, 3H), 2.42 (s, 3H) A δ 13.54 (br s, 1H), 9.02 (t, | 487.0 |
| | | | J = 6.0 Hz, 1H), 8.42 (s, 1H), | |
| | | | 8.32 (s, 1H), 7.93 (dd, J = 1.3, | |
| | | | 7.9 Hz, 1H), 7.80-7.65 (m, | |
| | | | 3H), 7.63-7.48 (m, 2H), 7.36 | |
| | | | (s, 1H), 4.78 (d, J = 5.9 Hz, | |
| | | | 2H), 3.75 (s, 3H), 3.36 (s, 3H), | |
| | | | 2.42 (s, 3H) | |
| B.57 | 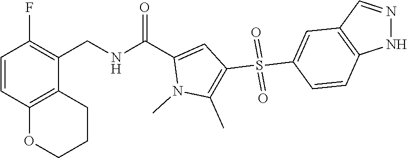 [SMILES: Cc1c(S(=O)(=O)c2ccc3[nH]ncc3c2)cc(C(=O)NCc2c(F)ccc3c2CCCO3)n1C] | <0.15 | A δ 13.56-13.48 (m, 1H), 8.45 (t, J = 4.9 Hz, 1H), 8.36 (s, 1H), 8.32-8.27 (m, 1H), 7.72- 7.64 (m, 2H), 7.25 (s, 1H), 6.93 (t, J = 9.3 Hz, 1H), 6.72 (dd, J = 4.8, 8.9 Hz, 1H), 4.35 (br d, J = 4.0 Hz, 2H), 4.09- 4.02 (m, 2H), 3.75 (s, 3H), 2.76 (t, J = 6.4 Hz, 2H), 2.39 (s, 3H), 1.96-1.83 (m, 2H) | :483.0 |
| B.58 | 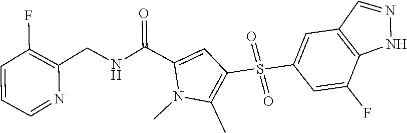 [SMILES: Cc1c(S(=O)(=O)c2cc(F)c3[nH]ncc3c2)cc(C(=O)NCc2ncccc2F)n1C] | 2.3 | A δ 14.04-13.72 (m, 1H), 8.77 (br t, J = 5.3 Hz, 1H), 8.42 (br d, J = 2.5 Hz, 1H), 8.37 (br d, J = 4.1 Hz, 1H), 8.26 (s, 1H), 7.68 (br t, J = 9.3 Hz, 1H), 7.49 (br d, J = 10.1 Hz, 1H), 7.39 (td, J = 4.2, 8.4 Hz, 1H), 7.28 (s, 1H), 4.54 (br d, J = 5.1 Hz, 2H), 3.73 (s, 3H), 2.41 (s, 3H) | 446.0 |
| B.59 | 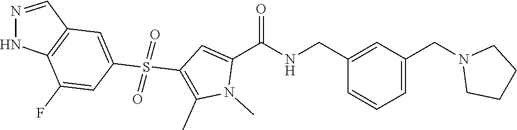 [SMILES: Cc1c(S(=O)(=O)c2cc(F)c3[nH]ncc3c2)cc(C(=O)NCc2cccc(CN3CCCC3)c2)n1C] | 41 | A & 14.29-14.03 (m, 1H), 10.78-10.46 (m, 1H), 8.87 (br t, J = 5.9 Hz, 1H), 8.42 (br s, 1H), 8.27 (s, 1H), 7.55-7.44 (m, 3H), 7.41 (t, J = 7.4 Hz, 1H), 7.38-7.33 (m, 1H), 7.29 (s, 1H), 4.40 (br d, J = 5.9 Hz, 2H), 4.31 (br d, J = 5.8 Hz, | 510.1 |
| | | | 2H), 3.76 (s, 3H), 3.36-3.27 | |
| | | | (m, 2H), 3.08-2.97 (m, 2H), | |
| | | | 2.42 (s, 3H), 2.05-1.94 (m, | |
| | | | 2H), 1.91-1.80 (m, 2H) | |
| B.60 | 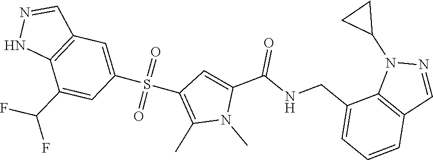 [SMILES: Cc1c(S(=O)(=O)c2cc(C(F)F)c3[nH]ncc3c2)cc(C(=O)NCc2cccc3cnn(C4CC4)c23)n1C] | 5.3 | A δ 13.95 (br d, J = 5.0 Hz, 1H), 8.87 (br t, J = 5.2 Hz, 1H), 8.58 (s, 1H), 8.45 (s, 1H), 7.99 (s, 1H), 7.95 (s, 1H), 7.64 (d, J = 7.8 Hz, 1H), 7.59-7.28 (m, 2H), 7.26 (d, J = 6.9 Hz, 1H), 7.10 (t, J = 7.5 Hz, 1H), 5.11 (br d, J = 5.3 Hz, 2H), 4.07-3.99 (m, 1H), 3.77 (s, 3H), 2.43 (s, 3H), 1.33-1.22 (m, 2H), 1.19-1.07 (m, 2H) | 539.1 |
| B.61 | 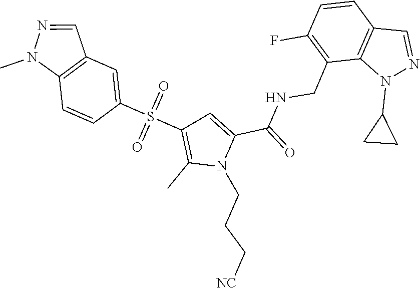 [SMILES: Cc1c(S(=O)(=O)c2ccc3c(cnn3C)c2)cc(C(=O)NCc2c(F)ccc3cnn(C4CC4)c23)n1CCCC#N] | 2.6 | A δ 8.62 (t, J = 4.3 Hz, 1H), 8.36-8.32 (m, 1H), 8.25 (d, J = 0.9 Hz, 1H), 8.03 (s, 1H), 7.82-7.74 (m, 2H), 7.73-7.67 (m, 1H), 7.32 (s, 1H), 7.08 (dd, J = 8.8, 10.1 Hz, 1H), 5.01 (br d, J = 2.6 Hz, 2H), 4.38-4.28 (m, 2H), 4.06 (s, 3H), 3.93 (td, J = 3.5, 7.1 Hz, 1H), 2.55-2.51 (m, 2H), 2.42 (s, 3H), 1.89 (quin, J = 7.4 Hz, 2H), 1.31-1.23 (m, 2H), 1.13- 1.03 (m, 2H) | 574.1 |
| B.62 | 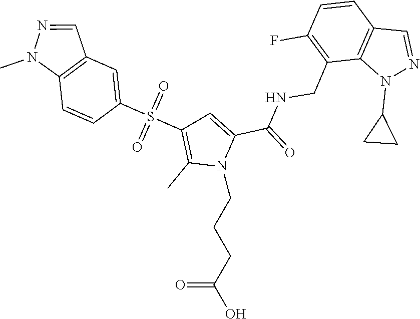 [SMILES: Cc1c(S(=O)(=O)c2ccc3c(cnn3C)c2)cc(C(=O)NCc2c(F)ccc3cnn(C4CC4)c23)n1CCCC(=O)O] | <0.15 | A δ 8.60 (t, J = 4.2 Hz, 1H), 8.34 (d, J = 1.0 Hz, 1H), 8.25 (s, 1H), 8.03 (s, 1H), 7.81-7.74 (m, 2H), 7.73-7.67 (m, 1H), 7.29 (s, 1H), 7.07 (dd, J = 8.9, 9.9 Hz, 1H), 5.00 (br d, J = 2.6 Hz, 2H), 4.34-4.24 (m, 2H), 4.06 (s, 3H), 3.90 (dt, J = 3.6, 7.0 Hz, 1H), 2.41 (s, 3H), 2.19 (t, J = 7.3 Hz, 2H), 1.77 (quin, J = 7.3 Hz, 2H), 1.32- 1.22 (m, 2H), 1.12-1.02 (m, 2H) | 593.2 |
| B.63 | 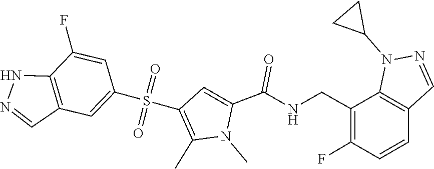 [SMILES: Cc1c(S(=O)(=O)c2cc(F)c3[nH]ncc3c2)cc(C(=O)NCc2c(F)ccc3cnn(C4CC4)c23)n1C] | <0.15 | A δ 8.61 (t, J = 4.3 Hz, 1H), 8.40 (d, J = 3.1 Hz, 1H), 8.22 (d, J = 0.9 Hz, 1H), 8.03 (s, 1H), 7.76 (dd, J = 5.2, 8.7 Hz, 1H), 7.44 (dd, J = 0.9, 10.4 Hz, 1H), 7.24 (s, 1H), 7.11- 7.03 (m, 1H), 5.00 (br d, J = 2.9 Hz, 2H), 4.00-3.92 (m, 1H), 3.77 (s, 3H), 2.40 (s, 3H), 1.31-1.24 (m, 2H), 1.11-1.04 (m, 2H) | 525.1 |
| B.64 | 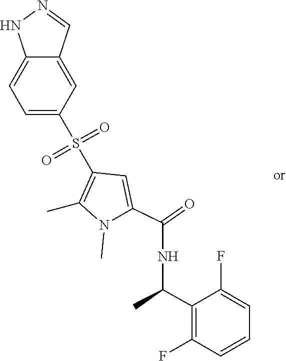 [SMILES: Cc1c(S(=O)(=O)c2ccc3[nH]ncc3c2)cc(C(=O)N[C@H](C)c2c(F)cccc2F)n1C] | 4 | B δ 8.45 (s, 1H), 8.23 (s, 1H), 7.81 (dd, J = 1.6, 8.8 Hz, 1H), 7.65 (d, J = 9.0 Hz, 1H), 7.30- 7.22 (m, 1H), 7.21 (s, 1H), 6.96-6.89 (m, 2H), 5.51-5.41 (m, 1H), 3.69 (s, 3H), 2.42 (s, 3H), 1.58 (d, J = 7.3 Hz, 3H) | 459.0 |
| | 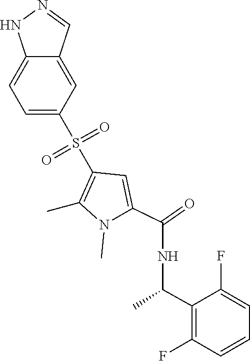 [SMILES: Cc1c(S(=O)(=O)c2ccc3[nH]ncc3c2)cc(C(=O)N[C@@H](C)c2c(F)cccc2F)n1C] | | | |
| | (faster eluting isomer) | | | |
| B.65 | 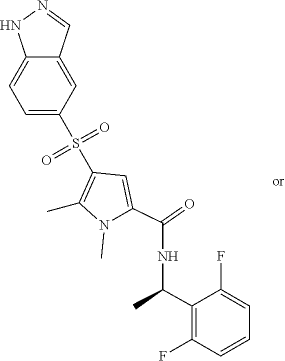 [SMILES: Cc1c(S(=O)(=O)c2ccc3[nH]ncc3c2)cc(C(=O)N[C@H](C)c2c(F)cccc2F)n1C] | 65 | B δ 8.46 (d, J = 0.9 Hz, 1H), 8.24 (s, 1H), 7.81 (dd, J = 1.6, 8.9 Hz, 1H), 7.66 (d, J = 9.0 Hz, 1H), 7.30-7.23 (m, 1H), 7.21 (s, 1H), 7.00-6.83 (m, 2H), 5.46 (q, J = 7.5 Hz, 1H), 3.69 (s, 3H), 2.42 (s, 3H), 1.58 (d, J = 7.3 Hz, 3H) | 459.1 |
| | 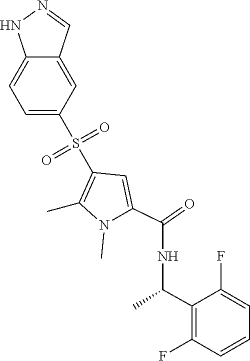 [SMILES: Cc1c(S(=O)(=O)c2ccc3[nH]ncc3c2)cc(C(=O)N[C@@H](C)c2c(F)cccc2F)n1C] | | | |
| | (slower eluting isomer) | | | |
| B.66 | 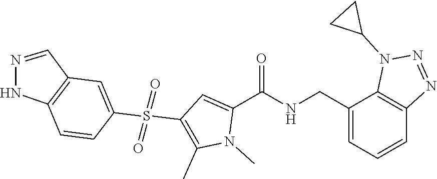 [SMILES: Cc1c(S(=O)(=O)c2ccc3[nH]ncc3c2)cc(C(=O)NCc2cccc3nnn(C4CC4)c23)n1C] | <0.15 | A δ 13.75-13.24 (m, 1H), 8.91 (t, J = 5.4 Hz, 1H), 8.40 (s, 1H), 8.31 (s, 1H), 7.93 (d, J = 8.1 Hz, 1H), 7.71 (s, 2H), 7.44-7.40 (m, 1H), 7.37 (d, J = 8.1 Hz, 1H), 7.34 (s, 1H), 5.07 (d, J = 5.4 Hz, 2H), 4.24 (tt, J = 3.6, 7.1 Hz, 1H), 3.76 (s, 3H), 2.41 (s, 3H), 1.46-1.39 (m, 2H), 1.33-1.25 (m, 2H) | 490.1 |
| B.67 | 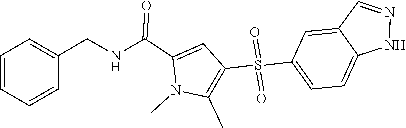 [SMILES: Cc1c(S(=O)(=O)c2ccc3[nH]ncc3c2)cc(C(=O)NCc2ccccc2)n1C] | 2.6 | A δ 13.95-13.04 (m, 1H), 8.84 (t, J = 6.0 Hz, 1H), 8.39 (t, J = 1.1 Hz, 1H), 8.30 (s, 1H), 7.70 (s, 2H), 7.34-7.21 (m, 6H), 4.36 (d, J = 6.0 Hz, 2H), 3.75 (s, 3H), 2.40 (s, 3H) | 409.0 |
| B.68 | 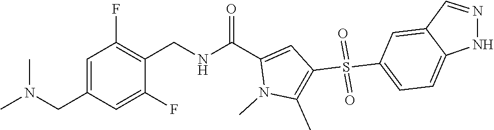 [SMILES: Cc1c(S(=O)(=O)c2ccc3[nH]ncc3c2)cc(C(=O)NCc2c(F)cc(CN(C)C)cc2F)n1C] | 4.1 | A δ 13.56 (br d, J = 4.3 Hz, 1H), 10.96 (br s, 1H), 8.72 (t, J = 5.3 Hz, 1H), 8.37 (t, J = 1.2 Hz, 1H), 8.30 (s, 1H), 7.69 (s, 2H), 7.38 (d, J = 7.9 Hz, 2H), 7.23 (s, 1H), 4.42 (br d, J = 5.1 Hz, 2H), 4.28 (d, J = 5.4 Hz, 2H), 3.73 (s, 3H), 2.69 (s, 3H), 2.68 (s, 3H), 2.38 (s, 3H) | 502.1 |
| B.69 | 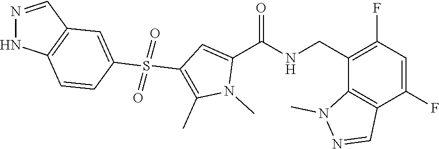 [SMILES: Cc1c(S(=O)(=O)c2ccc3[nH]ncc3c2)cc(C(=O)NCc2c(F)cc(F)c3cnn(C)c23)n1C] | <0.15 | A δ 13.73-13.33 (m, 1H), 8.65 (t, J = 4.4 Hz, 1H), 8.35 (t, J = 1.2 Hz, 1H), 8.28 (s, 1H), 8.20 (s, 1H), 7.67 (s, 2H), 7.25 (s, 1H), 7.08 (t, J = 10.2 Hz, 1H), 4.75 (br d, J = 2.5 Hz, 2H), 4.21 (s, 3H), 3.77 (s, 3H), 2.38 (s, 3H) | 499.0 |
| B.70 | 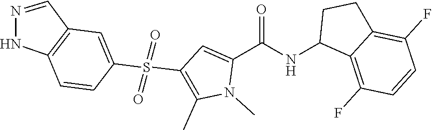 [SMILES: Cc1c(S(=O)(=O)c2ccc3[nH]ncc3c2)cc(C(=O)NC2CCc3c(F)ccc(F)c32)n1C] | 0.95 | A δ 13.52 (br dd, J = 2.7, 4.7 Hz, 1H), 8.68 (d, J = 8.6 Hz, 1H), 8.37 (s, 1H), 8.30 (s, 1H), 7.69 (s, 2H), 7.20 (s, 1H), 7.14 (dt, J = 3.5, 8.5 Hz, 1H), 7.04 (dt, J = 3.6, 8.7 Hz, 1H), 5.70- 5.61 (m, 1H), 3.77 (s, 3H), 3.13-3.01 (m, 1H), 2.91-2.81 | 471.1 |
| | | | (m, 1H), 2.47-2.43 (m, 1H), | |
| | racemic | | 2.40 (s, 3H), 2.03-1.92 (m, | |
| | | | 1H) | |
| B.71 | 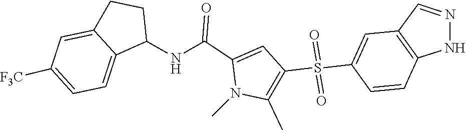 [SMILES: Cc1c(S(=O)(=O)c2ccc3[nH]ncc3c2)cc(C(=O)NC2CCc3cc(C(F)(F)F)ccc32)n1C] | 36 | B δ 8.46-8.43 (m, 1H), 8.23 (d, J = 0.9 Hz, 1H), 7.80 (dd, J = 1.6, 8.9 Hz, 1H), 7.65 (d, J = 8.9 Hz, 1H), 7.54 (s, 1H), 7.52-7.47 (m, 1H), 7.45-7.39 (m, 1H), 7.20 (s, 1H), 5.57 (t, J = 8.1 Hz, 1H), 3.83 (s, 3H), 3.16-3.06 (m, 1H), 3.02-2.90 | 503.0 |
| | | | (m, 1H), 2.58 (dtd, J = 3.1, | |
| | (racemic) | | 8.0, 12.7 Hz, 1H), 2.47 (s, | |
| | | | 3H), 2.11-1.98 (m, 1H) | |
| B.72 | 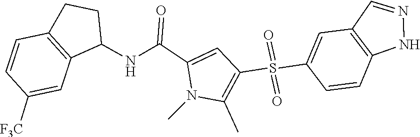 [SMILES: Cc1c(S(=O)(=O)c2ccc3[nH]ncc3c2)cc(C(=O)NC2CCc3ccc(C(F)(F)F)cc32)n1C] | 33 | B δ 8.45 (d, J = 1.0 Hz, 1H), 8.23 (d, J = 0.9 Hz, 1H), 7.80 (dd, J = 1.8, 8.9 Hz, 1H), 7.65 (d, J = 8.9 Hz, 1H), 7.52 (br d, J = 5.1 Hz, 2H), 7.45-7.40 (m, 1H), 7.20 (s, 1H), 5.57 (t, J = 7.9 Hz, 1H), 3.84 (s, 3H), 3.18-3.06 (m, 1H), 3.03-2.90 (m, 1H), 2.64-2.53 (m, 1H), | 503.0 |
| | | | 2.47 (s, 3H), 2.05 (qd, J = 8.6, | |
| | (racemic) | | 12.8 Hz, 1H) | |
| B.73 | 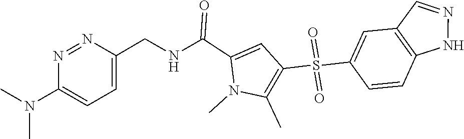 [SMILES: Cc1c(S(=O)(=O)c2ccc3[nH]ncc3c2)cc(C(=O)NCc2ccc(N(C)C)nn2)n1C] | 116 | B δ 8.46 (d, J = 1.0 Hz, 1H), 8.25 (s, 1H), 7.87-7.78 (m, 3H), 7.67 (d, J = 8.9 Hz, 1H), 7.25 (s, 1H), 4.56 (s, 2H), 3.78 (s, 3H), 3.32-3.31 (m, 3H), 3.30 (br s, 3H), 2.46 (s, 3H) | 454.1 |
| B.74 | 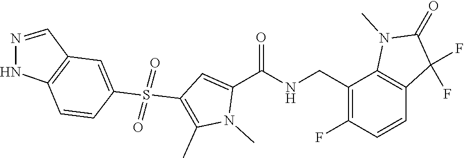 [SMILES: Cc1c(S(=O)(=O)c2ccc3[nH]ncc3c2)cc(C(=O)NCc2c(F)ccc3c2N(C)C(=O)C3(F)F)n1C] | 0.5 | B δ 8.43 (d, J = 1.0 Hz, 1H), 8.22 (d, J = 0.8 Hz, 1H), 7.78 (dd, J = 1.8, 8.9 Hz, 1H), 7.67- 7.56 (m, 2H), 7.15 (s, 1H), 7.00 (dd, J = 8.3, 10.2 Hz, 1H), 4.71 (d, J = 1.6 Hz, 2H), 3.78 (s, 3H), 3.56 (s, 3H), 2.44 (s, 3H) | 532.0 |
| B.75 | 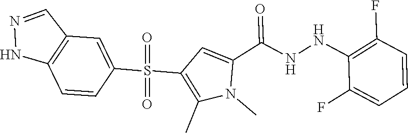 [SMILES: Cc1c(S(=O)(=O)c2ccc3[nH]ncc3c2)cc(C(=O)NNc2c(F)cccc2F)n1C] | 9.6 | B δ 8.47 (d, J = 0.9 Hz, 1H), 8.25 (s, 1H), 7.81 (dd, J = 1.8, 8.9 Hz, 1H), 7.67 (d, J = 8.9 Hz, 1H), 7.18 (s, 1H), 6.95- 6.81 (m, 3H), 3.74 (s, 3H), 2.45 (s, 3H) | 446.0 |
| B.76 | 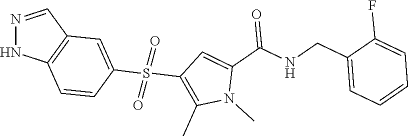 [SMILES: Cc1c(S(=O)(=O)c2ccc3[nH]ncc3c2)cc(C(=O)NCc2ccccc2F)n1C] | 1.6 | A δ 13.64-13.40 (m, 1H), 8.81 (quin, J = 5.4 Hz, 1H), 8.39 (s, 1H), 8.30 (s, 1H), 7.70 (s, 2H), 7.38-7.26 (m, 3H), 7.22-7.12 (m, 2H), 4.40 (d, J = 5.4 Hz, 2H), 3.74 (s, 3H), 2.40 (s, 3H) | 427.1 |
| B.77 | 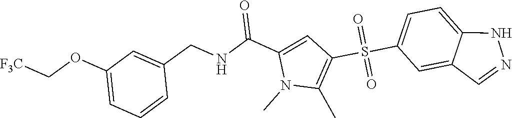 [SMILES: Cc1c(S(=O)(=O)c2ccc3[nH]ncc3c2)cc(C(=O)NCc2cccc(OCC(F)(F)F)c2)n1C] | 2.9 | A δ 13.51 (s, 1H), 8.81 (br t, J = 5.9 Hz, 1H), 8.39 (s, 1H), 8.30 (s, 1H), 7.70 (s, 2H), 7.33-7.24 (m, 2H), 6.99-6.90 (m, 3H), 4.73 (q, J = 8.8 Hz, 2H), 4.35 (d, J = 6.0 Hz, 2H), 3.75 (s, 3H), 2.40 (s, 3H) | 507.0 |
| B.78 | 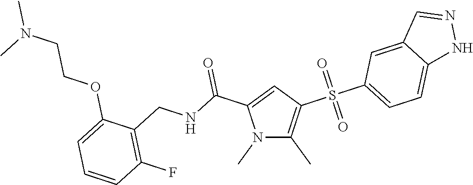 [SMILES: Cc1c(S(=O)(=O)c2ccc3[nH]ncc3c2)cc(C(=O)NCc2c(F)cccc2OCCN(C)C)n1C] | 2 | A δ 8.39-8.20 (m, 4H), 7.69 (s, 2H), 7.35-7.25 (m, 1H), 7.16 (s, 1H), 6.88 (d, J = 8.3 Hz, 1H), 6.80 (t, J = 8.8 Hz, 1H), 4.40 (br d, J = 4.6 Hz, 2H), 4.15 (br t, J = 5.2 Hz, 2H), 3.74 (s, 3H), 2.79 (br t, J = 5.1 Hz, 2H), 2.40 (s, 3H), 2.29 (s, 6H) | 514.1 |
| B.79 | 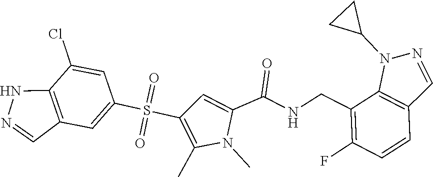 [SMILES: Cc1c(S(=O)(=O)c2cc(Cl)c3[nH]ncc3c2)cc(C(=O)NCc2c(F)ccc3cnn(C4CC4)c23)n1C] | 0.17 | B δ 8.38 (d, J = 1.4 Hz, 1H), 8.30 (s, 1H), 7.96 (s, 1H), 7.80-7.71 (m, 2H), 7.13 (s, 1H), 7.05 (dd, J = 8.9, 10.0 Hz, 1H), 5.18 (d, J = 1.6 Hz, 2H), 3.98 (tt, J = 3.6, 7.0 Hz, 1H), 3.81 (s, 3H), 2.46 (s, 3H), 1.37-1.30 (m, 2H), 1.23-1.16 (m, 2H) | 541.0 |
| B.80 | 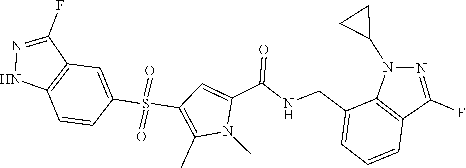 [SMILES: Cc1c(S(=O)(=O)c2ccc3[nH]nc(F)c3c2)cc(C(=O)NCc2cccc3c(F)nn(C4CC4)c23)n1C] | 1.5 | A δ 8.93-8.81 (m, 1H), 8.27 (s, 1H), 7.78 (dd, J = 1.6, 9.0 Hz, 1H), 7.71-7.64 (m, 1H), 7.59 (d, J = 8.1 Hz, 1H), 7.39-7.33 (m, 2H), 7.17 (t, J = 7.6 Hz, 1H), 5.06 (br d, J = 5.3 Hz, 2H), 3.94 (br s, 1H), 3.76 (s, 3H), 2.42 (s, 3H), 1.26-1.20 (m, 2H), 1.16-1.08 (m, 2H) | 525.0 |
| B.81 | 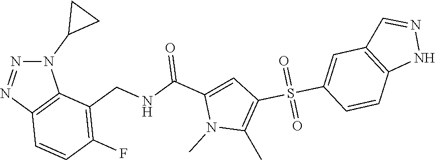 [SMILES: Cc1c(S(=O)(=O)c2ccc3[nH]ncc3c2)cc(C(=O)NCc2c(F)ccc3nnn(C4CC4)c23)n1C] | <0.15 | A δ 13.59-13.40 (m, 1H), 8.64 (br t, J = 4.2 Hz, 1H), 8.35 (s, 1H), 8.28 (s, 1H), 8.07 (dd, J = 4.6, 8.9 Hz, 1H), 7.70-7.62 (m, 2H), 7.34 (t, J = 9.6 Hz, 1H), 7.22 (s, 1H), 4.96 (br d, J = 3.5 Hz, 2H), 4.21-4.15 (m, 1H), 3.75 (s, 3H), 2.38 (s, 3H), 1.44-1.39 (m, 2H), 1.26-1.22 (m, 2H) | 508.1 |
| B.82 | 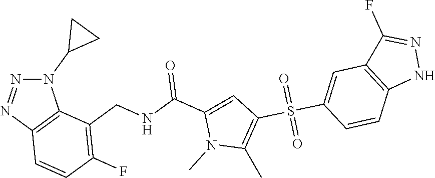 [SMILES: Cc1c(S(=O)(=O)c2ccc3[nH]nc(F)c3c2)cc(C(=O)NCc2c(F)ccc3nnn(C4CC4)c23)n1C] | <0.15 | A δ 13.07 (s, 1H), 8.64 (t, J = 4.3 Hz, 1H), 8.22 (s, 1H), 8.07 (dd, J = 4.6, 9.1 Hz, 1H), 7.75- 7.71 (m, 1H), 7.67-7.63 (m, 1H), 7.34 (t, J = 9.6 Hz, 1H), 7.23 (s, 1H), 4.96 (br d, J = 2.9 Hz, 2H), 4.18 (tt, J = 3.6, 7.1 Hz, 1H), 3.75 (s, 3H), 2.39 (s, 3H), 1.45-1.39 (m, 2H), 1.27- 1.20 (m, 2H) | 526.1 |
| B.83 | 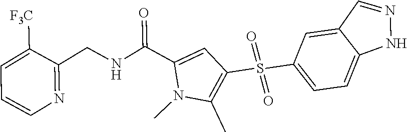 [SMILES: Cc1c(S(=O)(=O)c2ccc3[nH]ncc3c2)cc(C(=O)NCc2ncccc2C(F)(F)F)n1C] | 5.3 | A δ 8.83-8.75 (m, 2H), 8.40 (t, J = 1.2 Hz, 1H), 8.31 (s, 1H), 8.17 (d, J = 8.0 Hz, 1H), 7.72 (s, 2H), 7.53 (dd, J = 4.8, 7.8 Hz, 1H), 7.31 (s, 1H), 4.63 (br d, J = 5.4 Hz, 2H), 3.73 (s, 3H), 2.41 (s, 3H) A δ 8.83-8.75 (m, 2H), 8.40 (t, | 478.1 |
| | | | J = 1.2 Hz, 1H), 8.31 (s, 1H), | |
| | | | 8.17 (d, J = 8.0 Hz, 1H), 7.72 | |
| | | | (s, 2H), 7.53 (dd, J = 4.8, 7.8 | |
| | | | Hz, 1H), 7.31 (s, 1H), 4.63 (br | |
| | | | d, J = 5.4 Hz, 2H), 3.73 (s, | |
| | | | 3H), 2.41 (s, 3H) | |
| B.84 | 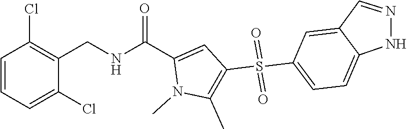 [SMILES: Cc1c(S(=O)(=O)c2ccc3[nH]ncc3c2)cc(C(=O)NCc2c(Cl)cccc2Cl)n1C] | 0.18 | A δ 13.59-13.46 (m, 1H), 8.45 (t, J = 4.6 Hz, 1H), 8.36 (s, 1H), 8.29 (s, 1H), 7.71-7.65 (m, 2H), 7.53-7.47 (m, 2H), 7.41-7.35 (m, 1H), 7.24 (s, 1H), 4.60 (d, J = 4.4 Hz, 2H), 3.76 (s, 3H), 2.39 (s, 3H). | 477.0 |
| B.85 | 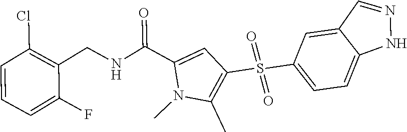 [SMILES: Cc1c(S(=O)(=O)c2ccc3[nH]ncc3c2)cc(C(=O)NCc2c(F)cccc2Cl)n1C] | 0.2 | B δ 8.43 (s, 1H), 8.23 (s, 1H), 7.78 (dd, J = 1.5, 8.9 Hz, 1H), 7.64 (d, J = 8.9 Hz, 1H), 7.36- 7.22 (m, 2H), 7.15-7.03 (m, 2H), 4.63 (s, 2H), 3.75 (s, 3H), 2.43 (s, 3H) | 461.0 |
| B.86 | 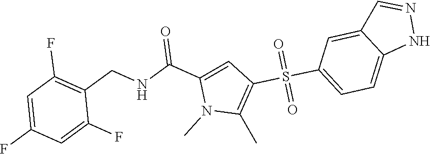 [SMILES: Cc1c(S(=O)(=O)c2ccc3[nH]ncc3c2)cc(C(=O)NCc2c(F)cc(F)cc2F)n1C] | 0.41 | B δ 8.44 (d, J = 1.0 Hz, 1H), 8.23 (d, J = 0.6 Hz, 1H), 7.79 (dd, J = 1.6, 8.9 Hz, 1H), 7.65 (d, J = 8.9 Hz, 1H), 7.11 (s, 1H), 6.92-6.80 (m, 2H), 4.50 (s, 2H), 3.76 (s, 3H), 2.43 (s, 3H) | 463.0 |
| B.87 | 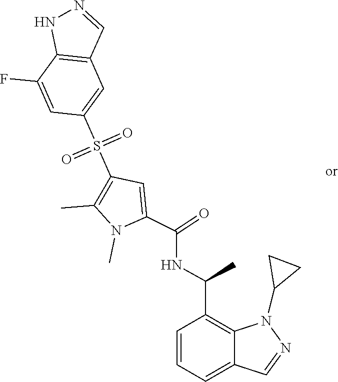 [SMILES: Cc1c(S(=O)(=O)c2cc(F)c3[nH]ncc3c2)cc(C(=O)N[C@@H](C)c2cccc3cnn(C4CC4)c23)n1C] | 0.82 | B δ 8.33-8.27 (m, 2H), 7.92 (s, 1H), 7.62 (d, J = 8.0 Hz, 1H), 7.57-7.50 (m, 2H), 7.26 (s, 1H), 7.16 (t, J = 7.6 Hz, 1H), 6.52 (d, J = 6.9 Hz, 1H), 4.06 (br d, J = 4.1 Hz, 1H), 3.70 (s, 3H), 2.44 (s, 3H), 1.66 (d, J = 6.9 Hz, 3H), 1.56-1.49 (m, 1H), 1.32-1.21 (m, 3H) | 521.1 |
| | 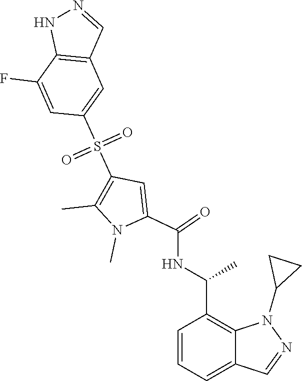 [SMILES: Cc1c(S(=O)(=O)c2cc(F)c3[nH]ncc3c2)cc(C(=O)N[C@H](C)c2cccc3cnn(C4CC4)c23)n1C] | | | |
| | (faster eluting isomer) | | | |
| B.88 | 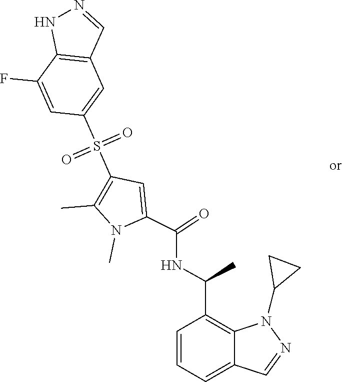 [SMILES: Cc1c(S(=O)(=O)c2cc(F)c3[nH]ncc3c2)cc(C(=O)N[C@@H](C)c2cccc3cnn(C4CC4)c23)n1C] | 22 | B δ 8.34-8.27 (m, 2H), 7.92 (s, 1H), 7.62 (d, J = 8.0 Hz, 1H), 7.57-7.50 (m, 2H), 7.26 (s, 1H), 7.16 (t, J = 7.6 Hz, 1H), 6.52 (q, J = 6.8 Hz, 1H), 4.09- 4.02 (m, 1H), 3.70 (s, 3H), 2.44 (s, 3H), 1.65 (d, J = 7.0 Hz, 3H), 1.53 (br d, J = 7.5 Hz, 1H), 1.29-1.22 (m, 3H) | 521.1 |
| | 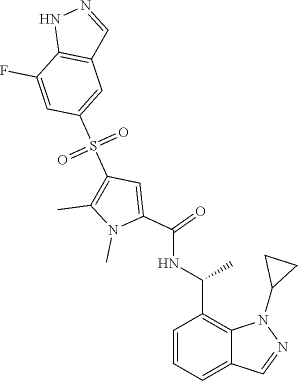 [SMILES: Cc1c(S(=O)(=O)c2cc(F)c3[nH]ncc3c2)cc(C(=O)N[C@H](C)c2cccc3cnn(C4CC4)c23)n1C] | | | |
| | (slower eluting isomer) | | | |
| B.89 | 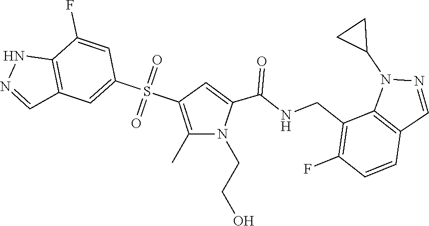 [SMILES: Cc1c(S(=O)(=O)c2cc(F)c3[nH]ncc3c2)cc(C(=O)NCc2c(F)ccc3cnn(C4CC4)c23)n1CCO] | 0.1 | B δ 8.33-8.18 (m, 2H), 7.94 (s, 1H), 7.72 (dd, J = 5.1, 8.8 Hz, 1H), 7.48 (br d, J = 10.1 Hz, 1H), 7.17 (s, 1H), 7.02 (dd, J = 8.9, 10.1 Hz, 1H), 5.15 (br d, J = 1.4 Hz, 2H), 4.42 (br t, J = 5.2 Hz, 2H), 3.94 (tt, J = 3.5, 6.8 Hz, 1H), 3.75 (t, J = 5.3 Hz, 2H), 2.51 (s, 3H), 1.33- 1.27 (m, 2H), 1.20-1.14 (m, 2H) | 555.1 |
| B.90 | 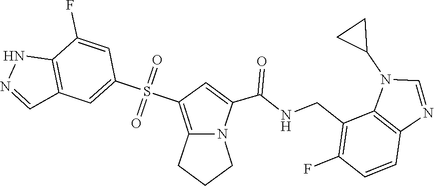 [SMILES: O=C(NCc1c(F)ccc2ncn(C3CC3)c12)c1cc(S(=O)(=O)c2cc(F)c3[nH]ncc3c2)c2n1CCC2] | 0.21 | A δ 8.45 (t, J = 4.3 Hz, 1H), 8.27 (d, J = 3.4 Hz, 1H), 8.20 (s, 1H), 8.07 (s, 1H), 7.60 (dd, J = 4.8, 8.8 Hz, 1H), 7.26-7.14 (m, 2H), 7.08 (dd, J = 8.8, 10.6 Hz, 1H), 4.94 (br d, J = 3.4 Hz, 2H), 4.21 (br t, J = 7.2 Hz, 2H), 3.73 (tt, J = 3.4, 7.0 Hz, 1H), 2.93 (br t, J = 7.5 Hz, 2H), 2.47-2.40 (m, 2H), 1.64 (s, 3H), 1.23-1.17 (m, 2H), 1.07-1.01 (m, 2H) | 537.1 |
| B.91 | 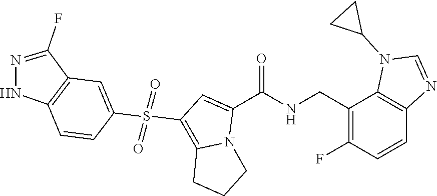 [SMILES: O=C(NCc1c(F)ccc2ncn(C3CC3)c12)c1cc(S(=O)(=O)c2ccc3[nH]nc(F)c3c2)c2n1CCC2] | 0.28 | A δ 13.23-13.11 (m, 1H), 9.22 (s, 1H), 8.60 (t, J = 4.6 Hz, 1H), 8.20 (s, 1H), 7.81-7.72 (m, 2H), 7.66 (dd, J = 1.6, 9.0 Hz, 1H), 7.38 (t, J = 9.6 Hz, 1H), 7.25 (s, 1H), 4.97 (br d, J = 3.8 Hz, 2H), 4.19 (br t, J = 7.3 Hz, 2H), 4.00 (br dd, J = 3.4, 7.0 Hz, 1H), 2.99-2.91 (m, 2H), 2.47-2.38 (m, 2H), 1.38- 1.27 (m, 2H), 1.20-1.12 (m, 2H) | 537.1 |
| B.92 | 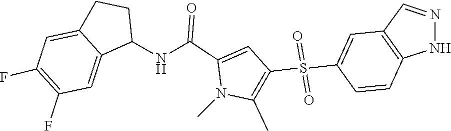 [SMILES: Cc1c(S(=O)(=O)c2ccc3[nH]ncc3c2)cc(C(=O)NC2CCc3cc(F)c(F)cc32)n1C] | 27 | B δ 8.44 (d, J = 0.9 Hz, 1H), 8.22 (s, 1H), 7.79 (dd, J = 1.6, 8.9 Hz, 1H), 7.64 (d, J = 8.9 Hz, 1H), 7.20 (s, 1H), 7.11 (br t, J = 8.9 Hz, 2H), 5.46 (br t, J = 7.6 Hz, 1H), 3.82 (s, 3H), 3.06-2.96 (m, 1H), 2.90-2.80 (m, 1H), 2.59-2.49 (m, 1H), | 471.0 |
| | | | 2.46 (s, 3H), 2.09-1.97 (m, | |
| | (racemic) | | 1H) | |
| B.93 | 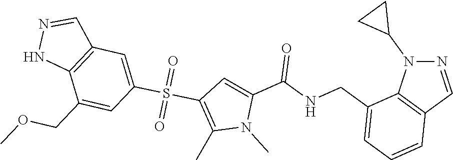 [SMILES: COCc1cc(S(=O)(=O)c2cc(C(=O)NCc3cccc4cnn(C5CC5)c34)n(C)c2C)cc2cn[nH]c12] | 68 | A δ 13.75-13.44 (m, 1H), 8.87 (t, J = 5.5 Hz, 1H), 8.35-8.31 (m, 2H), 7.99 (s, 1H), 7.68- 7.62 (m, 2H), 7.35 (s, 1H), 7.26 (d, J = 6.6 Hz, 1H), 7.13- 7.07 (m, 1H), 5.11 (d, J = 5.5 Hz, 2H), 4.75 (s, 2H), 4.03 (tt, J = 3.6, 7.1 Hz, 1H), 3.76 (s, 3H), 3.37 (s, 3H), 2.41 (s, 3H), 1.30-1.24 (m, 2H), 1.16-1.10 (m, 2H) | 533.2 |
| B.94 | 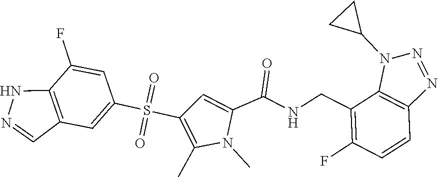 [SMILES: Cc1c(S(=O)(=O)c2cc(F)c3[nH]ncc3c2)cc(C(=O)NCc2c(F)ccc3nnn(C4CC4)c23)n1C] | <0.15 | A δ 10.73-9.71 (m, 1H), 8.65 (t, J = 4.3 Hz, 1H), 8.40 (d, J = 3.3 Hz, 1H), 8.22 (s, 1H), 8.07 (dd, J = 4.6, 9.1 Hz, 1H), 7.44 (d, J = 10.4 Hz, 1H), 7.34 (t, J = 9.6 Hz, 1H), 7.22 (s, 1H), 4.96 (br d, J = 3.8 Hz, 2H), 4.21-4.14 (m, 1H), 3.75 (s, 3H), 2.39 (s, 3H), 1.45-1.39 (m, 2H), 1.27-1.20 (m, 2H) | 526.1 |
| B.95 | 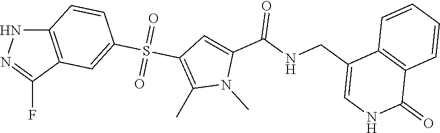 [SMILES: Cc1c(S(=O)(=O)c2ccc3[nH]nc(F)c3c2)cc(C(=O)NCc2c[nH]c(=O)c3ccccc23)n1C] | 2.2 | A δ 11.32-11.16 (m, 1H), 8.57 (t, J = 5.4 Hz, 1H), 8.23 (d, J = 7.0 Hz, 1H), 8.16 (s, 1H), 7.81-7.70 (m, 2H), 7.64-7.48 (m, 3H), 7.19 (s, 1H), 7.14 (s, 1H), 4.45 (d, J = 5.3 Hz, 2H), 3.77 (s, 3H), 2.39 (s, 3H). | 494.0 |
| B.96 | 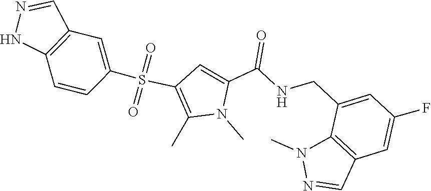 [SMILES: Cc1c(S(=O)(=O)c2ccc3[nH]ncc3c2)cc(C(=O)NCc2cc(F)cc3cnn(C)c23)n1C] | 1 | B δ 8.46 (d, J = 1.0 Hz, 1H), 8.23 (d, J = 0.9 Hz, 1H), 7.95 (s, 1H), 7.81 (dd, J = 1.8, 8.9 Hz, 1H), 7.66 (d, J = 8.9 Hz, 1H), 7.32 (dd, J = 2.2, 8.4 Hz, 1H), 7.24 (s, 1H), 7.14 (dd, J = 2.3, 9.8 Hz, 1H), 4.98 (s, 2H), 4.30 (s, 3H), 3.81 (s, 3H), 2.47 (s, 3H) | 481.1 |
| B.97 | 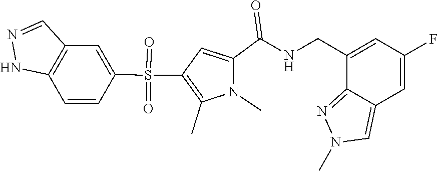 [SMILES: Cc1c(S(=O)(=O)c2ccc3[nH]ncc3c2)cc(C(=O)NCc2cc(F)cc3cn(C)nc23)n1C] | 11 | A δ 13.81-13.19 (m, 1H), 8.87 (t, J = 5.9 Hz, 1H), 8.41 (s, 1H), 8.33 (s, 1H), 8.31 (s, 1H), 7.75-7.70 (m, 2H), 7.34 (s, 1H), 7.31 (dd, J = 2.4, 9.4 Hz, 1H), 6.90 (dd, J = 1.6, 9.9 Hz, 1H), 4.71 (d, J = 5.6 Hz, 2H), 4.18 (s, 3H), 3.76 (s, 3H), 2.41 (s, 3H) | 481.2 |
| B.98 | 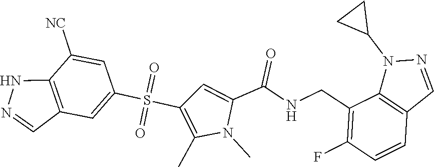 [SMILES: Cc1c(S(=O)(=O)c2cc(C#N)c3[nH]ncc3c2)cc(C(=O)NCc2c(F)ccc3cnn(C4CC4)c23)n1C] | 0.5 | B δ 8.69 (d, J = 1.38 Hz, 1H), 8.42-8.34 (m, 1H), 8.20 (d, J = 1.25 Hz, 1H), 7.95 (s, 1H), 7.73 (dd, J = 8.76, 5.13 Hz, 1H), 7.12 (s, 1H), 7.03 (dd, J = 10.19, 8.82 Hz, 1H), 5.16 (d, J = 1.75 Hz, 2H), 4.00-3.91 (m, 1H), 3.79 (s, 3H), 2.46 (s, 3H), 1.34-1.29 (m, 2H), 1.21- 1.14 (m, 2H) | 532.1 |
| B.99 | 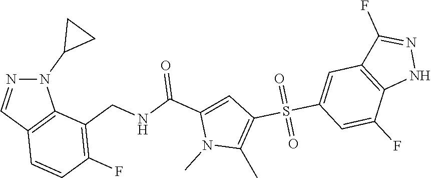 [SMILES: Cc1c(S(=O)(=O)c2cc(F)c3[nH]nc(F)c3c2)cc(C(=O)NCc2c(F)ccc3cnn(C4CC4)c23)n1C] | 0.15 | A δ 8.60 (br t, J = 4.1 Hz, 1H), 8.09 (s, 1H), 8.03 (s, 1H), 7.76 (dd, J = 5.1, 8.6 Hz, 1H), 7.57 (d, J = 9.9 Hz, 1H), 7.25 (s, 1H), 7.13-7.00 (m, 1H), 5.00 (br d, J = 3.0 Hz, 2H), 3.96 (tt, J = 3.6, 7.0 Hz, 1H), 3.77 (s, 3H), 2.41 (s, 3H), 1.34-1.20 (m, 2H), 1.11-0.95 (m, 2H) | 543.1 |
| B.100 | 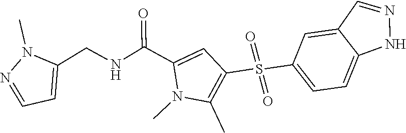 [SMILES: Cc1c(S(=O)(=O)c2ccc3[nH]ncc3c2)cc(C(=O)NCc2ccnn2C)n1C] | 12 | A δ 8.76 (t, J = 5.7 Hz, 1H), 8.38 (s, 1H), 8.30 (s, 1H), 7.70 (s, 2H), 7.32 (d, J = 1.8 Hz, 1H), 7.25 (s, 1H), 6.15 (d, J = 1.8 Hz, 1H), 4.41 (d, J = 5.6 Hz, 2H), 3.80 (s, 3H), 3.74 (s, 3H), 2.39 (s, 3H) | 413.1 |
| B.101 | 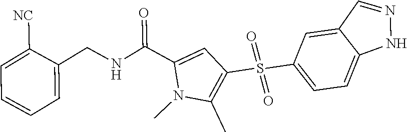 [SMILES: Cc1c(S(=O)(=O)c2ccc3[nH]ncc3c2)cc(C(=O)NCc2ccccc2C#N)n1C] | 192 | A δ 12.44-12.02 (m, 1H), 8.81-8.74 (m, 1H), 8.49 (d, J = 1.0 Hz, 1H), 8.35-8.30 (m, 1H), 7.99 (br s, 1H), 7.92-7.85 (m, 2H), 7.83-7.78 (m, 1H), 7.78-7.71 (m, 2H), 5.08 (s, 2H), 3.84 (s, 3H), 2.52 (s, 3H) | 434.0 |
| B.102 | 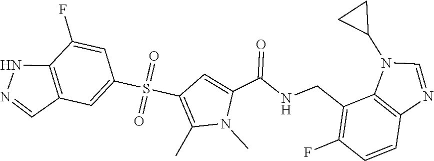 [SMILES: Cc1c(S(=O)(=O)c2cc(F)c3[nH]ncc3c2)cc(C(=O)NCc2c(F)ccc3ncn(C4CC4)c23)n1C] | <0.15 | A δ 9.10 (br s, 1H), 8.68 (t, J = 4.4 Hz, 1H), 8.41 (d, J = 3.0 Hz, 1H), 8.23 (d, J = 1.3 Hz, 1H), 7.76 (dd, J = 4.5, 8.9 Hz, 1H), 7.45 (dd, J = 0.8, 10.3 Hz, 1H), 7.37 (t, J = 9.7 Hz, 1H), 7.23 (s, 1H), 4.99 (br d, J = 3.6 Hz, 2H), 3.99 (td, J = 3.3, 6.8 Hz, 1H), 3.74 (s, 3H), 2.39 (s, 3H), 1.37-1.29 (m, 2H), 1.21-1.12 (m, 2H) | 525.1 |
| B.103 | 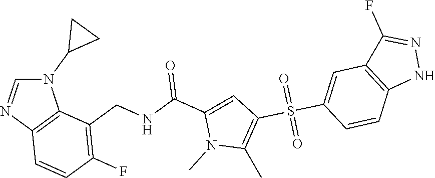 [SMILES: Cc1c(S(=O)(=O)c2ccc3[nH]nc(F)c3c2)cc(C(=O)NCc2c(F)ccc3ncn(C4CC4)c23)n1C] | <0.15 | A δ 13.11 (s, 1H), 9.06-8.90 (m, 1H), 8.66 (t, J = 4.3 Hz, 1H), 8.22 (s, 1H), 7.78-7.70 (m, 2H), 7.68-7.62 (m, 1H), 7.34 (t, J = 9.7 Hz, 1H), 7.24 (s, 1H), 4.98 (br d, J = 3.8 Hz, 2H), 3.96 (td, J = 3.3, 7.0 Hz, 1H), 3.74 (s, 3H), 2.39 (s, 3H), 1.37-1.27 (m, 2H), 1.20-1.11 (m, 2H) | 525.1 |
| B.104 | 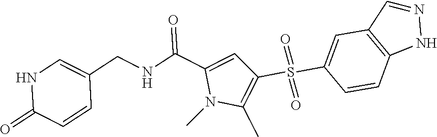 [SMILES: Cc1c(S(=O)(=O)c2ccc3[nH]ncc3c2)cc(C(=O)NCc2ccc(=O)[nH]c2)n1C] | 27 | B δ 8.45 (d, J = 1.0 Hz, 1H), 8.26-8.24 (m, 1H), 8.24-8.19 (m, 1H), 8.03 (d, J = 1.9 Hz, 1H), 7.80 (dd, J = 1.8, 8.9 Hz, 1H), 7.67 (d, J = 8.9 Hz, 1H), 7.20 (s, 1H), 7.14 (d, J = 9.0 Hz, 1H), 4.44 (s, 2H), 3.79 (s, 3H), 2.45 (s, 3H) | 426.1 |
| B.105 | 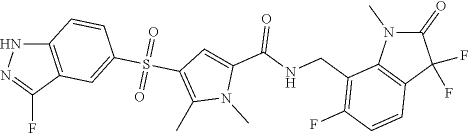 [SMILES: Cc1c(S(=O)(=O)c2ccc3[nH]nc(F)c3c2)cc(C(=O)NCc2c(F)ccc3c2N(C)C(=O)C3(F)F)n1C] | 0.15 | B δ 8.30 (s, 1H), 7.81 (dd, J = 1.6, 9.0 Hz, 1H), 7.64-7.52 (m, 2H), 7.15 (s, 1H), 7.00 (dd, J = 8.4, 10.3 Hz, 1H), 4.71 (d, J = 1.6 Hz, 2H), 3.78 (s, 3H), 3.56 (s, 3H), 2.45 (s, 3H) | 550.0 |
| B.106 | 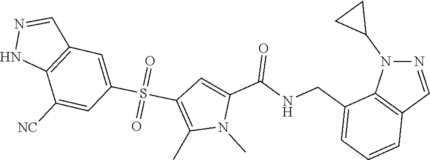 [SMILES: Cc1c(S(=O)(=O)c2cc(C#N)c3[nH]ncc3c2)cc(C(=O)NCc2cccc3cnn(C4CC4)c23)n1C] | 0.32 | B δ 8.73 (d, J = 1.4 Hz, 1H), 8.41 (s, 1H), 8.24 (d, J = 1.4 Hz, 1H), 7.95 (s, 1H), 7.65 (d, J = 8.0 Hz, 1H), 7.36 (d, J = 7.1 Hz, 1H), 7.25 (s, 1H), 7.13 (t, J = 7.6 Hz, 1H), 5.23 (s, 2H), 3.96 (tt, J = 3.6, 7.0 Hz, 1H), 3.81 (s, 3H), 2.49 (s, 3H), 1.38-1.30 (m, 2H), 1.26-1.16 (m, 2H) | 514.1 |
| B.107 | 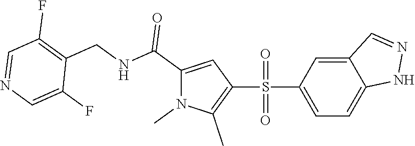 [SMILES: Cc1c(S(=O)(=O)c2ccc3[nH]ncc3c2)cc(C(=O)NCc2c(F)cncc2F)n1C] | 0.12 | A δ 13.53 (br s, 1H), 8.83 (s, 1H), 8.51 (s, 2H), 8.40 (s, 1H), 8.32 (s, 1H), 7.71 (s, 2H), 7.24 (s, 1H), 4.48 (d, J = 5.3 Hz, 2H), 3.74 (s, 3H), 2.40 (s, 3H) | 446.0 |
| B.108 | 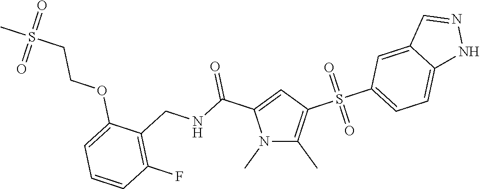 [SMILES: Cc1c(S(=O)(=O)c2ccc3[nH]ncc3c2)cc(C(=O)NCc2c(F)cccc2OCCS(C)(=O)=O)n1C] | <0.15 | A δ 13.84-13.17 (m, 1H), 8.36 (s, 1H), 8.30-8.24 (m, 2H), 7.73-7.64 (m, 2H), 7.33 (d, J = 6.9 Hz, 1H), 7.20 (s, 1H), 6.94 (d, J = 8.5 Hz, 1H), 6.84 (t, J = 8.8 Hz, 1H), 4.46-4.36 (m, 4H), 3.73 (s, 3H), 3.65 (br t, J = 5.3 Hz, 2H), 3.07 (s, 3H), 2.38 (s, 3H) | 549.1 |
| B.109 | 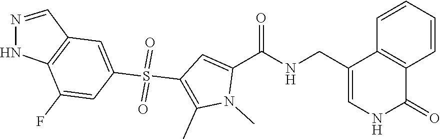 [SMILES: Cc1c(S(=O)(=O)c2cc(F)c3[nH]ncc3c2)cc(C(=O)NCc2c[nH]c(=O)c3ccccc23)n1C] | 0.24 | A δ 11.25-11.21 (m, 1H), 8.61-8.56 (m, 1H), 8.40 (br s, 1H), 8.25-8.21 (m, 2H), 7.81- 7.77 (m, 1H), 7.76-7.71 (m, 1H), 7.54-7.48 (m, 1H), 7.47- 7.43 (m, 1H), 7.19 (s, 1H), 7.14 (d, J = 6.3 Hz, 1H), 4.45 (d, J = 5.1 Hz, 2H), 3.78 (s, 3H), 2.40 (s, 3H) | 494.0 |
| B.110 | 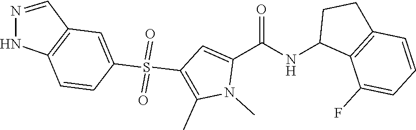 [SMILES: Cc1c(S(=O)(=O)c2ccc3[nH]ncc3c2)cc(C(=O)NC2CCc3cccc(F)c32)n1C] | 0.38 | B δ 8.44 (s, 1H), 8.26-8.20 (m, 1H), 7.79 (br d, J = 9.0 Hz, 1H), 7.64 (d, J = 8.9 Hz, 1H), 7.31-7.21 (m, 1H), 7.14-7.03 (m, 2H), 6.89 (t, J = 8.7 Hz, 1H), 5.70 (br t, J = 6.8 Hz, 1H), 3.80 (s, 3H), 3.18-3.08 (m, 1H), 2.96-2.85 (m, 1H), | 453.1 |
| | | | 2.59-2.50 (m, 1H), 2.45 (s, | |
| | (racemic) | | 3H), 2.09-1.97 (m, 1H) | |
| B.111 | 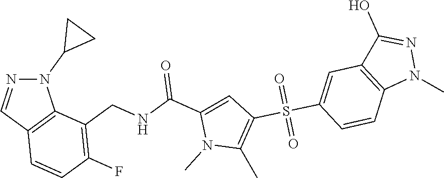 [SMILES: Cc1c(S(=O)(=O)c2ccc3c(c2)c(O)nn3C)cc(C(=O)NCc2c(F)ccc3cnn(C4CC4)c23)n1C] | 142 | A δ 11.29-10.72 (m, 1H), 8.61 (t, J = 3.9 Hz, 1H), 8.19 (s, 1H), 8.03 (s, 1H), 7.76 (dd, J = 5.1, 8.6 Hz, 1H), 7.64-7.58 (m, 1H), 7.57-7.52 (m, 1H), 7.23 (s, 1H), 7.07 (t, J = 9.5 Hz, 1H), 5.00 (br d, J = 2.9 Hz, 2H), 3.96 (tt, J = 3.3, 7.0 Hz, 1H), 3.79 (s, 3H), 3.76 (s, 3H), 2.37 (s, 3H), 1.30-1.24 (m, 2H), 1.11-1.04 (m, 2H) | 537.1 |
| B.112 | 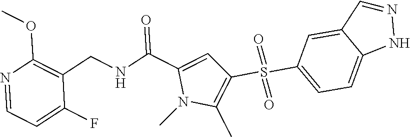 [SMILES: COc1nccc(F)c1CNC(=O)c1cc(S(=O)(=O)c2ccc3[nH]ncc3c2)c(C)n1C] | <0.15 | A δ 8.43 (t, J = 4.8 Hz, 1H), 8.36 (t, J = 1.1 Hz, 1H), 8.29 (s, 1H), 8.14 (dd, J = 5.9, 8.7 Hz, 1H), 7.71-7.65 (m, 2H), 7.20 (s, 1H), 6.95 (dd, J = 5.8, 8.7 Hz, 1H), 4.33 (d, J = 4.8 Hz, 2H), 3.90 (s, 3H), 3.74 (s, 3H), 2.38 (s, 3H) | 458.1 |
| B.113 | 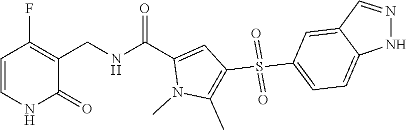 [SMILES: Cc1c(S(=O)(=O)c2ccc3[nH]ncc3c2)cc(C(=O)NCc2c(F)cc[nH]c2=O)n1C] | 0.34 | A δ 13.51 (br s, 1H), 11.83 (br s, 1H), 8.37 (s, 1H), 8.34-8.27 (m, 2H), 7.69 (s, 2H), 7.47 (t, J = 7.7 Hz, 1H), 7.22 (s, 1H), 6.22 (t, J = 7.3 Hz, 1H), 4.16 (d, J = 4.4 Hz, 2H), 3.74 (s, 3H), 2.38 (s, 3H) | 444.1 |
| B.114 | 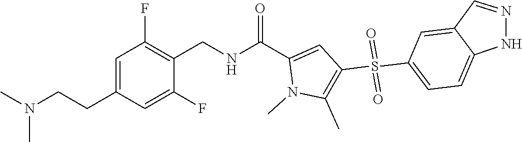 [SMILES: Cc1c(S(=O)(=O)c2ccc3[nH]ncc3c2)cc(C(=O)NCc2c(F)cc(CCN(C)C)cc2F)n1C] | 184 | A δ 13.53 (br s, 1H), 9.95-9.77 (m, 1H), 8.66 (t, J = 5.38 Hz, 1H), 8.37 (s, 1H), 8.29 (s, 1H), 7.69 (s, 2H), 7.21 (s, 1H), 7.10-7.01 (m, 2H), 4.38 (d, J = 5.00 Hz, 2H), 3.73 (s, 3H), 3.31-3.24 (m, 2H), 3.03- 2.96 (m, 2H), 2.80 (d, J = 4.75 Hz, 6H), 2.38 (s, 3H) | 516.1 |
| B.115 | 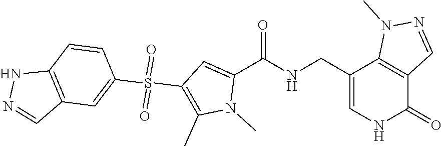 [SMILES: Cc1c(S(=O)(=O)c2ccc3[nH]ncc3c2)cc(C(=O)NCc2c[nH]c(=O)c3cnn(C)c23)n1C] | 0.17 | A δ 13.52 (s, 1H), 11.02 (br d, J = 5.9 Hz, 1H), 8.58 (t, J = 4.6 Hz, 1H), 8.37 (s, 1H), 8.29 (s, 1H), 8.01 (s, 1H), 7.69 (s, 2H), 7.29 (s, 1H), 7.11 (d, J = 6.1 Hz, 1H), 4.51 (d, J = 4.6 Hz, 2H), 4.05 (s, 3H), 3.76 (s, 3H), 2.39 (s, 3H) | 480.1 |
| B.116 | 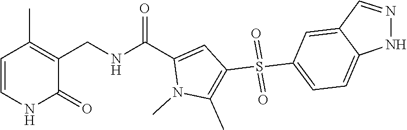 [SMILES: Cc1cc[nH]c(=O)c1CNC(=O)c1cc(S(=O)(=O)c2ccc3[nH]ncc3c2)c(C)n1C] | 4.6 | A δ 8.36 (s, 1H), 8.29 (s, 1H), 8.21 (t, J = 4.8 Hz, 1H), 7.68 (s, 2H), 7.21 (t, J = 3.3 Hz, 2H), 6.04 (d, J = 6.8 Hz, 1H), 4.22 (d, J = 4.8 Hz, 2H), 3.76- 3.72 (m, 3H), 2.38 (s, 3H), 2.18 (s, 3H) | 440.0 |
| B.117 | 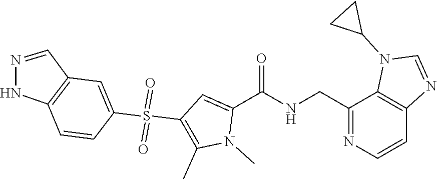 [SMILES: Cc1c(S(=O)(=O)c2ccc3[nH]ncc3c2)cc(C(=O)NCc2nccc3ncn(C4CC4)c23)n1C] | 0.18 | A δ 13.65-13.36 (m, 1H), 8.78 (br t, J = 5.0 Hz, 1H), 8.40 (d, J = 4.3 Hz, 2H), 8.30 (s, 1H), 8.26 (d, J = 5.5 Hz, 1H), 7.71 (s, 2H), 7.56 (d, J = 5.5 Hz, 1H), 7.31 (s, 1H), 5.11 (br d, J = 5.1 Hz, 2H), 3.87 (br d, J = 3.5 Hz, 1H), 3.75 (s, 3H), 2.41 (s, 3H), 1.26 (br d, J = 1.8 Hz, 2H), 1.16 (br d, J = 5.6 Hz, 2H) | 490.1 |
| B.118 | 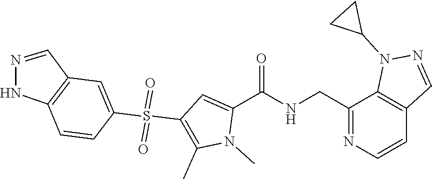 [SMILES: Cc1c(S(=O)(=O)c2ccc3[nH]ncc3c2)cc(C(=O)NCc2nccc3cnn(C4CC4)c23)n1C] | 0.48 | A δ 13.69-13.43 (m, 1H), 8.99 (br d, J = 3.3 Hz, 1H), 8.42 (s, 1H), 8.39-8.29 (m, 2H), 8.23 (d, J = 5.9 Hz, 1H), 8.00-7.91 (m, 1H), 7.73 (s, 2H), 7.38 (s, 1H), 5.32 (br d, J = 4.6 Hz, 2H), 4.24-4.15 (m, 1H), 3.75 (s, 3H), 2.43 (s, 3H), 1.39-1.34 (m, 2H), 1.28-1.23 (m, 2H) | 490.1 |
| B.119 | 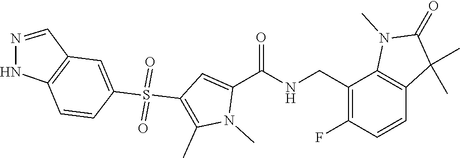 [SMILES: Cc1c(S(=O)(=O)c2ccc3[nH]ncc3c2)cc(C(=O)NCc2c(F)ccc3c2N(C)C(=O)C3(C)C)n1C] | 0.21 | B δ 8.42 (d, J = 1.5 Hz, 1H), 8.22 (s, 1H), 7.78 (dd, J = 1.5, 8.9 Hz, 1H), 7.64 (d, J = 8.9 Hz, 1H), 7.25 (dd, J = 5.3, 8.2 Hz, 1H), 7.13 (s, 1H), 6.86 (dd, J = 8.3, 10.6 Hz, 1H), 4.75 (d, J = 1.6 Hz, 2H), 3.78 (s, 3H), 3.53 (s, 3H), 2.44 (s, 3H), 1.33 (s, 6H) | 524.2 |
| B.120 | 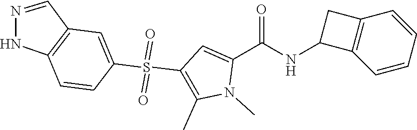 [SMILES: Cc1c(S(=O)(=O)c2ccc3[nH]ncc3c2)cc(C(=O)NC2Cc3ccccc32)n1C] | 1.2 | B δ 8.44 (d, J = 0.9 Hz, 1H), 8.22 (s, 1H), 7.80 (dd, J = 1.6, 8.9 Hz, 1H), 7.64 (d, J = 8.9 Hz, 1H), 7.32-7.16 (m, 4H), 7.13 (d, J = 7.0 Hz, 1H), 5.48 (dd, J = 2.4, 4.9 Hz, 1H), 3.82 (s, 3H), 3.70-3.63 (m, 1H), 3.16 (dd, J = 2.4, 14.2 Hz, 1H), 2.46 (s, 3H) | 421.1 |
| B.121 | 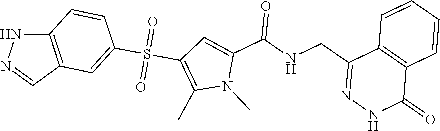 [SMILES: Cc1c(S(=O)(=O)c2ccc3[nH]ncc3c2)cc(C(=O)NCc2n[nH]c(=O)c3ccccc23)n1C] | <0.15 | B δ 8.43 (s, 1H), 8.39 (d, J = 8.3 Hz, 1H), 8.22 (s, 1H), 8.08 (d, J = 8.5 Hz, 1H), 7.97-7.91 (m, 1H), 7.90-7.84 (m, 1H), 7.78 (dd, J = 1.8, 8.8 Hz, 1H), 7.67-7.62 (m, 1H), 7.15 (s, 1H), 4.79 (br d, J = 2.8 Hz, 2H), 3.81 (s, 3H), 2.46 (s, 3H) | 477.1 |
| B.122 | 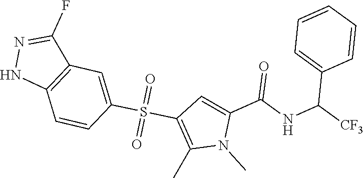 [SMILES: Cc1c(S(=O)(=O)c2ccc3[nH]nc(F)c3c2)cc(C(=O)NC(c2ccccc2)C(F)(F)F)n1C] | 70 | A δ 13.22-12.98 (m, 1H), 9.27 (d, J = 9.8 Hz, 1H), 8.29 (s, 1H), 7.80 (dd, J = 1.7, 8.9 Hz, 1H), 7.71-7.63 (m, 3H), 7.58 (s, 1H), 7.48-7.40 (m, 3H), 5.96 (t, J = 8.9 Hz, 1H), 3.73 (s, 3H), 2.41 (s, 3H) | 495.1 |
| B.123 | 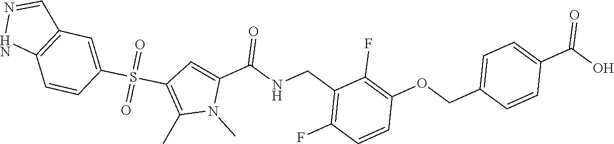 [SMILES: Cc1c(S(=O)(=O)c2ccc3[nH]ncc3c2)cc(C(=O)NCc2c(F)ccc(OCc3ccc(C(=O)O)cc3)c2F)n1C] | <0.15 | A δ 8.65 (br t, J = 5.1 Hz, 1H), 8.37 (s, 1H), 8.29 (s, 1H), 7.94 (d, J = 8.1 Hz, 2H), 7.68 (s, 2H), 7.51 (d, J = 8.1 Hz, 2H), 7.23 (s, 1H), 7.21-7.15 (m, 1H), 7.07-6.96 (m, 1H), 5.23 (s, 2H), 4.41 (br d, J = 4.9 Hz, 2H), 3.74 (s, 3H), 2.38 (s, 3H) | 595.1 |
| B.124 | 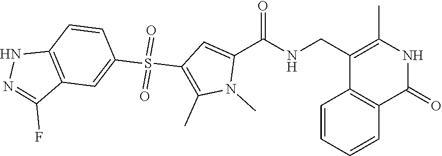 [SMILES: Cc1[nH]c(=O)c2ccccc2c1CNC(=O)c1cc(S(=O)(=O)c2ccc3[nH]nc(F)c3c2)c(C)n1C] | 0.36 | A δ 13.07 (s, 1H), 11.33-11.25 (m, 1H), 8.36 (t, J = 4.8 Hz, 1H), 8.23-8.15 (m, 2H), 7.79- 7.60 (m, 4H), 7.42 (t, J = 7.3 Hz, 1H), 7.17 (s, 1H), 4.49 (d, J = 4.9 Hz, 2H), 3.78 (s, 3H), 2.38 (d, J = 3.0 Hz, 6H) | 508.1 |
| B.125 | 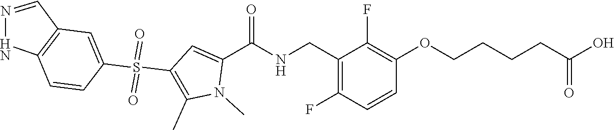 [SMILES: Cc1c(S(=O)(=O)c2ccc3[nH]ncc3c2)cc(C(=O)NCc2c(F)ccc(OCCCCC(=O)O)c2F)n1C] | <0.15 | A δ 8.68-8.59 (m, 1H), 8.36 (s, 1H), 8.29 (s, 1H), 7.74-7.63 (m, 2H), 7.23 (s, 1H), 7.16- 7.05 (m, 1H), 6.98 (br t, J = 9.2 Hz, 1H), 4.40 (br d, J = 4.3 Hz, 2H), 4.00 (br t, J = 6.0 Hz, 2H), 3.73 (s, 3H), 2.38 (s, 3H), 2.23 (br t, J = 7.1 Hz, 2H), | 561.1 |
| | | | 1.77-1.68 (m, 2H), 1.67-1.58 | |
| | | | (m, 2H) | |
| B.126 | 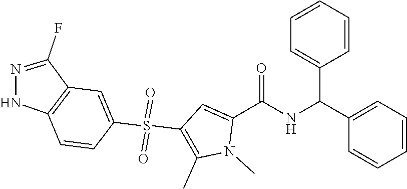 [SMILES: Cc1c(S(=O)(=O)c2ccc3[nH]nc(F)c3c2)cc(C(=O)NC(c2ccccc2)c2ccccc2)n1C] | 108 | A δ 9.08 (d, J = 8.8 Hz, 1H), 8.26 (s, 1H), 7.77 (dd, J = 1.4, 9.0 Hz, 1H), 7.66 (dd, J = 1.5, 9.0 Hz, 1H), 7.47 (s, 1H), 7.40-7.21 (m, 10H), 6.31 (d, J = 8.8 Hz, 1H), 3.74 (s, 3H), 2.40 (s, 3H) | 503.1 |
| B.127 | 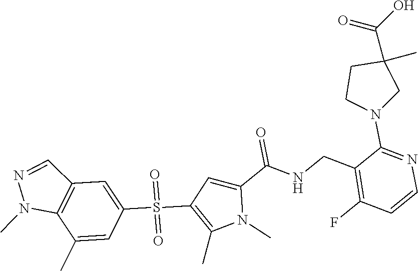 [SMILES: Cc1cc(S(=O)(=O)c2cc(C(=O)NCc3c(F)ccnc3N3CCC(C)(C(=O)O)C3)n(C)c2C)cc2cnn(C)c12] | 0.58 | B 8 8.21 (s, 1H), 8.10 (s, 1H), 8.03 (dd, J = 5.8, 8.6 Hz, 1H), 7.50 (s, 1H), 7.11 (s, 1H), 6.57 (dd, J = 5.8, 9.0 Hz, 1H), 4.59- 4.49 (m, 2H), 4.33 (s, 3H), 4.05 (d, J = 10.3 Hz, 1H), 3.78 (s, 3H), 3.69 (t, J = 6.9 Hz, 2H), 3.48-3.40 (m, 1H), 2.80 (s, 3H), 2.45 (s, 3H), 2.44-2.36 (m, 1H), 1.85 (td, J = 7.3, 12.4 Hz, 1H), 1.34 (s, 3H) | 583.1 |
| | (racemic) | | | |
| B.128 | 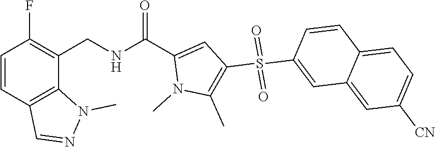 [SMILES: Cc1c(S(=O)(=O)c2ccc3ccc(C#N)cc3c2)cc(C(=O)NCc2c(F)ccc3cnn(C)c23)n1C] | 0.93 | A δ 8.86 (s, 1H), 8.73 (s, 1H), 8.67 (d, J = 1.5 Hz, 1H), 8.22 (d, J = 3.1 Hz, 1H), 8.20 (d, J = 3.0 Hz, 1H), 8.06 (s, 1H), 7.97 (dd, J = 1.6, 8.5 Hz, 1H), 7.93 (dd, J = 1.9, 8.8 Hz, 1H), 7.77 (dd, J = 5.1, 8.8 Hz, 1H), 7.31 (s, 1H), 7.05 (dd, J = 8.8, 10.1 Hz, 1H), 4.82-4.77 (m, | 516.1 |
| | | | 2H), 4.19 (s, 3H), 3.78 (s, 3H), | |
| | | | 2.42 (s, 3H) | |
| B.129 | 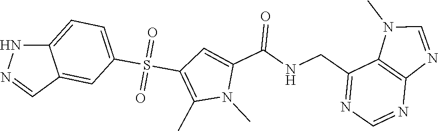 [SMILES: Cc1c(S(=O)(=O)c2ccc3[nH]ncc3c2)cc(C(=O)NCc2ncnc3ncn(C)c23)n1C] | <0.15 | A δ 8.93 (s, 1H), 8.85 (s, 1H), 8.59 (s, 1H), 8.39 (s, 1H), 8.31 (s, 1H), 7.71 (s, 2H), 7.33 (s, 1H), 4.91 (d, J = 5.5 Hz, 2H), 4.13 (s, 3H), 3.73 (s, 3H), 2.40 (s, 3H) | 465.1 |
| B.130 | 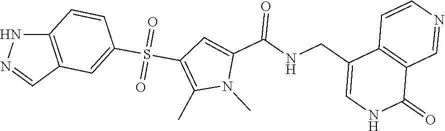 [SMILES: Cc1c(S(=O)(=O)c2ccc3[nH]ncc3c2)cc(C(=O)NCc2c[nH]c(=O)c3cnccc23)n1C] | <0.15 | A δ 9.33 (s, 1H), 8.75 (d, J = 5.6 Hz, 1H), 8.63-8.57 (m, 1H), 8.35 (s, 1H), 8.28 (s, 1H), 7.73-7.64 (m, 3H), 7.39 (d, J = 5.9 Hz, 1H), 7.18 (s, 1H), 4.42 (d, J = 5.4 Hz, 2H), 3.77 (s, 3H), 2.40-2.37 (m, 3H) | 477.0 |
| B.131 | 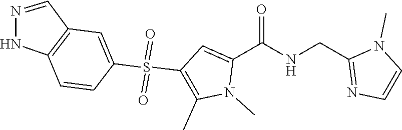 [SMILES: Cc1c(S(=O)(=O)c2ccc3[nH]ncc3c2)cc(C(=O)NCc2nccn2C)n1C] | 79 | B δ 8.45 (d, J = 1.0 Hz, 1H), 8.24 (s, 1H), 7.82-7.78 (m, 1H), 7.66 (d, J = 8.9 Hz, 1H), 7.47-7.37 (m, 1H), 7.25-7.20 (m, 2H), 7.10 (d, J = 1.1 Hz, 1H), 4.62 (s, 2H), 3.79 (d, J = 4.4 Hz, 6H), 2.46 (s, 3H) | 413.1 |
| B.132 | 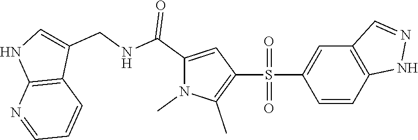 [SMILES: Cc1c(S(=O)(=O)c2ccc3[nH]ncc3c2)cc(C(=O)NCc2c[nH]c3ncccc23)n1C] | 0.6 | B δ 8.42 (d, J = 0.9 Hz, 1H), 8.21 (s, 1H), 8.17 (dd, J = 1.5, 4.9 Hz, 1H), 8.11 (dd, J = 1.5, 7.9 Hz, 1H), 7.77 (dd, J = 1.7, 8.8 Hz, 1H), 7.63 (d, J = 8.9 Hz, 1H), 7.38 (s, 1H), 7.12- 7.05 (m, 2H), 4.63 (s, 2H), 3.80 (s, 3H), 2.44 (s, 3H) | 449.0 |
| B.133 | 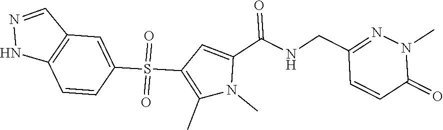 [SMILES: Cc1c(S(=O)(=O)c2ccc3[nH]ncc3c2)cc(C(=O)NCc2ccc(=O)n(C)n2)n1C] | 7.6 | B δ 8.48-8.44 (m, 1H), 8.24 (d, J = 0.9 Hz, 1H), 7.80 (dd, J = 1.6, 8 9 Hz, 1H), 7.66 (d, J = 8.9 Hz, 1H), 7.47 (d, J = 9.5 Hz, 1H), 7.20 (s, 1HD), 6 95 (d, J = 9.5 Hz, 1H), 4.40 (s, 2H), 3.79 (s, 3H), 3 74 (s, 3H), 2.46 (s, 3H) | 441.1 |
| B.134 | 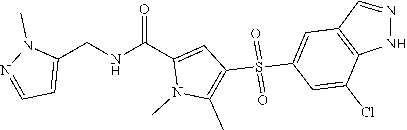 [SMILES: Cc1c(S(=O)(=O)c2cc(Cl)c3[nH]ncc3c2)cc(C(=O)NCc2ccnn2C)n1C] | 0.18 | A δ 8.76 (t, J = 5.7 Hz, 1H), 8.44 (s, 1H), 8.39 (d, J = 1.4 Hz, 1H), 7.72 (d, J = 1.4 Hz, 1H), 7.29 (d, J = 1.8 Hz, 1H), 7.26 (s, 1H), 6.14 (d, J = 1.8 Hz, 1H), 4.42 (d, J = 5.6 Hz, 2H), 3.79 (s, 3H), 3.75 (s, 3H), 2.40 (s, 3H) | 447.1 |
| B.135 | 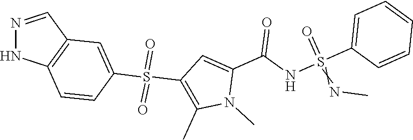 [SMILES: CN=S(=O)(NC(=O)c1cc(S(=O)(=O)c2ccc3[nH]ncc3c2)c(C)n1C)c1ccccc1] | 19 | A δ 8.42-8.39 (m, 1H), 8.31 (s, 1H), 7.86-7.81 (m, 2H), 7.75- 7.68 (m, 2H), 7.68-7.59 (m, 3H), 7.21 (s, 1H), 3.68 (s, 3H), 2.42 (s, 3H), 2.38 (s, 3H) | 472.0 |
| | (racemic) | | | |
| B.136 | 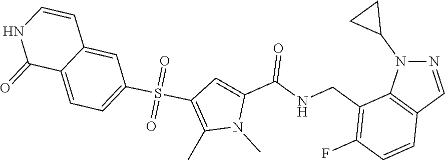 [SMILES: Cc1c(S(=O)(=O)c2ccc3c(=O)[nH]ccc3c2)cc(C(=O)NCc2c(F)ccc3cnn(C4CC4)c23)n1C] | <0.15 | A δ 11.53 (br d, J = 4.8 Hz, 1H), 8.66 (br t, J = 4.0 Hz, 1H), 8.30 (d, J = 8.4 Hz, 1H), 8.17 (s, 1H), 8.02 (s, 1H), 7.79-7.72 (m, 2H), 7.28 (br t, J = 6.4 Hz, 1H), 7.26 (s, 1H), 7.07 (t, J = 9.4 Hz, 1H), 6.73 (d, J = 7.0 Hz, 1H), 5.00 (br d, J = 3.0 Hz, 2H), 3.95 (dt, J = 3.6, 6.9 Hz, 1H), 3.78 (s, 3H), | 534.1 |
| | | | 2.41 (s, 3H), 1.30-1.24 (m, | |
| | | | 2H), 1.10-1.04 (m, 2H) | |
| B.137 | 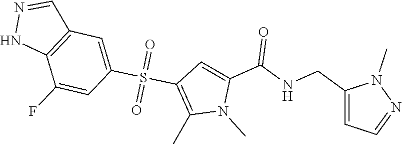 [SMILES: Cc1c(S(=O)(=O)c2cc(F)c3[nH]ncc3c2)cc(C(=O)NCc2ccnn2C)n1C] | 10 | A δ 8.75 (t, J = 5.8 Hz, 1H), 8.42 (d, J = 3.3 Hz, 1H), 8.26 (d, J = 1.3 Hz, 1H), 7.55-7.44 (m, 1H), 7.31-7.23 (m, 2H), 6.14 (d, J = 1.8 Hz, 1H), 4.41 (d, J = 5.6 Hz, 2H), 3.79 (s, 3H), 3.75 (s, 3H), 2.40 (s, 3H) | 431.1 |
| B.138 | 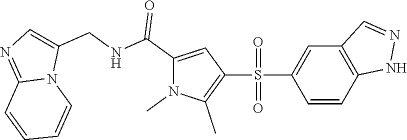 [SMILES: Cc1c(S(=O)(=O)c2ccc3[nH]ncc3c2)cc(C(=O)NCc2cnc3ccccn23)n1C] | 6.3 | B δ 8.93 (d, J = 6.88 Hz, 1H), 8.44-8.39 (m, 1H), 8.22 (d, J = 0.88 Hz, 1H), 8.05 (s, 1H), 8.03-7.99 (m, 1 H), 7.98-7.92 (m, 1H), 7.77 (dd, J = 8.88, 1.75 Hz, 1H), 7.64 (d, J = 8.88 Hz, 1H), 7.56 (td, J = 6.88, 1.25 Hz, 1H), 7.17 (s, 1H), 4.93 (s, 2H), 3.81 (s, 3H), 2.45 (s, 3H) | 449.0 |
| B.139 | 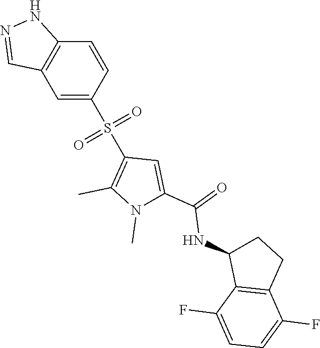 [SMILES: Cc1c(S(=O)(=O)c2ccc3[nH]ncc3c2)cc(C(=O)N[C@H]2CCc3c(F)ccc(F)c32)n1C] | 1.3 | B δ 8.44 (d, J = 0.9 Hz, 1H), 8.23 (s, 1H), 7.83-7.75 (m, 1H), 7.65 (d, J = 8.8 Hz, 1H), 7.11 (s, 1H), 7.03-6.96 (m, 1H), 6.95-6.88 (m, 1H), 5.78- 5.66 (m, 1H), 3.80 (s, 3H), 3.22-3.08 (m, 1H), 2.98-2.83 (m, 1H), 2.66-2.54 (m, 1H), 2.45 (s, 3H), 2.15-2.00 (m, 1H) | 471.1 |
| | or | | | |
| | 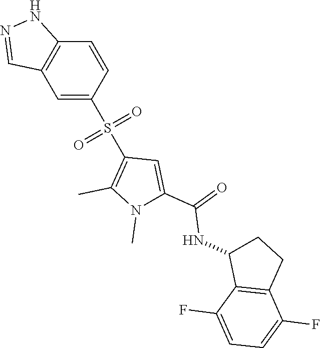 | | | |
| | (faster eluting isomer) | | | |
| B.140 | 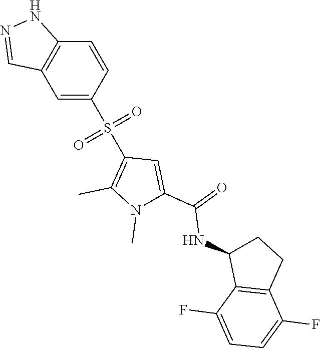 [SMILES: Cc1c(S(=O)(=O)c2ccc3[nH]ncc3c2)cc(C(=O)N[C@H]2CCc3c(F)ccc(F)c32)n1C] | 0.31 | B δ 8.44 (d, J = 1.0 Hz, 1H), 8.23 (s, 1H), 7.79 (dd, J = 1.6, 8.9 Hz, 1H), 7.65 (d, J = 8.9 Hz, 1H), 7.11 (s, 1H), 7.02- 6.95 (m, 1H), 6.95-6.88 (m, 1H), 5.76-5.66 (m, 1H), 3.80 (s, 3H), 3.20-3.09 (m, 1H), 2.97-2.84 (m, 1H), 2.66-2.53 (m, 1H), 2.45 (s, 3H), 2.14- 2.02 (m, 1H) | 471.0 |
| | or | | | |
| | 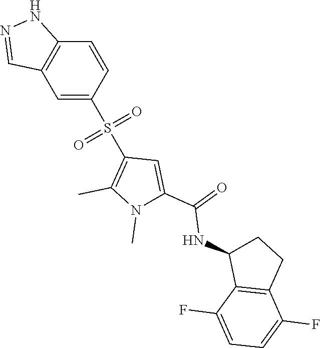 [SMILES: Cc1c(S(=O)(=O)c2ccc3[nH]ncc3c2)cc(C(=O)N[C@H]2CCc3c(F)ccc(F)c32)n1C] | | | |
| | (faster eluting isomer) | | | |
| B.141 | 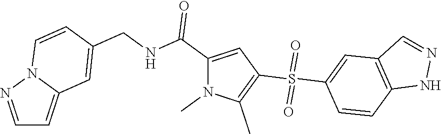 [SMILES: Cc1c(S(=O)(=O)c2ccc3[nH]ncc3c2)cc(C(=O)NCc2ccn3nccc3c2)n1C] | 16 | B δ 8.50-8.43 (m, 2H), 8.24 (s, 1H), 7.91 (d, J = 2.4 Hz, 1H), 7.81 (dd, J = 1.6, 8.9 Hz, 1H), 7.66 (d, J = 8.9 Hz, 1H), 7.53 (s, 1H), 7.23 (s, 1H), 6.85 (dd, J = 1.7, 7.2 Hz, 1H), 6.54 (d, J = 2.3 Hz, 1H), 4.50 (s, 2H), 3.80 (s, 3H), 2.46 (s, 3H) | 449.0 |
| B.142 | 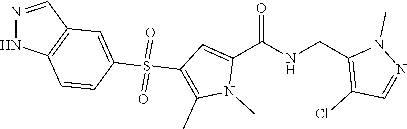 [SMILES: Cc1c(S(=O)(=O)c2ccc3[nH]ncc3c2)cc(C(=O)NCc2c(Cl)cnn2C)n1C] | 1.4 | A δ 13.62-13.39 (m, 1H), 8.71 (s, 1H), 8.38 (s, 1H), 8.30 (s, 1H), 7.69 (d, J = 0.6 Hz, 2H), 7.48 (s, 1H), 7.26 (s, 1H), 4.42 (d, J = 5.4 Hz, 2H), 3.82 (s, 3H), 3.75 (s, 3H), 2.39 (s, 3H) | 447.0 |
| B.143 | 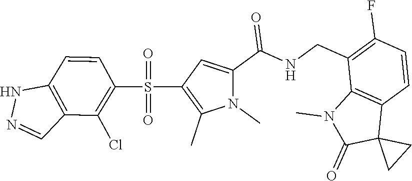 [SMILES: Cc1c(S(=O)(=O)c2ccc3[nH]ncc3c2Cl)cc(C(=O)NCc2c(F)ccc3c2N(C)C(=O)C32CC2)n1C] | 0.4 | A δ 14.03-13.61 (m, 1H), 8.60 (t, J = 4.2 Hz, 1H), 8.29 (s, 1H), 8.08 (d, J = 8.9 Hz, 1H), 7.71 (d, J = 8.8 Hz, 1H), 7.34 (s, 1H), 6.99 (dd, J = 5.4, 8.1 Hz, 1H), 6.85 (dd, J = 8.2, 10.6 Hz, 1H), 4.63 (br d, J = 2.3 Hz, 2H), 3.78 (s, 3H), 3.47 (s, 3H), 2.31 (s, 3H), 1.60 (br t, J = 3.4 Hz, 2H), 1.54 (t, J = 3.3 Hz, 2H) | 556.1 |
| B.144 | 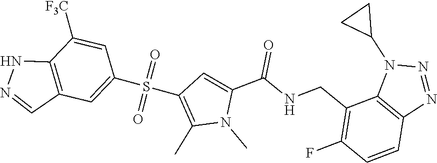 [SMILES: Cc1c(S(=O)(=O)c2cc(C(F)(F)F)c3[nH]ncc3c2)cc(C(=O)NCc2c(F)ccc3nnn(C4CC4)c23)n1C] | 1 | A δ 14.36-14.11 (m, 1H), 8.71-8.68 (m, 1H), 8.65 (t, J = 4.4 Hz, 1H), 8.53 (s, 1H), 8.07 (dd, J = 4.5, 9.0 Hz, 1H), 7.93 (s, 1H), 7.34 (t, J = 9.6 Hz, 1H), 7.27 (s, 1H), 4.96 (br d, J = 3.8 Hz, 2H), 4.21-4.12 (m, 1H), 3.76 (s, 3H), 2.41 (s, 3H), 1.45-1.38 (m, 2H), 1.26-1.18 (m, 2H) | 576.1 |
| B.145 | 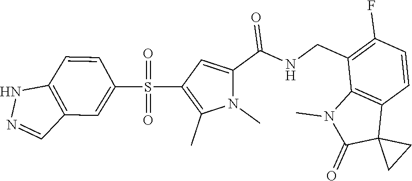 [SMILES: Cc1c(S(=O)(=O)c2ccc3[nH]ncc3c2)cc(C(=O)NCc2c(F)ccc3c2N(C)C(=O)C32CC2)n1C] | <0.15 | A δ 13.50 (br d, J = 3.5 Hz, 1H), 8.58 (br t, J = 3.9 Hz, 1H), 8.36 (s, 1H), 8.29 (s, 1H), 7.68 (s, 2H), 7.27 (s, 1H), 6.99 (dd, J = 5.4, 8.0 Hz, 1H), 6.90- 6.81 (m, 1H), 4.63 (br d, J = 2.8 Hz, 2H), 3.76 (s, 3H), 3.48 (s, 3H), 2.38 (s, 3H), 1.64-1.59 (m, 2H), 1.54 (br d, J = 3.5 Hz, 2H) | 522.2 |
| B.146 | 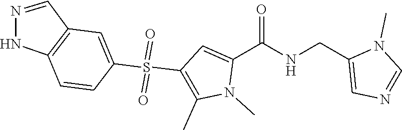 [SMILES: Cc1c(S(=O)(=O)c2ccc3[nH]ncc3c2)cc(C(=O)NCc2cncn2C)n1C] | 29 | B δ 8.47-8.39 (m, 1H), 8.26- 8.19 (m, 1H), 7.82-7.75 (m, 1H), 7.65 (d, J = 9.0 Hz, 1H), 7.56 (s, 1H), 7.13 (s, 1H), 6.92 (s, 1H), 4.48 (s, 2H), 3.78 (s, 3H), 3.68 (s, 3H), 2.44 (s, 3H) | 413.0 |
| B.147 | 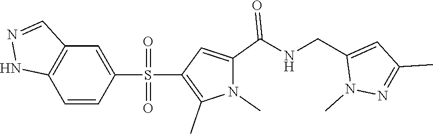 [SMILES: Cc1cc(CNC(=O)c2cc(S(=O)(=O)c3ccc4[nH]ncc4c3)c(C)n2C)n(C)n1] | 145 | B δ 8.45 (d, J = 1.0 Hz, 1H), 8.23 (s, 1H), 7.80 (dd, J = 1.5, 8.9 Hz, 1H), 7.66 (d, J = 8.9 Hz, 1H), 7.16 (s, 1H), 6.01 (s, 1H), 4.45 (s, 2H), 3.78 (d, J = 2.9 Hz, 6H), 2 45 (s, 3H), 2.17 (s, 3H) | 427.1 |
| B.148 | 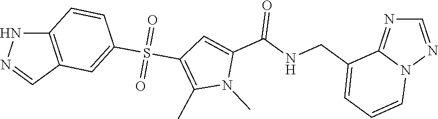 [SMILES: Cc1c(S(=O)(=O)c2ccc3[nH]ncc3c2)cc(C(=O)NCc2cccn3ncnc23)n1C] | 7.4 | A δ 13.62-13.43 (m, 1H), 8.99-8.92 (m, 1H), 8.86 (d, J = 6.4 Hz, 1H), 8.51 (s, 1H), 8.40 (s, 1H), 8.31 (s, 1H), 7.75-7.68 (m, 2H), 7.47 (dd, J = 1.0, 7.1 Hz, 1H), 7.34 (s, 1H), 7.17 (t, J = 7.0 Hz, 1H), 4.70 (d, J = 5.8 Hz, 2H), 3.75 (s, 3H), 2.41 (s, 3H) | 450.0 |
| B.149 | 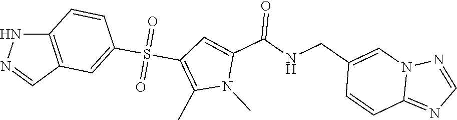 [SMILES: Cc1c(S(=O)(=O)c2ccc3[nH]ncc3c2)cc(C(=O)NCc2ccc3ncnn3c2)n1C] | 18 | A δ 13.66-13.38 (m, 1H), 8.93-8.82 (m, 2H), 8.48 (s, 1H), 8.39 (d, J = 1.1 Hz, 1H), 8.30 (s, 1H), 7.82 (d, J = 9.1 Hz, 1H), 7.70 (s, 2H), 7.63 (dd, J = 1.6, 9.2 Hz, 1H), 7.27 (s, 1H), 4.46 (d, J = 5.9 Hz, 2H), 3.75 (s, 3H), 2.40 (s, 3H) | 450.0 |
| B.150 | 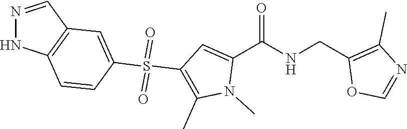 [SMILES: Cc1ncoc1CNC(=O)c1cc(S(=O)(=O)c2ccc3[nH]ncc3c2)c(C)n1C] | 14 | B δ 8.47-8.42 (m, 1H), 8.24 (s, 1H), 8.04 (s, 1H), 7.79 (dd, J = 1.6, 8.9 Hz, 1H), 7.65 (d, J = 8.9 Hz, 1H), 7.14 (s, 1H), 4.48 (s, 2H), 3.78 (s, 3H), 2.44 (s, 3H), 2.20 (s, 3H) | 414.1 |
| B.151 | 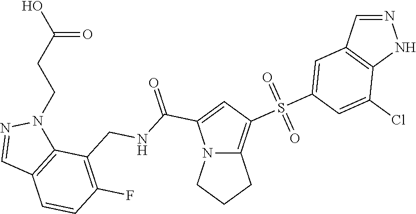 [SMILES: O=C(O)CCn1ncc2ccc(F)c(CNC(=O)c3cc(S(=O)(=O)c4cc(Cl)c5[nH]ncc5c4)c4n3CCC4)c21] | <0.15 | B δ 8.35 (d, J = 1.3 Hz, 1H), 8.29 (s, 1H), 8.02 (s, 1H), 7.79 (s, 1H), 7.71 (dd, J = 5.1, 8.7 Hz, 1H), 7.20 (s, 1H), 7.02- 6.95 (m, 1H), 4.95 (br s, 2H), 4.75 (s, 2H), 4.32 (t, J = 7.2 Hz, 2H), 3.02 (t, J = 7.4 Hz, 2H), 2.85 (t, J = 7.1 Hz, 2H), 2.53 (br t, J = 7.0 Hz, 2H) | 585.0 |
| B.152 | 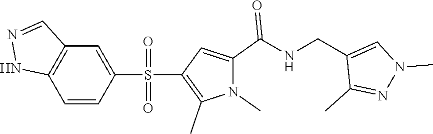 [SMILES: Cc1nn(C)cc1CNC(=O)c1cc(S(=O)(=O)c2ccc3[nH]ncc3c2)c(C)n1C] | 20 | B δ 8.44 (dd, J = 0.6, 1.6 Hz, 1H), 8.23 (d, J = 0.9 Hz, 1H), 7.79 (dd, J = 1.6, 8.9 Hz, 1H), 7.65 (d, J = 8.9 Hz, 1H), 7.57 (s, 1H), 7.10 (s, 1H), 4.28 (s, 2H), 3.82 (s, 3H), 3.78 (s, 3H), 2.44 (s, 3H), 2.27 (s, 3H) | 427.0 |
| B.153 | 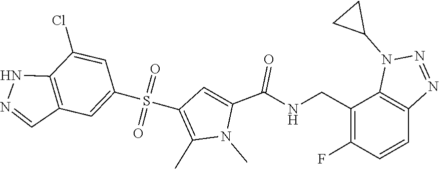 [SMILES: Cc1c(S(=O)(=O)c2cc(Cl)c3[nH]ncc3c2)cc(C(=O)NCc2c(F)ccc3nnn(C4CC4)c23)n1C] | <0.15 | B δ 8.36 (d, J = 1.3 Hz, 1H), 8.29 (s, 1H), 7.95 (dd, J = 4.4, 9.1 Hz, 1H), 7.75 (s, 1H), 7.28 (t, J = 9.6 Hz, 1H), 7.13 (s, 1H), 5.11 (d, J = 1.0 Hz, 2H), 4.25 (tt, J = 3.7, 7.2 Hz, 1H), 3.77 (s, 3H), 2.44 (s, 3H), 1.51-1.44 (m, 2H), 1.36-1.28 (m, 2H) | 542.1 |
| B.154 | 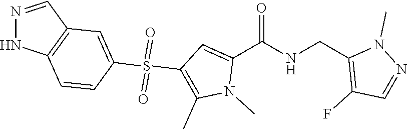 [SMILES: Cc1c(S(=O)(=O)c2ccc3[nH]ncc3c2)cc(C(=O)NCc2c(F)cnn2C)n1C] | 0.56 | A δ 8.76 (s, 1H), 8.38 (s, 1H), 8.30 (s, 1H), 7.69 (s, 2H), 7.38 (d, J = 4.5 Hz, 1H), 7.25 (s, 1H), 4.40 (d, J = 5.3 Hz, 2H), 3.76 (s, 3H), 3.74 (s, 3H), 2.39 (s, 3H) | 431.0 |
| B.155 | 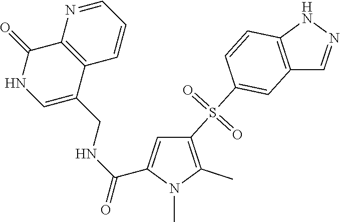 [SMILES: Cc1c(S(=O)(=O)c2ccc3[nH]ncc3c2)cc(C(=O)NCc2c[nH]c(=O)c3ncccc23)n1C] | 0.3 | A δ 13.57-13.45 (m, 1H), 11.56-11.44 (m, 1H), 8.80- 8.75 (m, 1H), 8.63-8.53 (m, 1H), 8.35 (s, 1H), 8.30-8.27 (m, 1H), 8.25-8.19 (m, 1H), 7.72 (dd, J = 4.3, 8.3 Hz, 1H), 7.67 (s, 2H), 7.26-7.20 (m, 1H), 7.17 (s, 1H), 4.44 (d, J = 5.4 Hz, 2H), 3.77 (s, 3H), 2.42-2.36 (m, 3H) | 477.0 |
| B.156 | 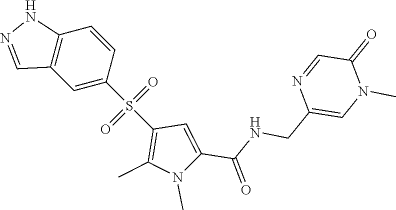 [SMILES: Cc1c(S(=O)(=O)c2ccc3[nH]ncc3c2)cc(C(=O)NCc2cn(C)c(=O)cn2)n1C] | 6 | A δ 13.60-13.43 (m, 1H), 8.74-8.64 (m, 1H), 8.42-8.35 (m, 1H), 8.34-8.26 (m, 1H), 7.97 (d, J = 1.0 Hz, 1H), 7.70 (s, 2H), 7.59 (s, 1H), 7.26 (s, 1H), 4.20 (d, J = 5.6 Hz, 2H), 3.75 (s, 3H), 3.42 (s, 3H), 2.40 (s, 3H) | 441.0 |
| B.157 | 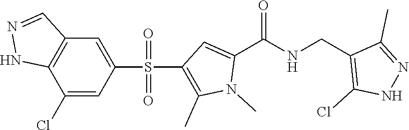 [SMILES: Cc1n[nH]c(Cl)c1CNC(=O)c1cc(S(=O)(=O)c2cc(Cl)c3[nH]ncc3c2)c(C)n1C] | 7.6 | A δ 8.38-8.33 (m, 2H), 8.29 (s, 1H), 7.68 (s, 2H), 7.18 (s, 1H), 4.11 (d, J = 5.4 Hz, 2H), 3.74 (s, 3H), 2.38 (s, 3H), 2.13 (s, 6H) | 427.0 |
| B.158 | 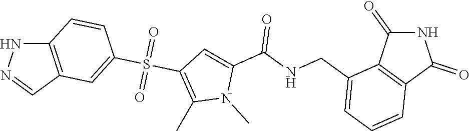 [SMILES: Cc1c(S(=O)(=O)c2ccc3[nH]ncc3c2)cc(C(=O)NCc2cccc3c2C(=O)NC3=O)n1C] | 0.2 | A δ 8.92-8.84 (m, 1H), 8.40 (t, J = 1.19 Hz, 1H), 8.31 (s, 1H), 7.78-7.69 (m, 4H), 7.68-7.62 (m, 1H), 7.32 (s, 1H), 4.83 (d, J = 5.88 Hz, 2H), 3.75 (s, 3H), 3.04 (s, 3H), 2.41 (s, 3H) | 492.0 |
| B.159 | 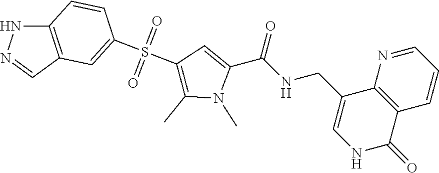 [SMILES: Cc1c(S(=O)(=O)c2ccc3[nH]ncc3c2)cc(C(=O)NCc2c[nH]c(=O)c3cccnc23)n1C] | 0.43 | A δ 13.63-13.35 (m, 1H), 11.45 (br d, J = 5.8 Hz, 1H), 8.98 (dd, J = 1.9, 4.5 Hz, 1H), 8.60-8.49 (m, 2H), 8.38 (t, J = 1.2 Hz, 1H), 8.30 (s, 1H), 7.69 (s, 2H), 7.55 (dd, J = 4.6, 8.1 Hz, 1H), 7.29 (d, J = 6.1 Hz, 1H), 7.25 (s, 1H), 4.55 (d, J = 5.3 Hz, 2H), 3.76 (s, 3H), 2.40 (s, 3H) | 477.0 |
| B.160 | 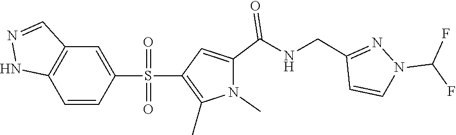 [SMILES: Cc1c(S(=O)(=O)c2ccc3[nH]ncc3c2)cc(C(=O)NCc2ccn(C(F)F)n2)n1C] | 149 | A δ 13.65-13.39 (m, 1H), 8.81 (t, J = 5.9 Hz, 1H), 8.39 (s, 1H), 8.30 (s, 1H), 8.12 (d, J = 2.6 Hz, 1H), 7.92-7.56 (m, 3H), 7.25 (s, 1H), 6.41 (d, J = 2.6 Hz, 1H), 4.37 (d, J = 5.9 Hz, 2H), 3.75 (s, 3H), 2.40 (s, 3H) | 449.0 |
| B.161 | 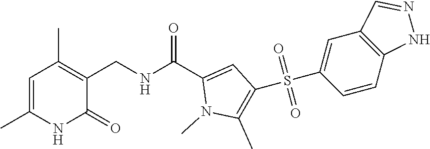 [SMILES: Cc1cc(C)c(CNC(=O)c2cc(S(=O)(=O)c3ccc4[nH]ncc4c3)c(C)n2C)c(=O)[nH]1] | 2.7 | B δ 8.45 (d, J = 0.9 Hz, 1H), 8.24 (d, J = 0.9 Hz, 1H), 7.80 (dd, J = 1.8, 8.9 Hz, 1H), 7.66 (d, J = 8.9 Hz, 1H), 7.26 (s, 1H), 6.69 (s, 1H), 4.46 (s, 2H), 3.79 (s, 3H), 2.51 (s, 3H), 2.46 (s, 3H), 2.42 (s, 3H) | 454.0 |
| B.162 | 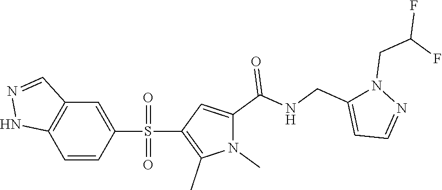 [SMILES: Cc1c(S(=O)(=O)c2ccc3[nH]ncc3c2)cc(C(=O)NCc2ccnn2CC(F)F)n1C] | 2.2 | H NMR A δ 8.82 (t, J = 5.8 Hz, 1H), 8.39 (s, 1H), 8.30 (s, 1H), 7.70 (d, J = 0.9 Hz, 2H), 7.43 (d, J = 1.8 Hz, 1H), 7.23 (s, 1H), 6.52-6.22 (m, 1H), 6.21-6.18 (m, 1H), 4.67 (dt, J = 3.9, 14.9 Hz, 2H), 4.44 (d, J = 5.9 Hz, 2H), 3.74 (s, 3H), 2.39 (s, 3H) | 463.0 |
| B.163 | 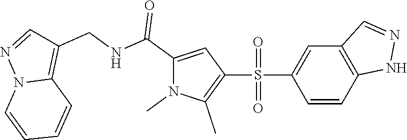 [SMILES: Cc1c(S(=O)(=O)c2ccc3[nH]ncc3c2)cc(C(=O)NCc2cnn3ccccc23)n1C] | 3 | A δ 8.72 (t, J = 5.8 Hz, 1H), 8.65-8.58 (m, 1H), 8.36 (t, J = 1.1 Hz, 1H), 8.28 (s, 1H), 7.93 (s, 1H), 7.77-7.72 (m, 1H), 7.67 (d, J = 0.8 Hz, 2H), 7.23- 7.13 (m, 2H), 6.85 (dt, J = 1.3, 6.9 Hz, 1H), 4.50 (d, J = 5.8 Hz, 2H), 3.76 (s, 3H), 2.38 (s, 3H) | 449.1 |
| B.164 | 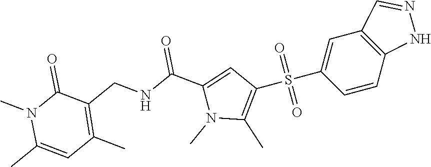 [SMILES: Cc1cc(C)n(C)c(=O)c1CNC(=O)c1cc(S(=O)(=O)c2ccc3[nH]ncc3c2)c(C)n1C] | 0.85 | A δ 13.58-13.41 (m, 1H), 8.36 (s, 1H), 8.29 (s, 1H), 8.14 (t, J = 4.4 Hz, 1H), 7.69 (d, J = 1.6 Hz, 2H), 7.19 (s, 1H), 6.01 (s, 1H), 4.22 (d, J = 5.0 Hz, 2H), 3.73 (s, 3H), 3.42 (s, 3H), 2.38 (s, 3H), 2.30 (s, 3H), 2.15 (s, 3H) | 468.1 |
| B.165 | 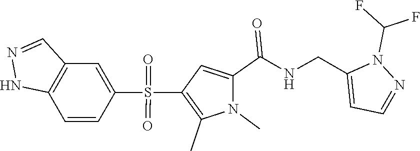 [SMILES: Cc1c(S(=O)(=O)c2ccc3[nH]ncc3c2)cc(C(=O)NCc2ccnn2C(F)F)n1C] | 14 | A δ 13.71-13.32 (m, 1H), 8.91 (t, J = 5.8 Hz, 1H), 8.40 (s, 1H), 8.31 (s, 1H), 8.06-7.63 (m, 4H), 7.25 (s, 1H), 6.37 (s, 1H), 4.57 (d, J = 5.8 Hz, 2H), 3.74 (s, 3H), 2.40 (s, 3H) | 449.0 |
| B.166 | 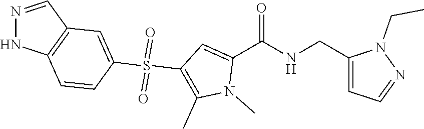 [SMILES: CCn1nccc1CNC(=O)c1cc(S(=O)(=O)c2ccc3[nH]ncc3c2)c(C)n1C] | 0.95 | A δ 8.77 (t, J = 5.8 Hz, 1H), 8.38 (t, J = 1.2 Hz, 1H), 8.30 (s, 1H), 7.70 (d, J = 0.8 Hz, 2H), 7.32 (d, J = 1.8 Hz, 1H), 7.24 (s, 1H), 6.12 (d, J = 1.8 Hz, 1H), 4.42 (d, J = 5.6 Hz, 2H), 4.12 (d, J = 7.1 Hz, 2H), 3.74 (s, 3H), 2.39 (s, 3H), 1.28 (s, 3H) | 427.1 |
| B.167 | 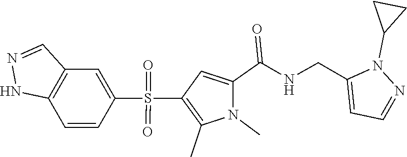 [SMILES: Cc1c(S(=O)(=O)c2ccc3[nH]ncc3c2)cc(C(=O)NCc2ccnn2C2CC2)n1C] | 7.8 | A δ 13.51 (s, 1H), 8.76 (t, J = 5.6 Hz, 1H), 8.39 (s, 1H), 8.30 (s, 1H), 7.70 (s, 2H), 7.28-7.24 (m, 2H), 6.13 (d, J = 1.6 Hz, 1H), 4.53 (d, J = 5.6 Hz, 2H), 3.74 (s, 3H), 3.58 (td, J = 3.5, 7.3 Hz, 1H), 2.40 (s, 3H), 1.07-1.02 (m, 2H), 0.99-0.93 (m, 2H) | 439.1 |
| B.168 | 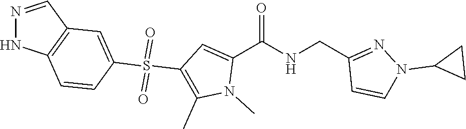 [SMILES: Cc1c(S(=O)(=O)c2ccc3[nH]ncc3c2)cc(C(=O)NCc2ccn(C3CC3)n2)n1C] | 172 | A δ 13.57-13.43 (m, 1H), 8.66 (br t, J = 5.7 Hz, 1H), 8.38 (s, 1H), 8.30 (s, 1H), 7.70 (s, 2H), 7.64 (d, J = 2.1 Hz, 1H), 7.22 (s, 1H), 6.08 (d, J = 2.1 Hz, 1H), 4.27 (d, J = 5.9 Hz, 2H), 3.75 (s, 3H), 3.62 (td, J = 3.5, 7.3 Hz, 1H), 2.40 (s, 3H), 0.97 (br s, 2H), 0.94-0.88 (m, 2H) | 439.1 |
| B.169 | 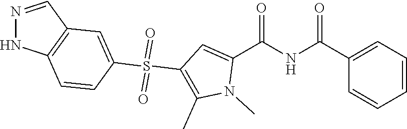 [SMILES: Cc1c(S(=O)(=O)c2ccc3[nH]ncc3c2)cc(C(=O)NC(=O)c2ccccc2)n1C] | 3.2 | A δ 13.63-13.41 (m, 1H), 11.22-10.88 (m, 1H), 8.43 (s, 1H), 8.32 (s, 1H), 7.88-7.80 (m, 2H), 7.78-7.68 (m, 2H), 7.67-7.58 (m, 2H), 7.56-7.48 (m, 2H), 3.74 (s, 3H), 2.45 (s, 3H) | 423.0 |
| B.170 | 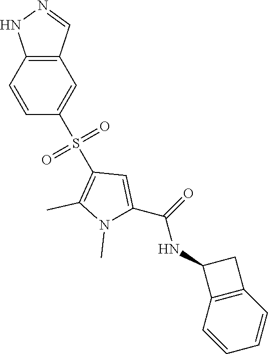 [SMILES: Cc1c(S(=O)(=O)c2ccc3[nH]ncc3c2)cc(C(=O)N[C@H]2Cc3ccccc32)n1C] | 2.5 | A & 8.94 (br d, J = 7.4 Hz, 1H), 8.37 (s, 1H), 8.29 (s, 1H), 7.69 (s, 2H), 7.32 (s, 1H), 7.31-7.20 (m, 2H), 7.20-7.15 (m, 2H), 5.41 (br t, J = 4.8 Hz, 1H), 3.79 (s, 3H), 3.58 (br dd, J = 5.1, 14.0 Hz, 1H), 3.15 (br d, J = 14.1 Hz, 1H), 2.41 (s, 3H) | 421.0 |
| | or | | | |
| | 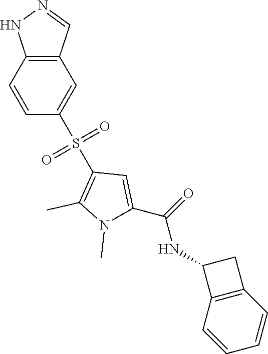 [SMILES: Cc1c(S(=O)(=O)c2ccc3[nH]ncc3c2)cc(C(=O)N[C@@H]2Cc3ccccc32)n1C] | | | |
| | (faster eluting isomer) | | | |
| B.171 | 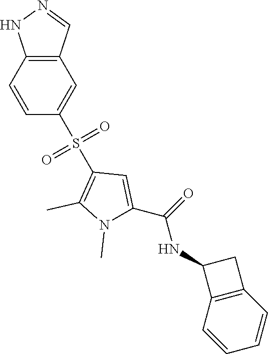 [SMILES: Cc1c(S(=O)(=O)c2ccc3[nH]ncc3c2)cc(C(=O)N[C@H]2Cc3ccccc32)n1C] | 2.7 | B δ 8.44 (d, J = 0.8 Hz, 1H), 8.22 (s, 1H), 7.79 (dd, J = 1.5, 8.9 Hz, 1H), 7.64 (d, J = 9.0 Hz, 1H), 7.30-7.21 (m, 2H), 7.21-7.16 (m, 2H), 7.12 (d, J = 7.0 Hz, 1H), 5.47 (dd, J = 2.2, 4.8 Hz, 1H), 3.81 (s, 3H), 3.66 (dd, J = 5.1, 14.2 Hz, 1H), 3.16 (dd, J = 2.2, 14.2 Hz, 1H), 2.45 (s, 3H) | 421.1 |
| | or | | | |
| | 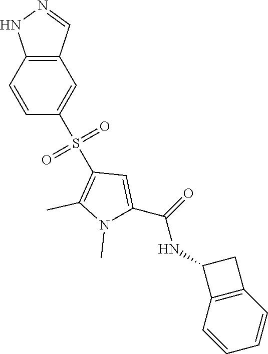 [SMILES: Cc1c(S(=O)(=O)c2ccc3[nH]ncc3c2)cc(C(=O)N[C@@H]2Cc3ccccc32)n1C] | | | |
| | (slower eluting isomer) | | | |
| B.172 | 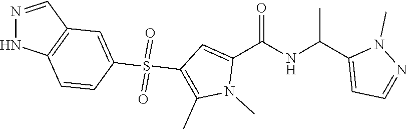 [SMILES: Cc1c(S(=O)(=O)c2ccc3[nH]ncc3c2)cc(C(=O)NC(C)c2ccnn2C)n1C] | 24 | A δ 8.61 (d, J = 8.4 Hz, 1H), 8.38 (s. 1H), 8.30 (s, 1H), 7.70 (d, J = 0.8 Hz, 2H), 7.34-7.26 (m, 2H), 6.22 (d, J = 1.8 Hz, 1H), 5.24 (quin, J = 7.2 Hz, 1H), 3.74 (s, 6H), 2.39 (s, 3H), 1.45 (d. J = 6.9 Hz, 3H) | 427.1 |
| | (racemic) | | | |
| B.173 | 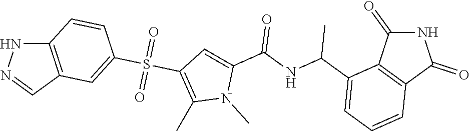 [SMILES: Cc1c(S(=O)(=O)c2ccc3[nH]ncc3c2)cc(C(=O)NC(C)c2cccc3c2C(=O)NC3=O)n1C] | 0.68 | A δ 13.76-13.38 (m, 1H), 11.35 (s, 1H), 8.89 (t, J = 5.9 Hz, 1H), 8.42-8.29 (m, 2H), 7.79-7.62 (m, 5H), 7.32 (s, 1H), 4.81 (d, J = 5.8 Hz, 2H), 3.75 (s, 3H), 2.41 (s, 3H) | 478.0 |
| B.174 | 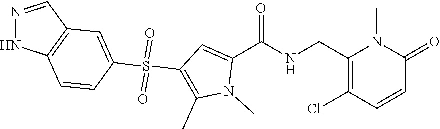 [SMILES: Cc1c(S(=O)(=O)c2ccc3[nH]ncc3c2)cc(C(=O)NCc2c(Cl)ccc(=O)n2C)n1C] | 0.7 | A δ 8.60 (t, J = 4.6 Hz, 1H), 8.38 (s, 1H), 8.30 (s, 1H), 7.70 (s, 2H), 7.51 (d, J = 9.8 Hz, 1H), 7.28 (s, 1H), 6.47 (d, J = 9.6 Hz, 1H), 4.57 (d, J = 4.5 Hz, 2H), 3.75 (s, 3H), 3.46 (s, 3H), 2.39 (s, 3H) | 474.0 |
| B.175 | 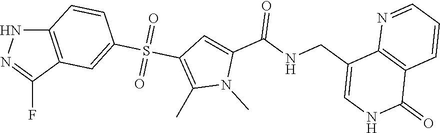 [SMILES: Cc1c(S(=O)(=O)c2ccc3[nH]nc(F)c3c2)cc(C(=O)NCc2c[nH]c(=O)c3cccnc23)n1C] | 3.3 | A δ 13.32-12.83 (m, 1H), 11.45 (br d, J = 0.9 Hz, 1H), 8.98 (dd, J = 1.8, 4.6 Hz, 1H), 8.62-8.47 (m, 2H), 8.24 (d, J = 0.9 Hz, 1H), 7.76 (dd, J = 1.6, 9.0 Hz, 1H), 7.66 (dd, J = 1.5, 9.0 Hz, 1H), 7.55 (dd, J = 4.6, 8.1 Hz, 1H), 7.29 (br s, 1H), | 495.0 |
| | | | 7.26 (s, 1H), 4.56 (d, J = 5.3 | |
| | | | Hz, 2H), 3.76 (s, 3H), 2.41 (s, | |
| | | | 3H) | |
| B.176 | 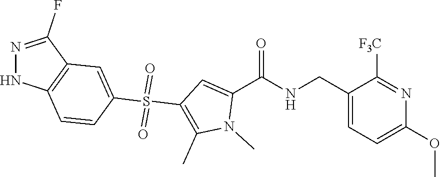 [SMILES: COc1ccc(CNC(=O)c2cc(S(=O)(=O)c3ccc4[nH]nc(F)c4c3)c(C)n2C)c(C(F)(F)F)n1] | 7 | A δ 8.83 (t, J = 5.6 Hz, 1H), 8.26 (s, 1H), 7.87 (d, J = 8.6 Hz, 1H), 7.79-7.74 (m, 1H), 7.70-7.64 (m, 1H), 7.30 (s, 1H), 7.16-7.10 (m, 1H), 4.51- 4.46 (m, 2H), 3.89 (s, 3H), 3.78-3.72 (m, 3H), 2.41 (s, 3H) | 526.1 |
| B.177 | 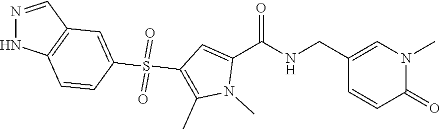 [SMILES: Cc1c(S(=O)(=O)c2ccc3[nH]ncc3c2)cc(C(=O)NCc2ccc(=O)n(C)c2)n1C] | 138 | A δ 13.52 (br s, 1H), 8.65 (br t, J = 5.5 Hz, 1H), 8.38 (s, 1H), 8.29 (s, 1H), 7.70 (s, 2H), 7.59 (d, J = 2.1 Hz, 1H), 7.38 (dd, J = 2.5, 9.3 Hz, 1H), 7.21 (s, 1H), 6.36 (d, J = 9.3 Hz, 1H), 5.75 (s, 1H), 4.08 (d, J = 5.8 Hz, 2H), 3.75 (s, 3H), 3.39 (s, 3H), 2.39 (s, 3H) | 440.1 |
| B.178 | 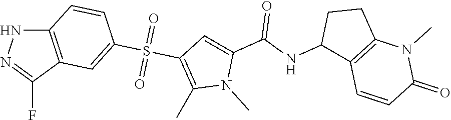 [SMILES: Cc1c(S(=O)(=O)c2ccc3[nH]nc(F)c3c2)cc(C(=O)NC2CCc3c2ccc(=O)n3C)n1C] | 21 | A δ 13.30-12.92 (m, 1H), 8.47 (d, J = 7.9 Hz, 1H), 8.24 (s, 1H), 7.76 (dd, J = 1.3, 9.1 Hz, 1H), 7.66 (br d, J = 8.0 Hz, 1H), 7.30 (d, J = 9.0 Hz, 1H), 7.24 (s, 1H), 6.23 (d, J = 9.2 Hz, 1H), 5.31-5.17 (m, 1H), 3.77 (s, 3H), 3.38 (s, 3H), | 484.0 |
| | | | 3.04-2.98 (m, 1H), 2.94-2.80 | |
| | (racemic) | | (m, 1H), 2.41 (s, 4H), 2.01- | |
| | | | 1.89 (m, 1H) | |
| B.179 | 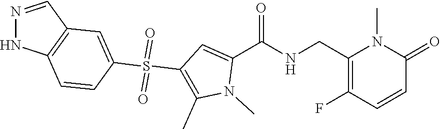 [SMILES: Cc1c(S(=O)(=O)c2ccc3[nH]ncc3c2)cc(C(=O)NCc2c(F)ccc(=O)n2C)n1C] | <0.15 | A δ 13.65-13.40 (m, 1H), 8.66 (t, J = 4.9 Hz, 1H), 8.39 (s, 1H), 8.30 (s, 1H), 7.70 (s, 2H), 7.63-7.52 (m, 1H), 7.28 (s, 1H), 6.45 (dd, J = 5.3, 9.9 Hz, 1H), 4.54-4.42 (m, 2H), 3.75 (s, 3H), 3.41 (s, 3H), 2.40 (s, 3H) | 458.1 |
| B.180 | 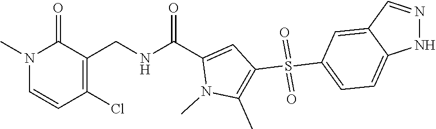 [SMILES: Cc1c(S(=O)(=O)c2ccc3[nH]ncc3c2)cc(C(=O)NCc2c(Cl)ccn(C)c2=O)n1C] | <0.15 | A δ 13.50 (br s, 1H), 8.36 (s, 1H), 8.29 (s, 1H), 8.20 (t, J = 4.5 Hz, 1H), 7.74 (d, J = 7.4 Hz, 1H), 7.69 (s, 2H), 7.21 (s, 1H), 6.38 (d, J = 7.3 Hz, 1H), 4.32 (d, J = 4.6 Hz, 2H), 3.75 (s, 3H), 3.45 (s, 3H), 2.38 (s, 3H) | 474.0 |
| B.181 | 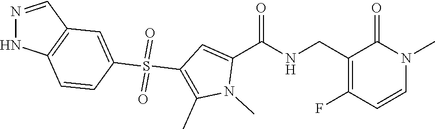 [SMILES: Cc1c(S(=O)(=O)c2ccc3[nH]ncc3c2)cc(C(=O)NCc2c(F)ccn(C)c2=O)n1C] | 0.23 | B δ 8.44 (s, 1H), 8.23 (s, 1H), 7.58-7.85 (m, 3H), 7.11 (s, 1H), 6.32 (t, J = 7.4 Hz, 1H), 4.39 (s, 2H), 3.76 (s, 3H), 3.56 (s, 3H), 2.44 (s, 3H) | 458.1 |
| B.182 | 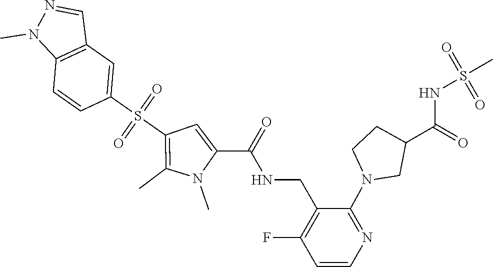 | <0.15 | B δ 8.41 (s, 1H), 8.18 (s, 1H), 7.99-8.06 (m, 1H), 7.79-7.85 (m, 1H), 7.69 (d, J = 8.5 Hz, 1H), 7.12 (s, 1H), 6.59 (dd, J = 8.9, 5.5 Hz, 1H), 4.60 (s, 2H), 4.54-4.57 (m, 1H), 4.09 (s, 3H), 3.82-3.88 (m, 1H), 3.75- 3.78 (m, 3H), 3.56-3.73 (m, 3H), 3.08-3.13 (m, 3H), 2.41- 2.46 (m, 3H), 2.12-2.23 (m, 2H) | 632.1 |
| B.183 | 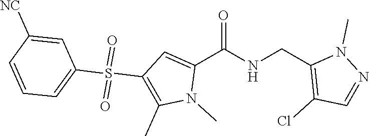 [SMILES: Cc1c(S(=O)(=O)c2cccc(C#N)c2)cc(C(=O)NCc2c(Cl)cnn2C)n1C] | 17 | A δ 8.74 (t, J = 5.3 Hz, 1H), 8.25 (t, J = 1.5 Hz, 1H), 8.12 (ddd, J = 1.6, 5.8, 7.3 Hz, 2H), 7.80 (t, J = 7.9 Hz, 1H), 7.48 (s, 1H), 7.26 (s, 1H), 4.43 (d, J = 5.3 Hz, 2H), 3.82 (s, 3H), 3.76 (s, 3H), 2.41 (s, 3H) | 432.0 |
| B.184 | 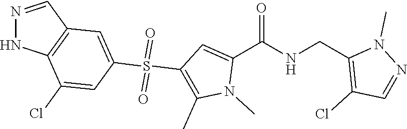 [SMILES: Cc1c(S(=O)(=O)c2cc(Cl)c3[nH]ncc3c2)cc(C(=O)NCc2c(Cl)cnn2C)n1C] | <0.15 | A δ 8.72 (t, J = 5.0 Hz, 1H), 8.49-8.30 (m, 2H), 7.71 (s, 1H), 7.48 (s, 1H), 7.27 (s, 1H), 4.47-4.39 (m, 2H), 3.82 (s, 3H), 3.75 (s, 3H), 2.40 (s, 3H) | 481.0 |
| B.185 | 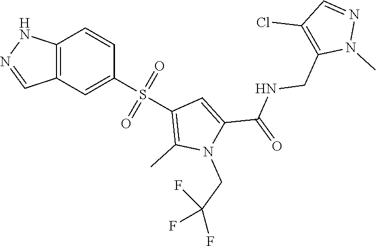 [SMILES: Cc1c(S(=O)(=O)c2ccc3[nH]ncc3c2)cc(C(=O)NCc2c(Cl)cnn2C)n1CC(F)(F)F] | 3.9 | B δ 8.48 (d, J = 1.0 Hz, 1H), 8.25 (s, 1H), 7.80 (dd, J = 1.8, 8.9 Hz, 1H), 7.68 (d, J = 8.9 Hz, 1H), 7.40 (s, 1H), 7.25 (s, 1H), 5.49-5.26 (m, 2H), 4.55 (s, 2H), 3.88 (s, 3H), 2.50 (s, 3H) | 515.0 |
| B.186 | 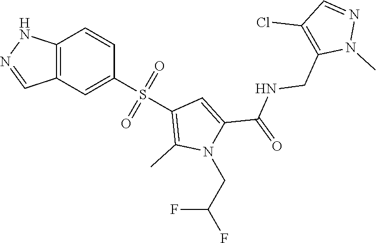 [SMILES: Cc1c(S(=O)(=O)c2ccc3[nH]ncc3c2)cc(C(=O)NCc2c(Cl)cnn2C)n1CC(F)F] | 1.1 | A δ 13.75-13.39 (m, 1H), 8.97-8.74 (m, 1H), 8.63-8.19 (m, 2H), 7.70 (s, 2H), 7.49 (s, 1H), 7.35 (s, 1H), 6.59-6.11 (m, 1H), 4.99-4.71 (m, 2H), 4.44 (br d, J = 5.1 Hz, 2H), 3.80 (s, 3H), 2.43 (s, 3H) | 497.0 |
| B.187 | 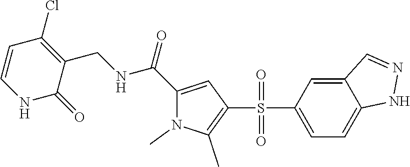 [SMILES: Cc1c(S(=O)(=O)c2ccc3[nH]ncc3c2)cc(C(=O)NCc2c(Cl)cc[nH]c2=O)n1C] | 0.61 | A δ 8.54-8.45 (m, 1H), 8.37 (s, 1H), 8.30 (s, 1H), 7.69 (s, 2H), 7.42 (d, J = 6.5 Hz, 1H), 7.17 (s, 1H), 6.25 (d, J = 7.0 Hz, 1H), 4.30 (br d, J = 4.1 Hz, 2H), 3.75 (s, 3H), 2.39 (s, 3H) | 460.0 |
| B.188 | 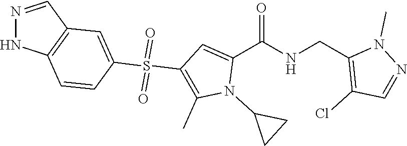 [SMILES: Cc1c(S(=O)(=O)c2ccc3[nH]ncc3c2)cc(C(=O)NCc2c(Cl)cnn2C)n1C1CC1] | 1.6 | B δ 8.44 (d, J = 1.0 Hz, 1H), 8.24 (s, 1H), 7.79 (dd, J = 1.6, 8.9 Hz, 1H), 7.65 (d, J = 9.0 Hz, 1H), 7.41 (s, 1H), 6.89 (s, 1H), 4.56 (s, 2H), 3.93 (s, 3H), 3.25 (tt, J = 3.8, 7.3 Hz, 1H), 2.53 (s, 3H), 1.08-1.00 (m, 2H), 0.72-0.65 (m, 2H) | 473.0 |
| B.189 | 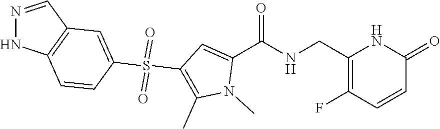 [SMILES: Cc1c(S(=O)(=O)c2ccc3[nH]ncc3c2)cc(C(=O)NCc2[nH]c(=O)ccc2F)n1C] | 0.37 | A δ 13.64-13.35 (m, 1H), 10.56 (br d, J = 7.9 Hz, 1H), 8.61 (br t, J = 5.3 Hz, 1H), 8.39 (s, 1H), 8.30 (s, 1H), 7.70 (s, 2H), 7.51 (t, J = 9.1 Hz, 1H), 7.26 (s, 1H), 6.53-6.38 (m, 1H), 4.34 (br d, J = 4.1 Hz, 2H), 3.74 (s, 3H), 2.40 (s, 3H) | 443.7 |
| B.190 | 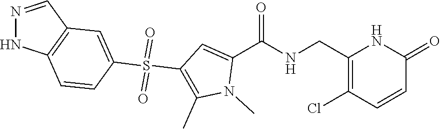 [SMILES: Cc1c(S(=O)(=O)c2ccc3[nH]ncc3c2)cc(C(=O)NCc2[nH]c(=O)ccc2Cl)n1C] | 3.3 | B δ 8.46 (d, J = 0.88 Hz, 1H), 8.24 (s, 1H), 7.81 (dd, J = 8.88, 1.75 Hz, 1H), 7.66 (d, J = 9.01 Hz, 1H), 7.56 (d, J = 9.51 Hz, 1H), 7.24 (s, 1H), 6.47 (d, J = 9.51 Hz, 1H), 4.49 (s, 2H), 3.80 (s, 3H), 2.46 (s, 3H) | 460.0 |
| B.191 | 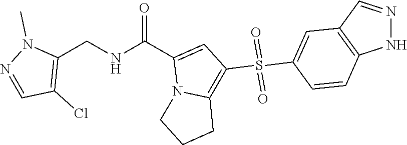 [SMILES: Cn1ncc(Cl)c1CNC(=O)c1cc(S(=O)(=O)c2ccc3[nH]ncc3c2)c2n1CCC2] | 8.6 | A δ 13.51 (br d, J = 5.4 Hz, 1H), 8.61 (t, J = 5.3 Hz, 1H), 8.37 (s, 1H), 8.30 (s, 1H), 7.75-7.68 (m, 2H), 7.47 (s, 1H), 7.25 (s, 1H), 4.41 (d, J = 5.4 Hz, 2H), 4.21 (t, J = 7.3 Hz, 2H), 3.79 (s, 3H), 2.95 (t, J = 7.5 Hz, 2H), 2.47-2.41 (m, 2H) | 459.0 |
| B.192 | 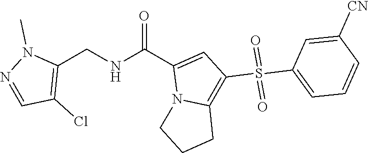 [SMILES: Cn1ncc(Cl)c1CNC(=O)c1cc(S(=O)(=O)c2cccc(C#N)c2)c2n1CCC2] | 98 | A δ 8.65 (br t, J = 5.3 Hz, 1H), 8.25 (s, 1H), 8.13 (br t, J = 9.1 Hz, 2H), 7.84-7.77 (m, 1H), 7.47 (s, 1H), 7.26 (s, 1H), 4.42 (br d, J = 5.3 Hz, 2H), 4.23 (br t, J = 7.2 Hz, 2H), 3.80 (s, 3H), 2.99 (br t, J = 7.5 Hz, 2H), 2.48-2.43 (m, 2H) | 444.0 |
| B.193 | 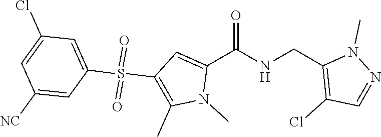 [SMILES: Cc1c(S(=O)(=O)c2cc(Cl)cc(C#N)c2)cc(C(=O)NCc2c(Cl)cnn2C)n1C] | 6.4 | A δ 8.75 (t, J = 5.3 Hz, 1H), 8.35 (t, J = 1.6 Hz, 1H), 8.26 (t, J = 1.4 Hz, 1H), 8.12 (t, J = 1.8 Hz, 1H), 7.48 (s, 1H), 7.27 (s, 1H), 4.44 (d, J = 5.3 Hz, 2H), 3.83 (s, 3H), 3.76 (s, 3H), 2.42 (s, 3H) | 466.0 |
| B.194 | 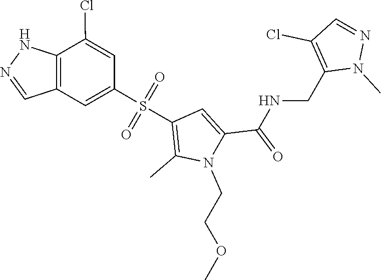 [SMILES: COCCn1c(C(=O)NCc2c(Cl)cnn2C)cc(S(=O)(=O)c2cc(Cl)c3[nH]ncc3c2)c1C] | 1.1 | B δ 8.39 (d, J = 1.4 Hz, 1H), 8.34-8.29 (m, 1H), 7.78 (d, J = 1.3 Hz, 1H), 7.40 (s, 1H), 7.19 (s, 1H), 4.54 (s, 2H), 4.49 (t, J = 5.1 Hz, 2H), 3.91 (s, 3H), 3.58 (t, J = 5.1 Hz, 2H), 3.16 (s, 3H), 2.48 (s, 3H) | 525.1 |
| B.195 | 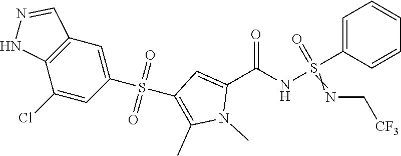 [SMILES: Cc1c(S(=O)(=O)c2cc(Cl)c3[nH]ncc3c2)cc(C(=O)NS(=O)(=NCC(F)(F)F)c2ccccc2)n1C] | 12 | A δ 9.02 (t, J = 6.63 Hz, 1H), 8.48-8.38 (m, 2H), 7.93-7.85 (m, 2H), 7.77 (s, 1H), 7.71- 7.57 (m, 3H), 7.32 (s, 1H), 3.84-3.71 (m, 2H), 3.68 (s, 3H), 2.39 (s, 3H) | 574.0 |
| B.196 | 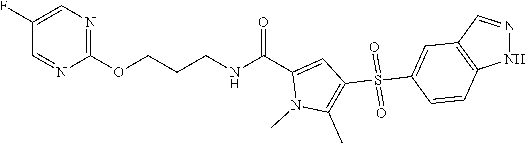 [SMILES: Cc1c(S(=O)(=O)c2ccc3[nH]ncc3c2)cc(C(=O)NCCCOc2ncc(F)cn2)n1C] | 18 | A δ 13.83-13.16 (m, 1H), 8.68 (s, 2H), 8.38 (s, 1H), 8.35 (br t, J = 5.6 Hz, 1H), 8.30 (s, 1H), 7.70 (s, 2H), 7.18 (s, 1H), 4.31 (t, J = 6.3 Hz, 2H), 3.72 (s, 3H), 3.28 (br s, 2H), 2.39 (s, 3H), 1.94 (quin, J = 6.5 Hz, 2H) | 473.2 |
| B.197 | 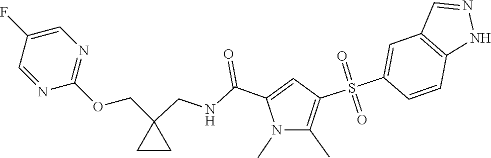 [SMILES: Cc1c(S(=O)(=O)c2ccc3[nH]ncc3c2)cc(C(=O)NCC2(COc3ncc(F)cn3)CC2)n1C] | 2.7 | B δ 8.48 (s, 3H), 8.25 (s, 1H), 7.84 (br d, J = 8.8 Hz, 1H), 7.67 (br d, J = 8.5 Hz, 1H), 7.20 (s, 1H), 4.30 (s, 2H), 3.72 (s, 3H), 3.43 (s, 2H), 2.45 (s, 3H), 0.72 (br s, 2H), 0.69 (br s, 2H) | 499.2 |
| B.198 | 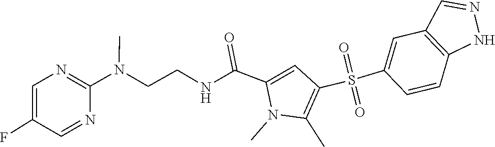 [SMILES: Cc1c(S(=O)(=O)c2ccc3[nH]ncc3c2)cc(C(=O)NCCN(C)c2ncc(F)cn2)n1C] | 16 | A δ 13.51 (br s, 1H), 8.40 (s, 2H), 8.38-8.33 (m, 2H), 8.30 (s, 1H), 7.69 (d, J = 1.4 Hz, 2H), 7.04 (s, 1H), 3.73-3.67 (m, 5H), 3.41-3.37 (m, 2H), 3.08 (s, 3H), 2.38 (s, 3H) | 472.0 |

NMR solvent:
A = DMSO-d6;
B = METHANOL-d4;
C = CHLOROFORM-d

### SARS-CoV-2/Huh7.5 Screening Assay

Huh7.5 cells are seeded in 96-well plates (Corning, Product Number 353072) in 10% FBS-containing media at a density of 1.0×10⁴ cells per well. Plates are incubated for 24 h at 37° C., 5% CO₂. After addition of compounds (100× in DMSO) to cells, plates are transported to the BSL3 facility (laboratory of Dr. Charles Rice, RU), where SARS-CoV-2 (strain USA-WA1/2020 propagated in Vero E6 cells) diluted in assay media is added to achieve ~30-50% infected cells. Plates are incubated for 24 h at 37° C., 5% CO₂, and then fixed with 3.5% formaldehyde. Fixed cells are analyzed for viral infection by immunostaining for SARS-CoV-2 nucleocapsid protein using SARS-CoV-2 (COVID-19) nucleocapsid antibody (Genetex, GTX135357) as the primary antibody and Alexa Fluor 488-conjugated goat anti-rabbit IgG (H+L, Invitrogen by Thermo Fisher Scientific, A11008) as the secondary antibody, and antifade-46-diamidino-2-phenylindole (DAPI; Thermo Fisher Scientific D1306) to stain DNA, with PBS 0.05% Tween-20 washes in between fixation and subsequent primary and secondary antibody staining.

Plates are imaged using the ImageXpress Micro Confocal High-Content Imaging System (Molecular Devices) with a 10× objective, with 4 fields imaged per well. Images are analyzed using the Multi-Wavelength Cell Scoring Application Module (MetaXpress), with DAPI staining identifying the host-cell nuclei (the total number of cells in the images) and the SARS-CoV-2 immunofluorescence signal leading to identification of infected cells.

For each condition, the percentage of infection is calculated as the ratio of the number of infected cells stained for coronavirus NP to number of cells stained with DAPI. Treatment of cells with DMSO serves as negative control; treatment of cells with 200 nM Remdesivir serves as positive control.

For dose response experiments, compounds are tested in technical triplicates on different assay plates and dose response curves are fitted with the four parameter Hill Equation.

Representative cellular activities of examples in the SARS-CoV-2/Huh7.5 screening assay are shown below in Table 4.

TABLE 4

| | IC₅₀ |
|---|---|
| Example | (μM) |
| A.1 | 0.005 |
| A.2 | 0.017 |
| A.3 | 0.068 |
| A.4 | 0.076 |
| A.5 | 0.21 |
| A.6 | 0.23 |
| A.8 | 0.49 |
| A.34 | 0.021 |
| A.35 | 0.48 |
| A.37 | 0.058 |
| A.46 | 0.08 |
| A.54 | 0.0030 |
| A.55 | 0.43 |
| A.57 | 0.18 |
| A.59 | 0.21 |
| A.60 | 0.35 |
| A.61 | 0.12 |
| A.66 | 0.12 |
| A.72 | 0.11 |
| A.73 | 0.093 |
| A.79 | 0.40 |
| A.82 | 0.071 |
| A.88 | 0.35 |
| A.90 | 0.35 |
| A.95 | 0.10 |
| B.2 | 0.052 |
| B.12 | 0.038 |
| B.13 | 0.056 |
| B.16 | 0.002 |
| B.18 | 0.0012 |
| B.22 | 0.0087 |
| B.24 | 0.287 |
| B.25 | 0.057 |
| B.27 | 0.026 |
| B.32 | 0.033 |
| B.33 | 0.26 |
| B.34 | 0.064 |
| B.43 | 0.0064 |
| B.49 | 0.057 |
| B.51 | 0.013 |
| B.52 | 0.0082 |
| B.63 | 0.0068 |
| B.66 | 0.15 |
| B.81 | 0.013 |
| B.87 | 0.28 |
| B.89 | 0.062 |
| B.134 | 0.35 |
| B.136 | 0.084 |
| B.142 | 0.19 |
| B.143 | 0.024 |
| B.145 | 0.0056 |
| B.151 | 0.25 |
| B.153 | 0.0042 |
| B.174 | 0.1 |
| B.179 | 0.22 |

### Computational Modeling

Models of the inhibitors bound to nsp14 were generated from SARS-CoV-2 nsp14 structures (PDB IDs 7R2V, 7TW8) and homology models built from SARS-CoV nsp14 structures (PDB IDs 5C8S, 5C8T, 5C8U, 5NFY, 7NOB, 7NOC, 7NOD). Models were built with and without SAH. The model that aligned with the SAR and validated with quantitative accuracy using FEP+ came from the structure with PDB ID 7R2V with SAH bound (Refolding of lid subdomain of SARS-CoV-2 nsp14 upon nsp10 interaction releases exonuclease activity, Czarna et al, Structure, 2022).

What is claimed is:

1. A compound of formula I: 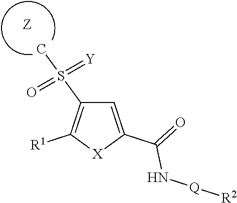
wherein
X is chosen from N(R^{a}) and S;
R^{a} is chosen from (C₁-C₆)hydrocarbon, (C₁-C₆)oxoalkyl, and hydrogen, wherein said (C₁-C₆)hydrocarbon and said (C₁-C₆)oxoalkyl is optionally substituted with one or more hydrogen, alkyl, alkoxy, hydroxyl, oxo, carbonyl, halogen, amino, cyano, or aminoacyl alkyl;
R¹ is chosen from methyl and ethyl; or
R^{a} and R¹ taken together with the atoms to which they are attached form a 5- or 6-member heterocycle optionally substituted with methyl; and
Y is chosen from O and N(R^{b});
R^{b} is chosen from hydrogen, (C₁-C₆)hydrocarbon, and hydroxyalkyl;
Z is chosen from aryl or heteroaryl, wherein said aryl and said heteroaryl is optionally substituted with one or more (C₁-C₆)alkyl, cyano, halogen, haloalkyl, cyanoalkyl, alkoxyalkyl, alkylsulfonyl, oxo, carbonyl, alkylcarbonyl, carboxamide, alkoxy, acetamide, cycloalkyl, hydroxyalkyl, or amino;
Q is chosen from direct bond, (C₁-C₆)hydrocarbon, (C₁-C₆)heteroalkyl, heterocycle, NH, 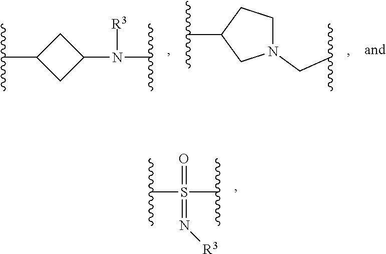
wherein said (C₁-C₆)hydrocarbon, said (C₁-C₆)heteroalkyl, and said heterocycle, is optionally substituted with one or more (C₁-C₁₀)hydrocarbyl, methylazetidine, oxoalkyl, hydroxyalkyl, halogen, oxo, hydroxyl, or haloalkyl;
R³ is chosen from hydrogen, alkyl, and haloalkyl;
R² is chosen from HetA, CarbA, —O-HetA, N(R^{c})₂, and C₁₃ hydrocarbon, said HetA and said CarbA is optionally substituted with one or more HetB, CarbB, halogen, alkoxy, oxoalkyl, cyano, COOH, hydroxyl, oxo, carbonyl, N(R^{c})₂, pyrrolidinylmethyl, azaalkyl, alkyl, thioalkyl, haloalkyl, alkoxylalkyl, haloalkylalkoxy, dimethylaminoalkyl, oxotetrahydrofuran, alkylsulfonyl, dimethylaminoalkylalkoxy, alkylsulfonylalkylalkoxy, —O-CarbB, or —O-HetB,
wherein said HetB, said alkoxy, said CarbB, said oxoalkyl, said azaalkyl, and said thioalkyl, is optionally substituted with one or more halogen, hydroxyl, (C₁-C₆)alkoxy, (C₁-C₆)hydrocarbyl, COOH, cyano, C(O)OC(CH₃)₂, N(R^{c})₂, (C₁-C₆)alkoxy(C₁-C₆)alkyl, (C₁-C₆)alkyl, oxo, carbonyl, C(O)CH₃, or alkylsulfonylcarboxamide,
wherein R^{c} is independently chosen in each instance from hydrogen, hydrocarbyl, haloalkyl, cyano, cyanoalkyl, alkoxyalkyl, carbonylhaloalkyl, oxoalkyl, carbonylalkyl, alkylsulfonyl, and HetA, said HetA optionally substituted with one or more halogen, and
wherein HetA is a 3- to 12-member heterocycle, HetB is a 3- to 11-member heterocycle, CarbA is a 6- to 10-member carbocycle, and CarbB is a 3- to 5-member carbocycle.

2. The compound of claim 1, wherein
R^{a} is chosen from (C₁-C₃)hydrocarbon, (C₁-C₄)oxoalkyl, and hydrogen, wherein said (C₁-C₃)hydrocarbon and said (C₁-C₄)oxoalkyl is optionally substituted with one or more methyl, hydroxyl, methoxy, fluoro, oxo, NH₂, ethyl, or NH—C(O)—CH₃;
Z is chosen from
(i) 5- or 6-member nitrogen-containing monocycle optionally substituted with one or more methyl, C(O)CH₃, haloalkyl, ethyl, cyano, cyanomethyl, or methoxymethyl, and
(ii) fused nitrogen-containing bicycle optionally substituted with one or more methyl, cyano, halogen, haloalkyl, methylsulfonyl, NH₂, oxo, or carbonyl;
Q is chosen from (C₁-C₂)alkyl and direct bond, wherein said (C₁-C₂)alkyl is optionally substituted with one or more (C₁-C₁₀)hydrocarbyl or methylazetidine; and
R² is chosen from
(i) heteroaryl and aryl, said heteroaryl and said aryl is optionally substituted with one or more 3- to 11-member heterocycle, halogen, (C₁-C₁₀)alkoxy, (C₁-C₁₀)hydrocarbyl, (C₁-C₁₀)oxoalkyl, cyano, COOH, hydroxyl, oxo, carbonyl, N(R^{c})₂, pyrrolidinylmethyl, (C₁-C₁₀)azaalkyl, difluoromethyl, or (C₁-C₁₀)thioalkyl,
wherein said 3- to 11-member heterocycle, said (C₁-C₁₀)alkoxy, said (C₁-C₁₀)hydrocarbyl, said (C₁-C₁₀)oxoalkyl, said (C₁-C₁₀)azaalkyl, and said (C₁-C₁₀)thioalkyl, are each independently optionally substituted with one or more halogen, hydroxyl, (C₁-C₆)alkoxy, (C₁-C₆)hydrocarbyl, COOH, cyano, C(O)OC(CH₃)₂, or N(R^{c})₂, and
wherein R^{c} in each instance is independently chosen from hydrogen, (C₁-C₁₀)hydrocarbyl, C(O)haloalkyl, oxo(C₁-C₆)alkyl, and alkylsulfonyl; and
(ii) 6:5 or 6:6 bicyclic heterocycle, 5- to 10-member heterocycle, and phenyl,
wherein said phenyl is optionally substituted with one or more haloalkyl, alkoxyalkyl, cyano, COOH, alkoxy, or haloalkylalkoxy,
wherein said 6:5 or 6:6 bicyclic heterocycle and said 5- to 10-member heterocycle is optionally substituted with one or more 3- to 10-member heterocycle, (C₁-C₆)alkyl, dimethylaminemethyl, pyrrolidinylmethyl, oxo, carbonyl, halogen, hydroxyl, N(R^{d})₂, oxotetrahydrofuran, alkylsulfonyl, (C₁-C₆)hydrocarbon, cyano, COOH, or (C₁-C₃)alkoxy,
wherein R^{d} in each instance is independently chosen from hydrogen, (C₁-C₆)hydrocarbon, and (C₁-C₆)oxoalkyl,
wherein said 3- to 10-member heterocycle is optionally substituted with one or more alkoxyalkyl, alkyl, hydroxyl, oxo, carbonyl, or C(O)CH₃, and
wherein said (C₁-C₆)hydrocarbon is optionally substituted with one or more halogen.

3. The compound of claim 1 or claim 2, wherein X is N(R^{a}).

4. The compound of claim 1 or claim 2, wherein X is S.

5. The compound of any one of claims 1-4, wherein R^{a} is hydrogen, methyl, or cyano.

6. The compound of any one of claims 1-5, wherein R¹ is methyl.

7. The compound of any one of claims 1-4, wherein R^{a} and R¹ taken together with the atoms to which they are attached form a 5- or 6-member heterocycle optionally substituted with methyl.

8. The compound of any one of claims 1-7, wherein Y is O.

9. The compound of any one of claims 1-7, wherein Y is N(R^{b}).

10. The compound of any one of claims 1-9, wherein R^{b} is hydrogen, methyl, ethyl, or hydroxyethyl.

11. The compound of any one of claims 1-10, wherein Z is a 5- to 6-member nitrogen-containing monocycle optionally substituted with one or more methyl, C(O)CH₃, haloalkyl, ethyl, cyano, cyanomethyl, or methoxymethyl.

12. The compound of any one of claims 1-10, wherein Z is a 6- to 12-member nitrogen-containing bicycle optionally substituted with one or more methyl, cyano, halogen, haloalkyl, methylsulfonyl, NH₂, oxo, or carbonyl.

13. The compound of any one of claims 1-12, wherein Q is direct bond.

14. The compound of any one of claims 1-12, wherein Q is CH₂.

15. The compound of any one of claims 1-12, wherein Q is CH₂CH₂.

16. The compound of any one of claims 1-12, wherein Q is CH₂-azetidine.

17. The compound of any one of claims 1-14, wherein Q is CH₂ substituted with one or more (C₁-C₁₀)alkyl.

18. The compound of claim 1, wherein Q is heterocycle optionally substituted with one or more (C₁-C₁₀)hydrocarbyl.

19. The compound of claim 1, wherein Q is

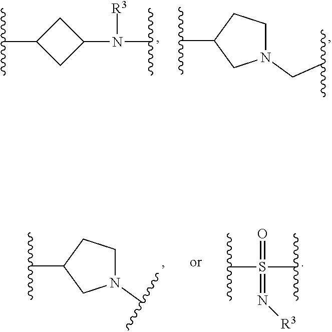

20. The compound of claim 19, wherein Q is

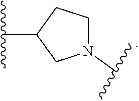 [SMILES: *C1CCN(*)C1]

21. The compound of any one of claims 1-20, wherein R² is a 3- to 12-member heterocycle.

22. The compound of claim 21, wherein R² is a 3- to 12-member nitrogen- or oxygen-containing monocycle or bicycle.

23. The compound of claim 21, wherein R² is chosen from pyridine, triazole, pyrazole, azetidine, benzofuran, dihydrobenzofuran, benzo[1.3]dioxole, indazole, pyrazolopyridine, imidazopyridine, triazolopyridine, dihydroindene, chromane, quinoline, hexahydrocyclopentapyrrole, hexahydropyrrolopyrrole, tetrahydrofuropyrrole, indolene, isoindoline, pyrrolopyrimidine, piperidine, piperazine, and pyridazine, wherein each of the foregoing is optionally substituted with one or more methyl, methoxy, hydroxyl, oxo, or fluoro.

24. The compound of any one of claims 1-20, wherein R² is a 6- to 10-member carbocycle.

25. The compound of claim 24, wherein R² is aromatic and optionally substituted with one or more methoxy, ethoxy, difluoromethyl, OCH(CH₃)₂, O-cyclopropyl, phenyl, cyano, cyclopropyl, COOH, pyridine optionally substituted with pyrrolidine optionally substituted with one or more fluoro.

26. The compound of claim 1, wherein R² is HetA or CarbA.

27. The compound of claim 26, wherein HetA is a 6-membered heteroaryl.

28. The compound of claim 26, wherein CarbA is a 6-membered aryl.

29. The compound of any one of claims 1-28, wherein R^{c} is independently chosen in each instance from hydrogen, methyl, haloalkyl, ethyl, cyano, hydrogen, cyanomethyl, or methoxymethyl.

30. The compound of any one of claims 1-29, wherein R^{d} is independently chosen in each instance from hydrogen, methyl, and methoxymethyl.

31. The compound of claim 1, with the formula:

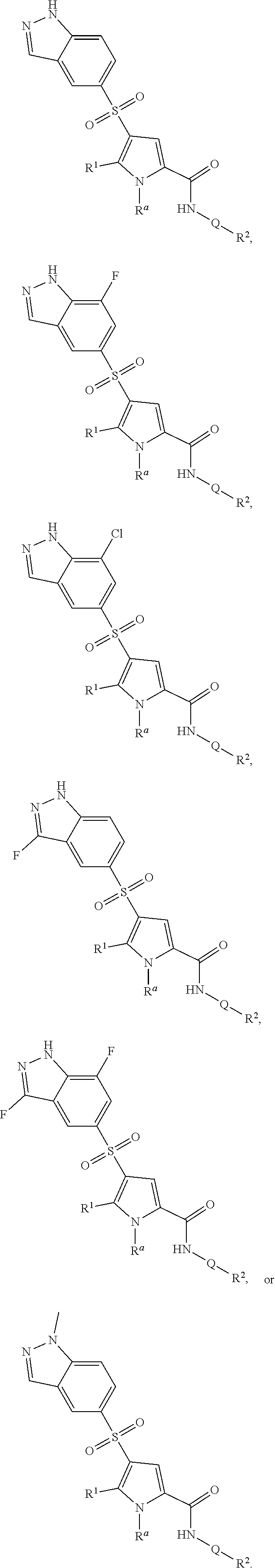

32. The compound of claim 1, with the formula:

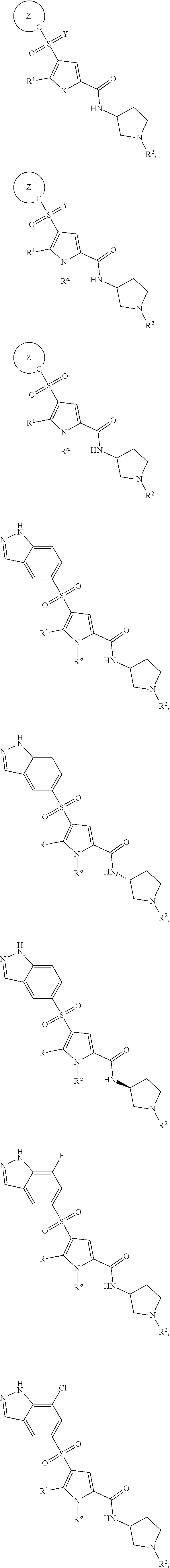

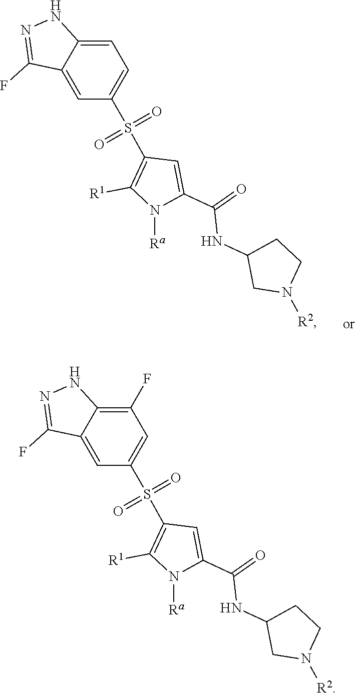

33. The compound of claim 1, with the formula:

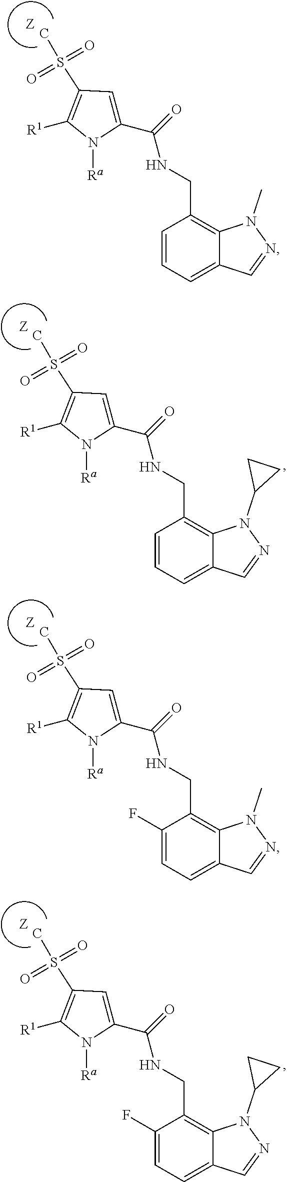

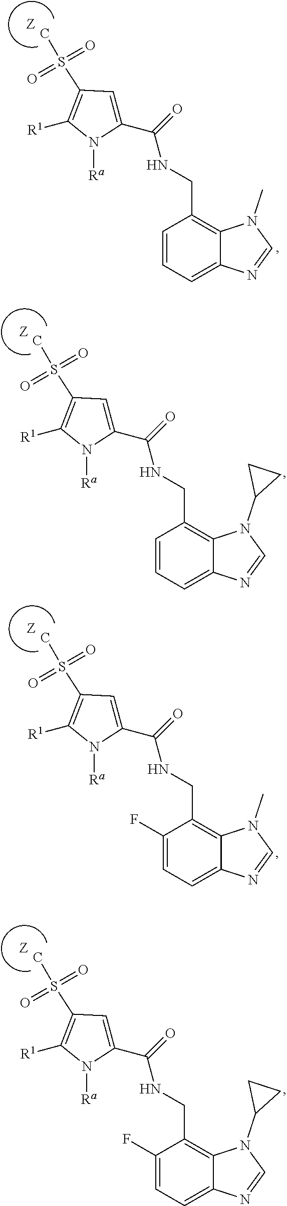

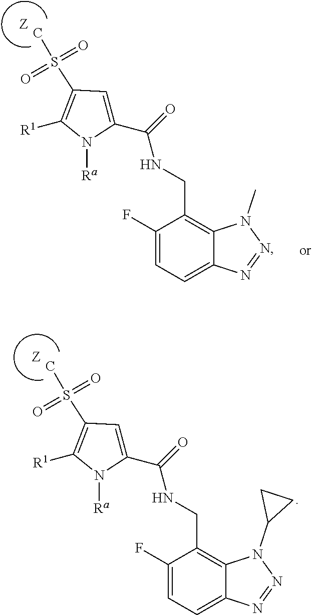

34. The compound of claim 1, with the formula:

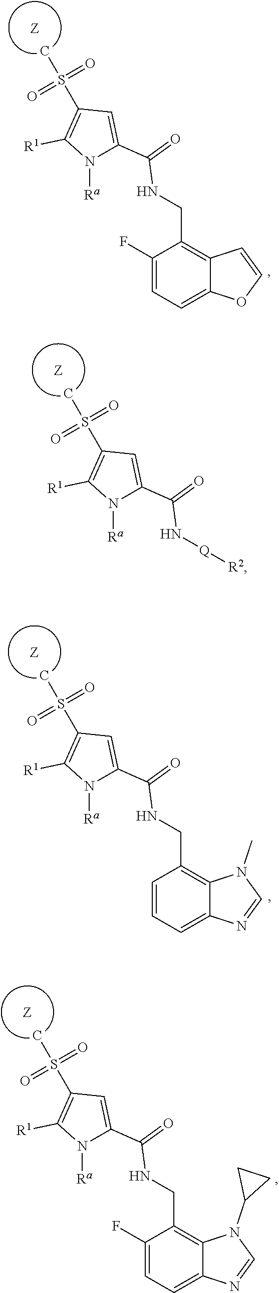

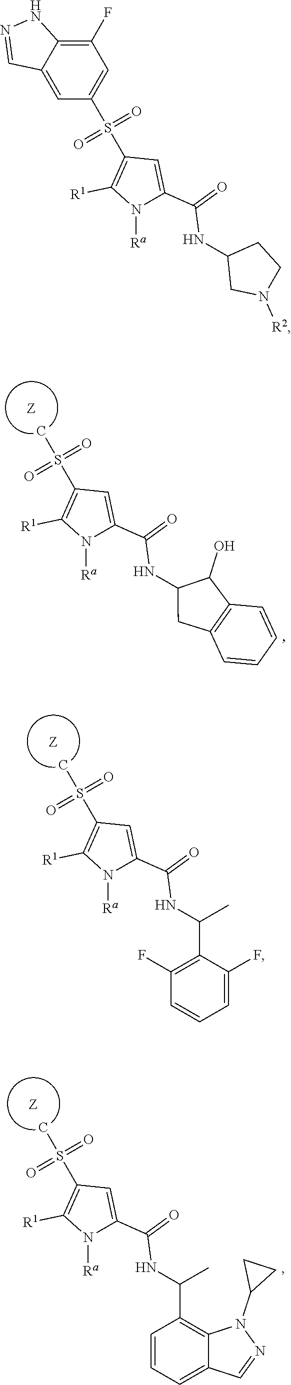

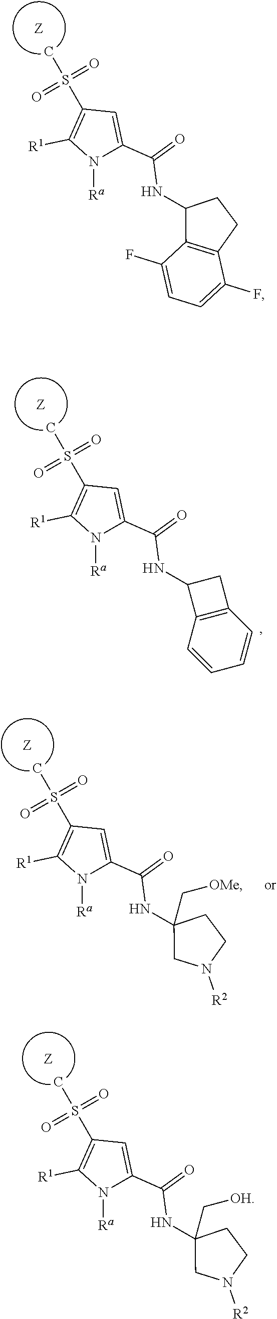

35. The compound of claim 34, with the structure:

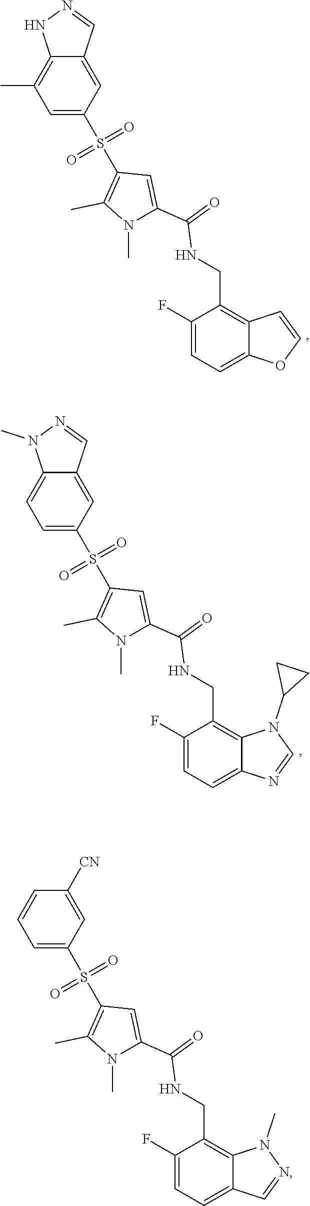 [SMILES: Cc1cc(S(=O)(=O)c2cc(C(=O)NCc3c(F)ccc4occc34)n(C)c2C)cc2cn[nH]c12 ; Cc1c(S(=O)(=O)c2ccc3c(cnn3C)c2)cc(C(=O)NCc2c(F)ccc3ncn(C4CC4)c23)n1C ; Cc1c(S(=O)(=O)c2cccc(C#N)c2)cc(C(=O)NCc2c(F)ccc3cnn(C)c23)n1C]

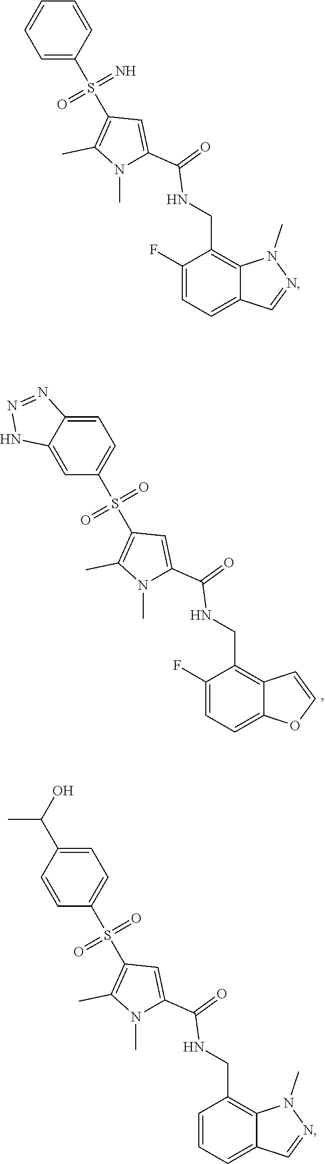 [SMILES: Cc1c(S(=N)(=O)c2ccccc2)cc(C(=O)NCc2c(F)ccc3cnn(C)c23)n1C ; Cc1c(S(=O)(=O)c2ccc3nn[nH]c3c2)cc(C(=O)NCc2c(F)ccc3occc23)n1C ; Cc1c(S(=O)(=O)c2ccc(C(C)O)cc2)cc(C(=O)NCc2cccc3cnn(C)c23)n1C]

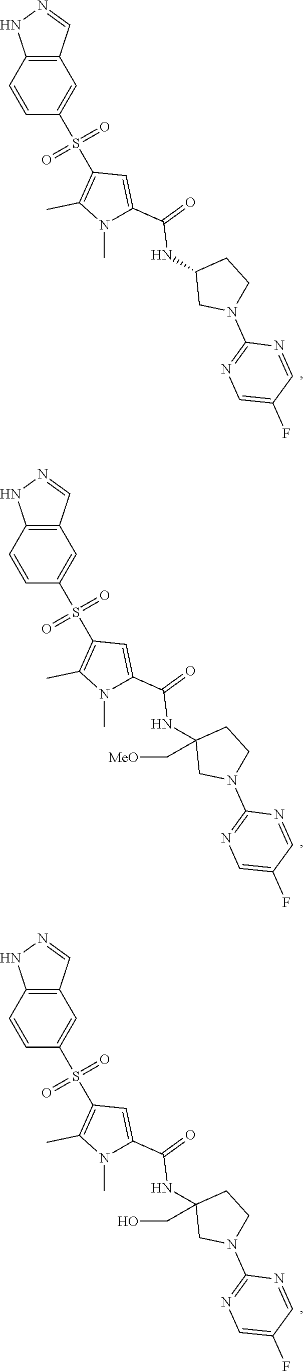 [SMILES: Cc1c(S(=O)(=O)c2ccc3[nH]ncc3c2)cc(C(=O)N[C@@H]2CCN(c3ncc(F)cn3)C2)n1C ; COCC1(NC(=O)c2cc(S(=O)(=O)c3ccc4[nH]ncc4c3)c(C)n2C)CCN(c2ncc(F)cn2)C1 ; Cc1c(S(=O)(=O)c2ccc3[nH]ncc3c2)cc(C(=O)NC2(CO)CCN(c3ncc(F)cn3)C2)n1C]

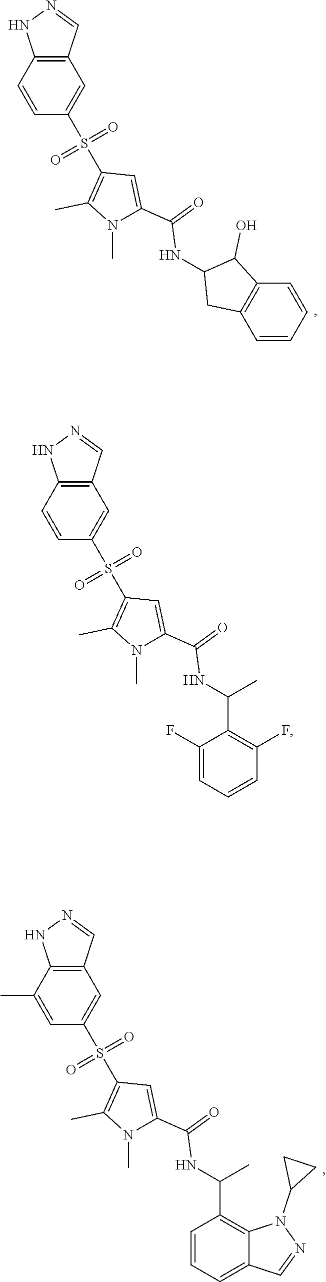 [SMILES: Cc1c(S(=O)(=O)c2ccc3[nH]ncc3c2)cc(C(=O)NC2Cc3ccccc3C2O)n1C ; Cc1c(S(=O)(=O)c2ccc3[nH]ncc3c2)cc(C(=O)NC(C)c2c(F)cccc2F)n1C ; Cc1cc(S(=O)(=O)c2cc(C(=O)NC(C)c3cccc4cnn(C5CC5)c34)n(C)c2C)cc2cn[nH]c12]

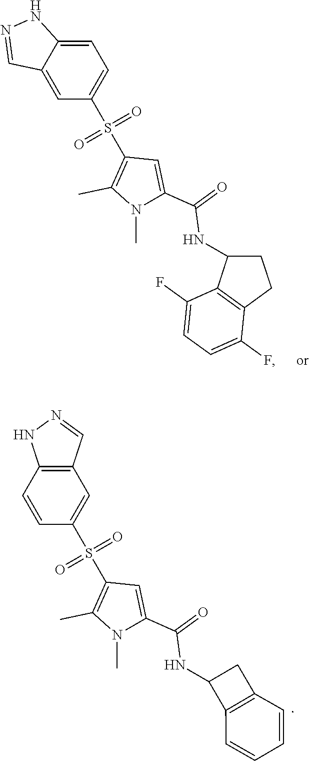 [SMILES: Cc1c(S(=O)(=O)c2ccc3[nH]ncc3c2)cc(C(=O)NC2CCc3c(F)ccc(F)c32)n1C ; Cc1c(S(=O)(=O)c2ccc3[nH]ncc3c2)cc(C(=O)NC2Cc3ccccc32)n1C]

36. The compound of claim 1, with the structure:

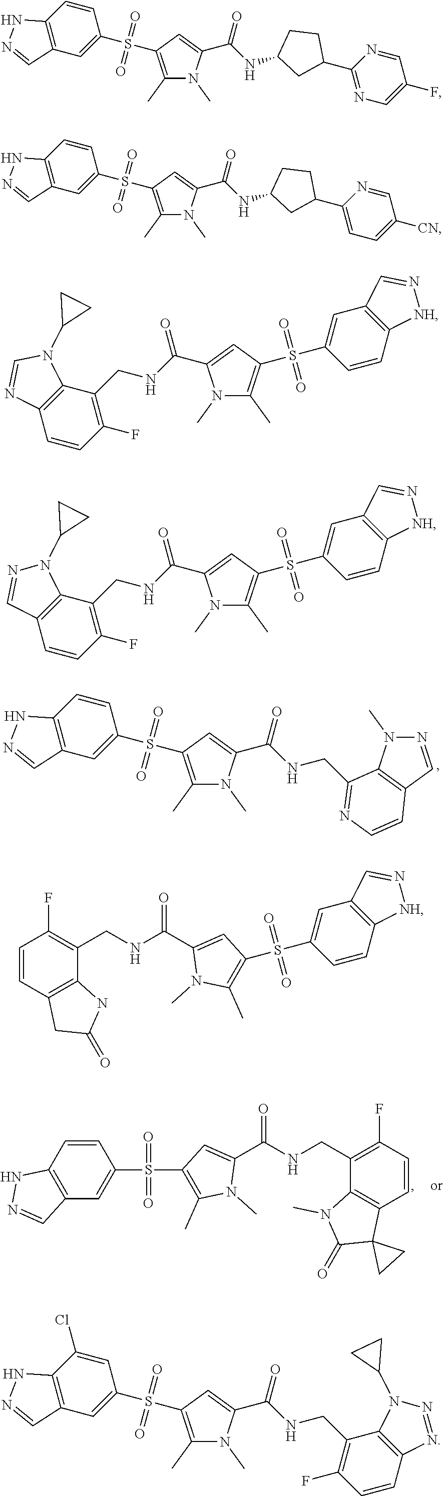

37. A method of inhibiting SARS-CoV-2 NSP14 protein in a patient comprising administering a compound according to any one of claims 1-36.

38. A method of treating Covid-19 in a patient comprising administering a compound according to any one of claims 1-36.

39. A pharmaceutical formulation comprising a pharmaceutically acceptable carrier and a compound according to any one of claims 1-36.
